(19) United States
(12) Patent Application Publication
(10) Pub. No.: US 2023/0181566 A1
(43) Pub. Date: Jun. 15, 2023

(54) THERAPEUTIC COMBINATIONS OF ORALLY ADMINISTERED PACLITAXEL AND A P-gp INHIBITOR FOR THE TREATMENT OF ANGIOSARCOMA

(71) Applicant: Athenex Therapeutics Limited, Hong Kong (CN)

(72) Inventors: Min-Fun Rudolf KWAN, Summit, NJ (US); Johnson Yiu-Nam LAU, Houston, TX (US); Wing Kai CHAN, Hong Kong (CN); Michael P. SMOLINSKI, Amherst, NY (US)

(21) Appl. No.: 17/963,504

(22) Filed: Oct. 11, 2022

Related U.S. Application Data
(63) Continuation of application No. 16/383,026, filed on Apr. 12, 2019, now abandoned.
(60) Provisional application No. 62/657,444, filed on Apr. 13, 2018.

(51) Int. Cl. A61K 31/4725; A61P 35/00; A61K 31/337

(52) U.S. Cl. CPC A61K 31/4725; A61K 31/337; A61P 35/00; A61K 9/0053

(57) ABSTRACT

The application pertains to pharmaceutical combinations of orally administered paclitaxel and a P-gp inhibitor. The pharmaceutical combinations are suitable for the treatment of an angiosarcoma in a subject and for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

### RELATED APPLICATION

This application is a continuation of U.S. Application No. 16/383,026, filed on Apr. 12, 2019, which claims priority to, and the benefit of, U.S. Provisional Application No. 62/657,444, filed on Apr. 13, 2018, the contents of each of which are incorporated herein by reference in their entireties.

### BACKGROUND

Paclitaxel is indicated to treat many types of cancer. However, the affinity of paclitaxel for the p-glycoprotein pump (P-gp) leads to efflux of paclitaxel back into the intestinal lumen, thereby making the drug non-bioavailable when taken orally. Its poor absorption through the intestinal epithelium, along with its unfavorable solubility, has necessitated IV administration of paclitaxel. Excipients, such as Cremophor®, used for IV administration often cause tolerability problems such as hypersensitivity-type infusion reactions. Intravenously administered paclitaxel (e.g., Taxol® or paclitaxel formulated with Cremophor®) requires premedication which has its own set of side effects. Additionally, premedication introduces potential drug-drug interactions. Further, intravenously administered paclitaxel may also be associated with increased incidence or severity of neurotoxicity.

Angiosarcomas are an uncommon type of soft tissue sarcoma characterized by rapidly proliferating, extensively infiltrating anaplastic cells derived from blood and lymphatic vessels. Angiosarcomas arise in various body sites, including cutaneous, soft tissue, and visceral locations. Angiosarcomas are frequently metastatic at diagnosis and are complicated by local recurrence, distant metastases, and poor overall survival. Angiosarcomas comprise about 2% of soft tissue sarcomas and 5.4% of cutaneous soft tissue sarcomas. Currently, there is no approved treatment for angiosarcomas. Treatment of angiosarcomas with IV paclitaxel has been investigated. However, the limitations and side effects associated with IV paclitaxel limit the effectiveness of the drug in treating angiosarcoma.

Therefore, an effective therapeutic regimen including an oral formulation of paclitaxel along with oral administration of a P-gp inhibitor may be beneficial and may be expected to improve the treatment outcome of angiosarcomas. By inhibiting paclitaxel efflux, oral bioavailability would be enhanced allowing delivery of therapeutically relevant concentrations of the drug and avoids the use of excipients such as polyethoxylated castor oil, e.g., Cremophor^{®}, thus leading to a wide therapeutic window that promotes antitumor response while mitigating or avoiding the reactions and toxicities associated directly with the drug or the excipients. In addition, oral administration of paclitaxel provides a more convenient and safe method. These benefits are attractive to patients, many of whom are elderly and would be disinclined to, or physically incapable of, receving treatment through IV paclitaxel due to the associated toxicities and side effects. The present application addresses the needs for orally administering paclitaxel for the treatment of angiosarcomas.

### SUMMARY

The application relates to methods, compositions, medicaments, therapeutic combinations, combinational therapies, and uses for treating an angiosarcoma in a subject in need thereof.

The application pertains to a compound for use in the treatment of an angiosarcoma in a subject in need thereof, wherein the subject is administered paclitaxel orally at an amount of about 100 mg/m² to about 400 mg/m² once a day and for 1-7 times a week; and

wherein the compound is Compound A:

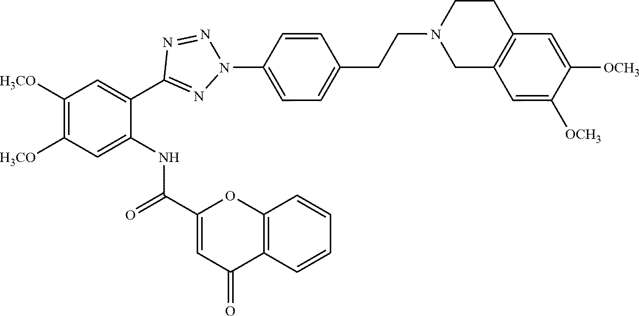 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

, which is administered to the subject once a day and for 1-7 times a week,

wherein Compound A is administered simultaneously with or prior to the paclitaxel.

The application also pertains to Compound A for use in reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and wherein Compound A is administered to the subject once a day and for 1-7 times a week,

wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

The application also pertains to Compound A for use in reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application also pertains to a method for treating an angiosarcoma in a subject in need thereof, comprising:
a. oral administration of paclitaxel at an amount of about 100 mg/m² to about 400 mg/m² to the subject once a day and for 1-7 times a week; and
b. oral administration of Compound A to the subject once a day and for 1-7 times a week, wherein Compound A is administered simultaneously with or prior to the paclitaxel.

The application also pertains to a method for reducing hematologic toxicity and/or neurotoxicity in a subject suffering from an angiosarcoma, comprising:
a. oral administration of paclitaxel at an amount of about 100 mg/m² to about 400 mg/m² to the subject once a day and for 1-7 times a week; and
b. oral administration of Compound A to the subject once a day and for 1-7 times a week,
   wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

The application also pertains to a method for reducing or preventing hypersensitivity-type infusion reactions associated with intravenously administered paclitaxel (e.g., Taxol® or paclitaxel formulated with Cremophor®) in a subject suffering from an angiosarcoma, comprising:

a. oral administration of paclitaxel at an amount of about 100 mg/m² to about 400 mg/m² to the subject once a day and for 1-7 times a week; and
b. oral administration of Compound A to the subject once a day and for 1-7 times a week,
   wherein the orally administered paclitaxel reaches therapeutic blood or plasma levels in the subject, and wherein Compound A is administered simultaneously with or prior to the paclitaxel.

The application also pertains to paclitaxel for oral administration for use in combination with Compound A, as described herein, in treating an angiosarcoma in a subject in need thereof, in reducing hematologic toxicity and/or neurotoxicity, and/or in reducing or preventing hypersensitivity-type infusion reactions associated with intravenously administered paclitaxel (e.g., Taxol® or paclitaxel formulated with Cremophor®).

This application also pertains to paclitaxel for oral administration for use with Compound A, as described herein, in a combinational therapy for treating an angiosarcoma in a subject in need thereof, for reducing hematologic toxicity and/or neurotoxicity, and/or for reducing or preventing hypersensitivity-type infusion reactions associated with intravenously administered paclitaxel (e.g., Taxol® or paclitaxel formulated with Cremophor®).

The application also pertains to the use of paclitaxel for oral administration in combination with Compound A, as described herein, in the manufacture of a medicament for treating an angiosarcoma in a subject in need thereof, for reducing hematologic toxicity and/or neurotoxicity, and/or for reducing or preventing hypersensitivity-type infusion reactions associated with intravenously administered paclitaxel (e.g., Taxol® or paclitaxel formulated with Cremophor®).

This application also pertains to Compound A for use in combination with orally administered paclitaxel, as described herein, in treating an angiosarcoma in a subject in need thereof, in reducing hematologic toxicity and/or neurotoxicity, and/or in reducing or preventing hypersensitivity-type infusion reactions associated with intravenously administered paclitaxel (e.g., Taxol® or paclitaxel formulated with Cremophor®).

This application also pertains to Compound A for use with orally administered paclitaxel, as described herein, in a combinational therapy for treating an angiosarcoma in a subject in need thereof, for reducing hematologic toxicity and/or neurotoxicity, and/or for reducing or preventing hypersensitivity-type infusion reactions associated with intravenously administered paclitaxel (e.g., Taxol® or paclitaxel formulated with Cremophor®).

This application also pertains to the use of Compound A, as described herein, in the manufacture of a medicament for treating an angiosarcoma in a subject in need thereof, for reducing hematologic toxicity and/or neurotoxicity, and/or for reducing or preventing hypersensitivity-type infusion reactions associated with intravenously administered paclitaxel (e.g., Taxol® or paclitaxel formulated with Cremophor®), wherein the subject is administered paclitaxel orally and Compound A, as described herein.

This application also pertains to a medicament for use in a combinational therapy for treating an angiosarcoma in a subject in need thereof, for reducing hematologic toxicity and/or neurotoxicity, and/or for reducing or preventing hypersensitivity-type infusion reactions associated with intravenously administered paclitaxel (e.g., Taxol® or paclitaxel formulated with Cremophor®), wherein the medicament comprises Compound A, which is administered to the subject once a day and for 1-7 times a week, wherein the subject is administered paclitaxel orally as described herein, and wherein the medicament is administered simultaneously with or prior to the paclitaxel.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

FIG. 1A - FIG. 1C are graphs showing in vitro cytotoxicity of paclitaxel in murine angiosarcoma cell lines: MS1 (FIG. 1A), SVRA221a (FIG. 1B), and SVRbag4 (FIG. 1C), each of which were treated with paclitaxel (0.1 - 2178 nM) for 72 hours. Symbols represent the mean of each individual experimental observation and lines depict the fit of the sigmoid Eₘₐₓ pharmacodynamic model using nonlinear regression. All data are representative of n = 3.

FIG. 2 is a graph showing in vitro cytotoxicity of paclitaxel in a human endothelium cell line treated with paclitaxel (0.1 - 2178 nM) for 72 hours. Symbols represent the mean of each individual experimental observation and lines depict the fit of the sigmoid Eₘₐₓ pharmacodynamic model using nonlinear regression. All data are representative of n = 3.

FIG. 3A - FIG. 3C are representative images (n = 2) of immunofluorescence microscopy of tubulin in MS1 (FIG. 3A), SVRA221a (FIG. 3B), and SVRbag4 (FIG. 3C), each of which were treated with paclitaxel (indicated in the images as “PTX”, 1 - 100 nM) in the presence and absence of 100 nM Compound A (indicated in the images as “HM”) for 3 hours.

FIG. 4 is a graph showing the effect of treatment with Compound A and paclitaxel on body weight of SCID mice implanted with SVRA221a tumor. Mouse weights were collected three times per week. Data are presented as the mean ± SEM.

FIG. 5 is a Kaplan-Meier survival curves showing the effect of treatment with Compound A and paclitaxel on survival of SCID mice implanted with SVRA221a tumor.

FIG. 6 is a graph showing the effect of treatment with Compound A and paclitaxel on SVRA221a tumor growth in SCID mice. Black arrows indicate treatment days. Data are presented as the mean ± SEM (n ≥ 7 tumors per data point).

FIG. 7 is an illustration of the pilot study with the therapeutic combination of the present application in subjects with cutaneous angiosarcoma.

FIG. 8A - FIG. 8C are photographs of Subject 1′s response to oral administration of paclitaxel in combination with Compound A in the treatment of cutaneous angiosarcoma at baseline (FIG. 8A), week 2 (FIG. 8B), and week 7 (FIG. 8C).

FIG. 9A - FIG. 9D are photographs of Subject 2′s response to oral administration of paclitaxel in combination with Compound A in the treatment of cutaneous angiosarcoma at baseline (FIG. 9A), week 7 (FIG. 9B), and week 10 (FIG. 9C and FIG. 9D).

FIG. 10A and FIG. 10B are photographs of Subject 3′s response to oral administration of paclitaxel in combination with Compound A in the treatment of cutaneous angiosarcoma at baseline (FIG. 10A) and week 7 (FIG. 10B).

FIG. 11A and FIG. 11B are PET scans of Subject 3′s response to oral administration of paclitaxel in combination with Compound A in the treatment of cutaneous angiosarcoma at baseline (FIG. 11A) and week 7 (FIG. 11B).

FIG. 12A and FIG. 12B are photographs of Subject 5′s response to oral administration of paclitaxel in combination with Compound A in the treatment of cutaneous angiosarcoma at baseline (FIG. 12A) and week 7 (FIG. 12B).

FIG. 13A - FIG. 13C are photographs of Subject 6′s response to oral administration of paclitaxel in combination with Compound A in the treatment of cutaneous angiosarcoma at baseline (FIG. 13A), week 2 (FIG. 13B), and week 7 (FIG. 13C).

FIG. 14A and FIG. 14B are photographs of Subject 7′s response to oral administration of paclitaxel in combination with Compound A in the treatment of cutaneous angiosarcoma at baseline (FIG. 14A) and week 7 (FIG. 14B).

### DETAILED DESCRIPTION

The application pertains, at least in part, to methods for treating an angiosarcoma in a subject in need thereof.

The application pertains, at least in part, to methods for reducing or preventing toxicity, hypersensitivity-type infusion reactions, and other negative outcomes resulting from or associated with intravenously administered paclitaxel (e.g., Taxol® or paclitaxel formulated with Cremophor®) in a subject suffering from an angiosarcoma.

In some embodiments, the application pertains to a method for treating an angiosarcoma in a subject in need thereof, comprising:
a. oral administration of paclitaxel at an amount of about 100 mg/m² to about 400 mg/m² to the subject once a day and for 1-7 times a week; and
b. oral administration of Compound A:

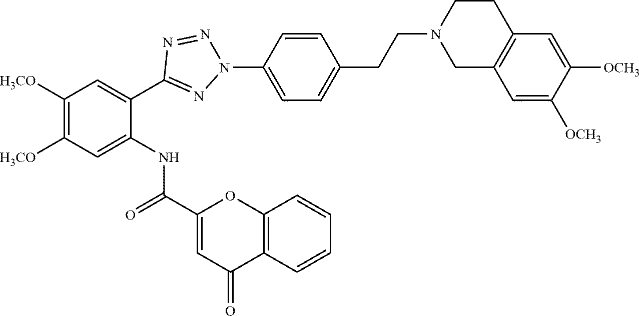 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

, to the subject once a day and for 1-7 times a week, wherein Compound A is administered simultaneously with or prior to the paclitaxel.

In some embodiments, the application pertains to a method for treating an angiosarcoma in a subject in need thereof, comprising:
a. oral administration of paclitaxel at an amount of about 100 mg/m² to about 400 mg/m² to the subject once a day and for 3 times a week; and
b. oral administration of Compound A to the subject once a day and for 3 times a week, wherein Compound A is administered simultaneously with or prior to the paclitaxel.

In some embodiments, the application pertains to a method for treating an angiosarcoma in a subject in need thereof, comprising:
a. oral administration of paclitaxel at an amount of about 100 mg/m² to about 400 mg/m² to the subject once a day and for 3 times a week; and
b. oral administration of Compound A to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 100 mg/m² to about 400 mg/m² to the subject once a day and for 3 times a week; and
b. oral administration of Compound A at an amount of about 15 mg to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 205 mg/m² to the subject once a day and for 3 times a week; and
b. oral administration of Compound A to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 205 mg/m² to the subject once a day and for 3 times a week; and
b. oral administration of Compound A at an amount of about 15 mg to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 205 mg/m² to the subject once a day and for 3 times a week; and
b. oral administration of Compound A at an amount of about 15 mg to the subject once a day and for 3 times a week, wherein Compound A is administered one hour prior to the paclitaxel.

In some embodiments, the application pertains to a method for treating an angiosarcoma in a subject in need thereof, comprising:
a. oral administration of paclitaxel at an amount of about 165 mg/m² to the subject once a day and for 3 times a week; and
b. oral administration of Compound A to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 165 mg/m² to the subject once a day and for 3 times a week; and
b. oral administration of Compound A at an amount of about 15 mg to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 165 mg/m² to the subject once a day and for 3 times a week; and
b. oral administration of Compound A at an amount of about 15 mg to the subject once a day and for 3 times a week, wherein Compound A is administered one hour prior to the paclitaxel.

In some embodiments, the application pertains to a method for treating an angiosarcoma in a subject in need thereof, comprising:
a. oral administration of paclitaxel at an amount of about 130 mg/m² to the subject once a day and for 3 times a week; and
b. oral administration of Compound A to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 130 mg/m² to the subject once a day and for 3 times a week; and
b. oral administration of Compound A at an amount of about 15 mg to the subject once a day and for 3 times a week, wherein Compound A is administered prior to the paclitaxel.

In some embodiments, the application pertains to a method for treating an angiosarcoma in a subject in need thereof, comprising:
a. oral administration of paclitaxel at an amount of about 130 mg/m² to the subject once a day and for 3 times a week; and
b. oral administration of Compound A at an amount of about 15 mg to the subject once a day and for 3 times a week, wherein Compound A is administered one hour prior to the paclitaxel.

In some embodiments, the application pertains to a method for reducing hematologic toxicity and/or neurotoxicity in a subject suffering from an angiosarcoma, comprising:
a. oral administration of paclitaxel at an amount of about 100 mg/m² to about 400 mg/m² to the subject once a day and for 1-7 times a week; and
b. oral administration of Compound A to the subject once a day and for 1-7 times a week,
   wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to a method for reducing or preventing hypersensitivity-type infusion reactions associated with intravenously administered paclitaxel (e.g., Taxol® or paclitaxel formulated with Cremophor®) in a subject suffering from an angiosarcoma, comprising:
a. oral administration of paclitaxel at an amount of about 100 mg/m² to about 400 mg/m² to the subject once a day and for 1-7 times a week; and
b. oral administration of Compound A to the subject once a day and for 1-7 times a week,
   wherein the orally administered paclitaxel reaches therapeutic blood or plasma levels in the subject, and wherein Compound A is administered simultaneously with or prior to the paclitaxel.

The application pertains, at least in part, to Compound A for use in the treatment of an angiosarcoma in a subject in need thereof.

The application pertains, at least in part, to Compound A for use in reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to Compound A for use in the treatment of an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to Compound A for use in reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to Compound A for use in reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to the use of Compound A in the manufacture of a medicament for the treatment of an angiosarcoma in a subject in need thereof.

The application pertains, at least in part, to the use of Compound A in the manufacture of a medicament for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to the use of Compound A in the manufacture of a medicament for treating an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to the use of Compound A in the manufacture of a medicament for reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to the use of Compound A in the manufacture of a medicament for reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to Compound A for use with paclitaxel in a combinational therapy for treating an angiosarcoma in a subject in need thereof.

The application pertains, at least in part, to Compound A for use with paclitaxel in a combinational therapy for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to Compound A for use with orally administered paclitaxel in a combinational therapy for treating an angiosarcoma in a subject in need thereof, wherein Compound A is administered to the subject once a day and for 1-7 times a week; and
wherein the subject is also administered paclitaxel orally at an amount of about 100 mg/m² to about 400 mg/m² once a day and for 1-7 times a week,
wherein Compound A is administered simultaneously with or prior to the paclitaxel.

In some embodiments, the application pertains to Compound A for use with orally administered paclitaxel in a combinational therapy for reducing hematologic toxicity and/or neurotoxicity in a subject suffering from an angiosarcoma, wherein Compound A is administered to the subject once a day and for 1-7 times a week; and
wherein the subject is also administered paclitaxel orally at an amount of about 100 mg/m² to about 400 mg/m² once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to Compound A for use with orally administered paclitaxel in a combinational therapy for reducing or preventing hypersensitivity-type infusion reactions associated with intravenously administered paclitaxel (e.g., Taxol® or paclitaxel formulated with Cremophor®) in a subject suffering from an angiosarcoma, wherein Compund A is administered to the subject once a day and for 1-7 times a week; and
wherein the subject is also administered paclitaxel orally at an amount of about 100 mg/m² to about 400 mg/m² once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to Compound A for use in combination with orally administered paclitaxel in treating an angiosarcoma in a subject in need thereof.

The application pertains, at least in part, to Compound A for use in combination with orally administered paclitaxel in reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to Compound A for use in combination with orally administered paclitaxel in treating an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to Compound A for use in combination with orally administered paclitaxel in reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, wherein Compound A is administered simultaneously with or prior to the paclitaxel.

In some embodiments, the application pertains to Compound A for use in combination with orally administered paclitaxel in reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to a medicament for use in a combinational therapy for treating an angiosarcoma in a subject in need thereof.

The application pertains, at least in part, to a medicament for use in a combinational therapy for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to a medicament for use in a combinational therapy for treating an angiosarcoma in a subject in need thereof, wherein the medicament comprises Compound A, which is administered to the subject once a day and for 1-7 times a week, wherein the subject is administered paclitaxel orally at an amount of about 100 mg/m² to about 400 mg/m² once a day and for 1-7 times a week, and wherein Compound A is administered simultaneously with or prior to the paclitaxel.

In some embodiments, the application pertains to a medicament for use in a combinational therapy for reducing hematologic toxicity and/or neurotoxicity in a subject suffering from an angiosarcoma, wherein the medicament comprises Compound A, which is administered to the subject once a day and for 1-7 times a week, wherein the subject is administered paclitaxel orally at an amount of about 100 mg/m² to about 400 mg/m² once a day and for 1-7 times a week, and

wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to a medicament for use in a combinational therapy for reducing or preventing hypersensitivity-type infusion reactions associated with intravenously administered paclitaxel (e.g., Taxol® or paclitaxel formulated with Cremophor®) in a subject suffering from an angiosarcoma, wherein the medicament comprises Compound A, which is administered to the subject once a day and for 1-7 times a week, wherein the subject is administered paclitaxel orally at an amount of about 100 mg/m² to about 400 mg/m² once a day and for 1-7 times a week,

wherein the orally administered paclitaxel reaches therapeutic blood or plasma levels in the subject, and wherein Compound A is administered simultaneously with or prior to the paclitaxel.

The application pertains, at least in part, to paclitaxel for oral administration for use in combination with Compound A in treating an angiosarcoma in a subject in need thereof.

The application pertains, at least in part, to paclitaxel for oral administration for use in combination with Compound A in reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to paclitaxel for oral administration for use in combination with Compound A in treating an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to paclitaxel for oral administration for use in combination with Compound A in reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to paclitaxel for oral administration for use in combination with Compound A in reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to paclitaxel for oral administration for use with Compound A in a combinational therapy for treating an angiosarcoma in a subject in need thereof.

The application pertains, at least in part, to paclitaxel for oral administration for use with Compound A in a combinational therapy for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to paclitaxel for oral administration for use with Compound A in a combinational therapy for treating an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to paclitaxel for oral administration for use with Compound A in a combinational therapy for reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to paclitaxel for oral administration for use with Compound A in a combinational therapy for reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

The application pertains, at least in part, to the use of paclitaxel for oral administration in combination with Compound A in the manufacture of a medicament for treating an angiosarcoma in a subject in need thereof.

The application pertains, at least in part, to the use of paclitaxel for oral administration in combination with Compound A in the manufacture of a medicament for reducing or preventing toxicity, hypersensitivity-type infusion reactions, and other negative outcomes resulting from or associated with intravenously administered paclitaxel (e.g., Taxol® or paclitaxel formulated with Cremophor®) therapy in a subject suffering from an angiosarcoma.

In some embodiments, the application pertains to the use of paclitaxel for oral administration in combination with Compound A in the manufacture of a medicament for treating an angiosarcoma in a subject in need thereof, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein Compound A is administered simultaneously with or prior to the paclitaxel.

In some embodiments, the application pertains to the use of paclitaxel for oral administration in combination with Compound A in the manufacture of a medicament for reducing hematologic toxicity and/or neurotoxicity in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the plasma exposure of the orally administered paclitaxel, as measured by AUC_{(0→∞)}, is equal to or greater than the plasma exposure, as measured by AUC_{(0→∞)}, of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks, at an amount of about 125 mg/m² to about 175 mg/m² over a period of about 0.5 hours to about 3 hours once every 3 weeks, or at an amount of about 80 mg/m² over a period of about 1 hour weekly, and wherein Compound A is administered simultaneously with or prior to the paclitaxel.

In some embodiments, the application pertains to the use of paclitaxel for oral administration in combination with Compound A in the manufacture of a medicament for reducing or preventing hypersensitivity-type infusion reactions associated with intravenously administered paclitaxel (e.g., Taxol® or paclitaxel formulated with Cremophor®) in a subject suffering from an angiosarcoma, wherein the subject is administered paclitaxel orally at an amount of about 100 mg/m² to about 400 mg/m² once a day and for 1-7 times a week; and
wherein Compound A is administered to the subject once a day and for 1-7 times a week,
wherein the orally administered paclitaxel reaches therapeutic blood or plasma levels in the subject, and wherein Compound A is administered simultaneously with or prior to the paclitaxel.

Unless otherwise indicated, the paclitaxel and Compound A of the present application are administered orally, i.e., through oral administration.

For the methods, compositions, medicaments, therapeutic combinations, combinational therapies, and uses described herein (e.g., Compound A for use, use of Compound A in the manufacture of a medicament, Compound A for use with orally administered paclitaxel in a combinational therapy, Compound A for use in combination with orally administered paclitaxel, medicament for use in a combinational therapy, paclitaxel for oral administration for use in combination with Compound A, paclitaxel for oral administration for use with Compound A in a combinational therapy, or use of paclitaxel for oral administration in combination with Compound A in the manufacture of a medicament), various embodiments are described below.

In some embodiments, the paclitaxel is orally administred at an amount of about 100 mg/m² to about 400 mg/m² once a day and for 1-7 times a week

In some embodiments, paclitaxel in the present application is formulated for oral administration at the various doses in the methods, compositions, medicaments, therapeutic combinations, combinational therapies, and uses, described herein.

In some embodiments, the paclitaxel is administered at an amount of about 100 mg/m² to about 400 mg/m².

In some embodiments, the paclitaxel is administered at an amount of about 100 mg/m² to about 350 mg/m².

In some embodiments, the paclitaxel is administered at an amount of about 100 mg/m² to about 300 mg/m².

In some embodiments, the paclitaxel is administered at an amount of about 150 mg/m² to about 250 mg/m².

In some embodiments, the paclitaxel is administered at an amount of about 100 mg/m² to about 245 mg/m².

In some embodiments, the paclitaxel is administered at an amount of about 100 mg/m² to about 240 mg/m².

In some embodiments, the paclitaxel is administered at an amount of about 100 mg/m² to about 235 mg/m².

In some embodiments, the paclitaxel is administered at an amount of about 100 mg/m² to about 230 mg/m².

In some embodiments, the paclitaxel is administered at an amount of about 100 mg/m² to about 225 mg/m².

In some embodiments, the paclitaxel is administered at an amount of about 100 mg/m² to about 220 mg/m².

In some embodiments, the paclitaxel is administered at an amount of about 100 mg/m² to about 215 mg/m².

In some embodiments, the paclitaxel is administered at an amount of about 100 mg/m² to about 210 mg/m².

In some embodiments, the paclitaxel is administered at an amount of about 100 mg/m² to about 205 mg/m².

In some embodiments, the paclitaxel is administered at an amount of about 100 mg/m² to about 200 mg/m².

In some embodiments, the paclitaxel is administered at an amount of about 100 mg/m² to about 195 mg/m².

In some embodiments, the paclitaxel is administered at an amount of about 100 mg/m² to about 190 mg/m².

In some embodiments, the paclitaxel is administered at an amount of about 100 mg/m² to about 185 mg/m².

In some embodiments, the paclitaxel is administered at an amount of about 100 mg/m² to about 180 mg/m².

In some embodiments, the paclitaxel is administered at an amount of about 100 mg/m² to about 175 mg/m².

In some embodiments, the paclitaxel is administered at an amount of about 100 mg/m² to about 170 mg/m².

In some embodiments, the paclitaxel is administered at an amount of about 100 mg/m² to about 165 mg/m².

In some embodiments, the paclitaxel is administered at an amount of about 100 mg/m² to about 160 mg/m².

In some embodiments, the paclitaxel is administered at an amount of about 100 mg/m² to about 155 mg/m².

In some embodiments, the paclitaxel is administered at an amount of about 100 mg/m² to about 150 mg/m².

In some embodiments, the paclitaxel is administered at an amount of about 100 mg/m² to about 145 mg/m².

In some embodiments, the paclitaxel is administered at an amount of about 100 mg/m² to about 140 mg/m².

In some embodiments, the paclitaxel is administered at an amount of about 100 mg/m² to about 135 mg/m².

In some embodiments, the paclitaxel is administered at an amount of about 100 mg/m² to about 130 mg/m².

In some embodiments, the paclitaxel is administered at an amount of about 100 mg/m² to about 125 mg/m².

In some embodiments, the paclitaxel is administered at an amount of about 100 mg/m² to about 120 mg/m².

In some embodiments, the paclitaxel is administered at an amount of about 100 mg/m² to about 115 mg/m².

In some embodiments, the paclitaxel is administered at an amount of about 100 mg/m² to about 110 mg/m².

In some embodiments, the paclitaxel is administered at an amount of about 100 mg/m² to about 105 mg/m².

In some embodiments, the paclitaxel is administered at an amount of about 150 mg/m² to about 350 mg/m².

In some embodiments, the paclitaxel is administered at an amount of about 150 mg/m² to about 300 mg/m².

In some embodiments, the paclitaxel is administered at an amount of about 150 mg/m² to about 250 mg/m².

In some embodiments, the paclitaxel is administered at an amount of about 150 mg/m² to about 245 mg/m².

In some embodiments, the paclitaxel is administered at an amount of about 150 mg/m² to about 240 mg/m².

In some embodiments, the paclitaxel is administered at an amount of about 150 mg/m² to about 235 mg/m².

In some embodiments, the paclitaxel is administered at an amount of about 150 mg/m² to about 230 mg/m².

In some embodiments, the paclitaxel is administered at an amount of about 150 mg/m² to about 225 mg/m².

In some embodiments, the paclitaxel is administered at an amount of about 150 mg/m² to about 220 mg/m².

In some embodiments, the paclitaxel is administered at an amount of about 150 mg/m² to about 215 mg/m².

In some embodiments, the paclitaxel is administered at an amount of about 150 mg/m² to about 210 mg/m².

In some embodiments, the paclitaxel is administered at an amount of about 150 mg/m² to about 205 mg/m².

In some embodiments, the paclitaxel is administered at an amount of about 150 mg/m² to about 200 mg/m².

In some embodiments, the paclitaxel is administered at an amount of about 150 mg/m² to about 195 mg/m².

In some embodiments, the paclitaxel is administered at an amount of about 150 mg/m² to about 190 mg/m².

In some embodiments, the paclitaxel is administered at an amount of about 150 mg/m² to about 185 mg/m².

In some embodiments, the paclitaxel is administered at an amount of about 150 mg/m² to about 180 mg/m².

In some embodiments, the paclitaxel is administered at an amount of about 150 mg/m² to about 175 mg/m².

In some embodiments, the paclitaxel is administered at an amount of about 150 mg/m² to about 170 mg/m².

In some embodiments, the paclitaxel is administered at an amount of about 150 mg/m² to about 165 mg/m².

In some embodiments, the paclitaxel is administered at an amount of about 150 mg/m² to about 160 mg/m².

In some embodiments, the paclitaxel is administered at an amount of about 150 mg/m² to about 155 mg/m².

In some embodiments, the paclitaxel is administered at an amount of about 200 mg/m² to about 300 mg/m².

In some embodiments, the paclitaxel is administered at an amount of about 200 mg/m² to about 295 mg/m².

In some embodiments, the paclitaxel is administered at an amount of about 200 mg/m² to about 290 mg/m².

In some embodiments, the paclitaxel is administered at an amount of about 200 mg/m² to about 285 mg/m².

In some embodiments, the paclitaxel is administered at an amount of about 200 mg/m² to about 280 mg/m².

In some embodiments, the paclitaxel is administered at an amount of about 200 mg/m² to about 275 mg/m².

In some embodiments, the paclitaxel is administered at an amount of about 200 mg/m² to about 270 mg/m².

In some embodiments, the paclitaxel is administered at an amount of about 200 mg/m² to about 265 mg/m².

In some embodiments, the paclitaxel is administered at an amount of about 200 mg/m² to about 260 mg/m².

In some embodiments, the paclitaxel is administered at an amount of about 200 mg/m² to about 255 mg/m².

In some embodiments, the paclitaxel is administered at an amount of about 200 mg/m² to about 250 mg/m².

In some embodiments, the paclitaxel is administered at an amount of about 200 mg/m² to about 245 mg/m².

In some embodiments, the paclitaxel is administered at an amount of about 200 mg/m² to about 240 mg/m².

In some embodiments, the paclitaxel is administered at an amount of about 200 mg/m² to about 235 mg/m².

In some embodiments, the paclitaxel is administered at an amount of about 200 mg/m² to about 230 mg/m².

In some embodiments, the paclitaxel is administered at an amount of about 200 mg/m² to about 225 mg/m².

In some embodiments, the paclitaxel is administered at an amount of about 200 mg/m² to about 220 mg/m².

In some embodiments, the paclitaxel is administered at an amount of about 200 mg/m² to about 215 mg/m².

In some embodiments, the paclitaxel is administered at an amount of about 200 mg/m² to about 210 mg/m².

In some embodiments, the paclitaxel is administered at an amount of about 200 mg/m² to about 205 mg/m².

In some embodiments, the paclitaxel is administered at an amount of about 250 mg/m² to about 350 mg/m².

In some embodiments, the paclitaxel is administered at an amount of about 250 mg/m² to about 300 mg/m².

In some embodiments, the paclitaxel is administered at an amount of about 120 mg/m² to about 235 mg/m².

In some embodiments, the paclitaxel is administered at an amount of about 120 mg/m² to about 230 mg/m².

In some embodiments, the paclitaxel is administered at an amount of about 120 mg/m² to about 225 mg/m².

In some embodiments, the paclitaxel is administered at an amount of about 120 mg/m² to about 220 mg/m².

In some embodiments, the paclitaxel is administered at an amount of about 120 mg/m² to about 215 mg/m².

In some embodiments, the paclitaxel is administered at an amount of about 120 mg/m² to about 210 mg/m².

In some embodiments, the paclitaxel is administered at an amount of about 120 mg/m² to about 205 mg/m².

In some embodiments, the paclitaxel is administered at an amount of about 120 mg/m² to about 200 mg/m².

In some embodiments, the paclitaxel is administered at an amount of about 120 mg/m² to about 195 mg/m².

In some embodiments, the paclitaxel is administered at an amount of about 120 mg/m² to about 190 mg/m².

In some embodiments, the paclitaxel is administered at an amount of about 120 mg/m² to about 185 mg/m².

In some embodiments, the paclitaxel is administered at an amount of about 120 mg/m² to about 180 mg/m².

In some embodiments, the paclitaxel is administered at an amount of about 120 mg/m² to about 175 mg/m².

In some embodiments, the paclitaxel is administered at an amount of about 120 mg/m² to about 170 mg/m².

In some embodiments, the paclitaxel is administered at an amount of about 120 mg/m² to about 165 mg/m².

In some embodiments, the paclitaxel is administered at an amount of about 120 mg/m² to about 160 mg/m².

In some embodiments, the paclitaxel is administered at an amount of about 120 mg/m² to about 155 mg/m².

In some embodiments, the paclitaxel is administered at an amount of about 120 mg/m² to about 150 mg/m².

In some embodiments, the paclitaxel is administered at an amount of about 120 mg/m² to about 145 mg/m².

In some embodiments, the paclitaxel is administered at an amount of about 120 mg/m² to about 140 mg/m².

In some embodiments, the paclitaxel is administered at an amount of about 120 mg/m² to about 135 mg/m².

In some embodiments, the paclitaxel is administered at an amount of about 120 mg/m² to about 130 mg/m².

In some embodiments, the paclitaxel is administered at an amount of about 120 mg/m² to about 125 mg/m².

In some embodiments, the paclitaxel is administered at an amount of about 125 mg/m² to about 235 mg/m².

In some embodiments, the paclitaxel is administered at an amount of about 125 mg/m² to about 230 mg/m².

In some embodiments, the paclitaxel is administered at an amount of about 125 mg/m² to about 225 mg/m².

In some embodiments, the paclitaxel is administered at an amount of about 125 mg/m² to about 220 mg/m².

In some embodiments, the paclitaxel is administered at an amount of about 125 mg/m² to about 215 mg/m².

In some embodiments, the paclitaxel is administered at an amount of about 125 mg/m² to about 210 mg/m².

In some embodiments, the paclitaxel is administered at an amount of about 125 mg/m² to about 205 mg/m².

In some embodiments, the paclitaxel is administered at an amount of about 125 mg/m² to about 200 mg/m².

In some embodiments, the paclitaxel is administered at an amount of about 125 mg/m² to about 195 mg/m².

In some embodiments, the paclitaxel is administered at an amount of about 125 mg/m² to about 190 mg/m².

In some embodiments, the paclitaxel is administered at an amount of about 125 mg/m² to about 185 mg/m².

In some embodiments, the paclitaxel is administered at an amount of about 125 mg/m² to about 180 mg/m².

In some embodiments, the paclitaxel is administered at an amount of about 125 mg/m² to about 175 mg/m².

In some embodiments, the paclitaxel is administered at an amount of about 125 mg/m² to about 170 mg/m².

In some embodiments, the paclitaxel is administered at an amount of about 125 mg/m² to about 165 mg/m².

In some embodiments, the paclitaxel is administered at an amount of about 125 mg/m² to about 160 mg/m².

In some embodiments, the paclitaxel is administered at an amount of about 125 mg/m² to about 155 mg/m².

In some embodiments, the paclitaxel is administered at an amount of about 125 mg/m² to about 150 mg/m².

In some embodiments, the paclitaxel is administered at an amount of about 125 mg/m² to about 145 mg/m².

In some embodiments, the paclitaxel is administered at an amount of about 125 mg/m² to about 140 mg/m².

In some embodiments, the paclitaxel is administered at an amount of about 125 mg/m² to about 135 mg/m².

In some embodiments, the paclitaxel is administered at an amount of about 125 mg/m² to about 130 mg/m².

In some embodiments, the paclitaxel is administered at an amount of about 130 mg/m² to about 235 mg/m².

In some embodiments, the paclitaxel is administered at an amount of about 130 mg/m² to about 230 mg/m².

In some embodiments, the paclitaxel is administered at an amount of about 130 mg/m² to about 225 mg/m².

In some embodiments, the paclitaxel is administered at an amount of about 130 mg/m² to about 220 mg/m².

In some embodiments, the paclitaxel is administered at an amount of about 130 mg/m² to about 215 mg/m².

In some embodiments, the paclitaxel is administered at an amount of about 130 mg/m² to about 210 mg/m².

In some embodiments, the paclitaxel is administered at an amount of about 130 mg/m² to about 205 mg/m².

In some embodiments, the paclitaxel is administered at an amount of about 130 mg/m² to about 200 mg/m².

In some embodiments, the paclitaxel is administered at an amount of about 130 mg/m² to about 195 mg/m².

In some embodiments, the paclitaxel is administered at an amount of about 130 mg/m² to about 190 mg/m².

In some embodiments, the paclitaxel is administered at an amount of about 130 mg/m² to about 185 mg/m².

In some embodiments, the paclitaxel is administered at an amount of about 130 mg/m² to about 180 mg/m².

In some embodiments, the paclitaxel is administered at an amount of about 130 mg/m² to about 175 mg/m².

In some embodiments, the paclitaxel is administered at an amount of about 130 mg/m² to about 170 mg/m².

In some embodiments, the paclitaxel is administered at an amount of about 130 mg/m² to about 165 mg/m².

In some embodiments, the paclitaxel is administered at an amount of about 130 mg/m² to about 160 mg/m².

In some embodiments, the paclitaxel is administered at an amount of about 130 mg/m² to about 155 mg/m².

In some embodiments, the paclitaxel is administered at an amount of about 130 mg/m² to about 150 mg/m².

In some embodiments, the paclitaxel is administered at an amount of about 130 mg/m² to about 145 mg/m².

In some embodiments, the paclitaxel is administered at an amount of about 130 mg/m² to about 140 mg/m².

In some embodiments, the paclitaxel is administered at an amount of about 130 mg/m² to about 135 mg/m².

In some embodiments, the paclitaxel is administered at an amount of about 100 mg/m².

In some embodiments, the paclitaxel is administered at an amount of about 105 mg/m².

In some embodiments, the paclitaxel is administered at an amount of about 110 mg/m².

In some embodiments, the paclitaxel is administered at an amount of about 115 mg/m².

In some embodiments, the paclitaxel is administered at an amount of about 120 mg/m².

In some embodiments, the paclitaxel is administered at an amount of about 125 mg/m².

In some embodiments, the paclitaxel is administered at an amount of about 130 mg/m².

In some embodiments, the paclitaxel is administered at an amount of about 135 mg/m².

In some embodiments, the paclitaxel is administered at an amount of about 140 mg/m².

In some embodiments, the paclitaxel is administered at an amount of about 145 mg/m².

In some embodiments, the paclitaxel is administered at an amount of about 150 mg/m².

In some embodiments, the paclitaxel is administered at an amount of about 155 mg/m².

In some embodiments, the paclitaxel is administered at an amount of about 160 mg/m².

In some embodiments, the paclitaxel is administered at an amount of about 165 mg/m².

In some embodiments, the paclitaxel is administered at an amount of about 170 mg/m².

In some embodiments, the paclitaxel is administered at an amount of about 175 mg/m².

In some embodiments, the paclitaxel is administered at an amount of about 180 mg/m².

In some embodiments, the paclitaxel is administered at an amount of about 185 mg/m².

In some embodiments, the paclitaxel is administered at an amount of about 190 mg/m².

In some embodiments, the paclitaxel is administered at an amount of about 195 mg/m².

In some embodiments, the paclitaxel is administered at an amount of about 200 mg/m².

In some embodiments, the paclitaxel is administered at an amount of about 205 mg/m².

In some embodiments, the paclitaxel is administered at an amount of about 210 mg/m².

In some embodiments, the paclitaxel is administered at an amount of about 215 mg/m².

In some embodiments, the paclitaxel is administered at an amount of about 220 mg/m².

In some embodiments, the paclitaxel is administered at an amount of about 225 mg/m².

In some embodiments, the paclitaxel is administered at an amount of about 230 mg/m².

In some embodiments, the paclitaxel is administered at an amount of about 235 mg/m².

In some embodiments, the paclitaxel is administered at an amount of about 240 mg/m².

In some embodiments, the paclitaxel is administered at an amount of about 245 mg/m².

In some embodiments, the paclitaxel is administered at an amount of about 250 mg/m².

In some embodiments, the paclitaxel is administered at an amount of about 255 mg/m².

In some embodiments, the paclitaxel is administered at an amount of about 260 mg/m².

In some embodiments, the paclitaxel is administered at an amount of about 265 mg/m².

In some embodiments, the paclitaxel is administered at an amount of about 270 mg/m².

In some embodiments, the paclitaxel is administered at an amount of about 275 mg/m².

In some embodiments, the paclitaxel is administered at an amount of about 280 mg/m².

In some embodiments, the paclitaxel is administered at an amount of about 285 mg/m².

In some embodiments, the paclitaxel is administered at an amount of about 290 mg/m².

In some embodiments, the paclitaxel is administered at an amount of about 295 mg/m².

In some embodiments, the paclitaxel is administered at an amount of about 300 mg/m².

In some embodiments, the paclitaxel is administered at an amount of about 305 mg/m².

In some embodiments, the paclitaxel is administered at an amount of about 310 mg/m².

In some embodiments, the paclitaxel is administered at an amount of about 315 mg/m².

In some embodiments, the paclitaxel is administered at an amount of about 320 mg/m².

In some embodiments, the paclitaxel is administered at an amount of about 325 mg/m².

In some embodiments, the paclitaxel is administered at an amount of about 330 mg/m².

In some embodiments, the paclitaxel is administered at an amount of about 335 mg/m².

In some embodiments, the paclitaxel is administered at an amount of about 340 mg/m².

In some embodiments, the paclitaxel is administered at an amount of about 345 mg/m².

In some embodiments, the paclitaxel is administered at an amount of about 350 mg/m².

In some embodiments, the paclitaxel is administered at an amount of about 355 mg/m².

In some embodiments, the paclitaxel is administered at an amount of about 360 mg/m².

In some embodiments, the paclitaxel is administered at an amount of about 365 mg/m².

In some embodiments, the paclitaxel is administered at an amount of about 370 mg/m².

In some embodiments, the paclitaxel is administered at an amount of about 375 mg/m².

In some embodiments, the paclitaxel is administered at an amount of about 380 mg/m².

In some embodiments, the paclitaxel is administered at an amount of about 385 mg/m².

In some embodiments, the paclitaxel is administered at an amount of about 390 mg/m².

In some embodiments, the paclitaxel is administered at an amount of about 395 mg/m².

In some embodiments, the paclitaxel is administered at an amount of about 400 mg/m².

In some embodiments, Compound A in the present application is formulated for oral administration at the various doses in the methods, compositions, medicaments, therapeutic combinations, combinational therapies, and uses, described herein.

In some embodiments, Compound A is administered at an amount of about 1 mg to about 500 mg.

In some embodiments, Compound A is administered at about 1 mg to about 400 mg.

In some embodiments, Compound A is administered at about 1 mg to about 300 mg.

In some embodiments, Compound A is administered at about 5 mg to about 200 mg.

In some embodiments, Compound A is administered at about 10 mg to about 100 mg.

In some embodiments, Compound A is administered at about 10 mg to about 95 mg.

In some embodiments, Compound A is administered at about 10 mg to about 90 mg.

In some embodiments, Compound A is administered at about 10 mg to about 85 mg.

In some embodiments, Compound A is administered at about 10 mg to about 80 mg.

In some embodiments, Compound A is administered at about 10 mg to about 75 mg.

In some embodiments, Compound A is administered at about 10 mg to about 70 mg.

In some embodiments, Compound A is administered at about 10 mg to about 65 mg.

In some embodiments, Compound A is administered at about 10 mg to about 60 mg.

In some embodiments, Compound A is administered at about 10 mg to about 55 mg.

In some embodiments, Compound A is administered at about 10 mg to about 50 mg.

In some embodiments, Compound A is administered at about 10 mg to about 45 mg.

In some embodiments, Compound A is administered at about 10 mg to about 40 mg.

In some embodiments, Compound A is administered at about 10 mg to about 35 mg.

In some embodiments, Compound A is administered at about 10 mg to about 30 mg.

In some embodiments, Compound A is administered at about 10 mg to about 25 mg.

In some embodiments, Compound A is administered at about 10 mg to about 20 mg.

In some embodiments, Compound A is administered at about 10 mg to about 15 mg.

In some embodiments, Compound A is administered at about 15 mg to about 50 mg.

In some embodiments, Compound A is administered at about 15 mg to about 45 mg.

In some embodiments, Compound A is administered at about 15 mg to about 40 mg.

In some embodiments, Compound A is administered at about 15 mg to about 35 mg.

In some embodiments, Compound A is administered at about 15 mg to about 30 mg.

In some embodiments, Compound A is administered at about 15 mg to about 25 mg.

In some embodiments, Compound A is administered at about 15 mg to about 20 mg.

In some embodiments, Compound A is administered at about 5 mg.

In some embodiments, Compound A is administered at about 10 mg.

In some embodiments, Compound A is administered at about 15 mg.

In some embodiments, Compound A is administered at about 20 mg.

In some embodiments, Compound A is administered at about 25 mg.

In some embodiments, Compound A is administered at about 30 mg

In some embodiments, Compound A is administered at about 35 mg.

In some embodiments, Compound A is administered at about 40 mg.

In some embodiments, Compound A is administered at about 45 mg.

In some embodiments, Compound A is administered at about 50 mg.

In some embodiments, paclitaxel in the present application is formulated for oral administration at the various doses described herein with the various dosing frequencies in the methods, compositions, medicaments, therapeutic combinations, combinational therapies, and uses, described herein.

In some embodiments, the paclitaxel is administered 1-7 times per week. In some embodiments, the paclitaxel is administered on consecutive days.

In some embodiments, the paclitaxel is administered 2-7 times per week. In some embodiments, the paclitaxel is administered on consecutive days.

In some embodiments, the paclitaxel is administered 3-7 times per week. In some embodiments, the paclitaxel is administered on consecutive days.

In some embodiments, the paclitaxel is administered 4-7 times per week. In some embodiments, the paclitaxel is administered on consecutive days.

In some embodiments, the paclitaxel is administered 5-7 times per week. In some embodiments, the paclitaxel is administered on consecutive days.

In some embodiments, the paclitaxel is administered 6-7 times per week. In some embodiments, the paclitaxel is administered on consecutive days.

In some embodiments, the paclitaxel is administered 2-6 times per week. In some embodiments, the paclitaxel is administered 2-5 times per week. In some embodiments, the paclitaxel is administered 2-4 times per week. In some embodiments, the paclitaxel is administered 2-3 times per week. In some embodiments, the paclitaxel is administered on consecutive days.

In some embodiments, the paclitaxel is administered 3-6 times per week. In some embodiments, the paclitaxel is administered 3-5 times per week. In some embodiments, the paclitaxel is administered 3-4 times per week. In some embodiments, the paclitaxel is administered on consecutive days.

In some embodiments, the paclitaxel is administered 4-6 times per week. In some embodiments, the paclitaxel is administered 4-5 times per week. In some embodiments, the paclitaxel is administered on consecutive days.

In some embodiments, the paclitaxel is administered 5-6 times per week. In some embodiments, the paclitaxel is administered on consecutive days.

In some embodiments, the paclitaxel is administered at least 1 time per week. In some embodiments, the paclitaxel is administered on consecutive days.

In some embodiments, the paclitaxel is administered at least 2 times per week. In some embodiments, the paclitaxel is administered on consecutive days.

In some embodiments, the paclitaxel is administered at least 3 times per week. In some embodiments, the paclitaxel is administered on consecutive days.

In some embodiments, the paclitaxel is administered at least 4 times per week. In some embodiments, the paclitaxel is administered on consecutive days.

In some embodiments, the paclitaxel is administered at least 5 times per week. In some embodiments, the paclitaxel is administered on consecutive days.

In some embodiments, the paclitaxel is administered at least 6 times per week. In some embodiments, the paclitaxel is administered on consecutive days.

In some embodiments, the paclitaxel is administered daily per week.

In some embodiments, the paclitaxel is administered at least 2 times per week at an amount of about 150 mg/m² to about 400 mg/m².

In some embodiments, the paclitaxel is administered at least 2 times per week at an amount of about 200 mg/m² to about 400 mg/m².

In some embodiments, the paclitaxel is administered at least 2 times per week at an amount of about 250 mg/m² to about 350 mg/m².

In some embodiments, the paclitaxel is administered at least 2 times per week at an amount of about 100 mg/m² to about 250 mg/m².

In some embodiments, the paclitaxel is administered at least 2 times per week at an amount of about 100 mg/m² to about 225 mg/m².

In some embodiments, the paclitaxel is administered at least 2 times per week at an amount of about 100 mg/m² to about 205 mg/m².

In some embodiments, the paclitaxel is administered at least 2 times per week at an amount of about 100 mg/m² to about 180 mg/m².

In some embodiments, the paclitaxel is administered at least 2 times per week at an amount of about 100 mg/m² to about 165 mg/m².

In some embodiments, the paclitaxel is administered at least 2 times per week at an amount of about 100 mg/m² to about 150 mg/m².

In some embodiments, the paclitaxel is administered at least 2 times per week at an amount of about 100 mg/m² to about 135 mg/m².

In some embodiments, the paclitaxel is administered at least 2 times per week at an amount of about 125 mg/m² to about 250 mg/m².

In some embodiments, the paclitaxel is administered at least 2 times per week at an amount of about 125 mg/m² to about 225 mg/m².

In some embodiments, the paclitaxel is administered at least 2 times per week at an amount of about 125 mg/m² to about 205 mg/m².

In some embodiments, the paclitaxel is administered at least 2 times per week at an amount of about 125 mg/m² to about 180 mg/m².

In some embodiments, the paclitaxel is administered at least 2 times per week at an amount of about 125 mg/m² to about 165 mg/m².

In some embodiments, the paclitaxel is administered at least 2 times per week at an amount of about 125 mg/m² to about 150 mg/m².

In some embodiments, the paclitaxel is administered at least 2 times per week at an amount of about 125 mg/m² to about 135 mg/m².

In some embodiments, the paclitaxel is administered at least 2 times per week at an amount of about 135 mg/m² to about 250 mg/m².

In some embodiments, the paclitaxel is administered at least 2 times per week at an amount of about 135 mg/m² to about 225 mg/m².

In some embodiments, the paclitaxel is administered at least 2 times per week at an amount of about 135 mg/m² to about 205 mg/m².

In some embodiments, the paclitaxel is administered at least 2 times per week at an amount of about 135 mg/m² to about 180 mg/m².

In some embodiments, the paclitaxel is administered at least 2 times per week at an amount of about 135 mg/m² to about 165 mg/m².

In some embodiments, the paclitaxel is administered at least 2 times per week at an amount of about 135 mg/m² to about 150 mg/m².

In some embodiments, the paclitaxel is administered at least 2 times per week at an amount of about 150 mg/m² to about 250 mg/m².

In some embodiments, the paclitaxel is administered at least 2 times per week at an amount of about 150 mg/m² to about 225 mg/m².

In some embodiments, the paclitaxel is administered at least 2 times per week at an amount of about 150 mg/m² to about 205 mg/m².

In some embodiments, the paclitaxel is administered at least 2 times per week at an amount of about 150 mg/m² to about 180 mg/m².

In some embodiments, the paclitaxel is administered at least 2 times per week at an amount of about 150 mg/m² to about 165 mg/m².

In some embodiments, the paclitaxel is administered at least 2 times per week at an amount of about 165 mg/m² to about 250 mg/m².

In some embodiments, the paclitaxel is administered at least 2 times per week at an amount of about 165 mg/m² to about 225 mg/m².

In some embodiments, the paclitaxel is administered at least 2 times per week at an amount of about 165 mg/m² to about 205 mg/m².

In some embodiments, the paclitaxel is administered at least 2 times per week at an amount of about 165 mg/m² to about 180 mg/m².

In some embodiments, the paclitaxel is administered at least 2 times per week at an amount of about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 250 mg/m², about 275 mg/m², about 300 mg/m², about 325 mg/m², about 350 mg/m², about 375 mg/m², or about 400 mg/m². In some embodiments, the paclitaxel is administered at least 2 times per week at an amount of about 250 mg/m², about 275 mg/m², about 300 mg/m², about 325 mg/m², or about 350 mg/m². In some embodiments, the paclitaxel is administered at least 2 times per week at an amount of about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 2 times per week at an amount of about 130 mg/m², about 135 mg/m², about 140 mg/m², about 145 mg/m², about 150 mg/m², about 155 mg/m², about 160 mg/m², about 165 mg/m², or about 170 mg/m². In some embodiments, the paclitaxel is administered at least 2 times per week at an amount of about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 2 times per week at an amount of about 135 mg/m², about 165 mg/m², or about 205 mg/m².

In some embodiments, the paclitaxel is administered at least 3 times per week at an amount of about 150 mg/m² to about 400 mg/m².

In some embodiments, the paclitaxel is administered at least 3 times per week at an amount of about 200 mg/m² to about 400 mg/m².

In some embodiments, the paclitaxel is administered at least 3 times per week at an amount of about 250 mg/m² to about 350 mg/m².

In some embodiments, the paclitaxel is administered at least 3 times per week at an amount of about 100 mg/m² to about 250 mg/m².

In some embodiments, the paclitaxel is administered at least 3 times per week at an amount of about 100 mg/m² to about 225 mg/m².

In some embodiments, the paclitaxel is administered at least 3 times per week at an amount of about 100 mg/m² to about 205 mg/m².

In some embodiments, the paclitaxel is administered at least 3 times per week at an amount of about 100 mg/m² to about 180 mg/m².

In some embodiments, the paclitaxel is administered at least 3 times per week at an amount of about 100 mg/m² to about 165 mg/m².

In some embodiments, the paclitaxel is administered at least 3 times per week at an amount of about 100 mg/m² to about 150 mg/m².

In some embodiments, the paclitaxel is administered at least 3 times per week at an amount of about 100 mg/m² to about 135 mg/m².

In some embodiments, the paclitaxel is administered at least 3 times per week at an amount of about 125 mg/m² to about 250 mg/m².

In some embodiments, the paclitaxel is administered at least 3 times per week at an amount of about 125 mg/m² to about 225 mg/m².

In some embodiments, the paclitaxel is administered at least 3 times per week at an amount of about 125 mg/m² to about 205 mg/m².

In some embodiments, the paclitaxel is administered at least 3 times per week at an amount of about 125 mg/m² to about 180 mg/m².

In some embodiments, the paclitaxel is administered at least 3 times per week at an amount of about 125 mg/m² to about 165 mg/m².

In some embodiments, the paclitaxel is administered at least 3 times per week at an amount of about 125 mg/m² to about 150 mg/m².

In some embodiments, the paclitaxel is administered at least 3 times per week at an amount of about 125 mg/m² to about 135 mg/m².

In some embodiments, the paclitaxel is administered at least 3 times per week at an amount of about 135 mg/m² to about 250 mg/m².

In some embodiments, the paclitaxel is administered at least 3 times per week at an amount of about 135 mg/m² to about 225 mg/m².

In some embodiments, the paclitaxel is administered at least 3 times per week at an amount of about 135 mg/m² to about 205 mg/m².

In some embodiments, the paclitaxel is administered at least 3 times per week at an amount of about 135 mg/m² to about 180 mg/m².

In some embodiments, the paclitaxel is administered at least 3 times per week at an amount of about 135 mg/m² to about 165 mg/m².

In some embodiments, the paclitaxel is administered at least 3 times per week at an amount of about 135 mg/m² to about 150 mg/m².

In some embodiments, the paclitaxel is administered at least 3 times per week at an amount of about 150 mg/m² to about 250 mg/m².

In some embodiments, the paclitaxel is administered at least 3 times per week at an amount of about 150 mg/m² to about 225 mg/m².

In some embodiments, the paclitaxel is administered at least 3 times per week at an amount of about 150 mg/m² to about 205 mg/m².

In some embodiments, the paclitaxel is administered at least 3 times per week at an amount of about 150 mg/m² to about 180 mg/m².

In some embodiments, the paclitaxel is administered at least 3 times per week at an amount of about 150 mg/m² to about 165 mg/m².

In some embodiments, the paclitaxel is administered at least 3 times per week at an amount of about 165 mg/m² to about 250 mg/m².

In some embodiments, the paclitaxel is administered at least 3 times per week at an amount of about 165 mg/m² to about 225 mg/m².

In some embodiments, the paclitaxel is administered at least 3 times per week at an amount of about 165 mg/m² to about 205 mg/m².

In some embodiments, the paclitaxel is administered at least 3 times per week at an amount of about 165 mg/m² to about 180 mg/m².

In some embodiments, the paclitaxel is administered at least 3 times per week at an amount of about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 250 mg/m², about 275 mg/m², about 300 mg/m², about 325 mg/m², about 350 mg/m², about 375 mg/m², or about 400 mg/m². In some embodiments, the paclitaxel is administered at least 3 times per week at an amount of about 250 mg/m², about 275 mg/m², about 300 mg/m², about 325 mg/m², or about 350 mg/m². In some embodiments, the paclitaxel is administered at least 3 times per week at an amount of about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 3 times per week at an amount of about 130 mg/m², about 135 mg/m², about 140 mg/m², about 145 mg/m², about 150 mg/m², about 155 mg/m², about 160 mg/m², about 165 mg/m², or about 170 mg/m². In some embodiments, the paclitaxel is administered at least 3 times per week at an amount of about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 3 times per week at an amount of about 135 mg/m², about 165 mg/m², or about 205 mg/m².

In some embodiments, the paclitaxel is administered at least 4 times per week at an amount of about 150 mg/m² to about 400 mg/m².

In some embodiments, the paclitaxel is administered at least 4 times per week at an amount of about 200 mg/m² to about 400 mg/m².

In some embodiments, the paclitaxel is administered at least 4 times per week at an amount of about 250 mg/m² to about 350 mg/m².

In some embodiments, the paclitaxel is administered at least 4 times per week at an amount of about 100 mg/m² to about 250 mg/m².

In some embodiments, the paclitaxel is administered at least 4 times per week at an amount of about 100 mg/m² to about 225 mg/m².

In some embodiments, the paclitaxel is administered at least 4 times per week at an amount of about 100 mg/m² to about 205 mg/m².

In some embodiments, the paclitaxel is administered at least 4 times per week at an amount of about 100 mg/m² to about 180 mg/m².

In some embodiments, the paclitaxel is administered at least 4 times per week at an amount of about 100 mg/m² to about 165 mg/m².

In some embodiments, the paclitaxel is administered at least 4 times per week at an amount of about 100 mg/m² to about 150 mg/m².

In some embodiments, the paclitaxel is administered at least 4 times per week at an amount of about 100 mg/m² to about 135 mg/m².

In some embodiments, the paclitaxel is administered at least 4 times per week at an amount of about 125 mg/m² to about 250 mg/m².

In some embodiments, the paclitaxel is administered at least 4 times per week at an amount of about 125 mg/m² to about 225 mg/m².

In some embodiments, the paclitaxel is administered at least 4 times per week at an amount of about 125 mg/m² to about 205 mg/m².

In some embodiments, the paclitaxel is administered at least 4 times per week at an amount of about 125 mg/m² to about 180 mg/m².

In some embodiments, the paclitaxel is administered at least 4 times per week at an amount of about 125 mg/m² to about 165 mg/m².

In some embodiments, the paclitaxel is administered at least 4 times per week at an amount of about 125 mg/m² to about 150 mg/m².

In some embodiments, the paclitaxel is administered at least 4 times per week at an amount of about 125 mg/m² to about 135 mg/m².

In some embodiments, the paclitaxel is administered at least 4 times per week at an amount of about 135 mg/m² to about 250 mg/m².

In some embodiments, the paclitaxel is administered at least 4 times per week at an amount of about 135 mg/m² to about 225 mg/m².

In some embodiments, the paclitaxel is administered at least 4 times per week at an amount of about 135 mg/m² to about 205 mg/m².

In some embodiments, the paclitaxel is administered at least 4 times per week at an amount of about 135 mg/m² to about 180 mg/m².

In some embodiments, the paclitaxel is administered at least 4 times per week at an amount of about 135 mg/m² to about 165 mg/m².

In some embodiments, the paclitaxel is administered at least 4 times per week at an amount of about 135 mg/m² to about 150 mg/m².

In some embodiments, the paclitaxel is administered at least 4 times per week at an amount of about 150 mg/m² to about 250 mg/m².

In some embodiments, the paclitaxel is administered at least 4 times per week at an amount of about 150 mg/m² to about 225 mg/m².

In some embodiments, the paclitaxel is administered at least 4 times per week at an amount of about 150 mg/m² to about 205 mg/m².

In some embodiments, the paclitaxel is administered at least 4 times per week at an amount of about 150 mg/m² to about 180 mg/m².

In some embodiments, the paclitaxel is administered at least 4 times per week at an amount of about 150 mg/m² to about 165 mg/m².

In some embodiments, the paclitaxel is administered at least 4 times per week at an amount of about 165 mg/m² to about 250 mg/m².

In some embodiments, the paclitaxel is administered at least 4 times per week at an amount of about 165 mg/m² to about 225 mg/m².

In some embodiments, the paclitaxel is administered at least 4 times per week at an amount of about 165 mg/m² to about 205 mg/m².

In some embodiments, the paclitaxel is administered at least 4 times per week at an amount of about 165 mg/m² to about 180 mg/m².

In some embodiments, the paclitaxel is administered at least 4 times per week at an amount of about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 250 mg/m², about 275 mg/m², about 300 mg/m², about 325 mg/m², about 350 mg/m², about 375 mg/m², or about 400 mg/m². In some embodiments, the paclitaxel is administered at least 4 times per week at an amount of about 250 mg/m², about 275 mg/m², about 300 mg/m², about 325 mg/m², or about 350 mg/m². In some embodiments, the paclitaxel is administered at least 4 times per week at an amount of about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 4 times per week at an amount of about 130 mg/m², about 135 mg/m², about 140 mg/m², about 145 mg/m², about 150 mg/m², about 155 mg/m², about 160 mg/m², about 165 mg/m², or about 170 mg/m². In some embodiments, the paclitaxel is administered at least 4 times per week at an amount of about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 4 times per week at an amount of about 100 mg/m², about 125 mg/m², about 135 mg/m², about 150 mg/m², about 165 mg/m², or about 205 mg/m².

In some embodiments, the paclitaxel is administered at least 5 times per week at an amount of about 150 mg/m² to about 400 mg/m².

In some embodiments, the paclitaxel is administered at least 5 times per week at an amount of about 200 mg/m² to about 400 mg/m².

In some embodiments, the paclitaxel is administered at least 5 times per week at an amount of about 250 mg/m² to about 350 mg/m².

In some embodiments, the paclitaxel is administered at least 5 times per week at an amount of about 100 mg/m² to about 250 mg/m².

In some embodiments, the paclitaxel is administered at least 5 times per week at an amount of about 100 mg/m² to about 225 mg/m².

In some embodiments, the paclitaxel is administered at least 5 times per week at an amount of about 100 mg/m² to about 205 mg/m².

In some embodiments, the paclitaxel is administered at least 5 times per week at an amount of about 100 mg/m² to about 180 mg/m².

In some embodiments, the paclitaxel is administered at least 5 times per week at an amount of about 100 mg/m² to about 165 mg/m².

In some embodiments, the paclitaxel is administered at least 5 times per week at an amount of about 100 mg/m² to about 150 mg/m².

In some embodiments, the paclitaxel is administered at least 5 times per week at an amount of about 100 mg/m² to about 135 mg/m².

In some embodiments, the paclitaxel is administered at least 5 times per week at an amount of about 125 mg/m² to about 250 mg/m².

In some embodiments, the paclitaxel is administered at least 5 times per week at an amount of about 125 mg/m² to about 225 mg/m².

In some embodiments, the paclitaxel is administered at least 5 times per week at an amount of about 125 mg/m² to about 205 mg/m².

In some embodiments, the paclitaxel is administered at least 5 times per week at an amount of about 125 mg/m² to about 180 mg/m².

In some embodiments, the paclitaxel is administered at least 5 times per week at an amount of about 125 mg/m² to about 165 mg/m².

In some embodiments, the paclitaxel is administered at least 5 times per week at an amount of about 125 mg/m² to about 150 mg/m².

In some embodiments, the paclitaxel is administered at least 5 times per week at an amount of about 125 mg/m² to about 135 mg/m².

In some embodiments, the paclitaxel is administered at least 5 times per week at an amount of about 135 mg/m² to about 250 mg/m².

In some embodiments, the paclitaxel is administered at least 5 times per week at an amount of about 135 mg/m² to about 225 mg/m².

In some embodiments, the paclitaxel is administered at least 5 times per week at an amount of about 135 mg/m² to about 205 mg/m².

In some embodiments, the paclitaxel is administered at least 5 times per week at an amount of about 135 mg/m² to about 180 mg/m².

In some embodiments, the paclitaxel is administered at least 5 times per week at an amount of about 135 mg/m² to about 165 mg/m².

In some embodiments, the paclitaxel is administered at least 5 times per week at an amount of about 135 mg/m² to about 150 mg/m².

In some embodiments, the paclitaxel is administered at least 5 times per week at an amount of about 150 mg/m² to about 250 mg/m².

In some embodiments, the paclitaxel is administered at least 5 times per week at an amount of about 150 mg/m² to about 225 mg/m².

In some embodiments, the paclitaxel is administered at least 5 times per week at an amount of about 150 mg/m² to about 205 mg/m².

In some embodiments, the paclitaxel is administered at least 5 times per week at an amount of about 150 mg/m² to about 180 mg/m².

In some embodiments, the paclitaxel is administered at least 5 times per week at an amount of about 150 mg/m² to about 165 mg/m².

In some embodiments, the paclitaxel is administered at least 5 times per week at an amount of about 165 mg/m² to about 250 mg/m².

In some embodiments, the paclitaxel is administered at least 5 times per week at an amount of about 165 mg/m² to about 225 mg/m².

In some embodiments, the paclitaxel is administered at least 5 times per week at an amount of about 165 mg/m² to about 205 mg/m².

In some embodiments, the paclitaxel is administered at least 5 times per week at an amount of about 165 mg/m² to about 180 mg/m².

In some embodiments, the paclitaxel is administered at least 5 times per week at an amount of about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 250 mg/m², about 275 mg/m², about 300 mg/m², about 325 mg/m², about 350 mg/m², about 375 mg/m², or about 400 mg/m². In some embodiments, the paclitaxel is administered at least 5 times per week at an amount of about 250 mg/m², about 275 mg/m², about 300 mg/m², about 325 mg/m², or about 350 mg/m². In some embodiments, the paclitaxel is administered at least 5 times per week at an amount of about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 5 times per week at an amount of about 130 mg/m², about 135 mg/m², about 140 mg/m², about 145 mg/m², about 150 mg/m², about 155 mg/m², about 160 mg/m², about 165 mg/m², or about 170 mg/m². In some embodiments, the paclitaxel is administered at least 5 times per week at an amount of about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 5 times per week at an amount of about 100 mg/m², about 125 mg/m², about 135 mg/m², about 150 mg/m², about 165 mg/m², or about 205 mg/m².

In some embodiments, the paclitaxel is administered at least 6 times per week at an amount of about 150 mg/m² to about 400 mg/m².

In some embodiments, the paclitaxel is administered at least 6 times per week at an amount of about 200 mg/m² to about 400 mg/m².

In some embodiments, the paclitaxel is administered at least 6 times per week at an amount of about 250 mg/m² to about 350 mg/m².

In some embodiments, the paclitaxel is administered at least 6 times per week at an amount of about 100 mg/m² to about 250 mg/m².

In some embodiments, the paclitaxel is administered at least 6 times per week at an amount of about 100 mg/m² to about 225 mg/m².

In some embodiments, the paclitaxel is administered at least 6 times per week at an amount of about 100 mg/m² to about 205 mg/m².

In some embodiments, the paclitaxel is administered at least 6 times per week at an amount of about 100 mg/m² to about 180 mg/m².

In some embodiments, the paclitaxel is administered at least 6 times per week at an amount of about 100 mg/m² to about 165 mg/m².

In some embodiments, the paclitaxel is administered at least 6 times per week at an amount of about 100 mg/m² to about 150 mg/m².

In some embodiments, the paclitaxel is administered at least 6 times per week at an amount of about 100 mg/m² to about 135 mg/m².

In some embodiments, the paclitaxel is administered at least 6 times per week at an amount of about 125 mg/m² to about 250 mg/m².

In some embodiments, the paclitaxel is administered at least 6 times per week at an amount of about 125 mg/m² to about 225 mg/m².

In some embodiments, the paclitaxel is administered at least 6 times per week at an amount of about 125 mg/m² to about 205 mg/m².

In some embodiments, the paclitaxel is administered at least 6 times per week at an amount of about 125 mg/m² to about 180 mg/m².

In some embodiments, the paclitaxel is administered at least 6 times per week at an amount of about 125 mg/m² to about 165 mg/m².

In some embodiments, the paclitaxel is administered at least 6 times per week at an amount of about 125 mg/m² to about 150 mg/m².

In some embodiments, the paclitaxel is administered at least 6 times per week at an amount of about 125 mg/m² to about 135 mg/m².

In some embodiments, the paclitaxel is administered at least 6 times per week at an amount of about 135 mg/m² to about 250 mg/m².

In some embodiments, the paclitaxel is administered at least 6 times per week at an amount of about 135 mg/m² to about 225 mg/m².

In some embodiments, the paclitaxel is administered at least 6 times per week at an amount of about 135 mg/m² to about 205 mg/m².

In some embodiments, the paclitaxel is administered at least 6 times per week at an amount of about 135 mg/m² to about 180 mg/m².

In some embodiments, the paclitaxel is administered at least 6 times per week at an amount of about 135 mg/m² to about 165 mg/m².

In some embodiments, the paclitaxel is administered at least 6 times per week at an amount of about 135 mg/m² to about 150 mg/m².

In some embodiments, the paclitaxel is administered at least 6 times per week at an amount of about 150 mg/m² to about 250 mg/m².

In some embodiments, the paclitaxel is administered at least 6 times per week at an amount of about 150 mg/m² to about 225 mg/m².

In some embodiments, the paclitaxel is administered at least 6 times per week at an amount of about 150 mg/m² to about 205 mg/m².

In some embodiments, the paclitaxel is administered at least 6 times per week at an amount of about 150 mg/m² to about 180 mg/m².

In some embodiments, the paclitaxel is administered at least 6 times per week at an amount of about 150 mg/m² to about 165 mg/m².

In some embodiments, the paclitaxel is administered at least 6 times per week at an amount of about 165 mg/m² to about 250 mg/m².

In some embodiments, the paclitaxel is administered at least 6 times per week at an amount of about 165 mg/m² to about 225 mg/m².

In some embodiments, the paclitaxel is administered at least 6 times per week at an amount of about 165 mg/m² to about 205 mg/m².

In some embodiments, the paclitaxel is administered at least 6 times per week at an amount of about 165 mg/m² to about 180 mg/m².

In some embodiments, the paclitaxel is administered at least 6 times per week at an amount of about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 250 mg/m², about 275 mg/m², about 300 mg/m², about 325 mg/m², about 350 mg/m², about 375 mg/m², or about 400 mg/m². In some embodiments, the paclitaxel is administered at least 6 times per week at an amount of about 250 mg/m², about 275 mg/m², about 300 mg/m², about 325 mg/m², or about 350 mg/m². In some embodiments, the paclitaxel is administered at least 6 times per week at an amount of about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 6 times per week at an amount of about 130 mg/m², about 135 mg/m², about 140 mg/m², about 145 mg/m², about 150 mg/m², about 155 mg/m², about 160 mg/m², about 165 mg/m², or about 170 mg/m². In some embodiments, the paclitaxel is administered at least 6 times per week at an amount of about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², or about 210 mg/m². In some embodiments, the paclitaxel is administered at least 6 times per week at an amount of about 100 mg/m², about 125 mg/m², about 135 mg/m², about 150 mg/m², about 165 mg/m², or about 205 mg/m².

In some embodiments, Compound A in the present application is formulated for oral administration at the various doses described herein with the various dosing frequencies in the methods, compositions, medicaments, therapeutic combinations, combinational therapies, and uses, described herein.

In some embodiments, Compound A is administered 1-7 times per week. In some embodiments, Compound A is administered on consecutive days.

In some embodiments, Compound A is administered 2-7 times per week. In some embodiments, Compound A is administered on consecutive days.

In some embodiments, Compound A is administered 3-7 times per week. In some embodiments, Compound A is administered on consecutive days.

In some embodiments, Compound A is administered 4-7 times per week. In some embodiments, Compound A is administered on consecutive days.

In some embodiments, Compound A is administered 5-7 times per week. In some embodiments, Compound A is administered on consecutive days.

In some embodiments, Compound A is administered 6-7 times per week. In some embodiments, Compound A is administered on consecutive days.

In some embodiments, Compound A is administered 1-6 times per week. In some embodiments, Compound A is administered 1-5 times per week. In some embodiments, Compound A is administered 1-4 times per week. In some embodiments, Compound A is administered 1-3 times per week. In some embodiments, Compound A is administered 1-2 times per week. In some embodiments, Compound A is administered on consecutive days.

In some embodiments, Compound A is administered 2-6 times per week. In some embodiments, Compound A is administered 2-5 times per week. In some embodiments, Compound A is administered 2-4 times per week. In some embodiments, Compound A is administered 2-3 times per week. In some embodiments, Compound A is administered on consecutive days.

In some embodiments, Compound A is administered 3-6 times per week. In some embodiments, Compound A is administered 3-5 times per week. In some embodiments, Compound A is administered 3-4 times per week. In some embodiments, Compound A is administered on consecutive days.

In some embodiments, Compound A is administered 4-6 times per week. In some embodiments, Compound A is administered 4-5 times per week. In some embodiments, Compound A is administered on consecutive days.

In some embodiments, Compound A is administered 5-6 times per week. In some embodiments, Compound A is administered on consecutive days.

In some embodiments, Compound A is administered at least 1 time per week. In some embodiments, Compound A is administered on consecutive days.

In some embodiments, Compound A is administered at least 2 times per week. In some embodiments, Compound A is administered on consecutive days.

In some embodiments, Compound A is administered at least 3 times per week. In some embodiments, Compound A is administered on consecutive days.

In some embodiments, Compound A is administered at least 4 times per week. In some embodiments, Compound A is administered on consecutive days.

In some embodiments, Compound A is administered at least 5 times per week. In some embodiments, Compound A is administered on consecutive days.

In some embodiments, Compound A is administered at least 6 times per week. In some embodiments, Compound A is administered on consecutive days.

In some embodiments, Compound A is administered daily per week.

In some embodiments, Compound A is administered at least 1 time per week at an amount of about 1 mg to about 500 mg.

In some embodiments, Compound A is administered at least 1 time per week at an amount of about 1 mg to about 400 mg.

In some embodiments, Compound A is administered at least 1 time per week at an amount of about 1 mg to about 300 mg.

In some embodiments, Compound A is administered at least 1 time per week at an amount of about 5 mg to about 200 mg.

In some embodiments, Compound A is administered at least 1 time per week at an amount of about 10 mg to about 100 mg.

In some embodiments, Compound A is administered at least 1 time per week at an amount of about 10 mg to about 95 mg.

In some embodiments, Compound A is administered at least 1 time per week at an amount of about 10 mg to about 90 mg.

In some embodiments, Compound A is administered at least 1 time per week at an amount of about 10 mg to about 85 mg.

In some embodiments, Compound A is administered at least 1 time per week at an amount of about 10 mg to about 80 mg.

In some embodiments, Compound A is administered at least 1 time per week at an amount of about 10 mg to about 75 mg.

In some embodiments, Compound A is administered at least 1 time per week at an amount of about 10 mg to about 70 mg.

In some embodiments, Compound A is administered at least 1 time per week at an amount of about 10 mg to about 65 mg.

In some embodiments, Compound A is administered at least 1 time per week at an amount of about 10 mg to about 60 mg.

In some embodiments, Compound A is administered at least 1 time per week at an amount of about 10 mg to about 55 mg.

In some embodiments, Compound A is administered at least 1 time per week at an amount of about 10 mg to about 50 mg.

In some embodiments, Compound A is administered at least 1 time per week at an amount of about 10 mg to about 45 mg.

In some embodiments, Compound A is administered at least 1 time per week at an amount of about 10 mg to about 40 mg.

In some embodiments, Compound A is administered at least 1 time per week at an amount of about 10 mg to about 35 mg.

In some embodiments, Compound A is administered at least 1 time per week at an amount of about 10 mg to about 30 mg.

In some embodiments, Compound A is administered at least 1 time per week at an amount of about 10 mg to about 25 mg.

In some embodiments, Compound A is administered at least 1 time per week at an amount of about 10 mg to about 20 mg.

In some embodiments, Compound A is administered at least 1 time per week at an amount of about 10 mg to about 15 mg.

In some embodiments, Compound A is administered at least 1 time per week at an amount of about 15 mg to about 50 mg.

In some embodiments, Compound A is administered at least 1 time per week at an amount of about 15 mg to about 45 mg.

In some embodiments, Compound A is administered at least 1 time per week at an amount of about 15 mg to about 40 mg.

In some embodiments, Compound A is administered at least 1 time per week at an amount of about 15 mg to about 35 mg.

In some embodiments, Compound A is administered at least 1 time per week at an amount of about 15 mg to about 30 mg.

In some embodiments, Compound A is administered at least 1 time per week at an amount of about 15 mg to about 25 mg.

In some embodiments, Compound A is administered at least 1 time per week at an amount of about 15 mg to about 20 mg.

In some embodiments, Compound A is administered at least 1 time per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 1 time per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 1 time per week at an amount of about 55 mg, about 60 mg, about 65 mg, about 70 mg, or about 75 mg. In some embodiments, Compound A is administered at least 1 time per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 1 time per week at an amount of about 5 mg, about 10 mg, about 15 mg, about 20 mg, or about 25 mg. In some embodiments, Compound A is administered at least 1 time per week at an amount of about 30 mg, about 35 mg, about 40 mg, about 45 mg, or about 50 mg. In some embodiments, Compound A is administered at least 1 time per week at an amount of about 10 mg, about 15 mg, or about 20 mg. In some embodiments, Compound A is administered at least 1 time per week at an amount of about 15 mg.

In some embodiments, Compound A is administered at least 2 times per week at an amount of about 1 mg to about 500 mg.

In some embodiments, Compound A is administered at least 2 times per week at an amount of about 1 mg to about 400 mg.

In some embodiments, Compound A is administered at least 2 times per week at an amount of about 1 mg to about 300 mg.

In some embodiments, Compound A is administered at least 2 times per week at an amount of about 5 mg to about 200 mg.

In some embodiments, Compound A is administered at least 2 times per week at an amount of about 10 mg to about 100 mg.

In some embodiments, Compound A is administered at least 2 times per week at an amount of about 10 mg to about 95 mg.

In some embodiments, Compound A is administered at least 2 times per week at an amount of about 10 mg to about 90 mg.

In some embodiments, Compound A is administered at least 2 times per week at an amount of about 10 mg to about 85 mg.

In some embodiments, Compound A is administered at least 2 times per week at an amount of about 10 mg to about 80 mg.

In some embodiments, Compound A is administered at least 2 times per week at an amount of about 10 mg to about 75 mg.

In some embodiments, Compound A is administered at least 2 times per week at an amount of about 10 mg to about 70 mg.

In some embodiments, Compound A is administered at least 2 times per week at an amount of about 10 mg to about 65 mg.

In some embodiments, Compound A is administered at least 2 times per week at an amount of about 10 mg to about 60 mg.

In some embodiments, Compound A is administered at least 2 times per week at an amount of about 10 mg to about 55 mg.

In some embodiments, Compound A is administered at least 2 times per week at an amount of about 10 mg to about 50 mg.

In some embodiments, Compound A is administered at least 2 times per week at an amount of about 10 mg to about 45 mg.

In some embodiments, Compound A is administered at least 2 times per week at an amount of about 10 mg to about 40 mg.

In some embodiments, Compound A is administered at least 2 times per week at an amount of about 10 mg to about 35 mg.

In some embodiments, Compound A is administered at least 2 times per week at an amount of about 10 mg to about 30 mg.

In some embodiments, Compound A is administered at least 2 times per week at an amount of about 10 mg to about 25 mg.

In some embodiments, Compound A is administered at least 2 times per week at an amount of about 10 mg to about 20 mg.

In some embodiments, Compound A is administered at least 2 times per week at an amount of about 10 mg to about 15 mg.

In some embodiments, Compound A is administered at least 2 times per week at an amount of about 15 mg to about 50 mg.

In some embodiments, Compound A is administered at least 2 times per week at an amount of about 15 mg to about 45 mg.

In some embodiments, Compound A is administered at least 2 times per week at an amount of about 15 mg to about 40 mg.

In some embodiments, Compound A is administered at least 2 times per week at an amount of about 15 mg to about 35 mg.

In some embodiments, Compound A is administered at least 2 times per week at an amount of about 15 mg to about 30 mg.

In some embodiments, Compound A is administered at least 2 times per week at an amount of about 15 mg to about 25 mg.

In some embodiments, Compound A is administered at least 2 times per week at an amount of about 15 mg to about 20 mg.

In some embodiments, Compound A is administered at least 2 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 2 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 2 times per week at an amount of about 55 mg, about 60 mg, about 65 mg, about 70 mg, or about 75 mg. In some embodiments, Compound A is administered at least 2 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 2 times per week at an amount of about 5 mg, about 10 mg, about 15 mg, about 20 mg, or about 25 mg. In some embodiments, Compound A is administered at least 2 times per week at an amount of about 30 mg, about 35 mg, about 40 mg, about 45 mg, or about 50 mg. In some embodiments, Compound A is administered at least 2 times per week at an amount of about 10 mg, about 15 mg, or about 20 mg. In some embodiments, Compound A is administered at least 2 times per week at an amount of about 15 mg.

In some embodiments, Compound A is administered at least 3 times per week at an amount of about 1 mg to about 500 mg.

In some embodiments, Compound A is administered at least 3 times per week at an amount of about 1 mg to about 400 mg.

In some embodiments, Compound A is administered at least 3 times per week at an amount of about 1 mg to about 300 mg.

In some embodiments, Compound A is administered at least 3 times per week at an amount of about 5 mg to about 200 mg.

In some embodiments, Compound A is administered at least 3 times per week at an amount of about 10 mg to about 100 mg.

In some embodiments, Compound A is administered at least 3 times per week at an amount of about 10 mg to about 95 mg.

In some embodiments, Compound A is administered at least 3 times per week at an amount of about 10 mg to about 90 mg.

In some embodiments, Compound A is administered at least 3 times per week at an amount of about 10 mg to about 85 mg.

In some embodiments, Compound A is administered at least 3 times per week at an amount of about 10 mg to about 80 mg.

In some embodiments, Compound A is administered at least 3 times per week at an amount of about 10 mg to about 75 mg.

In some embodiments, Compound A is administered at least 3 times per week at an amount of about 10 mg to about 70 mg.

In some embodiments, Compound A is administered at least 3 times per week at an amount of about 10 mg to about 65 mg.

In some embodiments, Compound A is administered at least 3 times per week at an amount of about 10 mg to about 60 mg.

In some embodiments, Compound A is administered at least 3 times per week at an amount of about 10 mg to about 55 mg.

In some embodiments, Compound A is administered at least 3 times per week at an amount of about 10 mg to about 50 mg.

In some embodiments, Compound A is administered at least 3 times per week at an amount of about 10 mg to about 45 mg.

In some embodiments, Compound A is administered at least 3 times per week at an amount of about 10 mg to about 40 mg.

In some embodiments, Compound A is administered at least 3 times per week at an amount of about 10 mg to about 35 mg.

In some embodiments, Compound A is administered at least 3 times per week at an amount of about 10 mg to about 30 mg.

In some embodiments, Compound A is administered at least 3 times per week at an amount of about 10 mg to about 25 mg.

In some embodiments, Compound A is administered at least 3 times per week at an amount of about 10 mg to about 20 mg.

In some embodiments, Compound A is administered at least 3 times per week at an amount of about 10 mg to about 15 mg.

In some embodiments, Compound A is administered at least 3 times per week at an amount of about 15 mg to about 50 mg.

In some embodiments, Compound A is administered at least 3 times per week at an amount of about 15 mg to about 45 mg.

In some embodiments, Compound A is administered at least 3 times per week at an amount of about 15 mg to about 40 mg.

In some embodiments, Compound A is administered at least 3 times per week at an amount of about 15 mg to about 35 mg.

In some embodiments, Compound A is administered at least 3 times per week at an amount of about 15 mg to about 30 mg.

In some embodiments, Compound A is administered at least 3 times per week at an amount of about 15 mg to about 25 mg.

In some embodiments, Compound A is administered at least 3 times per week at an amount of about 15 mg to about 20 mg.

In some embodiments, Compound A is administered at least 3 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 3 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 3 times per week at an amount of about 55 mg, about 60 mg, about 65 mg, about 70 mg, or about 75 mg. In some embodiments, Compound A is administered at least 3 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 3 times per week at an amount of about 5 mg, about 10 mg, about 15 mg, about 20 mg, or about 25 mg. In some embodiments, Compound A is administered at least 3 times per week at an amount of about 30 mg, about 35 mg, about 40 mg, about 45 mg, or about 50 mg. In some embodiments, Compound A is administered at least 3 times per week at an amount of about 10 mg, about 15 mg, or about 20 mg. In some embodiments, Compound A is administered at least 3 times per week at an amount of about 15 mg.

In some embodiments, Compound A is administered at least 4 times per week at an amount of about 1 mg to about 500 mg.

In some embodiments, Compound A is administered at least 4 times per week at an amount of about 1 mg to about 400 mg.

In some embodiments, Compound A is administered at least 4 times per week at an amount of about 1 mg to about 300 mg.

In some embodiments, Compound A is administered at least 4 times per week at an amount of about 5 mg to about 200 mg.

In some embodiments, Compound A is administered at least 4 times per week at an amount of about 10 mg to about 100 mg.

In some embodiments, Compound A is administered at least 4 times per week at an amount of about 10 mg to about 95 mg.

In some embodiments, Compound A is administered at least 4 times per week at an amount of about 10 mg to about 90 mg.

In some embodiments, Compound A is administered at least 4 times per week at an amount of about 10 mg to about 85 mg.

In some embodiments, Compound A is administered at least 4 times per week at an amount of about 10 mg to about 80 mg.

In some embodiments, Compound A is administered at least 4 times per week at an amount of about 10 mg to about 75 mg.

In some embodiments, Compound A is administered at least 4 times per week at an amount of about 10 mg to about 70 mg.

In some embodiments, Compound A is administered at least 4 times per week at an amount of about 10 mg to about 65 mg.

In some embodiments, Compound A is administered at least 4 times per week at an amount of about 10 mg to about 60 mg.

In some embodiments, Compound A is administered at least 4 times per week at an amount of about 10 mg to about 55 mg.

In some embodiments, Compound A is administered at least 4 times per week at an amount of about 10 mg to about 50 mg.

In some embodiments, Compound A is administered at least 4 times per week at an amount of about 10 mg to about 45 mg.

In some embodiments, Compound A is administered at least 4 times per week at an amount of about 10 mg to about 40 mg.

In some embodiments, Compound A is administered at least 4 times per week at an amount of about 10 mg to about 35 mg.

In some embodiments, Compound A is administered at least 4 times per week at an amount of about 10 mg to about 30 mg.

In some embodiments, Compound A is administered at least 4 times per week at an amount of about 10 mg to about 25 mg.

In some embodiments, Compound A is administered at least 4 times per week at an amount of about 10 mg to about 20 mg.

In some embodiments, Compound A is administered at least 4 times per week at an amount of about 10 mg to about 15 mg.

In some embodiments, Compound A is administered at least 4 times per week at an amount of about 15 mg to about 50 mg.

In some embodiments, Compound A is administered at least 4 times per week at an amount of about 15 mg to about 45 mg.

In some embodiments, Compound A is administered at least 4 times per week at an amount of about 15 mg to about 40 mg.

In some embodiments, Compound A is administered at least 4 times per week at an amount of about 15 mg to about 35 mg.

In some embodiments, Compound A is administered at least 4 times per week at an amount of about 15 mg to about 30 mg.

In some embodiments, Compound A is administered at least 4 times per week at an amount of about 15 mg to about 25 mg.

In some embodiments, Compound A is administered at least 4 times per week at an amount of about 15 mg to about 20 mg.

In some embodiments, Compound A is administered at least 4 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 4 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 4 times per week at an amount of about 55 mg, about 60 mg, about 65 mg, about 70 mg, or about 75 mg. In some embodiments, Compound A is administered at least 4 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 4 times per week at an amount of about 5 mg, about 10 mg, about 15 mg, about 20 mg, or about 25 mg. In some embodiments, Compound A is administered at least 4 times per week at an amount of about 30 mg, about 35 mg, about 40 mg, about 45 mg, or about 50 mg. In some embodiments, Compound A is administered at least 4 times per week at an amount of about 10 mg, about 15 mg, or about 20 mg. In some embodiments, Compound A is administered at least 4 times per week at an amount of about 15 mg.

In some embodiments, Compound A is administered at least 5 times per week at an amount of about 1 mg to about 500 mg.

In some embodiments, Compound A is administered at least 5 times per week at an amount of about 1 mg to about 400 mg.

In some embodiments, Compound A is administered at least 5 times per week at an amount of about 1 mg to about 300 mg.

In some embodiments, Compound A is administered at least 5 times per week at an amount of about 5 mg to about 200 mg.

In some embodiments, Compound A is administered at least 5 times per week at an amount of about 10 mg to about 100 mg.

In some embodiments, Compound A is administered at least 5 times per week at an amount of about 10 mg to about 95 mg.

In some embodiments, Compound A is administered at least 5 times per week at an amount of about 10 mg to about 90 mg.

In some embodiments, Compound A is administered at least 5 times per week at an amount of about 10 mg to about 85 mg.

In some embodiments, Compound A is administered at least 5 times per week at an amount of about 10 mg to about 80 mg.

In some embodiments, Compound A is administered at least 5 times per week at an amount of about 10 mg to about 75 mg.

In some embodiments, Compound A is administered at least 5 times per week at an amount of about 10 mg to about 70 mg.

In some embodiments, Compound A is administered at least 5 times per week at an amount of about 10 mg to about 65 mg.

In some embodiments, Compound A is administered at least 5 times per week at an amount of about 10 mg to about 60 mg.

In some embodiments, Compound A is administered at least 5 times per week at an amount of about 10 mg to about 55 mg.

In some embodiments, Compound A is administered at least 5 times per week at an amount of about 10 mg to about 50 mg.

In some embodiments, Compound A is administered at least 5 times per week at an amount of about 10 mg to about 45 mg.

In some embodiments, Compound A is administered at least 5 times per week at an amount of about 10 mg to about 40 mg.

In some embodiments, Compound A is administered at least 5 times per week at an amount of about 10 mg to about 35 mg.

In some embodiments, Compound A is administered at least 5 times per week at an amount of about 10 mg to about 30 mg.

In some embodiments, Compound A is administered at least 5 times per week at an amount of about 10 mg to about 25 mg.

In some embodiments, Compound A is administered at least 5 times per week at an amount of about 10 mg to about 20 mg.

In some embodiments, Compound A is administered at least 5 times per week at an amount of about 10 mg to about 15 mg.

In some embodiments, Compound A is administered at least 5 times per week at an amount of about 15 mg to about 50 mg.

In some embodiments, Compound A is administered at least 5 times per week at an amount of about 15 mg to about 45 mg.

In some embodiments, Compound A is administered at least 5 times per week at an amount of about 15 mg to about 40 mg.

In some embodiments, Compound A is administered at least 5 times per week at an amount of about 15 mg to about 35 mg.

In some embodiments, Compound A is administered at least 5 times per week at an amount of about 15 mg to about 30 mg.

In some embodiments, Compound A is administered at least 5 times per week at an amount of about 15 mg to about 25 mg.

In some embodiments, Compound A is administered at least 5 times per week at an amount of about 15 mg to about 20 mg.

In some embodiments, Compound A is administered at least 5 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 5 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 5 times per week at an amount of about 55 mg, about 60 mg, about 65 mg, about 70 mg, or about 75 mg. In some embodiments, Compound A is administered at least 5 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 5 times per week at an amount of about 5 mg, about 10 mg, about 15 mg, about 20 mg, or about 25 mg. In some embodiments, Compound A is administered at least 5 times per week at an amount of about 30 mg, about 35 mg, about 40 mg, about 45 mg, or about 50 mg. In some embodiments, Compound A is administered at least 5 times per week at an amount of about 10 mg, about 15 mg, or about 20 mg. In some embodiments, Compound A is administered at least 5 times per week at an amount of about 15 mg.

In some embodiments, Compound A is administered at least 6 times per week at an amount of about 1 mg to about 500 mg.

In some embodiments, Compound A is administered at least 6 times per week at an amount of about 1 mg to about 400 mg.

In some embodiments, Compound A is administered at least 6 times per week at an amount of about 1 mg to about 300 mg.

In some embodiments, Compound A is administered at least 6 times per week at an amount of about 5 mg to about 200 mg.

In some embodiments, Compound A is administered at least 6 times per week at an amount of about 10 mg to about 100 mg.

In some embodiments, Compound A is administered at least 6 times per week at an amount of about 10 mg to about 95 mg.

In some embodiments, Compound A is administered at least 6 times per week at an amount of about 10 mg to about 90 mg.

In some embodiments, Compound A is administered at least 6 times per week at an amount of about 10 mg to about 85 mg.

In some embodiments, Compound A is administered at least 6 times per week at an amount of about 10 mg to about 80 mg.

In some embodiments, Compound A is administered at least 6 times per week at an amount of about 10 mg to about 75 mg.

In some embodiments, Compound A is administered at least 6 times per week at an amount of about 10 mg to about 70 mg.

In some embodiments, Compound A is administered at least 6 times per week at an amount of about 10 mg to about 65 mg.

In some embodiments, Compound A is administered at least 6 times per week at an amount of about 10 mg to about 60 mg.

In some embodiments, Compound A is administered at least 6 times per week at an amount of about 10 mg to about 55 mg.

In some embodiments, Compound A is administered at least 6 times per week at an amount of about 10 mg to about 50 mg.

In some embodiments, Compound A is administered at least 6 times per week at an amount of about 10 mg to about 45 mg.

In some embodiments, Compound A is administered at least 6 times per week at an amount of about 10 mg to about 40 mg.

In some embodiments, Compound A is administered at least 6 times per week at an amount of about 10 mg to about 35 mg.

In some embodiments, Compound A is administered at least 6 times per week at an amount of about 10 mg to about 30 mg.

In some embodiments, Compound A is administered at least 6 times per week at an amount of about 10 mg to about 25 mg.

In some embodiments, Compound A is administered at least 6 times per week at an amount of about 10 mg to about 20 mg.

In some embodiments, Compound A is administered at least 6 times per week at an amount of about 10 mg to about 15 mg.

In some embodiments, Compound A is administered at least 6 times per week at an amount of about 15 mg to about 50 mg.

In some embodiments, Compound A is administered at least 6 times per week at an amount of about 15 mg to about 45 mg.

In some embodiments, Compound A is administered at least 6 times per week at an amount of about 15 mg to about 40 mg.

In some embodiments, Compound A is administered at least 6 times per week at an amount of about 15 mg to about 35 mg.

In some embodiments, Compound A is administered at least 6 times per week at an amount of about 15 mg to about 30 mg.

In some embodiments, Compound A is administered at least 6 times per week at an amount of about 15 mg to about 25 mg.

In some embodiments, Compound A is administered at least 6 times per week at an amount of about 15 mg to about 20 mg.

In some embodiments, Compound A is administered at least 6 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 6 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 6 times per week at an amount of about 55 mg, about 60 mg, about 65 mg, about 70 mg, or about 75 mg. In some embodiments, Compound A is administered at least 6 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 6 times per week at an amount of about 5 mg, about 10 mg, about 15 mg, about 20 mg, or about 25 mg. In some embodiments, Compound A is administered at least 6 times per week at an amount of about 30 mg, about 35 mg, about 40 mg, about 45 mg, or about 50 mg. In some embodiments, Compound A is administered at least 6 times per week at an amount of about 10 mg, about 15 mg, or about 20 mg. In some embodiments, Compound A is administered at least 6 times per week at an amount of about 15 mg.

In some embodiments, any dose described herein for paclitaxel can be combined with any dose described herein for Compound A, which combination can be combined with any dosing frequency described herein for paclitaxel and for Compound A.

In some embodiments, the paclitaxel is administered at an amount of about 100 mg/m² to about 400 mg/m² in combination with Compound A.

In some embodiments, the paclitaxel is administered at an amount of about 100 mg/m² to about 350 mg/m² in combination with Compound A.

In some embodiments, the paclitaxel is administered at an amount of about 100 mg/m² to about 300 mg/m² in combination with Compound A.

In some embodiments, the paclitaxel is administered at an amount of about 150 mg/m² to about 250 mg/m² in combination with Compound A.

In some embodiments, the paclitaxel is administered at an amount of about 100 mg/m² to about 245 mg/m² in combination with Compound A.

In some embodiments, the paclitaxel is administered at an amount of about 100 mg/m² to about 240 mg/m² in combination with Compound A.

In some embodiments, the paclitaxel is administered at an amount of about 100 mg/m² to about 235 mg/m² in combination with Compound A.

In some embodiments, the paclitaxel is administered at an amount of about 100 mg/m² to about 230 mg/m² in combination with Compound A.

In some embodiments, the paclitaxel is administered at an amount of about 100 mg/m² to about 225 mg/m² in combination with Compound A.

In some embodiments, the paclitaxel is administered at an amount of about 100 mg/m² to about 220 mg/m² in combination with Compound A.

In some embodiments, the paclitaxel is administered at an amount of about 100 mg/m² to about 215 mg/m² in combination with Compound A.

In some embodiments, the paclitaxel is administered at an amount of about 100 mg/m² to about 210 mg/m² in combination with Compound A.

In some embodiments, the paclitaxel is administered at an amount of about 100 mg/m² to about 205 mg/m² in combination with Compound A.

In some embodiments, the paclitaxel is administered at an amount of about 100 mg/m² to about 200 mg/m² in combination with Compound A.

In some embodiments, the paclitaxel is administered at an amount of about 100 mg/m² to about 195 mg/m² in combination with Compound A.

In some embodiments, the paclitaxel is administered at an amount of about 100 mg/m² to about 190 mg/m² in combination with Compound A.

In some embodiments, the paclitaxel is administered at an amount of about 100 mg/m² to about 185 mg/m² in combination with Compound A.

In some embodiments, the paclitaxel is administered at an amount of about 100 mg/m² to about 180 mg/m² in combination with Compound A.

In some embodiments, the paclitaxel is administered at an amount of about 100 mg/m² to about 175 mg/m² in combination with Compound A.

In some embodiments, the paclitaxel is administered at an amount of about 100 mg/m² to about 170 mg/m² in combination with Compound A.

In some embodiments, the paclitaxel is administered at an amount of about 100 mg/m² to about 165 mg/m² in combination with Compound A.

In some embodiments, the paclitaxel is administered at an amount of about 100 mg/m² to about 160 mg/m² in combination with Compound A.

In some embodiments, the paclitaxel is administered at an amount of about 100 mg/m² to about 155 mg/m² in combination with Compound A

In some embodiments, the paclitaxel is administered at an amount of about 100 mg/m² to about 150 mg/m² in combination with Compound A.

In some embodiments, the paclitaxel is administered at an amount of about 100 mg/m² to about 145 mg/m² in combination with Compound A.

In some embodiments, the paclitaxel is administered at an amount of about 100 mg/m² to about 140 mg/m² in combination with Compound A.

In some embodiments, the paclitaxel is administered at an amount of about 100 mg/m² to about 135 mg/m² in combination with Compound A.

In some embodiments, the paclitaxel is administered at an amount of about 100 mg/m² to about 130 mg/m² in combination with Compound A.

In some embodiments, the paclitaxel is administered at an amount of about 100 mg/m² to about 125 mg/m² in combination with Compound A.

In some embodiments, the paclitaxel is administered at an amount of about 100 mg/m² to about 120 mg/m² in combination with Compound A.

In some embodiments, the paclitaxel is administered at an amount of about 100 mg/m² to about 115 mg/m² in combination with Compound A.

In some embodiments, the paclitaxel is administered at an amount of about 100 mg/m² to about 110 mg/m² in combination with Compound A.

In some embodiments, the paclitaxel is administered at an amount of about 100 mg/m² to about 105 mg/m² in combination with Compound A.

In some embodiments, the paclitaxel is administered at an amount of about 150 mg/m² to about 350 mg/m² in combination with Compound A.

In some embodiments, the paclitaxel is administered at an amount of about 150 mg/m² to about 300 mg/m² in combination with Compound A.

In some embodiments, the paclitaxel is administered at an amount of about 150 mg/m² to about 250 mg/m² in combination with Compound A.

In some embodiments, the paclitaxel is administered at an amount of about 150 mg/m² to about 245 mg/m² in combination with Compound A.

In some embodiments, the paclitaxel is administered at an amount of about 150 mg/m² to about 240 mg/m² in combination with Compound A.

In some embodiments, the paclitaxel is administered at an amount of about 150 mg/m² to about 235 mg/m² in combination with Compound A.

In some embodiments, the paclitaxel is administered at an amount of about 150 mg/m² to about 230 mg/m² in combination with Compound A.

In some embodiments, the paclitaxel is administered at an amount of about 150 mg/m² to about 225 mg/m² in combination with Compound A.

In some embodiments, the paclitaxel is administered at an amount of about 150 mg/m² to about 220 mg/m² in combination with Compound A.

In some embodiments, the paclitaxel is administered at an amount of about 150 mg/m² to about 215 mg/m² in combination with Compound A.

In some embodiments, the paclitaxel is administered at an amount of about 150 mg/m² to about 210 mg/m² in combination with Compound A.

In some embodiments, the paclitaxel is administered at an amount of about 150 mg/m² to about 205 mg/m² in combination with Compound A.

In some embodiments, the paclitaxel is administered at an amount of about 150 mg/m² to about 200 mg/m² in combination with Compound A.

In some embodiments, the paclitaxel is administered at an amount of about 150 mg/m² to about 195 mg/m² in combination with Compound A.

In some embodiments, the paclitaxel is administered at an amount of about 150 mg/m² to about 190 mg/m² in combination with Compound A.

In some embodiments, the paclitaxel is administered at an amount of about 150 mg/m² to about 185 mg/m² in combination with Compound A.

In some embodiments, the paclitaxel is administered at an amount of about 150 mg/m² to about 180 mg/m² in combination with Compound A.

In some embodiments, the paclitaxel is administered at an amount of about 150 mg/m² to about 175 mg/m² in combination with Compound A.

In some embodiments, the paclitaxel is administered at an amount of about 150 mg/m² to about 170 mg/m² in combination with Compound A.

In some embodiments, the paclitaxel is administered at an amount of about 150 mg/m² to about 165 mg/m² in combination with Compound A.

In some embodiments, the paclitaxel is administered at an amount of about 150 mg/m² to about 160 mg/m² in combination with Compound A.

In some embodiments, the paclitaxel is administered at an amount of about 150 mg/m² to about 155 mg/m² in combination with Compound A.

In some embodiments, the paclitaxel is administered at an amount of about 200 mg/m² to about 300 mg/m² in combination with Compound A.

In some embodiments, the paclitaxel is administered at an amount of about 200 mg/m² to about 295 mg/m² in combination with Compound A.

In some embodiments, the paclitaxel is administered at an amount of about 200 mg/m² to about 290 mg/m² in combination with Compound A.

In some embodiments, the paclitaxel is administered at an amount of about 200 mg/m² to about 285 mg/m² in combination with Compound A.

In some embodiments, the paclitaxel is administered at an amount of about 200 mg/m² to about 280 mg/m² in combination with Compound A.

In some embodiments, the paclitaxel is administered at an amount of about 200 mg/m² to about 275 mg/m² in combination with Compound A.

In some embodiments, the paclitaxel is administered at an amount of about 200 mg/m² to about 270 mg/m² in combination with Compound A.

In some embodiments, the paclitaxel is administered at an amount of about 200 mg/m² to about 265 mg/m² in combination with Compound A.

In some embodiments, the paclitaxel is administered at an amount of about 200 mg/m² to about 260 mg/m² in combination with Compound A.

In some embodiments, the paclitaxel is administered at an amount of about 200 mg/m² to about 255 mg/m² in combination with Compound A.

In some embodiments, the paclitaxel is administered at an amount of about 200 mg/m² to about 250 mg/m² in combination with Compound A.

In some embodiments, the paclitaxel is administered at an amount of about 200 mg/m² to about 245 mg/m² in combination with Compound A.

In some embodiments, the paclitaxel is administered at an amount of about 200 mg/m² to about 240 mg/m² in combination with Compound A.

In some embodiments, the paclitaxel is administered at an amount of about 200 mg/m² to about 235 mg/m² in combination with Compound A.

In some embodiments, the paclitaxel is administered at an amount of about 200 mg/m² to about 230 mg/m² in combination with Compound A.

In some embodiments, the paclitaxel is administered at an amount of about 200 mg/m² to about 225 mg/m² in combination with Compound A.

In some embodiments, the paclitaxel is administered at an amount of about 200 mg/m² to about 220 mg/m² in combination with Compound A.

In some embodiments, the paclitaxel is administered at an amount of about 200 mg/m² to about 215 mg/m² in combination with Compound A.

In some embodiments, the paclitaxel is administered at an amount of about 200 mg/m² to about 210 mg/m² in combination with Compound A.

In some embodiments, the paclitaxel is administered at an amount of about 200 mg/m² to about 205 mg/m² in combination with Compound A.

In some embodiments, the paclitaxel is administered at an amount of about 250 mg/m² to about 350 mg/m² in combination with Compound A.

In some embodiments, the paclitaxel is administered at an amount of about 250 mg/m² to about 300 mg/m² in combination with Compound A.

In some embodiments, the paclitaxel is administered at an amount of about 120 mg/m² to about 235 mg/m² in combination with Compound A.

In some embodiments, the paclitaxel is administered at an amount of about 120 mg/m² to about 230 mg/m² in combination with Compound A.

In some embodiments, the paclitaxel is administered at an amount of about 120 mg/m² to about 225 mg/m² in combination with Compound A.

In some embodiments, the paclitaxel is administered at an amount of about 120 mg/m² to about 220 mg/m² in combination with Compound A.

In some embodiments, the paclitaxel is administered at an amount of about 120 mg/m² to about 215 mg/m² in combination with Compound A.

In some embodiments, the paclitaxel is administered at an amount of about 120 mg/m² to about 210 mg/m² in combination with Compound A.

In some embodiments, the paclitaxel is administered at an amount of about 120 mg/m² to about 205 mg/m² in combination with Compound A.

In some embodiments, the paclitaxel is administered at an amount of about 120 mg/m² to about 200 mg/m² in combination with Compound A.

In some embodiments, the paclitaxel is administered at an amount of about 120 mg/m² to about 195 mg/m² in combination with Compound A.

In some embodiments, the paclitaxel is administered at an amount of about 120 mg/m² to about 190 mg/m² in combination with Compound A.

In some embodiments, the paclitaxel is administered at an amount of about 120 mg/m² to about 185 mg/m² in combination with Compound A.

In some embodiments, the paclitaxel is administered at an amount of about 120 mg/m² to about 180 mg/m² in combination with Compound A.

In some embodiments, the paclitaxel is administered at an amount of about 120 mg/m² to about 175 mg/m² in combination with Compound A.

In some embodiments, the paclitaxel is administered at an amount of about 120 mg/m² to about 170 mg/m² in combination with Compound A.

In some embodiments, the paclitaxel is administered at an amount of about 120 mg/m² to about 165 mg/m² in combination with Compound A.

In some embodiments, the paclitaxel is administered at an amount of about 120 mg/m² to about 160 mg/m² in combination with Compound A.

In some embodiments, the paclitaxel is administered at an amount of about 120 mg/m² to about 155 mg/m² in combination with Compound A.

In some embodiments, the paclitaxel is administered at an amount of about 120 mg/m² to about 150 mg/m² in combination with Compound A.

In some embodiments, the paclitaxel is administered at an amount of about 120 mg/m² to about 145 mg/m² in combination with Compound A.

In some embodiments, the paclitaxel is administered at an amount of about 120 mg/m² to about 140 mg/m² in combination with Compound A.

In some embodiments, the paclitaxel is administered at an amount of about 120 mg/m² to about 135 mg/m² in combination with Compound A.

In some embodiments, the paclitaxel is administered at an amount of about 120 mg/m² to about 130 mg/m² in combination with Compound A.

In some embodiments, the paclitaxel is administered at an amount of about 120 mg/m² to about 125 mg/m² in combination with Compound A.

In some embodiments, the paclitaxel is administered at an amount of about 125 mg/m² to about 235 mg/m² in combination with Compound A.

In some embodiments, the paclitaxel is administered at an amount of about 125 mg/m² to about 230 mg/m² in combination with Compound A.

In some embodiments, the paclitaxel is administered at an amount of about 125 mg/m² to about 225 mg/m² in combination with Compound A.

In some embodiments, the paclitaxel is administered at an amount of about 125 mg/m² to about 220 mg/m² in combination with Compound A.

In some embodiments, the paclitaxel is administered at an amount of about 125 mg/m² to about 215 mg/m² in combination with Compound A.

In some embodiments, the paclitaxel is administered at an amount of about 125 mg/m² to about 210 mg/m² in combination with Compound A.

In some embodiments, the paclitaxel is administered at an amount of about 125 mg/m² to about 205 mg/m² in combination with Compound A.

In some embodiments, the paclitaxel is administered at an amount of about 125 mg/m² to about 200 mg/m² in combination with Compound A.

In some embodiments, the paclitaxel is administered at an amount of about 125 mg/m² to about 195 mg/m² in combination with Compound A.

In some embodiments, the paclitaxel is administered at an amount of about 125 mg/m² to about 190 mg/m² in combination with Compound A.

In some embodiments, the paclitaxel is administered at an amount of about 125 mg/m² to about 185 mg/m² in combination with Compound A.

In some embodiments, the paclitaxel is administered at an amount of about 125 mg/m² to about 180 mg/m² in combination with Compound A.

In some embodiments, the paclitaxel is administered at an amount of about 125 mg/m² to about 175 mg/m² in combination with Compound A.

In some embodiments, the paclitaxel is administered at an amount of about 125 mg/m² to about 170 mg/m² in combination with Compound A.

In some embodiments, the paclitaxel is administered at an amount of about 125 mg/m² to about 165 mg/m² in combination with Compound A.

In some embodiments, the paclitaxel is administered at an amount of about 125 mg/m² to about 160 mg/m² in combination with Compound A.

In some embodiments, the paclitaxel is administered at an amount of about 125 mg/m² to about 155 mg/m² in combination with Compound A.

In some embodiments, the paclitaxel is administered at an amount of about 125 mg/m² to about 150 mg/m² in combination with Compound A.

In some embodiments, the paclitaxel is administered at an amount of about 125 mg/m² to about 145 mg/m² in combination with Compound A.

In some embodiments, the paclitaxel is administered at an amount of about 125 mg/m² to about 140 mg/m² in combination with Compound A.

In some embodiments, the paclitaxel is administered at an amount of about 125 mg/m² to about 135 mg/m² in combination with Compound A.

In some embodiments, the paclitaxel is administered at an amount of about 125 mg/m² to about 130 mg/m² in combination with Compound A.

In some embodiments, the paclitaxel is administered at an amount of about 130 mg/m² to about 235 mg/m² in combination with Compound A.

In some embodiments, the paclitaxel is administered at an amount of about 130 mg/m² to about 230 mg/m² in combination with Compound A.

In some embodiments, the paclitaxel is administered at an amount of about 130 mg/m² to about 225 mg/m² in combination with Compound A.

In some embodiments, the paclitaxel is administered at an amount of about 130 mg/m² to about 220 mg/m² in combination with Compound A.

In some embodiments, the paclitaxel is administered at an amount of about 130 mg/m² to about 215 mg/m² in combination with Compound A.

In some embodiments, the paclitaxel is administered at an amount of about 130 mg/m² to about 210 mg/m² in combination with Compound A.

In some embodiments, the paclitaxel is administered at an amount of about 130 mg/m² to about 205 mg/m² in combination with Compound A.

In some embodiments, the paclitaxel is administered at an amount of about 130 mg/m² to about 200 mg/m² in combination with Compound A.

In some embodiments, the paclitaxel is administered at an amount of about 130 mg/m² to about 195 mg/m² in combination with Compound A.

In some embodiments, the paclitaxel is administered at an amount of about 130 mg/m² to about 190 mg/m² in combination with Compound A.

In some embodiments, the paclitaxel is administered at an amount of about 130 mg/m² to about 185 mg/m² in combination with Compound A.

In some embodiments, the paclitaxel is administered at an amount of about 130 mg/m² to about 180 mg/m² in combination with Compound A.

In some embodiments, the paclitaxel is administered at an amount of about 130 mg/m² to about 175 mg/m² in combination with Compound A.

In some embodiments, the paclitaxel is administered at an amount of about 130 mg/m² to about 170 mg/m² in combination with Compound A.

In some embodiments, the paclitaxel is administered at an amount of about 130 mg/m² to about 165 mg/m² in combination with Compound A.

In some embodiments, the paclitaxel is administered at an amount of about 130 mg/m² to about 160 mg/m² in combination with Compound A.

In some embodiments, the paclitaxel is administered at an amount of about 130 mg/m² to about 155 mg/m² in combination with Compound A.

In some embodiments, the paclitaxel is administered at an amount of about 130 mg/m² to about 150 mg/m² in combination with Compound A.

In some embodiments, the paclitaxel is administered at an amount of about 130 mg/m² to about 145 mg/m² in combination with Compound A.

In some embodiments, the paclitaxel is administered at an amount of about 130 mg/m² to about 140 mg/m² in combination with Compound A.

In some embodiments, the paclitaxel is administered at an amount of about 130 mg/m² to about 135 mg/m² in combination with Compound A.

In some embodiments, the paclitaxel is administered at an amount of about 100 mg/m² in combination with Compound A.

In some embodiments, the paclitaxel is administered at an amount of about 105 mg/m² in combination with Compound A.

In some embodiments, the paclitaxel is administered at an amount of about 110 mg/m² in combination with Compound A.

In some embodiments, the paclitaxel is administered at an amount of about 115 mg/m² in combination with Compound A.

In some embodiments, the paclitaxel is administered at an amount of about 120 mg/m² in combination with Compound A.

In some embodiments, the paclitaxel is administered at an amount of about 125 mg/m² in combination with Compound A.

In some embodiments, the paclitaxel is administered at an amount of about 130 mg/m² in combination with Compound A.

In some embodiments, the paclitaxel is administered at an amount of about 135 mg/m² in combination with Compound A.

In some embodiments, the paclitaxel is administered at an amount of about 140 mg/m² in combination with Compound A.

In some embodiments, the paclitaxel is administered at an amount of about 145 mg/m² in combination with Compound A.

In some embodiments, the paclitaxel is administered at an amount of about 150 mg/m² in combination with Compound A.

In some embodiments, the paclitaxel is administered at an amount of about 155 mg/m² in combination with Compound A.

In some embodiments, the paclitaxel is administered at an amount of about 160 mg/m² in combination with Compound A.

In some embodiments, the paclitaxel is administered at an amount of about 165 mg/m² in combination with Compound A.

In some embodiments, the paclitaxel is administered at an amount of about 170 mg/m² in combination with Compound A.

In some embodiments, the paclitaxel is administered at an amount of about 175 mg/m² in combination with Compound A.

In some embodiments, the paclitaxel is administered at an amount of about 180 mg/m² in combination with Compound A.

In some embodiments, the paclitaxel is administered at an amount of about 185 mg/m² in combination with Compound A.

In some embodiments, the paclitaxel is administered at an amount of about 190 mg/m² in combination with Compound A.

In some embodiments, the paclitaxel is administered at an amount of about 195 mg/m² in combination with Compound A.

In some embodiments, the paclitaxel is administered at an amount of about 200 mg/m² in combination with Compound A.

In some embodiments, the paclitaxel is administered at an amount of about 205 mg/m² in combination with Compound A.

In some embodiments, the paclitaxel is administered at an amount of about 210 mg/m² in combination with Compound A.

In some embodiments, the paclitaxel is administered at an amount of about 215 mg/m² in combination with Compound A.

In some embodiments, the paclitaxel is administered at an amount of about 220 mg/m² in combination with Compound A.

In some embodiments, the paclitaxel is administered at an amount of about 225 mg/m² in combination with Compound A.

In some embodiments, the paclitaxel is administered at an amount of about 230 mg/m² in combination with Compound A.

In some embodiments, the paclitaxel is administered at an amount of about 235 mg/m² in combination with Compound A.

In some embodiments, the paclitaxel is administered at an amount of about 240 mg/m² in combination with Compound A.

In some embodiments, the paclitaxel is administered at an amount of about 245 mg/m² in combination with Compound A.

In some embodiments, the paclitaxel is administered at an amount of about 250 mg/m² in combination with Compound A.

In some embodiments, the paclitaxel is administered at an amount of about 255 mg/m² in combination with Compound A.

In some embodiments, the paclitaxel is administered at an amount of about 260 mg/m² in combination with Compound A.

In some embodiments, the paclitaxel is administered at an amount of about 265 mg/m² in combination with Compound A.

In some embodiments, the paclitaxel is administered at an amount of about 270 mg/m² in combination with Compound A.

In some embodiments, the paclitaxel is administered at an amount of about 275 mg/m² in combination with Compound A.

In some embodiments, the paclitaxel is administered at an amount of about 280 mg/m² in combination with Compound A.

In some embodiments, the paclitaxel is administered at an amount of about 285 mg/m² in combination with Compound A.

In some embodiments, the paclitaxel is administered at an amount of about 290 mg/m² in combination with Compound A.

In some embodiments, the paclitaxel is administered at an amount of about 295 mg/m² in combination with Compound A.

In some embodiments, the paclitaxel is administered at an amount of about 300 mg/m² in combination with Compound A.

In some embodiments, the paclitaxel is administered at an amount of about 305 mg/m² in combination with Compound A.

In some embodiments, the paclitaxel is administered at an amount of about 310 mg/m² in combination with Compound A.

In some embodiments, the paclitaxel is administered at an amount of about 315 mg/m² in combination with Compound A.

In some embodiments, the paclitaxel is administered at an amount of about 320 mg/m² in combination with Compound A.

In some embodiments, the paclitaxel is administered at an amount of about 325 mg/m² in combination with Compound A.

In some embodiments, the paclitaxel is administered at an amount of about 330 mg/m² in combination with Compound A.

In some embodiments, the paclitaxel is administered at an amount of about 335 mg/m² in combination with Compound A.

In some embodiments, the paclitaxel is administered at an amount of about 340 mg/m² in combination with Compound A.

In some embodiments, the paclitaxel is administered at an amount of about 345 mg/m² in combination with Compound A.

In some embodiments, the paclitaxel is administered at an amount of about 350 mg/m² in combination with Compound A.

In some embodiments, the paclitaxel is administered at an amount of about 355 mg/m² in combination with Compound A.

In some embodiments, the paclitaxel is administered at an amount of about 360 mg/m² in combination with Compound A.

In some embodiments, the paclitaxel is administered at an amount of about 365 mg/m² in combination with Compound A.

In some embodiments, the paclitaxel is administered at an amount of about 370 mg/m² in combination with Compound A.

In some embodiments, the paclitaxel is administered at an amount of about 375 mg/m² in combination with Compound A.

In some embodiments, the paclitaxel is administered at an amount of about 380 mg/m² in combination with Compound A.

In some embodiments, the paclitaxel is administered at an amount of about 385 mg/m² in combination with Compound A.

In some embodiments, the paclitaxel is administered at an amount of about 390 mg/m² in combination with Compound A.

In some embodiments, the paclitaxel is administered at an amount of about 395 mg/m² in combination with Compound A.

In some embodiments, the paclitaxel is administered at an amount of about 400 mg/m² in combination with Compound A.

In some embodiments, Compound A is administered at an amount of about 1 mg to about 500 mg in combination with paclitaxel.

In some embodiments, Compound A is administered at about 1 mg to about 400 mg in combination with paclitaxel.

In some embodiments, Compound A is administered at about 1 mg to about 300 mg in combination with paclitaxel.

In some embodiments, Compound A is administered at about 5 mg to about 200 mg in combination with paclitaxel.

In some embodiments, Compound A is administered at about 10 mg to about 100 mg in combination with paclitaxel.

In some embodiments, Compound A is administered at about 10 mg to about 95 mg in combination with paclitaxel.

In some embodiments, Compound A is administered at about 10 mg to about 90 mg in combination with paclitaxel.

In some embodiments, Compound A is administered at about 10 mg to about 85 mg in combination with paclitaxel.

In some embodiments, Compound A is administered at about 10 mg to about 80 mg in combination with paclitaxel.

In some embodiments, Compound A is administered at about 10 mg to about 75 mg in combination with paclitaxel.

In some embodiments, Compound A is administered at about 10 mg to about 70 mg in combination with paclitaxel.

In some embodiments, Compound A is administered at about 10 mg to about 65 mg in combination with paclitaxel.

In some embodiments, Compound A is administered at about 10 mg to about 60 mg in combination with paclitaxel.

In some embodiments, Compound A is administered at about 10 mg to about 55 mg in combination with paclitaxel.

In some embodiments, Compound A is administered at about 10 mg to about 50 mg in combination with paclitaxel.

In some embodiments, Compound A is administered at about 10 mg to about 45 mg in combination with paclitaxel.

In some embodiments, Compound A is administered at about 10 mg to about 40 mg in combination with paclitaxel.

In some embodiments, Compound A is administered at about 10 mg to about 35 mg in combination with paclitaxel.

In some embodiments, Compound A is administered at about 10 mg to about 30 mg in combination with paclitaxel.

In some embodiments, Compound A is administered at about 10 mg to about 25 mg in combination with paclitaxel.

In some embodiments, Compound A is administered at about 10 mg to about 20 mg in combination with paclitaxel.

In some embodiments, Compound A is administered at about 10 mg to about 15 mg in combination with paclitaxel.

In some embodiments, Compound A is administered at about 15 mg to about 50 mg in combination with paclitaxel.

In some embodiments, Compound A is administered at about 15 mg to about 45 mg in combination with paclitaxel.

In some embodiments, Compound A is administered at about 15 mg to about 40 mg in combination with paclitaxel.

In some embodiments, Compound A is administered at about 15 mg to about 35 mg in combination with paclitaxel.

In some embodiments, Compound A is administered at about 15 mg to about 30 mg in combination with paclitaxel.

In some embodiments, Compound A is administered at about 15 mg to about 25 mg in combination with paclitaxel.

In some embodiments, Compound A is administered at about 15 mg to about 20 mg in combination with paclitaxel.

In some embodiments, Compound A is administered at about 5 mg in combination with paclitaxel.

In some embodiments, Compound A is administered at about 10 mg in combination with paclitaxel.

In some embodiments, Compound A is administered at about 15 mg in combination with paclitaxel.

In some embodiments, Compound A is administered at about 20 mg in combination with paclitaxel.

In some embodiments, Compound A is administered at about 25 mg in combination with paclitaxel.

In some embodiments, Compound A is administered at about 30 mg in combination with paclitaxel.

In some embodiments, Compound A is administered at about 35 mg in combination with paclitaxel.

In some embodiments, Compound A is administered at about 40 mg in combination with paclitaxel.

In some embodiments, Compound A is administered at about 45 mg in combination with paclitaxel.

In some embodiments, Compound A is administered at about 50 mg in combination with paclitaxel.

In some embodiments, the paclitaxel is administered in combination with Compound A 1-7 times per week. In some embodiments, the paclitaxel is administered in combination with Compound A on consecutive days.

In some embodiments, the paclitaxel is administered in combination with Compound A 2-7 times per week. In some embodiments, the paclitaxel is administered in combination with Compound A on consecutive days.

In some embodiments, the paclitaxel is administered in combination with Compound A 3-7 times per week. In some embodiments, the paclitaxel is administered in combination with Compound A on consecutive days.

In some embodiments, the paclitaxel is administered in combination with Compound A 4-7 times per week. In some embodiments, the paclitaxel is administered in combination with Compound A on consecutive days.

In some embodiments, the paclitaxel is administered in combination with Compound A 5-7 times per week. In some embodiments, the paclitaxel is administered in combination with Compound A on consecutive days.

In some embodiments, the paclitaxel is administered in combination with Compound A 6-7 times per week. In some embodiments, the paclitaxel is administered in combination with Compound A on consecutive days.

In some embodiments, the paclitaxel is administered in combination with Compound A 1-6 times per week. In some embodiments, the paclitaxel is administered in combination with Compound A 1-5 times per week. In some embodiments, the paclitaxel is administered in combination with Compound A 1-4 times per week. In some embodiments, the paclitaxel is administered in combination with Compound A 1-3 times per week. In some embodiments, the paclitaxel is administered in combination with Compound A 1-2 times per week. In some embodiments, the paclitaxel is administered in combination with Compound A on consecutive days.

In some embodiments, the paclitaxel is administered in combination with Compound A 2-6 times per week. In some embodiments, the paclitaxel is administered in combination with Compound A 2-5 times per week. In some embodiments, the paclitaxel is administered in combination with Compound A 2-4 times per week. In some embodiments, the paclitaxel is administered in combination with Compound A 2-3 times per week. In some embodiments, the paclitaxel is administered in combination with Compound A on consecutive days.

In some embodiments, the paclitaxel is administered in combination with Compound A 3-6 times per week. In some embodiments, the paclitaxel is administered in combination with Compound A 3-5 times per week. In some embodiments, the paclitaxel is administered in combination with Compound A 3-4 times per week. In some embodiments, the paclitaxel is administered in combination with Compound A on consecutive days.

In some embodiments, the paclitaxel is administered in combination with Compound A 4-6 times per week. In some embodiments, the paclitaxel is administered in combination with Compound A 4-5 times per week. In some embodiments, the paclitaxel is administered in combination with Compound A on consecutive days.

In some embodiments, the paclitaxel is administered in combination with Compound A 5-6 times per week. In some embodiments, the paclitaxel is administered in combination with Compound A on consecutive days.

In some embodiments, the paclitaxel is administered in combination with Compound A at least 1 time per week. In some embodiments, the paclitaxel is administered in combination with Compound A on consecutive days.

In some embodiments, the paclitaxel is administered in combination with Compound A at least 2 times per week. In some embodiments, the paclitaxel is administered in combination with Compound A on consecutive days.

In some embodiments, the paclitaxel is administered in combination with Compound A at least 3 times per week. In some embodiments, the paclitaxel is administered in combination with Compound A on consecutive days.

In some embodiments, the paclitaxel is administered in combination with Compound A at least 4 times per week. In some embodiments, the paclitaxel is administered in combination with Compound A on consecutive days.

In some embodiments, the paclitaxel is administered in combination with Compound A at least 5 times per week. In some embodiments, the paclitaxel is administered in combination with Compound A on consecutive days.

In some embodiments, the paclitaxel is administered in combination with Compound A at least 6 times per week. In some embodiments, the paclitaxel is administered in combination with Compound A on consecutive days.

In some embodiments, the paclitaxel is administered in combination with Compound A daily per week.

In some embodiments, the paclitaxel is administered in combination with Compound A at least 1 time a week.

In some embodiments, the paclitaxel is administered at an amount of about 100 mg/m² to about 400 mg/m² in combination with Compound A at least 1 time a week.

In some embodiments, the paclitaxel is administered at an amount of about 100 mg/m² to about 350 mg/m² in combination with Compound A at least 1 time a week.

In some embodiments, the paclitaxel is administered at an amount of about 100 mg/m² to about 300 mg/m² in combination with Compound A at least 1 time a week.

In some embodiments, the paclitaxel is administered at an amount of about 150 mg/m² to about 250 mg/m² in combination with Compound A at least 1 time a week.

In some embodiments, the paclitaxel is administered at an amount of about 100 mg/m² to about 245 mg/m² in combination with Compound A at least 1 time a week.

In some embodiments, the paclitaxel is administered at an amount of about 100 mg/m² to about 240 mg/m² in combination with Compound A at least 1 time a week.

In some embodiments, the paclitaxel is administered at an amount of about 100 mg/m² to about 235 mg/m² in combination with Compound A at least 1 time a week.

In some embodiments, the paclitaxel is administered at an amount of about 100 mg/m² to about 230 mg/m² in combination with Compound A at least 1 time a week.

In some embodiments, the paclitaxel is administered at an amount of about 100 mg/m² to about 225 mg/m² in combination with Compound A at least 1 time a week.

In some embodiments, the paclitaxel is administered at an amount of about 100 mg/m² to about 220 mg/m² in combination with Compound A at least 1 time a week.

In some embodiments, the paclitaxel is administered at an amount of about 100 mg/m² to about 215 mg/m² in combination with Compound A at least 1 time a week.

In some embodiments, the paclitaxel is administered at an amount of about 100 mg/m² to about 210 mg/m² in combination with Compound A at least 1 time a week.

In some embodiments, the paclitaxel is administered at an amount of about 100 mg/m² to about 205 mg/m² in combination with Compound A at least 1 time a week.

In some embodiments, the paclitaxel is administered at an amount of about 100 mg/m² to about 200 mg/m² in combination with Compound A at least 1 time a week.

In some embodiments, the paclitaxel is administered at an amount of about 100 mg/m² to about 195 mg/m² in combination with Compound A at least 1 time a week.

In some embodiments, the paclitaxel is administered at an amount of about 100 mg/m² to about 190 mg/m² in combination with Compound A at least 1 time a week.

In some embodiments, the paclitaxel is administered at an amount of about 100 mg/m² to about 185 mg/m² in combination with Compound A at least 1 time a week.

In some embodiments, the paclitaxel is administered at an amount of about 100 mg/m² to about 180 mg/m² in combination with Compound A at least 1 time a week.

In some embodiments, the paclitaxel is administered at an amount of about 100 mg/m² to about 175 mg/m² in combination with Compound A at least 1 time a week.

In some embodiments, the paclitaxel is administered at an amount of about 100 mg/m² to about 170 mg/m² in combination with Compound A at least 1 time a week.

In some embodiments, the paclitaxel is administered at an amount of about 100 mg/m² to about 165 mg/m² in combination with Compound A at least 1 time a week.

In some embodiments, the paclitaxel is administered at an amount of about 100 mg/m² to about 160 mg/m² in combination with Compound A at least 1 time a week.

In some embodiments, the paclitaxel is administered at an amount of about 100 mg/m² to about 155 mg/m² in combination with Compound A at least 1 time a week.

In some embodiments, the paclitaxel is administered at an amount of about 100 mg/m² to about 150 mg/m² in combination with Compound A at least 1 time a week.

In some embodiments, the paclitaxel is administered at an amount of about 100 mg/m² to about 145 mg/m² in combination with Compound A at least 1 time a week.

In some embodiments, the paclitaxel is administered at an amount of about 100 mg/m² to about 140 mg/m² in combination with Compound A at least 1 time a week.

In some embodiments, the paclitaxel is administered at an amount of about 100 mg/m² to about 135 mg/m² in combination with Compound A at least 1 time a week.

In some embodiments, the paclitaxel is administered at an amount of about 100 mg/m² to about 130 mg/m² in combination with Compound A at least 1 time a week.

In some embodiments, the paclitaxel is administered at an amount of about 100 mg/m² to about 125 mg/m² in combination with Compound A at least 1 time a week.

In some embodiments, the paclitaxel is administered at an amount of about 100 mg/m² to about 120 mg/m² in combination with Compound A at least 1 time a week.

In some embodiments, the paclitaxel is administered at an amount of about 100 mg/m² to about 115 mg/m² in combination with Compound A at least 1 time a week.

In some embodiments, the paclitaxel is administered at an amount of about 100 mg/m² to about 110 mg/m² in combination with Compound A at least 1 time a week.

In some embodiments, the paclitaxel is administered at an amount of about 100 mg/m² to about 105 mg/m² in combination with Compound A at least 1 time a week.

In some embodiments, the paclitaxel is administered at an amount of about 150 mg/m² to about 350 mg/m² in combination with Compound A at least 1 time a week.

In some embodiments, the paclitaxel is administered at an amount of about 150 mg/m² to about 300 mg/m² in combination with Compound A at least 1 time a week.

In some embodiments, the paclitaxel is administered at an amount of about 150 mg/m² to about 250 mg/m² in combination with Compound A at least 1 time a week.

In some embodiments, the paclitaxel is administered at an amount of about 150 mg/m² to about 245 mg/m² in combination with Compound A at least 1 time a week.

In some embodiments, the paclitaxel is administered at an amount of about 150 mg/m² to about 240 mg/m² in combination with Compound A at least 1 time a week.

In some embodiments, the paclitaxel is administered at an amount of about 150 mg/m² to about 235 mg/m² in combination with Compound A at least 1 time a week.

In some embodiments, the paclitaxel is administered at an amount of about 150 mg/m² to about 230 mg/m² in combination with Compound A at least 1 time a week.

In some embodiments, the paclitaxel is administered at an amount of about 150 mg/m² to about 225 mg/m² in combination with Compound A at least 1 time a week.

In some embodiments, the paclitaxel is administered at an amount of about 150 mg/m² to about 220 mg/m² in combination with Compound A at least 1 time a week.

In some embodiments, the paclitaxel is administered at an amount of about 150 mg/m² to about 215 mg/m² in combination with Compound A at least 1 time a week.

In some embodiments, the paclitaxel is administered at an amount of about 150 mg/m² to about 210 mg/m² in combination with Compound A at least 1 time a week.

In some embodiments, the paclitaxel is administered at an amount of about 150 mg/m² to about 205 mg/m² in combination with Compound A at least 1 time a week.

In some embodiments, the paclitaxel is administered at an amount of about 150 mg/m² to about 200 mg/m² in combination with Compound A at least 1 time a week.

In some embodiments, the paclitaxel is administered at an amount of about 150 mg/m² to about 195 mg/m² in combination with Compound A at least 1 time a week.

In some embodiments, the paclitaxel is administered at an amount of about 150 mg/m² to about 190 mg/m² in combination with Compound A at least 1 time a week.

In some embodiments, the paclitaxel is administered at an amount of about 150 mg/m² to about 185 mg/m² in combination with Compound A at least 1 time a week.

In some embodiments, the paclitaxel is administered at an amount of about 150 mg/m² to about 180 mg/m² in combination with Compound A at least 1 time a week.

In some embodiments, the paclitaxel is administered at an amount of about 150 mg/m² to about 175 mg/m² in combination with Compound A at least 1 time a week.

In some embodiments, the paclitaxel is administered at an amount of about 150 mg/m² to about 170 mg/m² in combination with Compound A at least 1 time a week.

In some embodiments, the paclitaxel is administered at an amount of about 150 mg/m² to about 165 mg/m² in combination with Compound A at least 1 time a week.

In some embodiments, the paclitaxel is administered at an amount of about 150 mg/m² to about 160 mg/m² in combination with Compound A at least 1 time a week.

In some embodiments, the paclitaxel is administered at an amount of about 150 mg/m² to about 155 mg/m² in combination with Compound A at least 1 time a week.

In some embodiments, the paclitaxel is administered at an amount of about 200 mg/m² to about 300 mg/m² in combination with Compound A at least 1 time a week.

In some embodiments, the paclitaxel is administered at an amount of about 200 mg/m² to about 295 mg/m² in combination with Compound A at least 1 time a week.

In some embodiments, the paclitaxel is administered at an amount of about 200 mg/m² to about 290 mg/m² in combination with Compound A at least 1 time a week.

In some embodiments, the paclitaxel is administered at an amount of about 200 mg/m² to about 285 mg/m² in combination with Compound A at least 1 time a week.

In some embodiments, the paclitaxel is administered at an amount of about 200 mg/m² to about 280 mg/m² in combination with Compound A at least 1 time a week.

In some embodiments, the paclitaxel is administered at an amount of about 200 mg/m² to about 275 mg/m² in combination with Compound A at least 1 time a week.

In some embodiments, the paclitaxel is administered at an amount of about 200 mg/m² to about 270 mg/m² in combination with Compound A at least 1 time a week.

In some embodiments, the paclitaxel is administered at an amount of about 200 mg/m² to about 265 mg/m² in combination with Compound A at least 1 time a week.

In some embodiments, the paclitaxel is administered at an amount of about 200 mg/m² to about 260 mg/m² in combination with Compound A at least 1 time a week.

In some embodiments, the paclitaxel is administered at an amount of about 200 mg/m² to about 255 mg/m² in combination with Compound A at least 1 time a week.

In some embodiments, the paclitaxel is administered at an amount of about 200 mg/m² to about 250 mg/m² in combination with Compound A at least 1 time a week.

In some embodiments, the paclitaxel is administered at an amount of about 200 mg/m² to about 245 mg/m² in combination with Compound A at least 1 time a week.

In some embodiments, the paclitaxel is administered at an amount of about 200 mg/m² to about 240 mg/m² in combination with Compound A at least 1 time a week.

In some embodiments, the paclitaxel is administered at an amount of about 200 mg/m² to about 235 mg/m² in combination with Compound A at least 1 time a week.

In some embodiments, the paclitaxel is administered at an amount of about 200 mg/m² to about 230 mg/m² in combination with Compound A at least 1 time a week.

In some embodiments, the paclitaxel is administered at an amount of about 200 mg/m² to about 225 mg/m² in combination with Compound A at least 1 time a week.

In some embodiments, the paclitaxel is administered at an amount of about 200 mg/m² to about 220 mg/m² in combination with Compound A at least 1 time a week.

In some embodiments, the paclitaxel is administered at an amount of about 200 mg/m² to about 215 mg/m² in combination with Compound A at least 1 time a week.

In some embodiments, the paclitaxel is administered at an amount of about 200 mg/m² to about 210 mg/m² in combination with Compound A at least 1 time a week.

In some embodiments, the paclitaxel is administered at an amount of about 200 mg/m² to about 205 mg/m² in combination with Compound A at least 1 time a week.

In some embodiments, the paclitaxel is administered at an amount of about 250 mg/m² to about 350 mg/m² in combination with Compound A at least 1 time a week.

In some embodiments, the paclitaxel is administered at an amount of about 250 mg/m² to about 300 mg/m² in combination with Compound A at least 1 time a week.

In some embodiments, the paclitaxel is administered at an amount of about 120 mg/m² to about 235 mg/m² in combination with Compound A at least 1 time a week.

In some embodiments, the paclitaxel is administered at an amount of about 120 mg/m² to about 230 mg/m² in combination with Compound A at least 1 time a week.

In some embodiments, the paclitaxel is administered at an amount of about 120 mg/m² to about 225 mg/m² in combination with Compound A at least 1 time a week.

In some embodiments, the paclitaxel is administered at an amount of about 120 mg/m² to about 220 mg/m² in combination with Compound A at least 1 time a week.

In some embodiments, the paclitaxel is administered at an amount of about 120 mg/m² to about 215 mg/m² in combination with Compound A at least 1 time a week.

In some embodiments, the paclitaxel is administered at an amount of about 120 mg/m² to about 210 mg/m² in combination with Compound A at least 1 time a week.

In some embodiments, the paclitaxel is administered at an amount of about 120 mg/m² to about 205 mg/m² in combination with Compound A at least 1 time a week.

In some embodiments, the paclitaxel is administered at an amount of about 120 mg/m² to about 200 mg/m² in combination with Compound A at least 1 time a week.

In some embodiments, the paclitaxel is administered at an amount of about 120 mg/m² to about 195 mg/m² in combination with Compound A at least 1 time a week.

In some embodiments, the paclitaxel is administered at an amount of about 120 mg/m² to about 190 mg/m² in combination with Compound A at least 1 time a week.

In some embodiments, the paclitaxel is administered at an amount of about 120 mg/m² to about 185 mg/m² in combination with Compound A at least 1 time a week.

In some embodiments, the paclitaxel is administered at an amount of about 120 mg/m² to about 180 mg/m² in combination with Compound A at least 1 time a week.

In some embodiments, the paclitaxel is administered at an amount of about 120 mg/m² to about 175 mg/m² in combination with Compound A at least 1 time a week.

In some embodiments, the paclitaxel is administered at an amount of about 120 mg/m² to about 170 mg/m² in combination with Compound A at least 1 time a week.

In some embodiments, the paclitaxel is administered at an amount of about 120 mg/m² to about 165 mg/m² in combination with Compound A at least 1 time a week.

In some embodiments, the paclitaxel is administered at an amount of about 120 mg/m² to about 160 mg/m² in combination with Compound A at least 1 time a week.

In some embodiments, the paclitaxel is administered at an amount of about 120 mg/m² to about 155 mg/m² in combination with Compound A at least 1 time a week.

In some embodiments, the paclitaxel is administered at an amount of about 120 mg/m² to about 150 mg/m² in combination with Compound A at least 1 time a week.

In some embodiments, the paclitaxel is administered at an amount of about 120 mg/m² to about 145 mg/m² in combination with Compound A at least 1 time a week.

In some embodiments, the paclitaxel is administered at an amount of about 120 mg/m² to about 140 mg/m² in combination with Compound A at least 1 time a week.

In some embodiments, the paclitaxel is administered at an amount of about 120 mg/m² to about 135 mg/m² in combination with Compound A at least 1 time a week.

In some embodiments, the paclitaxel is administered at an amount of about 120 mg/m² to about 130 mg/m² in combination with Compound A at least 1 time a week.

In some embodiments, the paclitaxel is administered at an amount of about 120 mg/m² to about 125 mg/m² in combination with Compound A at least 1 time a week.

In some embodiments, the paclitaxel is administered at an amount of about 125 mg/m² to about 235 mg/m² in combination with Compound A at least 1 time a week.

In some embodiments, the paclitaxel is administered at an amount of about 125 mg/m² to about 230 mg/m² in combination with Compound A at least 1 time a week.

In some embodiments, the paclitaxel is administered at an amount of about 125 mg/m² to about 225 mg/m² in combination with Compound A at least 1 time a week.

In some embodiments, the paclitaxel is administered at an amount of about 125 mg/m² to about 220 mg/m² in combination with Compound A at least 1 time a week.

In some embodiments, the paclitaxel is administered at an amount of about 125 mg/m² to about 215 mg/m² in combination with Compound A at least 1 time a week.

In some embodiments, the paclitaxel is administered at an amount of about 125 mg/m² to about 210 mg/m² in combination with Compound A at least 1 time a week.

In some embodiments, the paclitaxel is administered at an amount of about 125 mg/m² to about 205 mg/m² in combination with Compound A at least 1 time a week.

In some embodiments, the paclitaxel is administered at an amount of about 125 mg/m² to about 200 mg/m² in combination with Compound A at least 1 time a week.

In some embodiments, the paclitaxel is administered at an amount of about 125 mg/m² to about 195 mg/m² in combination with Compound A at least 1 time a week.

In some embodiments, the paclitaxel is administered at an amount of about 125 mg/m² to about 190 mg/m² in combination with Compound A at least 1 time a week.

In some embodiments, the paclitaxel is administered at an amount of about 125 mg/m² to about 185 mg/m² in combination with Compound A at least 1 time a week.

In some embodiments, the paclitaxel is administered at an amount of about 125 mg/m² to about 180 mg/m² in combination with Compound A at least 1 time a week.

In some embodiments, the paclitaxel is administered at an amount of about 125 mg/m² to about 175 mg/m² in combination with Compound A at least 1 time a week.

In some embodiments, the paclitaxel is administered at an amount of about 125 mg/m² to about 170 mg/m² in combination with Compound A at least 1 time a week.

In some embodiments, the paclitaxel is administered at an amount of about 125 mg/m² to about 165 mg/m² in combination with Compound A at least 1 time a week.

In some embodiments, the paclitaxel is administered at an amount of about 125 mg/m² to about 160 mg/m² in combination with Compound A at least 1 time a week.

In some embodiments, the paclitaxel is administered at an amount of about 125 mg/m² to about 155 mg/m² in combination with Compound A at least 1 time a week.

In some embodiments, the paclitaxel is administered at an amount of about 125 mg/m² to about 150 mg/m² in combination with Compound A at least 1 time a week.

In some embodiments, the paclitaxel is administered at an amount of about 125 mg/m² to about 145 mg/m² in combination with Compound A at least 1 time a week.

In some embodiments, the paclitaxel is administered at an amount of about 125 mg/m² to about 140 mg/m² in combination with Compound A at least 1 time a week.

In some embodiments, the paclitaxel is administered at an amount of about 125 mg/m² to about 135 mg/m² in combination with Compound A at least 1 time a week.

In some embodiments, the paclitaxel is administered at an amount of about 125 mg/m² to about 130 mg/m² in combination with Compound A at least 1 time a week.

In some embodiments, the paclitaxel is administered at an amount of about 130 mg/m² to about 235 mg/m² in combination with Compound A at least 1 time a week.

In some embodiments, the paclitaxel is administered at an amount of about 130 mg/m² to about 230 mg/m² in combination with Compound A at least 1 time a week.

In some embodiments, the paclitaxel is administered at an amount of about 130 mg/m² to about 225 mg/m² in combination with Compound A at least 1 time a week.

In some embodiments, the paclitaxel is administered at an amount of about 130 mg/m² to about 220 mg/m² in combination with Compound A at least 1 time a week.

In some embodiments, the paclitaxel is administered at an amount of about 130 mg/m² to about 215 mg/m² in combination with Compound A at least 1 time a week.

In some embodiments, the paclitaxel is administered at an amount of about 130 mg/m² to about 210 mg/m² in combination with Compound A at least 1 time a week.

In some embodiments, the paclitaxel is administered at an amount of about 130 mg/m² to about 205 mg/m² in combination with Compound A at least 1 time a week.

In some embodiments, the paclitaxel is administered at an amount of about 130 mg/m² to about 200 mg/m² in combination with Compound A at least 1 time a week.

In some embodiments, the paclitaxel is administered at an amount of about 130 mg/m² to about 195 mg/m² in combination with Compound A at least 1 time a week.

In some embodiments, the paclitaxel is administered at an amount of about 130 mg/m² to about 190 mg/m² in combination with Compound A at least 1 time a week.

In some embodiments, the paclitaxel is administered at an amount of about 130 mg/m² to about 185 mg/m² in combination with Compound A at least 1 time a week.

In some embodiments, the paclitaxel is administered at an amount of about 130 mg/m² to about 180 mg/m² in combination with Compound A at least 1 time a week.

In some embodiments, the paclitaxel is administered at an amount of about 130 mg/m² to about 175 mg/m² in combination with Compound A at least 1 time a week.

In some embodiments, the paclitaxel is administered at an amount of about 130 mg/m² to about 170 mg/m² in combination with Compound A at least 1 time a week.

In some embodiments, the paclitaxel is administered at an amount of about 130 mg/m² to about 165 mg/m² in combination with Compound A at least 1 time a week.

In some embodiments, the paclitaxel is administered at an amount of about 130 mg/m² to about 160 mg/m² in combination with Compound A at least 1 time a week.

In some embodiments, the paclitaxel is administered at an amount of about 130 mg/m² to about 155 mg/m² in combination with Compound A at least 1 time a week.

In some embodiments, the paclitaxel is administered at an amount of about 130 mg/m² to about 150 mg/m² in combination with Compound A at least 1 time a week.

In some embodiments, the paclitaxel is administered at an amount of about 130 mg/m² to about 145 mg/m² in combination with Compound A at least 1 time a week.

In some embodiments, the paclitaxel is administered at an amount of about 130 mg/m² to about 140 mg/m² in combination with Compound A at least 1 time a week.

In some embodiments, the paclitaxel is administered at an amount of about 130 mg/m² to about 135 mg/m² in combination with Compound A at least 1 time a week.

In some embodiments, the paclitaxel is administered at an amount of about 100 mg/m² in combination with Compound A at least 1 time a week.

In some embodiments, the paclitaxel is administered at an amount of about 105 mg/m² in combination with Compound A at least 1 time a week.

In some embodiments, the paclitaxel is administered at an amount of about 110 mg/m² in combination with Compound A at least 1 time a week.

In some embodiments, the paclitaxel is administered at an amount of about 115 mg/m² in combination with Compound A at least 1 time a week.

In some embodiments, the paclitaxel is administered at an amount of about 120 mg/m² in combination with Compound A at least 1 time a week.

In some embodiments, the paclitaxel is administered at an amount of about 125 mg/m² in combination with Compound A at least 1 time a week.

In some embodiments, the paclitaxel is administered at an amount of about 130 mg/m² in combination with Compound A at least 1 time a week.

In some embodiments, the paclitaxel is administered at an amount of about 135 mg/m² in combination with Compound A at least 1 time a week.

In some embodiments, the paclitaxel is administered at an amount of about 140 mg/m² in combination with Compound A at least 1 time a week.

In some embodiments, the paclitaxel is administered at an amount of about 145 mg/m² in combination with Compound A at least 1 time a week.

In some embodiments, the paclitaxel is administered at an amount of about 150 mg/m² in combination with Compound A at least 1 time a week.

In some embodiments, the paclitaxel is administered at an amount of about 155 mg/m² in combination with Compound A at least 1 time a week.

In some embodiments, the paclitaxel is administered at an amount of about 160 mg/m² in combination with Compound A at least 1 time a week.

In some embodiments, the paclitaxel is administered at an amount of about 165 mg/m² in combination with Compound A at least 1 time a week.

In some embodiments, the paclitaxel is administered at an amount of about 170 mg/m² in combination with Compound A at least 1 time a week.

In some embodiments, the paclitaxel is administered at an amount of about 175 mg/m² in combination with Compound A at least 1 time a week.

In some embodiments, the paclitaxel is administered at an amount of about 180 mg/m² in combination with Compound A at least 1 time a week.

In some embodiments, the paclitaxel is administered at an amount of about 185 mg/m² in combination with Compound A at least 1 time a week.

In some embodiments, the paclitaxel is administered at an amount of about 190 mg/m² in combination with Compound A at least 1 time a week.

In some embodiments, the paclitaxel is administered at an amount of about 195 mg/m² in combination with Compound A at least 1 time a week.

In some embodiments, the paclitaxel is administered at an amount of about 200 mg/m² in combination with Compound A at least 1 time a week.

In some embodiments, the paclitaxel is administered at an amount of about 205 mg/m² in combination with Compound A at least 1 time a week.

In some embodiments, the paclitaxel is administered at an amount of about 210 mg/m² in combination with Compound A at least 1 time a week.

In some embodiments, the paclitaxel is administered at an amount of about 215 mg/m² in combination with Compound A at least 1 time a week.

In some embodiments, the paclitaxel is administered at an amount of about 220 mg/m² in combination with Compound A at least 1 time a week.

In some embodiments, the paclitaxel is administered at an amount of about 225 mg/m² in combination with Compound A at least 1 time a week.

In some embodiments, the paclitaxel is administered at an amount of about 230 mg/m² in combination with Compound A at least 1 time a week.

In some embodiments, the paclitaxel is administered at an amount of about 235 mg/m² in combination with Compound A at least 1 time a week.

In some embodiments, the paclitaxel is administered at an amount of about 240 mg/m² in combination with Compound A at least 1 time a week.

In some embodiments, the paclitaxel is administered at an amount of about 245 mg/m² in combination with Compound A at least 1 time a week.

In some embodiments, the paclitaxel is administered at an amount of about 250 mg/m² in combination with Compound A at least 1 time a week.

In some embodiments, the paclitaxel is administered at an amount of about 255 mg/m² in combination with Compound A at least 1 time a week.

In some embodiments, the paclitaxel is administered at an amount of about 260 mg/m² in combination with Compound A at least 1 time a week.

In some embodiments, the paclitaxel is administered at an amount of about 265 mg/m² in combination with Compound A at least 1 time a week.

In some embodiments, the paclitaxel is administered at an amount of about 270 mg/m² in combination with Compound A at least 1 time a week.

In some embodiments, the paclitaxel is administered at an amount of about 275 mg/m² in combination with Compound A at least 1 time a week.

In some embodiments, the paclitaxel is administered at an amount of about 280 mg/m² in combination with Compound A at least 1 time a week.

In some embodiments, the paclitaxel is administered at an amount of about 285 mg/m² in combination with Compound A at least 1 time a week.

In some embodiments, the paclitaxel is administered at an amount of about 290 mg/m² in combination with Compound A at least 1 time a week.

In some embodiments, the paclitaxel is administered at an amount of about 295 mg/m² in combination with Compound A at least 1 time a week.

In some embodiments, the paclitaxel is administered at an amount of about 300 mg/m² in combination with Compound A at least 1 time a week.

In some embodiments, the paclitaxel is administered at an amount of about 305 mg/m² in combination with Compound A at least 1 time a week.

In some embodiments, the paclitaxel is administered at an amount of about 310 mg/m² in combination with Compound A at least 1 time a week.

In some embodiments, the paclitaxel is administered at an amount of about 315 mg/m² in combination with Compound A at least 1 time a week.

In some embodiments, the paclitaxel is administered at an amount of about 320 mg/m² in combination with Compound A at least 1 time a week.

In some embodiments, the paclitaxel is administered at an amount of about 325 mg/m² in combination with Compound A at least 1 time a week.

In some embodiments, the paclitaxel is administered at an amount of about 330 mg/m² in combination with Compound A at least 1 time a week.

In some embodiments, the paclitaxel is administered at an amount of about 335 mg/m² in combination with Compound A at least 1 time a week.

In some embodiments, the paclitaxel is administered at an amount of about 340 mg/m² in combination with Compound A at least 1 time a week.

In some embodiments, the paclitaxel is administered at an amount of about 345 mg/m² in combination with Compound A at least 1 time a week.

In some embodiments, the paclitaxel is administered at an amount of about 350 mg/m² in combination with Compound A at least 1 time a week.

In some embodiments, the paclitaxel is administered at an amount of about 355 mg/m² in combination with Compound A at least 1 time a week.

In some embodiments, the paclitaxel is administered at an amount of about 360 mg/m² in combination with Compound A at least 1 time a week.

In some embodiments, the paclitaxel is administered at an amount of about 365 mg/m² in combination with Compound A at least 1 time a week.

In some embodiments, the paclitaxel is administered at an amount of about 370 mg/m² in combination with Compound A at least 1 time a week.

In some embodiments, the paclitaxel is administered at an amount of about 375 mg/m² in combination with Compound A at least 1 time a week.

In some embodiments, the paclitaxel is administered at an amount of about 380 mg/m² in combination with Compound A at least 1 time a week.

In some embodiments, the paclitaxel is administered at an amount of about 385 mg/m² in combination with Compound A at least 1 time a week.

In some embodiments, the paclitaxel is administered at an amount of about 390 mg/m² in combination with Compound A at least 1 time a week.

In some embodiments, the paclitaxel is administered at an amount of about 395 mg/m² in combination with Compound A at least 1 time a week.

In some embodiments, the paclitaxel is administered at an amount of about 400 mg/m² in combination with Compound A at least 1 time a week.

In some embodiments, Compound A is administered in combination with paclitaxel at least 1 time a week.

In some embodiments, Compound A is administered at an amount of about 1 mg to about 500 mg in combination with paclitaxel at least 1 time a week.

In some embodiments, Compound A is administered at about 1 mg to about 400 mg in combination with paclitaxel at least 1 time a week.

In some embodiments, Compound A is administered at about 1 mg to about 300 mg in combination with paclitaxel at least 1 time a week.

In some embodiments, Compound A is administered at about 5 mg to about 200 mg in combination with paclitaxel at least 1 time a week.

In some embodiments, Compound A is administered at about 10 mg to about 100 mg in combination with paclitaxel at least 1 time a week.

In some embodiments, Compound A is administered at about 10 mg to about 95 mg in combination with paclitaxel at least 1 time a week.

In some embodiments, Compound A is administered at about 10 mg to about 90 mg in combination with paclitaxel at least 1 time a week.

In some embodiments, Compound A is administered at about 10 mg to about 85 mg in combination with paclitaxel at least 1 time a week.

In some embodiments, Compound A is administered at about 10 mg to about 80 mg in combination with paclitaxel at least 1 time a week.

In some embodiments, Compound A is administered at about 10 mg to about 75 mg in combination with paclitaxel at least 1 time a week.

In some embodiments, Compound A is administered at about 10 mg to about 70 mg in combination with paclitaxel at least 1 time a week.

In some embodiments, Compound A is administered at about 10 mg to about 65 mg in combination with paclitaxel at least 1 time a week.

In some embodiments, Compound A is administered at about 10 mg to about 60 mg in combination with paclitaxel at least 1 time a week.

In some embodiments, Compound A is administered at about 10 mg to about 55 mg in combination with paclitaxel at least 1 time a week.

In some embodiments, Compound A is administered at about 10 mg to about 50 mg in combination with paclitaxel at least 1 time a week.

In some embodiments, Compound A is administered at about 10 mg to about 45 mg in combination with paclitaxel at least 1 time a week.

In some embodiments, Compound A is administered at about 10 mg to about 40 mg in combination with paclitaxel at least 1 time a week.

In some embodiments, Compound A is administered at about 10 mg to about 35 mg in combination with paclitaxel at least 1 time a week.

In some embodiments, Compound A is administered at about 10 mg to about 30 mg in combination with paclitaxel at least 1 time a week.

In some embodiments, Compound A is administered at about 10 mg to about 25 mg in combination with paclitaxel at least 1 time a week.

In some embodiments, Compound A is administered at about 10 mg to about 20 mg in combination with paclitaxel at least 1 time a week.

In some embodiments, Compound A is administered at about 10 mg to about 15 mg in combination with paclitaxel at least 1 time a week.

In some embodiments, Compound A is administered at about 15 mg to about 50 mg in combination with paclitaxel at least 1 time a week.

In some embodiments, Compound A is administered at about 15 mg to about 45 mg in combination with paclitaxel at least 1 time a week.

In some embodiments, Compound A is administered at about 15 mg to about 40 mg in combination with paclitaxel at least 1 time a week.

In some embodiments, Compound A is administered at about 15 mg to about 35 mg in combination with paclitaxel at least 1 time a week.

In some embodiments, Compound A is administered at about 15 mg to about 30 mg in combination with paclitaxel at least 1 time a week.

In some embodiments, Compound A is administered at about 15 mg to about 25 mg in combination with paclitaxel at least 1 time a week.

In some embodiments, Compound A is administered at about 15 mg to about 20 mg in combination with paclitaxel at least 1 time a week.

In some embodiments, Compound A is administered at about 5 mg in combination with paclitaxel at least 1 time a week.

In some embodiments, Compound A is administered at about 10 mg in combination with paclitaxel at least 1 time a week.

In some embodiments, Compound A is administered at about 15 mg in combination with paclitaxel at least 1 time a week.

In some embodiments, Compound A is administered at about 20 mg in combination with paclitaxel at least 1 time a week.

In some embodiments, Compound A is administered at about 25 mg in combination with paclitaxel at least 1 time a week.

In some embodiments, Compound A is administered at about 30 mg in combination with paclitaxel at least 1 time a week.

In some embodiments, Compound A is administered at about 35 mg in combination with paclitaxel at least 1 time a week.

In some embodiments, Compound A is administered at about 40 mg in combination with paclitaxel at least 1 time a week.

In some embodiments, Compound A is administered at about 45 mg in combination with paclitaxel at least 1 time a week.

In some embodiments, Compound A is administered at about 50 mg in combination with paclitaxel at least 1 time a week.

In some embodiments, the paclitaxel is administered in combination with Compound A at least 1 time a week.

In some embodiments, the paclitaxel is administered in combination with Compound A at least 2 times a week.

In some embodiments, the paclitaxel is administered at an amount of about 100 mg/m² to about 400 mg/m² in combination with Compound A at least 2 times a week.

In some embodiments, the paclitaxel is administered at an amount of about 100 mg/m² to about 350 mg/m² in combination with Compound A at least 2 times a week.

In some embodiments, the paclitaxel is administered at an amount of about 100 mg/m² to about 300 mg/m² in combination with Compound A at least 2 times a week.

In some embodiments, the paclitaxel is administered at an amount of about 150 mg/m² to about 250 mg/m² in combination with Compound A at least 2 times a week.

In some embodiments, the paclitaxel is administered at an amount of about 100 mg/m² to about 245 mg/m² in combination with Compound A at least 2 times a week.

In some embodiments, the paclitaxel is administered at an amount of about 100 mg/m² to about 240 mg/m² in combination with Compound A at least 2 times a week.

In some embodiments, the paclitaxel is administered at an amount of about 100 mg/m² to about 235 mg/m² in combination with Compound A at least 2 times a week.

In some embodiments, the paclitaxel is administered at an amount of about 100 mg/m² to about 230 mg/m² in combination with Compound A at least 2 times a week.

In some embodiments, the paclitaxel is administered at an amount of about 100 mg/m² to about 225 mg/m² in combination with Compound A at least 2 times a week.

In some embodiments, the paclitaxel is administered at an amount of about 100 mg/m² to about 220 mg/m² in combination with Compound A at least 2 times a week.

In some embodiments, the paclitaxel is administered at an amount of about 100 mg/m² to about 215 mg/m² in combination with Compound A at least 2 times a week.

In some embodiments, the paclitaxel is administered at an amount of about 100 mg/m² to about 210 mg/m² in combination with Compound A at least 2 times a week.

In some embodiments, the paclitaxel is administered at an amount of about 100 mg/m² to about 205 mg/m² in combination with Compound A at least 2 times a week.

In some embodiments, the paclitaxel is administered at an amount of about 100 mg/m² to about 200 mg/m² in combination with Compound A at least 2 times a week.

In some embodiments, the paclitaxel is administered at an amount of about 100 mg/m² to about 195 mg/m² in combination with Compound A at least 2 times a week.

In some embodiments, the paclitaxel is administered at an amount of about 100 mg/m² to about 190 mg/m² in combination with Compound A at least 2 times a week.

In some embodiments, the paclitaxel is administered at an amount of about 100 mg/m² to about 185 mg/m² in combination with Compound A at least 2 times a week.

In some embodiments, the paclitaxel is administered at an amount of about 100 mg/m² to about 180 mg/m² in combination with Compound A at least 2 times a week.

In some embodiments, the paclitaxel is administered at an amount of about 100 mg/m² to about 175 mg/m² in combination with Compound A at least 2 times a week.

In some embodiments, the paclitaxel is administered at an amount of about 100 mg/m² to about 170 mg/m² in combination with Compound A at least 2 times a week.

In some embodiments, the paclitaxel is administered at an amount of about 100 mg/m² to about 165 mg/m² in combination with Compound A at least 2 times a week.

In some embodiments, the paclitaxel is administered at an amount of about 100 mg/m² to about 160 mg/m² in combination with Compound A at least 2 times a week.

In some embodiments, the paclitaxel is administered at an amount of about 100 mg/m² to about 155 mg/m² in combination with Compound A at least 2 times a week.

In some embodiments, the paclitaxel is administered at an amount of about 100 mg/m² to about 150 mg/m² in combination with Compound A at least 2 times a week.

In some embodiments, the paclitaxel is administered at an amount of about 100 mg/m² to about 145 mg/m² in combination with Compound A at least 2 times a week.

In some embodiments, the paclitaxel is administered at an amount of about 100 mg/m² to about 140 mg/m² in combination with Compound A at least 2 times a week.

In some embodiments, the paclitaxel is administered at an amount of about 100 mg/m² to about 135 mg/m² in combination with Compound A at least 2 times a week.

In some embodiments, the paclitaxel is administered at an amount of about 100 mg/m² to about 130 mg/m² in combination with Compound A at least 2 times a week.

In some embodiments, the paclitaxel is administered at an amount of about 100 mg/m² to about 125 mg/m² in combination with Compound A at least 2 times a week.

In some embodiments, the paclitaxel is administered at an amount of about 100 mg/m² to about 120 mg/m² in combination with Compound A at least 2 times a week.

In some embodiments, the paclitaxel is administered at an amount of about 100 mg/m² to about 115 mg/m² in combination with Compound A at least 2 times a week.

In some embodiments, the paclitaxel is administered at an amount of about 100 mg/m² to about 110 mg/m² in combination with Compound A at least 2 times a week.

In some embodiments, the paclitaxel is administered at an amount of about 100 mg/m² to about 105 mg/m² in combination with Compound A at least 2 times a week.

In some embodiments, the paclitaxel is administered at an amount of about 150 mg/m² to about 350 mg/m² in combination with Compound A at least 2 times a week.

In some embodiments, the paclitaxel is administered at an amount of about 150 mg/m² to about 300 mg/m² in combination with Compound A at least 2 times a week.

In some embodiments, the paclitaxel is administered at an amount of about 150 mg/m² to about 250 mg/m² in combination with Compound A at least 2 times a week.

In some embodiments, the paclitaxel is administered at an amount of about 150 mg/m² to about 245 mg/m² in combination with Compound A at least 2 times a week.

In some embodiments, the paclitaxel is administered at an amount of about 150 mg/m² to about 240 mg/m² in combination with Compound A at least 2 times a week.

In some embodiments, the paclitaxel is administered at an amount of about 150 mg/m² to about 235 mg/m² in combination with Compound A at least 2 times a week.

In some embodiments, the paclitaxel is administered at an amount of about 150 mg/m² to about 230 mg/m² in combination with Compound A at least 2 times a week.

In some embodiments, the paclitaxel is administered at an amount of about 150 mg/m² to about 225 mg/m² in combination with Compound A at least 2 times a week.

In some embodiments, the paclitaxel is administered at an amount of about 150 mg/m² to about 220 mg/m² in combination with Compound A at least 2 times a week.

In some embodiments, the paclitaxel is administered at an amount of about 150 mg/m² to about 215 mg/m² in combination with Compound A at least 2 times a week.

In some embodiments, the paclitaxel is administered at an amount of about 150 mg/m² to about 210 mg/m² in combination with Compound A at least 2 times a week.

In some embodiments, the paclitaxel is administered at an amount of about 150 mg/m² to about 205 mg/m² in combination with Compound A at least 2 times a week.

In some embodiments, the paclitaxel is administered at an amount of about 150 mg/m² to about 200 mg/m² in combination with Compound A at least 2 times a week.

In some embodiments, the paclitaxel is administered at an amount of about 150 mg/m² to about 195 mg/m² in combination with Compound A at least 2 times a week.

In some embodiments, the paclitaxel is administered at an amount of about 150 mg/m² to about 190 mg/m² in combination with Compound A at least 2 times a week.

In some embodiments, the paclitaxel is administered at an amount of about 150 mg/m² to about 185 mg/m² in combination with Compound A at least 2 times a week.

In some embodiments, the paclitaxel is administered at an amount of about 150 mg/m² to about 180 mg/m² in combination with Compound A at least 2 times a week.

In some embodiments, the paclitaxel is administered at an amount of about 150 mg/m² to about 175 mg/m² in combination with Compound A at least 2 times a week.

In some embodiments, the paclitaxel is administered at an amount of about 150 mg/m² to about 170 mg/m² in combination with Compound A at least 2 times a week.

In some embodiments, the paclitaxel is administered at an amount of about 150 mg/m² to about 165 mg/m² in combination with Compound A at least 2 times a week.

In some embodiments, the paclitaxel is administered at an amount of about 150 mg/m² to about 160 mg/m² in combination with Compound A at least 2 times a week.

In some embodiments, the paclitaxel is administered at an amount of about 150 mg/m² to about 155 mg/m² in combination with Compound A at least 2 times a week.

In some embodiments, the paclitaxel is administered at an amount of about 200 mg/m² to about 300 mg/m² in combination with Compound A at least 2 times a week.

In some embodiments, the paclitaxel is administered at an amount of about 200 mg/m² to about 295 mg/m² in combination with Compound A at least 2 times a week.

In some embodiments, the paclitaxel is administered at an amount of about 200 mg/m² to about 290 mg/m² in combination with Compound A at least 2 times a week.

In some embodiments, the paclitaxel is administered at an amount of about 200 mg/m² to about 285 mg/m² in combination with Compound A at least 2 times a week.

In some embodiments, the paclitaxel is administered at an amount of about 200 mg/m² to about 280 mg/m² in combination with Compound A at least 2 times a week.

In some embodiments, the paclitaxel is administered at an amount of about 200 mg/m² to about 275 mg/m² in combination with Compound A at least 2 times a week.

In some embodiments, the paclitaxel is administered at an amount of about 200 mg/m² to about 270 mg/m² in combination with Compound A at least 2 times a week.

In some embodiments, the paclitaxel is administered at an amount of about 200 mg/m² to about 265 mg/m² in combination with Compound A at least 2 times a week.

In some embodiments, the paclitaxel is administered at an amount of about 200 mg/m² to about 260 mg/m² in combination with Compound A at least 2 times a week.

In some embodiments, the paclitaxel is administered at an amount of about 200 mg/m² to about 255 mg/m² in combination with Compound A at least 2 times a week.

In some embodiments, the paclitaxel is administered at an amount of about 200 mg/m² to about 250 mg/m² in combination with Compound A at least 2 times a week.

In some embodiments, the paclitaxel is administered at an amount of about 200 mg/m² to about 245 mg/m² in combination with Compound A at least 2 times a week.

In some embodiments, the paclitaxel is administered at an amount of about 200 mg/m² to about 240 mg/m² in combination with Compound A at least 2 times a week.

In some embodiments, the paclitaxel is administered at an amount of about 200 mg/m² to about 235 mg/m² in combination with Compound A at least 2 times a week.

In some embodiments, the paclitaxel is administered at an amount of about 200 mg/m² to about 230 mg/m² in combination with Compound A at least 2 times a week.

In some embodiments, the paclitaxel is administered at an amount of about 200 mg/m² to about 225 mg/m² in combination with Compound A at least 2 times a week.

In some embodiments, the paclitaxel is administered at an amount of about 200 mg/m² to about 220 mg/m² in combination with Compound A at least 2 times a week.

In some embodiments, the paclitaxel is administered at an amount of about 200 mg/m² to about 215 mg/m² in combination with Compound A at least 2 times a week.

In some embodiments, the paclitaxel is administered at an amount of about 200 mg/m² to about 210 mg/m² in combination with Compound A at least 2 times a week.

In some embodiments, the paclitaxel is administered at an amount of about 200 mg/m² to about 205 mg/m² in combination with Compound A at least 2 times a week.

In some embodiments, the paclitaxel is administered at an amount of about 250 mg/m² to about 350 mg/m² in combination with Compound A at least 2 times a week.

In some embodiments, the paclitaxel is administered at an amount of about 250 mg/m² to about 300 mg/m² in combination with Compound A at least 2 times a week.

In some embodiments, the paclitaxel is administered at an amount of about 120 mg/m² to about 235 mg/m² in combination with Compound A at least 2 times a week.

In some embodiments, the paclitaxel is administered at an amount of about 120 mg/m² to about 230 mg/m² in combination with Compound A at least 2 times a week.

In some embodiments, the paclitaxel is administered at an amount of about 120 mg/m² to about 225 mg/m² in combination with Compound A at least 2 times a week.

In some embodiments, the paclitaxel is administered at an amount of about 120 mg/m² to about 220 mg/m² in combination with Compound A at least 2 times a week.

In some embodiments, the paclitaxel is administered at an amount of about 120 mg/m² to about 215 mg/m² in combination with Compound A at least 2 times a week.

In some embodiments, the paclitaxel is administered at an amount of about 120 mg/m² to about 210 mg/m² in combination with Compound A at least 2 times a week.

In some embodiments, the paclitaxel is administered at an amount of about 120 mg/m² to about 205 mg/m² in combination with Compound A at least 2 times a week.

In some embodiments, the paclitaxel is administered at an amount of about 120 mg/m² to about 200 mg/m² in combination with Compound A at least 2 times a week.

In some embodiments, the paclitaxel is administered at an amount of about 120 mg/m² to about 195 mg/m² in combination with Compound A at least 2 times a week.

In some embodiments, the paclitaxel is administered at an amount of about 120 mg/m² to about 190 mg/m² in combination with Compound A at least 2 times a week.

In some embodiments, the paclitaxel is administered at an amount of about 120 mg/m² to about 185 mg/m² in combination with Compound A at least 2 times a week.

In some embodiments, the paclitaxel is administered at an amount of about 120 mg/m² to about 180 mg/m² in combination with Compound A at least 2 times a week.

In some embodiments, the paclitaxel is administered at an amount of about 120 mg/m² to about 175 mg/m² in combination with Compound A at least 2 times a week.

In some embodiments, the paclitaxel is administered at an amount of about 120 mg/m² to about 170 mg/m² in combination with Compound A at least 2 times a week.

In some embodiments, the paclitaxel is administered at an amount of about 120 mg/m² to about 165 mg/m² in combination with Compound A at least 2 times a week.

In some embodiments, the paclitaxel is administered at an amount of about 120 mg/m² to about 160 mg/m² in combination with Compound A at least 2 times a week.

In some embodiments, the paclitaxel is administered at an amount of about 120 mg/m² to about 155 mg/m² in combination with Compound A at least 2 times a week.

In some embodiments, the paclitaxel is administered at an amount of about 120 mg/m² to about 150 mg/m² in combination with Compound A at least 2 times a week.

In some embodiments, the paclitaxel is administered at an amount of about 120 mg/m² to about 145 mg/m² in combination with Compound A at least 2 times a week.

In some embodiments, the paclitaxel is administered at an amount of about 120 mg/m² to about 140 mg/m² in combination with Compound A at least 2 times a week.

In some embodiments, the paclitaxel is administered at an amount of about 120 mg/m² to about 135 mg/m² in combination with Compound A at least 2 times a week.

In some embodiments, the paclitaxel is administered at an amount of about 120 mg/m² to about 130 mg/m² in combination with Compound A at least 2 times a week.

In some embodiments, the paclitaxel is administered at an amount of about 120 mg/m² to about 125 mg/m² in combination with Compound A at least 2 times a week.

In some embodiments, the paclitaxel is administered at an amount of about 125 mg/m² to about 235 mg/m² in combination with Compound A at least 2 times a week.

In some embodiments, the paclitaxel is administered at an amount of about 125 mg/m² to about 230 mg/m² in combination with Compound A at least 2 times a week.

In some embodiments, the paclitaxel is administered at an amount of about 125 mg/m² to about 225 mg/m² in combination with Compound A at least 2 times a week.

In some embodiments, the paclitaxel is administered at an amount of about 125 mg/m² to about 220 mg/m² in combination with Compound A at least 2 times a week.

In some embodiments, the paclitaxel is administered at an amount of about 125 mg/m² to about 215 mg/m² in combination with Compound A at least 2 times a week.

In some embodiments, the paclitaxel is administered at an amount of about 125 mg/m² to about 210 mg/m² in combination with Compound A at least 2 times a week.

In some embodiments, the paclitaxel is administered at an amount of about 125 mg/m² to about 205 mg/m² in combination with Compound A at least 2 times a week.

In some embodiments, the paclitaxel is administered at an amount of about 125 mg/m² to about 200 mg/m² in combination with Compound A at least 2 times a week.

In some embodiments, the paclitaxel is administered at an amount of about 125 mg/m² to about 195 mg/m² in combination with Compound A at least 2 times a week.

In some embodiments, the paclitaxel is administered at an amount of about 125 mg/m² to about 190 mg/m² in combination with Compound A at least 2 times a week.

In some embodiments, the paclitaxel is administered at an amount of about 125 mg/m² to about 185 mg/m² in combination with Compound A at least 2 times a week.

In some embodiments, the paclitaxel is administered at an amount of about 125 mg/m² to about 180 mg/m² in combination with Compound A at least 2 times a week.

In some embodiments, the paclitaxel is administered at an amount of about 125 mg/m² to about 175 mg/m² in combination with Compound A at least 2 times a week.

In some embodiments, the paclitaxel is administered at an amount of about 125 mg/m² to about 170 mg/m² in combination with Compound A at least 2 times a week.

In some embodiments, the paclitaxel is administered at an amount of about 125 mg/m² to about 165 mg/m² in combination with Compound A at least 2 times a week.

In some embodiments, the paclitaxel is administered at an amount of about 125 mg/m² to about 160 mg/m² in combination with Compound A at least 2 times a week.

In some embodiments, the paclitaxel is administered at an amount of about 125 mg/m² to about 155 mg/m² in combination with Compound A at least 2 times a week.

In some embodiments, the paclitaxel is administered at an amount of about 125 mg/m² to about 150 mg/m² in combination with Compound A at least 2 times a week.

In some embodiments, the paclitaxel is administered at an amount of about 125 mg/m² to about 145 mg/m² in combination with Compound A at least 2 times a week.

In some embodiments, the paclitaxel is administered at an amount of about 125 mg/m² to about 140 mg/m² in combination with Compound A at least 2 times a week.

In some embodiments, the paclitaxel is administered at an amount of about 125 mg/m² to about 135 mg/m² in combination with Compound A at least 2 times a week.

In some embodiments, the paclitaxel is administered at an amount of about 125 mg/m² to about 130 mg/m² in combination with Compound A at least 2 times a week.

In some embodiments, the paclitaxel is administered at an amount of about 130 mg/m² to about 235 mg/m² in combination with Compound A at least 2 times a week.

In some embodiments, the paclitaxel is administered at an amount of about 130 mg/m² to about 230 mg/m² in combination with Compound A at least 2 times a week.

In some embodiments, the paclitaxel is administered at an amount of about 130 mg/m² to about 225 mg/m² in combination with Compound A at least 2 times a week.

In some embodiments, the paclitaxel is administered at an amount of about 130 mg/m² to about 220 mg/m² in combination with Compound A at least 2 times a week.

In some embodiments, the paclitaxel is administered at an amount of about 130 mg/m² to about 215 mg/m² in combination with Compound A at least 2 times a week.

In some embodiments, the paclitaxel is administered at an amount of about 130 mg/m² to about 210 mg/m² in combination with Compound A at least 2 times a week.

In some embodiments, the paclitaxel is administered at an amount of about 130 mg/m² to about 205 mg/m² in combination with Compound A at least 2 times a week.

In some embodiments, the paclitaxel is administered at an amount of about 130 mg/m² to about 200 mg/m² in combination with Compound A at least 2 times a week.

In some embodiments, the paclitaxel is administered at an amount of about 130 mg/m² to about 195 mg/m² in combination with Compound A at least 2 times a week.

In some embodiments, the paclitaxel is administered at an amount of about 130 mg/m² to about 190 mg/m² in combination with Compound A at least 2 times a week.

In some embodiments, the paclitaxel is administered at an amount of about 130 mg/m² to about 185 mg/m² in combination with Compound A at least 2 times a week.

In some embodiments, the paclitaxel is administered at an amount of about 130 mg/m² to about 180 mg/m² in combination with Compound A at least 2 times a week.

In some embodiments, the paclitaxel is administered at an amount of about 130 mg/m² to about 175 mg/m² in combination with Compound A at least 2 times a week.

In some embodiments, the paclitaxel is administered at an amount of about 130 mg/m² to about 170 mg/m² in combination with Compound A at least 2 times a week.

In some embodiments, the paclitaxel is administered at an amount of about 130 mg/m² to about 165 mg/m² in combination with Compound A at least 2 times a week.

In some embodiments, the paclitaxel is administered at an amount of about 130 mg/m² to about 160 mg/m² in combination with Compound A at least 2 times a week.

In some embodiments, the paclitaxel is administered at an amount of about 130 mg/m² to about 155 mg/m² in combination with Compound A at least 2 times a week.

In some embodiments, the paclitaxel is administered at an amount of about 130 mg/m² to about 150 mg/m² in combination with Compound A at least 2 times a week.

In some embodiments, the paclitaxel is administered at an amount of about 130 mg/m² to about 145 mg/m² in combination with Compound A at least 2 times a week.

In some embodiments, the paclitaxel is administered at an amount of about 130 mg/m² to about 140 mg/m² in combination with Compound A at least 2 times a week.

In some embodiments, the paclitaxel is administered at an amount of about 130 mg/m² to about 135 mg/m² in combination with Compound A at least 2 times a week.

In some embodiments, the paclitaxel is administered at an amount of about 100 mg/m² in combination with Compound A at least 2 times a week.

In some embodiments, the paclitaxel is administered at an amount of about 105 mg/m² in combination with Compound A at least 2 times a week.

In some embodiments, the paclitaxel is administered at an amount of about 110 mg/m² in combination with Compound A at least 2 times a week.

In some embodiments, the paclitaxel is administered at an amount of about 115 mg/m² in combination with Compound A at least 2 times a week.

In some embodiments, the paclitaxel is administered at an amount of about 120 mg/m² in combination with Compound A at least 2 times a week.

In some embodiments, the paclitaxel is administered at an amount of about 125 mg/m² in combination with Compound A at least 2 times a week.

In some embodiments, the paclitaxel is administered at an amount of about 130 mg/m² in combination with Compound A at least 2 times a week.

In some embodiments, the paclitaxel is administered at an amount of about 135 mg/m² in combination with Compound A at least 2 times a week.

In some embodiments, the paclitaxel is administered at an amount of about 140 mg/m² in combination with Compound A at least 2 times a week.

In some embodiments, the paclitaxel is administered at an amount of about 145 mg/m² in combination with Compound A at least 2 times a week.

In some embodiments, the paclitaxel is administered at an amount of about 150 mg/m² in combination with Compound A at least 2 times a week.

In some embodiments, the paclitaxel is administered at an amount of about 155 mg/m² in combination with Compound A at least 2 times a week.

In some embodiments, the paclitaxel is administered at an amount of about 160 mg/m² in combination with Compound A at least 2 times a week.

In some embodiments, the paclitaxel is administered at an amount of about 165 mg/m² in combination with Compound A at least 2 times a week.

In some embodiments, the paclitaxel is administered at an amount of about 170 mg/m² in combination with Compound A at least 2 times a week.

In some embodiments, the paclitaxel is administered at an amount of about 175 mg/m² in combination with Compound A at least 2 times a week.

In some embodiments, the paclitaxel is administered at an amount of about 180 mg/m² in combination with Compound A at least 2 times a week.

In some embodiments, the paclitaxel is administered at an amount of about 185 mg/m² in combination with Compound A at least 2 times a week.

In some embodiments, the paclitaxel is administered at an amount of about 190 mg/m² in combination with Compound A at least 2 times a week.

In some embodiments, the paclitaxel is administered at an amount of about 195 mg/m² in combination with Compound A at least 2 times a week.

In some embodiments, the paclitaxel is administered at an amount of about 200 mg/m² in combination with Compound A at least 2 times a week.

In some embodiments, the paclitaxel is administered at an amount of about 205 mg/m² in combination with Compound A at least 2 times a week.

In some embodiments, the paclitaxel is administered at an amount of about 210 mg/m² in combination with Compound A at least 2 times a week.

In some embodiments, the paclitaxel is administered at an amount of about 215 mg/m² in combination with Compound A at least 2 times a week.

In some embodiments, the paclitaxel is administered at an amount of about 220 mg/m² in combination with Compound A at least 2 times a week.

In some embodiments, the paclitaxel is administered at an amount of about 225 mg/m² in combination with Compound A at least 2 times a week.

In some embodiments, the paclitaxel is administered at an amount of about 230 mg/m² in combination with Compound A at least 2 times a week.

In some embodiments, the paclitaxel is administered at an amount of about 235 mg/m² in combination with Compound A at least 2 times a week.

In some embodiments, the paclitaxel is administered at an amount of about 240 mg/m² in combination with Compound A at least 2 times a week.

In some embodiments, the paclitaxel is administered at an amount of about 245 mg/m² in combination with Compound A at least 2 times a week.

In some embodiments, the paclitaxel is administered at an amount of about 250 mg/m² in combination with Compound A at least 2 times a week.

In some embodiments, the paclitaxel is administered at an amount of about 255 mg/m² in combination with Compound A at least 2 times a week.

In some embodiments, the paclitaxel is administered at an amount of about 260 mg/m² in combination with Compound A at least 2 times a week.

In some embodiments, the paclitaxel is administered at an amount of about 265 mg/m² in combination with Compound A at least 2 times a week.

In some embodiments, the paclitaxel is administered at an amount of about 270 mg/m² in combination with Compound A at least 2 times a week.

In some embodiments, the paclitaxel is administered at an amount of about 275 mg/m² in combination with Compound A at least 2 times a week.

In some embodiments, the paclitaxel is administered at an amount of about 280 mg/m² in combination with Compound A at least 2 times a week.

In some embodiments, the paclitaxel is administered at an amount of about 285 mg/m² in combination with Compound A at least 2 times a week.

In some embodiments, the paclitaxel is administered at an amount of about 290 mg/m² in combination with Compound A at least 2 times a week.

In some embodiments, the paclitaxel is administered at an amount of about 295 mg/m² in combination with Compound A at least 2 times a week.

In some embodiments, the paclitaxel is administered at an amount of about 300 mg/m² in combination with Compound A at least 2 times a week.

In some embodiments, the paclitaxel is administered at an amount of about 305 mg/m² in combination with Compound A at least 2 times a week.

In some embodiments, the paclitaxel is administered at an amount of about 310 mg/m² in combination with Compound A at least 2 times a week.

In some embodiments, the paclitaxel is administered at an amount of about 315 mg/m² in combination with Compound A at least 2 times a week.

In some embodiments, the paclitaxel is administered at an amount of about 320 mg/m² in combination with Compound A at least 2 times a week.

In some embodiments, the paclitaxel is administered at an amount of about 325 mg/m² in combination with Compound A at least 2 times a week.

In some embodiments, the paclitaxel is administered at an amount of about 330 mg/m² in combination with Compound A at least 2 times a week.

In some embodiments, the paclitaxel is administered at an amount of about 335 mg/m² in combination with Compound A at least 2 times a week.

In some embodiments, the paclitaxel is administered at an amount of about 340 mg/m² in combination with Compound A at least 2 times a week.

In some embodiments, the paclitaxel is administered at an amount of about 345 mg/m² in combination with Compound A at least 2 times a week.

In some embodiments, the paclitaxel is administered at an amount of about 350 mg/m² in combination with Compound A at least 2 times a week.

In some embodiments, the paclitaxel is administered at an amount of about 355 mg/m² in combination with Compound A at least 2 times a week.

In some embodiments, the paclitaxel is administered at an amount of about 360 mg/m² in combination with Compound A at least 2 times a week.

In some embodiments, the paclitaxel is administered at an amount of about 365 mg/m² in combination with Compound A at least 2 times a week.

In some embodiments, the paclitaxel is administered at an amount of about 370 mg/m² in combination with Compound A at least 2 times a week.

In some embodiments, the paclitaxel is administered at an amount of about 375 mg/m² in combination with Compound A at least 2 times a week.

In some embodiments, the paclitaxel is administered at an amount of about 380 mg/m² in combination with Compound A at least 2 times a week.

In some embodiments, the paclitaxel is administered at an amount of about 385 mg/m² in combination with Compound A at least 2 times a week.

In some embodiments, the paclitaxel is administered at an amount of about 390 mg/m² in combination with Compound A at least 2 times a week.

In some embodiments, the paclitaxel is administered at an amount of about 395 mg/m² in combination with Compound A at least 2 times a week.

In some embodiments, the paclitaxel is administered at an amount of about 400 mg/m² in combination with Compound A at least 2 times a week.

In some embodiments, Compound A is administered in combination with paclitaxel at least 2 times a week.

In some embodiments, Compound A is administered at an amount of about 1 mg to about 500 mg in combination with paclitaxel at least 2 times a week.

In some embodiments, Compound A is administered at about 1 mg to about 400 mg in combination with paclitaxel at least 2 times a week.

In some embodiments, Compound A is administered at about 1 mg to about 300 mg in combination with paclitaxel at least 2 times a week.

In some embodiments, Compound A is administered at about 5 mg to about 200 mg in combination with paclitaxel at least 2 times a week.

In some embodiments, Compound A is administered at about 10 mg to about 100 mg in combination with paclitaxel at least 2 times a week.

In some embodiments, Compound A is administered at about 10 mg to about 95 mg in combination with paclitaxel at least 2 times a week.

In some embodiments, Compound A is administered at about 10 mg to about 90 mg in combination with paclitaxel at least 2 times a week.

In some embodiments, Compound A is administered at about 10 mg to about 85 mg in combination with paclitaxel at least 2 times a week.

In some embodiments, Compound A is administered at about 10 mg to about 80 mg in combination with paclitaxel at least 2 times a week.

In some embodiments, Compound A is administered at about 10 mg to about 75 mg in combination with paclitaxel at least 2 times a week.

In some embodiments, Compound A is administered at about 10 mg to about 70 mg in combination with paclitaxel at least 2 times a week.

In some embodiments, Compound A is administered at about 10 mg to about 65 mg in combination with paclitaxel at least 2 times a week.

In some embodiments, Compound A is administered at about 10 mg to about 60 mg in combination with paclitaxel at least 2 times a week.

In some embodiments, Compound A is administered at about 10 mg to about 55 mg in combination with paclitaxel at least 2 times a week.

In some embodiments, Compound A is administered at about 10 mg to about 50 mg in combination with paclitaxel at least 2 times a week.

In some embodiments, Compound A is administered at about 10 mg to about 45 mg in combination with paclitaxel at least 2 times a week.

In some embodiments, Compound A is administered at about 10 mg to about 40 mg in combination with paclitaxel at least 2 times a week.

In some embodiments, Compound A is administered at about 10 mg to about 35 mg in combination with paclitaxel at least 2 times a week.

In some embodiments, Compound A is administered at about 10 mg to about 30 mg in combination with paclitaxel at least 2 times a week.

In some embodiments, Compound A is administered at about 10 mg to about 25 mg in combination with paclitaxel at least 2 times a week.

In some embodiments, Compound A is administered at about 10 mg to about 20 mg in combination with paclitaxel at least 2 times a week.

In some embodiments, Compound A is administered at about 10 mg to about 15 mg in combination with paclitaxel at least 2 times a week.

In some embodiments, Compound A is administered at about 15 mg to about 50 mg in combination with paclitaxel at least 2 times a week.

In some embodiments, Compound A is administered at about 15 mg to about 45 mg in combination with paclitaxel at least 2 times a week.

In some embodiments, Compound A is administered at about 15 mg to about 40 mg in combination with paclitaxel at least 2 times a week.

In some embodiments, Compound A is administered at about 15 mg to about 35 mg in combination with paclitaxel at least 2 times a week.

In some embodiments, Compound A is administered at about 15 mg to about 30 mg in combination with paclitaxel at least 2 times a week.

In some embodiments, Compound A is administered at about 15 mg to about 25 mg in combination with paclitaxel at least 2 times a week.

In some embodiments, Compound A is administered at about 15 mg to about 20 mg in combination with paclitaxel at least 2 times a week.

In some embodiments, Compound A is administered at about 5 mg in combination with paclitaxel at least 2 times a week.

In some embodiments, Compound A is administered at about 10 mg in combination with paclitaxel at least 2 times a week.

In some embodiments, Compound A is administered at about 15 mg in combination with paclitaxel at least 2 times a week.

In some embodiments, Compound A is administered at about 20 mg in combination with paclitaxel at least 2 times a week.

In some embodiments, Compound A is administered at about 25 mg in combination with paclitaxel at least 2 times a week.

In some embodiments, Compound A is administered at about 30 mg in combination with paclitaxel at least 2 times a week.

In some embodiments, Compound A is administered at about 35 mg in combination with paclitaxel at least 2 times a week.

In some embodiments, Compound A is administered at about 40 mg in combination with paclitaxel at least 2 times a week.

In some embodiments, Compound A is administered at about 45 mg in combination with paclitaxel at least 2 times a week.

In some embodiments, Compound A is administered at about 50 mg in combination with paclitaxel at least 2 times a week.

In some embodiments, the paclitaxel is administered in combination with Compound A at least 2 times a week, wherein the at least 2 days are consecutive.

In some embodiments, the paclitaxel is administered in combination with Compound A at least 3 times a week.

In some embodiments, the paclitaxel is administered at an amount of about 100 mg/m² to about 400 mg/m² in combination with Compound A at least 3 times a week.

In some embodiments, the paclitaxel is administered at an amount of about 100 mg/m² to about 350 mg/m² in combination with Compound A at least 3 times a week.

In some embodiments, the paclitaxel is administered at an amount of about 100 mg/m² to about 300 mg/m² in combination with Compound A at least 3 times a week.

In some embodiments, the paclitaxel is administered at an amount of about 150 mg/m² to about 250 mg/m² in combination with Compound A at least 3 times a week.

In some embodiments, the paclitaxel is administered at an amount of about 100 mg/m² to about 245 mg/m² in combination with Compound A at least 3 times a week.

In some embodiments, the paclitaxel is administered at an amount of about 100 mg/m² to about 240 mg/m² in combination with Compound A at least 3 times a week.

In some embodiments, the paclitaxel is administered at an amount of about 100 mg/m² to about 235 mg/m² in combination with Compound A at least 3 times a week.

In some embodiments, the paclitaxel is administered at an amount of about 100 mg/m² to about 230 mg/m² in combination with Compound A at least 3 times a week.

In some embodiments, the paclitaxel is administered at an amount of about 100 mg/m² to about 225 mg/m² in combination with Compound A at least 3 times a week.

In some embodiments, the paclitaxel is administered at an amount of about 100 mg/m² to about 220 mg/m² in combination with Compound A at least 3 times a week.

In some embodiments, the paclitaxel is administered at an amount of about 100 mg/m² to about 215 mg/m² in combination with Compound A at least 3 times a week.

In some embodiments, the paclitaxel is administered at an amount of about 100 mg/m² to about 210 mg/m² in combination with Compound A at least 3 times a week.

In some embodiments, the paclitaxel is administered at an amount of about 100 mg/m² to about 205 mg/m² in combination with Compound A at least 3 times a week.

In some embodiments, the paclitaxel is administered at an amount of about 100 mg/m² to about 200 mg/m² in combination with Compound A at least 3 times a week.

In some embodiments, the paclitaxel is administered at an amount of about 100 mg/m² to about 195 mg/m² in combination with Compound A at least 3 times a week.

In some embodiments, the paclitaxel is administered at an amount of about 100 mg/m² to about 190 mg/m² in combination with Compound A at least 3 times a week.

In some embodiments, the paclitaxel is administered at an amount of about 100 mg/m² to about 185 mg/m² in combination with Compound A at least 3 times a week.

In some embodiments, the paclitaxel is administered at an amount of about 100 mg/m² to about 180 mg/m² in combination with Compound A at least 3 times a week.

In some embodiments, the paclitaxel is administered at an amount of about 100 mg/m² to about 175 mg/m² in combination with Compound A at least 3 times a week.

In some embodiments, the paclitaxel is administered at an amount of about 100 mg/m² to about 170 mg/m² in combination with Compound A at least 3 times a week.

In some embodiments, the paclitaxel is administered at an amount of about 100 mg/m² to about 165 mg/m² in combination with Compound A at least 3 times a week.

In some embodiments, the paclitaxel is administered at an amount of about 100 mg/m² to about 160 mg/m² in combination with Compound A at least 3 times a week.

In some embodiments, the paclitaxel is administered at an amount of about 100 mg/m² to about 155 mg/m² in combination with Compound A at least 3 times a week.

In some embodiments, the paclitaxel is administered at an amount of about 100 mg/m² to about 150 mg/m² in combination with Compound A at least 3 times a week.

In some embodiments, the paclitaxel is administered at an amount of about 100 mg/m² to about 145 mg/m² in combination with Compound A at least 3 times a week.

In some embodiments, the paclitaxel is administered at an amount of about 100 mg/m² to about 140 mg/m² in combination with Compound A at least 3 times a week.

In some embodiments, the paclitaxel is administered at an amount of about 100 mg/m² to about 135 mg/m² in combination with Compound A at least 3 times a week.

In some embodiments, the paclitaxel is administered at an amount of about 100 mg/m² to about 130 mg/m² in combination with Compound A at least 3 times a week.

In some embodiments, the paclitaxel is administered at an amount of about 100 mg/m² to about 125 mg/m² in combination with Compound A at least 3 times a week.

In some embodiments, the paclitaxel is administered at an amount of about 100 mg/m² to about 120 mg/m² in combination with Compound A at least 3 times a week.

In some embodiments, the paclitaxel is administered at an amount of about 100 mg/m² to about 115 mg/m² in combination with Compound A at least 3 times a week.

In some embodiments, the paclitaxel is administered at an amount of about 100 mg/m² to about 110 mg/m² in combination with Compound A at least 3 times a week.

In some embodiments, the paclitaxel is administered at an amount of about 100 mg/m² to about 105 mg/m² in combination with Compound A at least 3 times a week.

In some embodiments, the paclitaxel is administered at an amount of about 150 mg/m² to about 350 mg/m² in combination with Compound A at least 3 times a week.

In some embodiments, the paclitaxel is administered at an amount of about 150 mg/m² to about 300 mg/m² in combination with Compound A at least 3 times a week.

In some embodiments, the paclitaxel is administered at an amount of about 150 mg/m² to about 250 mg/m² in combination with Compound A at least 3 times a week.

In some embodiments, the paclitaxel is administered at an amount of about 150 mg/m² to about 245 mg/m² in combination with Compound A at least 3 times a week.

In some embodiments, the paclitaxel is administered at an amount of about 150 mg/m² to about 240 mg/m² in combination with Compound A at least 3 times a week.

In some embodiments, the paclitaxel is administered at an amount of about 150 mg/m² to about 235 mg/m² in combination with Compound A at least 3 times a week.

In some embodiments, the paclitaxel is administered at an amount of about 150 mg/m² to about 230 mg/m² in combination with Compound A at least 3 times a week.

In some embodiments, the paclitaxel is administered at an amount of about 150 mg/m² to about 225 mg/m² in combination with Compound A at least 3 times a week.

In some embodiments, the paclitaxel is administered at an amount of about 150 mg/m² to about 220 mg/m² in combination with Compound A at least 3 times a week.

In some embodiments, the paclitaxel is administered at an amount of about 150 mg/m² to about 215 mg/m² in combination with Compound A at least 3 times a week.

In some embodiments, the paclitaxel is administered at an amount of about 150 mg/m² to about 210 mg/m² in combination with Compound A at least 3 times a week.

In some embodiments, the paclitaxel is administered at an amount of about 150 mg/m² to about 205 mg/m² in combination with Compound A at least 3 times a week.

In some embodiments, the paclitaxel is administered at an amount of about 150 mg/m² to about 200 mg/m² in combination with Compound A at least 3 times a week.

In some embodiments, the paclitaxel is administered at an amount of about 150 mg/m² to about 195 mg/m² in combination with Compound A at least 3 times a week.

In some embodiments, the paclitaxel is administered at an amount of about 150 mg/m² to about 190 mg/m² in combination with Compound A at least 3 times a week.

In some embodiments, the paclitaxel is administered at an amount of about 150 mg/m² to about 185 mg/m² in combination with Compound A at least 3 times a week.

In some embodiments, the paclitaxel is administered at an amount of about 150 mg/m² to about 180 mg/m² in combination with Compound A at least 3 times a week.

In some embodiments, the paclitaxel is administered at an amount of about 150 mg/m² to about 175 mg/m² in combination with Compound A at least 3 times a week.

In some embodiments, the paclitaxel is administered at an amount of about 150 mg/m² to about 170 mg/m² in combination with Compound A at least 3 times a week.

In some embodiments, the paclitaxel is administered at an amount of about 150 mg/m² to about 165 mg/m² in combination with Compound A at least 3 times a week.

In some embodiments, the paclitaxel is administered at an amount of about 150 mg/m² to about 160 mg/m² in combination with Compound A at least 3 times a week.

In some embodiments, the paclitaxel is administered at an amount of about 150 mg/m² to about 155 mg/m² in combination with Compound A at least 3 times a week.

In some embodiments, the paclitaxel is administered at an amount of about 200 mg/m² to about 300 mg/m² in combination with Compound A at least 3 times a week.

In some embodiments, the paclitaxel is administered at an amount of about 200 mg/m² to about 295 mg/m² in combination with Compound A at least 3 times a week.

In some embodiments, the paclitaxel is administered at an amount of about 200 mg/m² to about 290 mg/m² in combination with Compound A at least 3 times a week.

In some embodiments, the paclitaxel is administered at an amount of about 200 mg/m² to about 285 mg/m² in combination with Compound A at least 3 times a week.

In some embodiments, the paclitaxel is administered at an amount of about 200 mg/m² to about 280 mg/m² in combination with Compound A at least 3 times a week.

In some embodiments, the paclitaxel is administered at an amount of about 200 mg/m² to about 275 mg/m² in combination with Compound A at least 3 times a week.

In some embodiments, the paclitaxel is administered at an amount of about 200 mg/m² to about 270 mg/m² in combination with Compound A at least 3 times a week.

In some embodiments, the paclitaxel is administered at an amount of about 200 mg/m² to about 265 mg/m² in combination with Compound A at least 3 times a week.

In some embodiments, the paclitaxel is administered at an amount of about 200 mg/m² to about 260 mg/m² in combination with Compound A at least 3 times a week.

In some embodiments, the paclitaxel is administered at an amount of about 200 mg/m² to about 255 mg/m² in combination with Compound A at least 3 times a week.

In some embodiments, the paclitaxel is administered at an amount of about 200 mg/m² to about 250 mg/m² in combination with Compound A at least 3 times a week.

In some embodiments, the paclitaxel is administered at an amount of about 200 mg/m² to about 245 mg/m² in combination with Compound A at least 3 times a week.

In some embodiments, the paclitaxel is administered at an amount of about 200 mg/m² to about 240 mg/m² in combination with Compound A at least 3 times a week.

In some embodiments, the paclitaxel is administered at an amount of about 200 mg/m² to about 235 mg/m² in combination with Compound A at least 3 times a week.

In some embodiments, the paclitaxel is administered at an amount of about 200 mg/m² to about 230 mg/m² in combination with Compound A at least 3 times a week.

In some embodiments, the paclitaxel is administered at an amount of about 200 mg/m² to about 225 mg/m² in combination with Compound A at least 3 times a week.

In some embodiments, the paclitaxel is administered at an amount of about 200 mg/m² to about 220 mg/m² in combination with Compound A at least 3 times a week.

In some embodiments, the paclitaxel is administered at an amount of about 200 mg/m² to about 215 mg/m² in combination with Compound A at least 3 times a week.

In some embodiments, the paclitaxel is administered at an amount of about 200 mg/m² to about 210 mg/m² in combination with Compound A at least 3 times a week.

In some embodiments, the paclitaxel is administered at an amount of about 200 mg/m² to about 205 mg/m² in combination with Compound A at least 3 times a week.

In some embodiments, the paclitaxel is administered at an amount of about 250 mg/m² to about 350 mg/m² in combination with Compound A at least 3 times a week.

In some embodiments, the paclitaxel is administered at an amount of about 250 mg/m² to about 300 mg/m² in combination with Compound A at least 3 times a week.

In some embodiments, the paclitaxel is administered at an amount of about 120 mg/m² to about 235 mg/m² in combination with Compound A at least 3 times a week.

In some embodiments, the paclitaxel is administered at an amount of about 120 mg/m² to about 230 mg/m² in combination with Compound A at least 3 times a week.

In some embodiments, the paclitaxel is administered at an amount of about 120 mg/m² to about 225 mg/m² in combination with Compound A at least 3 times a week.

In some embodiments, the paclitaxel is administered at an amount of about 120 mg/m² to about 220 mg/m² in combination with Compound A at least 3 times a week.

In some embodiments, the paclitaxel is administered at an amount of about 120 mg/m² to about 215 mg/m² in combination with Compound A at least 3 times a week.

In some embodiments, the paclitaxel is administered at an amount of about 120 mg/m² to about 210 mg/m² in combination with Compound A at least 3 times a week.

In some embodiments, the paclitaxel is administered at an amount of about 120 mg/m² to about 205 mg/m² in combination with Compound A at least 3 times a week.

In some embodiments, the paclitaxel is administered at an amount of about 120 mg/m² to about 200 mg/m² in combination with Compound A at least 3 times a week.

In some embodiments, the paclitaxel is administered at an amount of about 120 mg/m² to about 195 mg/m² in combination with Compound A at least 3 times a week.

In some embodiments, the paclitaxel is administered at an amount of about 120 mg/m² to about 190 mg/m² in combination with Compound A at least 3 times a week.

In some embodiments, the paclitaxel is administered at an amount of about 120 mg/m² to about 185 mg/m² in combination with Compound A at least 3 times a week.

In some embodiments, the paclitaxel is administered at an amount of about 120 mg/m² to about 180 mg/m² in combination with Compound A at least 3 times a week.

In some embodiments, the paclitaxel is administered at an amount of about 120 mg/m² to about 175 mg/m² in combination with Compound A at least 3 times a week.

In some embodiments, the paclitaxel is administered at an amount of about 120 mg/m² to about 170 mg/m² in combination with Compound A at least 3 times a week.

In some embodiments, the paclitaxel is administered at an amount of about 120 mg/m² to about 165 mg/m² in combination with Compound A at least 3 times a week.

In some embodiments, the paclitaxel is administered at an amount of about 120 mg/m² to about 160 mg/m² in combination with Compound A at least 3 times a week.

In some embodiments, the paclitaxel is administered at an amount of about 120 mg/m² to about 155 mg/m² in combination with Compound A at least 3 times a week.

In some embodiments, the paclitaxel is administered at an amount of about 120 mg/m² to about 150 mg/m² in combination with Compound A at least 3 times a week.

In some embodiments, the paclitaxel is administered at an amount of about 120 mg/m² to about 145 mg/m² in combination with Compound A at least 3 times a week.

In some embodiments, the paclitaxel is administered at an amount of about 120 mg/m² to about 140 mg/m² in combination with Compound A at least 3 times a week.

In some embodiments, the paclitaxel is administered at an amount of about 120 mg/m² to about 135 mg/m² in combination with Compound A at least 3 times a week.

In some embodiments, the paclitaxel is administered at an amount of about 120 mg/m² to about 130 mg/m² in combination with Compound A at least 3 times a week.

In some embodiments, the paclitaxel is administered at an amount of about 120 mg/m² to about 125 mg/m² in combination with Compound A at least 3 times a week.

In some embodiments, the paclitaxel is administered at an amount of about 125 mg/m² to about 235 mg/m² in combination with Compound A at least 3 times a week.

In some embodiments, the paclitaxel is administered at an amount of about 125 mg/m² to about 230 mg/m² in combination with Compound A at least 3 times a week.

In some embodiments, the paclitaxel is administered at an amount of about 125 mg/m² to about 225 mg/m² in combination with Compound A at least 3 times a week.

In some embodiments, the paclitaxel is administered at an amount of about 125 mg/m² to about 220 mg/m² in combination with Compound A at least 3 times a week.

In some embodiments, the paclitaxel is administered at an amount of about 125 mg/m² to about 215 mg/m² in combination with Compound A at least 3 times a week.

In some embodiments, the paclitaxel is administered at an amount of about 125 mg/m² to about 210 mg/m² in combination with Compound A at least 3 times a week.

In some embodiments, the paclitaxel is administered at an amount of about 125 mg/m² to about 205 mg/m² in combination with Compound A at least 3 times a week.

In some embodiments, the paclitaxel is administered at an amount of about 125 mg/m² to about 200 mg/m² in combination with Compound A at least 3 times a week.

In some embodiments, the paclitaxel is administered at an amount of about 125 mg/m² to about 195 mg/m² in combination with Compound A at least 3 times a week.

In some embodiments, the paclitaxel is administered at an amount of about 125 mg/m² to about 190 mg/m² in combination with Compound A at least 3 times a week.

In some embodiments, the paclitaxel is administered at an amount of about 125 mg/m² to about 185 mg/m² in combination with Compound A at least 3 times a week.

In some embodiments, the paclitaxel is administered at an amount of about 125 mg/m² to about 180 mg/m² in combination with Compound A at least 3 times a week.

In some embodiments, the paclitaxel is administered at an amount of about 125 mg/m² to about 175 mg/m² in combination with Compound A at least 3 times a week.

In some embodiments, the paclitaxel is administered at an amount of about 125 mg/m² to about 170 mg/m² in combination with Compound A at least 3 times a week.

In some embodiments, the paclitaxel is administered at an amount of about 125 mg/m² to about 165 mg/m² in combination with Compound A at least 3 times a week.

In some embodiments, the paclitaxel is administered at an amount of about 125 mg/m² to about 160 mg/m² in combination with Compound A at least 3 times a week.

In some embodiments, the paclitaxel is administered at an amount of about 125 mg/m² to about 155 mg/m² in combination with Compound A at least 3 times a week.

In some embodiments, the paclitaxel is administered at an amount of about 125 mg/m² to about 150 mg/m² in combination with Compound A at least 3 times a week.

In some embodiments, the paclitaxel is administered at an amount of about 125 mg/m² to about 145 mg/m² in combination with Compound A at least 3 times a week.

In some embodiments, the paclitaxel is administered at an amount of about 125 mg/m² to about 140 mg/m² in combination with Compound A at least 3 times a week.

In some embodiments, the paclitaxel is administered at an amount of about 125 mg/m² to about 135 mg/m² in combination with Compound A at least 3 times a week.

In some embodiments, the paclitaxel is administered at an amount of about 125 mg/m² to about 130 mg/m² in combination with Compound A at least 3 times a week.

In some embodiments, the paclitaxel is administered at an amount of about 130 mg/m² to about 235 mg/m² in combination with Compound A at least 3 times a week.

In some embodiments, the paclitaxel is administered at an amount of about 130 mg/m² to about 230 mg/m² in combination with Compound A at least 3 times a week.

In some embodiments, the paclitaxel is administered at an amount of about 130 mg/m² to about 225 mg/m² in combination with Compound A at least 3 times a week.

In some embodiments, the paclitaxel is administered at an amount of about 130 mg/m² to about 220 mg/m² in combination with Compound A at least 3 times a week.

In some embodiments, the paclitaxel is administered at an amount of about 130 mg/m² to about 215 mg/m² in combination with Compound A at least 3 times a week.

In some embodiments, the paclitaxel is administered at an amount of about 130 mg/m² to about 210 mg/m² in combination with Compound A at least 3 times a week.

In some embodiments, the paclitaxel is administered at an amount of about 130 mg/m² to about 205 mg/m² in combination with Compound A at least 3 times a week.

In some embodiments, the paclitaxel is administered at an amount of about 130 mg/m² to about 200 mg/m² in combination with Compound A at least 3 times a week.

In some embodiments, the paclitaxel is administered at an amount of about 130 mg/m² to about 195 mg/m² in combination with Compound A at least 3 times a week.

In some embodiments, the paclitaxel is administered at an amount of about 130 mg/m² to about 190 mg/m² in combination with Compound A at least 3 times a week.

In some embodiments, the paclitaxel is administered at an amount of about 130 mg/m² to about 185 mg/m² in combination with Compound A at least 3 times a week.

In some embodiments, the paclitaxel is administered at an amount of about 130 mg/m² to about 180 mg/m² in combination with Compound A at least 3 times a week.

In some embodiments, the paclitaxel is administered at an amount of about 130 mg/m² to about 175 mg/m² in combination with Compound A at least 3 times a week.

In some embodiments, the paclitaxel is administered at an amount of about 130 mg/m² to about 170 mg/m² in combination with Compound A at least 3 times a week.

In some embodiments, the paclitaxel is administered at an amount of about 130 mg/m² to about 165 mg/m² in combination with Compound A at least 3 times a week.

In some embodiments, the paclitaxel is administered at an amount of about 130 mg/m² to about 160 mg/m² in combination with Compound A at least 3 times a week.

In some embodiments, the paclitaxel is administered at an amount of about 130 mg/m² to about 155 mg/m² in combination with Compound A at least 3 times a week.

In some embodiments, the paclitaxel is administered at an amount of about 130 mg/m² to about 150 mg/m² in combination with Compound A at least 3 times a week.

In some embodiments, the paclitaxel is administered at an amount of about 130 mg/m² to about 145 mg/m² in combination with Compound A at least 3 times a week.

In some embodiments, the paclitaxel is administered at an amount of about 130 mg/m² to about 140 mg/m² in combination with Compound A at least 3 times a week.

In some embodiments, the paclitaxel is administered at an amount of about 130 mg/m² to about 135 mg/m² in combination with Compound A at least 3 times a week.

In some embodiments, the paclitaxel is administered at an amount of about 100 mg/m² in combination with Compound A at least 3 times a week.

In some embodiments, the paclitaxel is administered at an amount of about 105 mg/m² in combination with Compound A at least 3 times a week.

In some embodiments, the paclitaxel is administered at an amount of about 110 mg/m² in combination with Compound A at least 3 times a week.

In some embodiments, the paclitaxel is administered at an amount of about 115 mg/m² in combination with Compound A at least 3 times a week.

In some embodiments, the paclitaxel is administered at an amount of about 120 mg/m² in combination with Compound A at least 3 times a week.

In some embodiments, the paclitaxel is administered at an amount of about 125 mg/m² in combination with Compound A at least 3 times a week.

In some embodiments, the paclitaxel is administered at an amount of about 130 mg/m² in combination with Compound A at least 3 times a week.

In some embodiments, the paclitaxel is administered at an amount of about 135 mg/m² in combination with Compound A at least 3 times a week.

In some embodiments, the paclitaxel is administered at an amount of about 140 mg/m² in combination with Compound A at least 3 times a week.

In some embodiments, the paclitaxel is administered at an amount of about 145 mg/m² in combination with Compound A at least 3 times a week.

In some embodiments, the paclitaxel is administered at an amount of about 150 mg/m² in combination with Compound A at least 3 times a week.

In some embodiments, the paclitaxel is administered at an amount of about 155 mg/m² in combination with Compound A at least 3 times a week.

In some embodiments, the paclitaxel is administered at an amount of about 160 mg/m² in combination with Compound A at least 3 times a week.

In some embodiments, the paclitaxel is administered at an amount of about 165 mg/m² in combination with Compound A at least 3 times a week.

In some embodiments, the paclitaxel is administered at an amount of about 170 mg/m² in combination with Compound A at least 3 times a week.

In some embodiments, the paclitaxel is administered at an amount of about 175 mg/m² in combination with Compound A at least 3 times a week.

In some embodiments, the paclitaxel is administered at an amount of about 180 mg/m² in combination with Compound A at least 3 times a week.

In some embodiments, the paclitaxel is administered at an amount of about 185 mg/m² in combination with Compound A at least 3 times a week.

In some embodiments, the paclitaxel is administered at an amount of about 190 mg/m² in combination with Compound A at least 3 times a week.

In some embodiments, the paclitaxel is administered at an amount of about 195 mg/m² in combination with Compound A at least 3 times a week.

In some embodiments, the paclitaxel is administered at an amount of about 200 mg/m² in combination with Compound A at least 3 times a week.

In some embodiments, the paclitaxel is administered at an amount of about 205 mg/m² in combination with Compound A at least 3 times a week.

In some embodiments, the paclitaxel is administered at an amount of about 210 mg/m² in combination with Compound A at least 3 times a week.

In some embodiments, the paclitaxel is administered at an amount of about 215 mg/m² in combination with Compound A at least 3 times a week.

In some embodiments, the paclitaxel is administered at an amount of about 220 mg/m² in combination with Compound A at least 3 times a week.

In some embodiments, the paclitaxel is administered at an amount of about 225 mg/m² in combination with Compound A at least 3 times a week.

In some embodiments, the paclitaxel is administered at an amount of about 230 mg/m² in combination with Compound A at least 3 times a week.

In some embodiments, the paclitaxel is administered at an amount of about 235 mg/m² in combination with Compound A at least 3 times a week.

In some embodiments, the paclitaxel is administered at an amount of about 240 mg/m² in combination with Compound A at least 3 times a week.

In some embodiments, the paclitaxel is administered at an amount of about 245 mg/m² in combination with Compound A at least 3 times a week.

In some embodiments, the paclitaxel is administered at an amount of about 250 mg/m² in combination with Compound A at least 3 times a week.

In some embodiments, the paclitaxel is administered at an amount of about 255 mg/m² in combination with Compound A at least 3 times a week.

In some embodiments, the paclitaxel is administered at an amount of about 260 mg/m² in combination with Compound A at least 3 times a week.

In some embodiments, the paclitaxel is administered at an amount of about 265 mg/m² in combination with Compound A at least 3 times a week.

In some embodiments, the paclitaxel is administered at an amount of about 270 mg/m² in combination with Compound A at least 3 times a week.

In some embodiments, the paclitaxel is administered at an amount of about 275 mg/m² in combination with Compound A at least 3 times a week.

In some embodiments, the paclitaxel is administered at an amount of about 280 mg/m² in combination with Compound A at least 3 times a week.

In some embodiments, the paclitaxel is administered at an amount of about 285 mg/m² in combination with Compound A at least 3 times a week.

In some embodiments, the paclitaxel is administered at an amount of about 290 mg/m² in combination with Compound A at least 3 times a week.

In some embodiments, the paclitaxel is administered at an amount of about 295 mg/m² in combination with Compound A at least 3 times a week.

In some embodiments, the paclitaxel is administered at an amount of about 300 mg/m² in combination with Compound A at least 3 times a week.

In some embodiments, the paclitaxel is administered at an amount of about 305 mg/m² in combination with Compound A at least 3 times a week.

In some embodiments, the paclitaxel is administered at an amount of about 310 mg/m² in combination with Compound A at least 3 times a week.

In some embodiments, the paclitaxel is administered at an amount of about 315 mg/m² in combination with Compound A at least 3 times a week.

In some embodiments, the paclitaxel is administered at an amount of about 320 mg/m² in combination with Compound A at least 3 times a week.

In some embodiments, the paclitaxel is administered at an amount of about 325 mg/m² in combination with Compound A at least 3 times a week.

In some embodiments, the paclitaxel is administered at an amount of about 330 mg/m² in combination with Compound A at least 3 times a week.

In some embodiments, the paclitaxel is administered at an amount of about 335 mg/m² in combination with Compound A at least 3 times a week.

In some embodiments, the paclitaxel is administered at an amount of about 340 mg/m² in combination with Compound A at least 3 times a week.

In some embodiments, the paclitaxel is administered at an amount of about 345 mg/m² in combination with Compound A at least 3 times a week.

In some embodiments, the paclitaxel is administered at an amount of about 350 mg/m² in combination with Compound A at least 3 times a week.

In some embodiments, the paclitaxel is administered at an amount of about 355 mg/m² in combination with Compound A at least 3 times a week.

In some embodiments, the paclitaxel is administered at an amount of about 360 mg/m² in combination with Compound A at least 3 times a week.

In some embodiments, the paclitaxel is administered at an amount of about 365 mg/m² in combination with Compound A at least 3 times a week.

In some embodiments, the paclitaxel is administered at an amount of about 370 mg/m² in combination with Compound A at least 3 times a week.

In some embodiments, the paclitaxel is administered at an amount of about 375 mg/m² in combination with Compound A at least 3 times a week.

In some embodiments, the paclitaxel is administered at an amount of about 380 mg/m² in combination with Compound A at least 3 times a week.

In some embodiments, the paclitaxel is administered at an amount of about 385 mg/m² in combination with Compound A at least 3 times a week.

In some embodiments, the paclitaxel is administered at an amount of about 390 mg/m² in combination with Compound A at least 3 times a week.

In some embodiments, the paclitaxel is administered at an amount of about 395 mg/m² in combination with Compound A at least 3 times a week.

In some embodiments, the paclitaxel is administered at an amount of about 400 mg/m² in combination with Compound A at least 3 times a week.

In some embodiments, Compound A is administered in combination with paclitaxel at least 3 times a week.

In some embodiments, Compound A is administered at an amount of about 1 mg to about 500 mg in combination with paclitaxel at least 3 times a week.

In some embodiments, Compound A is administered at about 1 mg to about 400 mg in combination with paclitaxel at least 3 times a week.

In some embodiments, Compound A is administered at about 1 mg to about 300 mg in combination with paclitaxel at least 3 times a week.

In some embodiments, Compound A is administered at about 5 mg to about 200 mg in combination with paclitaxel at least 3 times a week.

In some embodiments, Compound A is administered at about 10 mg to about 100 mg in combination with paclitaxel at least 3 times a week.

In some embodiments, Compound A is administered at about 10 mg to about 95 mg in combination with paclitaxel at least 3 times a week.

In some embodiments, Compound A is administered at about 10 mg to about 90 mg in combination with paclitaxel at least 3 times a week.

In some embodiments, Compound A is administered at about 10 mg to about 85 mg in combination with paclitaxel at least 3 times a week.

In some embodiments, Compound A is administered at about 10 mg to about 80 mg in combination with paclitaxel at least 3 times a week.

In some embodiments, Compound A is administered at about 10 mg to about 75 mg in combination with paclitaxel at least 3 times a week.

In some embodiments, Compound A is administered at about 10 mg to about 70 mg in combination with paclitaxel at least 3 times a week.

In some embodiments, Compound A is administered at about 10 mg to about 65 mg in combination with paclitaxel at least 3 times a week.

In some embodiments, Compound A is administered at about 10 mg to about 60 mg in combination with paclitaxel at least 3 times a week.

In some embodiments, Compound A is administered at about 10 mg to about 55 mg in combination with paclitaxel at least 3 times a week.

In some embodiments, Compound A is administered at about 10 mg to about 50 mg in combination with paclitaxel at least 3 times a week.

In some embodiments, Compound A is administered at about 10 mg to about 45 mg in combination with paclitaxel at least 3 times a week.

In some embodiments, Compound A is administered at about 10 mg to about 40 mg in combination with paclitaxel at least 3 times a week.

In some embodiments, Compound A is administered at about 10 mg to about 35 mg in combination with paclitaxel at least 3 times a week.

In some embodiments, Compound A is administered at about 10 mg to about 30 mg in combination with paclitaxel at least 3 times a week.

In some embodiments, Compound A is administered at about 10 mg to about 25 mg in combination with paclitaxel at least 3 times a week.

In some embodiments, Compound A is administered at about 10 mg to about 20 mg in combination with paclitaxel at least 3 times a week.

In some embodiments, Compound A is administered at about 10 mg to about 15 mg in combination with paclitaxel at least 3 times a week.

In some embodiments, Compound A is administered at about 15 mg to about 50 mg in combination with paclitaxel at least 3 times a week.

In some embodiments, Compound A is administered at about 15 mg to about 45 mg in combination with paclitaxel at least 3 times a week.

In some embodiments, Compound A is administered at about 15 mg to about 40 mg in combination with paclitaxel at least 3 times a week.

In some embodiments, Compound A is administered at about 15 mg to about 35 mg in combination with paclitaxel at least 3 times a week.

In some embodiments, Compound A is administered at about 15 mg to about 30 mg in combination with paclitaxel at least 3 times a week.

In some embodiments, Compound A is administered at about 15 mg to about 25 mg in combination with paclitaxel at least 3 times a week.

In some embodiments, Compound A is administered at about 15 mg to about 20 mg in combination with paclitaxel at least 3 times a week.

In some embodiments, Compound A is administered at about 5 mg in combination with paclitaxel at least 3 times a week.

In some embodiments, Compound A is administered at about 10 mg in combination with paclitaxel at least 3 times a week.

In some embodiments, Compound A is administered at about 15 mg in combination with paclitaxel at least 3 times a week.

In some embodiments, Compound A is administered at about 20 mg in combination with paclitaxel at least 3 times a week.

In some embodiments, Compound A is administered at about 25 mg in combination with paclitaxel at least 3 times a week.

In some embodiments, Compound A is administered at about 30 mg in combination with paclitaxel at least 3 times a week.

In some embodiments, Compound A is administered at about 35 mg in combination with paclitaxel at least 3 times a week.

In some embodiments, Compound A is administered at about 40 mg in combination with paclitaxel at least 3 times a week.

In some embodiments, Compound A is administered at about 45 mg in combination with paclitaxel at least 3 times a week.

In some embodiments, Compound A is administered at about 50 mg in combination with paclitaxel at least 3 times a week.

In some embodiments, the paclitaxel is administered in combination with Compound A at least 3 times a week, wherein the at least 3 days are consecutive.

In some embodiments, the paclitaxel is administered in combination with Compound A at least 4 times a week.

In some embodiments, the paclitaxel is administered at an amount of about 100 mg/m² to about 400 mg/m² in combination with Compound A at least 4 times a week.

In some embodiments, the paclitaxel is administered at an amount of about 100 mg/m² to about 350 mg/m² in combination with Compound A at least 4 times a week.

In some embodiments, the paclitaxel is administered at an amount of about 100 mg/m² to about 300 mg/m² in combination with Compound A at least 4 times a week.

In some embodiments, the paclitaxel is administered at an amount of about 150 mg/m² to about 250 mg/m² in combination with Compound A at least 4 times a week.

In some embodiments, the paclitaxel is administered at an amount of about 100 mg/m² to about 245 mg/m² in combination with Compound A at least 4 times a week.

In some embodiments, the paclitaxel is administered at an amount of about 100 mg/m² to about 240 mg/m² in combination with Compound A at least 4 times a week.

In some embodiments, the paclitaxel is administered at an amount of about 100 mg/m² to about 235 mg/m² in combination with Compound A at least 4 times a week.

In some embodiments, the paclitaxel is administered at an amount of about 100 mg/m² to about 230 mg/m² in combination with Compound A at least 4 times a week.

In some embodiments, the paclitaxel is administered at an amount of about 100 mg/m² to about 225 mg/m² in combination with Compound A at least 4 times a week.

In some embodiments, the paclitaxel is administered at an amount of about 100 mg/m² to about 220 mg/m² in combination with Compound A at least 4 times a week.

In some embodiments, the paclitaxel is administered at an amount of about 100 mg/m² to about 215 mg/m² in combination with Compound A at least 4 times a week.

In some embodiments, the paclitaxel is administered at an amount of about 100 mg/m² to about 210 mg/m² in combination with Compound A at least 4 times a week.

In some embodiments, the paclitaxel is administered at an amount of about 100 mg/m² to about 205 mg/m² in combination with Compound A at least 4 times a week.

In some embodiments, the paclitaxel is administered at an amount of about 100 mg/m² to about 200 mg/m² in combination with Compound A at least 4 times a week.

In some embodiments, the paclitaxel is administered at an amount of about 100 mg/m² to about 195 mg/m² in combination with Compound A at least 4 times a week.

In some embodiments, the paclitaxel is administered at an amount of about 100 mg/m² to about 190 mg/m² in combination with Compound A at least 4 times a week.

In some embodiments, the paclitaxel is administered at an amount of about 100 mg/m² to about 185 mg/m² in combination with Compound A at least 4 times a week.

In some embodiments, the paclitaxel is administered at an amount of about 100 mg/m² to about 180 mg/m² in combination with Compound A at least 4 times a week.

In some embodiments, the paclitaxel is administered at an amount of about 100 mg/m² to about 175 mg/m² in combination with Compound A at least 4 times a week.

In some embodiments, the paclitaxel is administered at an amount of about 100 mg/m² to about 170 mg/m² in combination with Compound A at least 4 times a week.

In some embodiments, the paclitaxel is administered at an amount of about 100 mg/m² to about 165 mg/m² in combination with Compound A at least 4 times a week.

In some embodiments, the paclitaxel is administered at an amount of about 100 mg/m² to about 160 mg/m² in combination with Compound A at least 4 times a week.

In some embodiments, the paclitaxel is administered at an amount of about 100 mg/m² to about 155 mg/m² in combination with Compound A at least 4 times a week.

In some embodiments, the paclitaxel is administered at an amount of about 100 mg/m² to about 150 mg/m² in combination with Compound A at least 4 times a week.

In some embodiments, the paclitaxel is administered at an amount of about 100 mg/m² to about 145 mg/m² in combination with Compound A at least 4 times a week.

In some embodiments, the paclitaxel is administered at an amount of about 100 mg/m² to about 140 mg/m² in combination with Compound A at least 4 times a week.

In some embodiments, the paclitaxel is administered at an amount of about 100 mg/m² to about 135 mg/m² in combination with Compound A at least 4 times a week.

In some embodiments, the paclitaxel is administered at an amount of about 100 mg/m² to about 130 mg/m² in combination with Compound A at least 4 times a week.

In some embodiments, the paclitaxel is administered at an amount of about 100 mg/m² to about 125 mg/m² in combination with Compound A at least 4 times a week.

In some embodiments, the paclitaxel is administered at an amount of about 100 mg/m² to about 120 mg/m² in combination with Compound A at least 4 times a week.

In some embodiments, the paclitaxel is administered at an amount of about 100 mg/m² to about 115 mg/m² in combination with Compound A at least 4 times a week.

In some embodiments, the paclitaxel is administered at an amount of about 100 mg/m² to about 110 mg/m² in combination with Compound A at least 4 times a week.

In some embodiments, the paclitaxel is administered at an amount of about 100 mg/m² to about 105 mg/m² in combination with Compound A at least 4 times a week.

In some embodiments, the paclitaxel is administered at an amount of about 150 mg/m² to about 350 mg/m² in combination with Compound A at least 4 times a week.

In some embodiments, the paclitaxel is administered at an amount of about 150 mg/m² to about 300 mg/m² in combination with Compound A at least 4 times a week.

In some embodiments, the paclitaxel is administered at an amount of about 150 mg/m² to about 250 mg/m² in combination with Compound A at least 4 times a week.

In some embodiments, the paclitaxel is administered at an amount of about 150 mg/m² to about 245 mg/m² in combination with Compound A at least 4 times a week.

In some embodiments, the paclitaxel is administered at an amount of about 150 mg/m² to about 240 mg/m² in combination with Compound A at least 4 times a week.

In some embodiments, the paclitaxel is administered at an amount of about 150 mg/m² to about 235 mg/m² in combination with Compound A at least 4 times a week.

In some embodiments, the paclitaxel is administered at an amount of about 150 mg/m² to about 230 mg/m² in combination with Compound A at least 4 times a week.

In some embodiments, the paclitaxel is administered at an amount of about 150 mg/m² to about 225 mg/m² in combination with Compound A at least 4 times a week.

In some embodiments, the paclitaxel is administered at an amount of about 150 mg/m² to about 220 mg/m² in combination with Compound A at least 4 times a week.

In some embodiments, the paclitaxel is administered at an amount of about 150 mg/m² to about 215 mg/m² in combination with Compound A at least 4 times a week.

In some embodiments, the paclitaxel is administered at an amount of about 150 mg/m² to about 210 mg/m² in combination with Compound A at least 4 times a week.

In some embodiments, the paclitaxel is administered at an amount of about 150 mg/m² to about 205 mg/m² in combination with Compound A at least 4 times a week.

In some embodiments, the paclitaxel is administered at an amount of about 150 mg/m² to about 200 mg/m² in combination with Compound A at least 4 times a week.

In some embodiments, the paclitaxel is administered at an amount of about 150 mg/m² to about 195 mg/m² in combination with Compound A at least 4 times a week.

In some embodiments, the paclitaxel is administered at an amount of about 150 mg/m² to about 190 mg/m² in combination with Compound A at least 4 times a week.

In some embodiments, the paclitaxel is administered at an amount of about 150 mg/m² to about 185 mg/m² in combination with Compound A at least 4 times a week.

In some embodiments, the paclitaxel is administered at an amount of about 150 mg/m² to about 180 mg/m² in combination with Compound A at least 4 times a week.

In some embodiments, the paclitaxel is administered at an amount of about 150 mg/m² to about 175 mg/m² in combination with Compound A at least 4 times a week.

In some embodiments, the paclitaxel is administered at an amount of about 150 mg/m² to about 170 mg/m² in combination with Compound A at least 4 times a week.

In some embodiments, the paclitaxel is administered at an amount of about 150 mg/m² to about 165 mg/m² in combination with Compound A at least 4 times a week.

In some embodiments, the paclitaxel is administered at an amount of about 150 mg/m² to about 160 mg/m² in combination with Compound A at least 4 times a week.

In some embodiments, the paclitaxel is administered at an amount of about 150 mg/m² to about 155 mg/m² in combination with Compound A at least 4 times a week.

In some embodiments, the paclitaxel is administered at an amount of about 200 mg/m² to about 300 mg/m² in combination with Compound A at least 4 times a week.

In some embodiments, the paclitaxel is administered at an amount of about 200 mg/m² to about 295 mg/m² in combination with Compound A at least 4 times a week.

In some embodiments, the paclitaxel is administered at an amount of about 200 mg/m² to about 290 mg/m² in combination with Compound A at least 4 times a week.

In some embodiments, the paclitaxel is administered at an amount of about 200 mg/m² to about 285 mg/m² in combination with Compound A at least 4 times a week.

In some embodiments, the paclitaxel is administered at an amount of about 200 mg/m² to about 280 mg/m² in combination with Compound A at least 4 times a week.

In some embodiments, the paclitaxel is administered at an amount of about 200 mg/m² to about 275 mg/m² in combination with Compound A at least 4 times a week.

In some embodiments, the paclitaxel is administered at an amount of about 200 mg/m² to about 270 mg/m² in combination with Compound A at least 4 times a week.

In some embodiments, the paclitaxel is administered at an amount of about 200 mg/m² to about 265 mg/m² in combination with Compound A at least 4 times a week.

In some embodiments, the paclitaxel is administered at an amount of about 200 mg/m² to about 260 mg/m² in combination with Compound A at least 4 times a week.

In some embodiments, the paclitaxel is administered at an amount of about 200 mg/m² to about 255 mg/m² in combination with Compound A at least 4 times a week.

In some embodiments, the paclitaxel is administered at an amount of about 200 mg/m² to about 250 mg/m² in combination with Compound A at least 4 times a week.

In some embodiments, the paclitaxel is administered at an amount of about 200 mg/m² to about 245 mg/m² in combination with Compound A at least 4 times a week.

In some embodiments, the paclitaxel is administered at an amount of about 200 mg/m² to about 240 mg/m² in combination with Compound A at least 4 times a week.

In some embodiments, the paclitaxel is administered at an amount of about 200 mg/m² to about 235 mg/m² in combination with Compound A at least 4 times a week.

In some embodiments, the paclitaxel is administered at an amount of about 200 mg/m² to about 230 mg/m² in combination with Compound A at least 4 times a week.

In some embodiments, the paclitaxel is administered at an amount of about 200 mg/m² to about 225 mg/m² in combination with Compound A at least 4 times a week.

In some embodiments, the paclitaxel is administered at an amount of about 200 mg/m² to about 220 mg/m² in combination with Compound A at least 4 times a week.

In some embodiments, the paclitaxel is administered at an amount of about 200 mg/m² to about 215 mg/m² in combination with Compound A at least 4 times a week.

In some embodiments, the paclitaxel is administered at an amount of about 200 mg/m² to about 210 mg/m² in combination with Compound A at least 4 times a week.

In some embodiments, the paclitaxel is administered at an amount of about 200 mg/m² to about 205 mg/m² in combination with Compound A at least 4 times a week.

In some embodiments, the paclitaxel is administered at an amount of about 250 mg/m² to about 350 mg/m² in combination with Compound A at least 4 times a week.

In some embodiments, the paclitaxel is administered at an amount of about 250 mg/m² to about 300 mg/m² in combination with Compound A at least 4 times a week.

In some embodiments, the paclitaxel is administered at an amount of about 120 mg/m² to about 235 mg/m² in combination with Compound A at least 4 times a week.

In some embodiments, the paclitaxel is administered at an amount of about 120 mg/m² to about 230 mg/m² in combination with Compound A at least 4 times a week.

In some embodiments, the paclitaxel is administered at an amount of about 120 mg/m² to about 225 mg/m² in combination with Compound A at least 4 times a week.

In some embodiments, the paclitaxel is administered at an amount of about 120 mg/m² to about 220 mg/m² in combination with Compound A at least 4 times a week.

In some embodiments, the paclitaxel is administered at an amount of about 120 mg/m² to about 215 mg/m² in combination with Compound A at least 4 times a week.

In some embodiments, the paclitaxel is administered at an amount of about 120 mg/m² to about 210 mg/m² in combination with Compound A at least 4 times a week.

In some embodiments, the paclitaxel is administered at an amount of about 120 mg/m² to about 205 mg/m² in combination with Compound A at least 4 times a week.

In some embodiments, the paclitaxel is administered at an amount of about 120 mg/m² to about 200 mg/m² in combination with Compound A at least 4 times a week.

In some embodiments, the paclitaxel is administered at an amount of about 120 mg/m² to about 195 mg/m² in combination with Compound A at least 4 times a week.

In some embodiments, the paclitaxel is administered at an amount of about 120 mg/m² to about 190 mg/m² in combination with Compound A at least 4 times a week.

In some embodiments, the paclitaxel is administered at an amount of about 120 mg/m² to about 185 mg/m² in combination with Compound A at least 4 times a week.

In some embodiments, the paclitaxel is administered at an amount of about 120 mg/m² to about 180 mg/m² in combination with Compound A at least 4 times a week.

In some embodiments, the paclitaxel is administered at an amount of about 120 mg/m² to about 175 mg/m² in combination with Compound A at least 4 times a week.

In some embodiments, the paclitaxel is administered at an amount of about 120 mg/m² to about 170 mg/m² in combination with Compound A at least 4 times a week.

In some embodiments, the paclitaxel is administered at an amount of about 120 mg/m² to about 165 mg/m² in combination with Compound A at least 4 times a week.

In some embodiments, the paclitaxel is administered at an amount of about 120 mg/m² to about 160 mg/m² in combination with Compound A at least 4 times a week.

In some embodiments, the paclitaxel is administered at an amount of about 120 mg/m² to about 155 mg/m² in combination with Compound A at least 4 times a week.

In some embodiments, the paclitaxel is administered at an amount of about 120 mg/m² to about 150 mg/m² in combination with Compound A at least 4 times a week.

In some embodiments, the paclitaxel is administered at an amount of about 120 mg/m² to about 145 mg/m² in combination with Compound A at least 4 times a week.

In some embodiments, the paclitaxel is administered at an amount of about 120 mg/m² to about 140 mg/m² in combination with Compound A at least 4 times a week.

In some embodiments, the paclitaxel is administered at an amount of about 120 mg/m² to about 135 mg/m² in combination with Compound A at least 4 times a week.

In some embodiments, the paclitaxel is administered at an amount of about 120 mg/m² to about 130 mg/m² in combination with Compound A at least 4 times a week.

In some embodiments, the paclitaxel is administered at an amount of about 120 mg/m² to about 125 mg/m² in combination with Compound A at least 4 times a week.

In some embodiments, the paclitaxel is administered at an amount of about 125 mg/m² to about 235 mg/m² in combination with Compound A at least 4 times a week.

In some embodiments, the paclitaxel is administered at an amount of about 125 mg/m² to about 230 mg/m² in combination with Compound A at least 4 times a week.

In some embodiments, the paclitaxel is administered at an amount of about 125 mg/m² to about 225 mg/m² in combination with Compound A at least 4 times a week.

In some embodiments, the paclitaxel is administered at an amount of about 125 mg/m² to about 220 mg/m² in combination with Compound A at least 4 times a week.

In some embodiments, the paclitaxel is administered at an amount of about 125 mg/m² to about 215 mg/m² in combination with Compound A at least 4 times a week.

In some embodiments, the paclitaxel is administered at an amount of about 125 mg/m² to about 210 mg/m² in combination with Compound A at least 4 times a week.

In some embodiments, the paclitaxel is administered at an amount of about 125 mg/m² to about 205 mg/m² in combination with Compound A at least 4 times a week.

In some embodiments, the paclitaxel is administered at an amount of about 125 mg/m² to about 200 mg/m² in combination with Compound A at least 4 times a week.

In some embodiments, the paclitaxel is administered at an amount of about 125 mg/m² to about 195 mg/m² in combination with Compound A at least 4 times a week.

In some embodiments, the paclitaxel is administered at an amount of about 125 mg/m² to about 190 mg/m² in combination with Compound A at least 4 times a week.

In some embodiments, the paclitaxel is administered at an amount of about 125 mg/m² to about 185 mg/m² in combination with Compound A at least 4 times a week.

In some embodiments, the paclitaxel is administered at an amount of about 125 mg/m² to about 180 mg/m² in combination with Compound A at least 4 times a week.

In some embodiments, the paclitaxel is administered at an amount of about 125 mg/m² to about 175 mg/m² in combination with Compound A at least 4 times a week.

In some embodiments, the paclitaxel is administered at an amount of about 125 mg/m² to about 170 mg/m² in combination with Compound A at least 4 times a week.

In some embodiments, the paclitaxel is administered at an amount of about 125 mg/m² to about 165 mg/m² in combination with Compound A at least 4 times a week.

In some embodiments, the paclitaxel is administered at an amount of about 125 mg/m² to about 160 mg/m² in combination with Compound A at least 4 times a week.

In some embodiments, the paclitaxel is administered at an amount of about 125 mg/m² to about 155 mg/m² in combination with Compound A at least 4 times a week.

In some embodiments, the paclitaxel is administered at an amount of about 125 mg/m² to about 150 mg/m² in combination with Compound A at least 4 times a week.

In some embodiments, the paclitaxel is administered at an amount of about 125 mg/m² to about 145 mg/m² in combination with Compound A at least 4 times a week.

In some embodiments, the paclitaxel is administered at an amount of about 125 mg/m² to about 140 mg/m² in combination with Compound A at least 4 times a week.

In some embodiments, the paclitaxel is administered at an amount of about 125 mg/m² to about 135 mg/m² in combination with Compound A at least 4 times a week.

In some embodiments, the paclitaxel is administered at an amount of about 125 mg/m² to about 130 mg/m² in combination with Compound A at least 4 times a week.

In some embodiments, the paclitaxel is administered at an amount of about 130 mg/m² to about 235 mg/m² in combination with Compound A at least 4 times a week.

In some embodiments, the paclitaxel is administered at an amount of about 130 mg/m² to about 230 mg/m² in combination with Compound A at least 4 times a week.

In some embodiments, the paclitaxel is administered at an amount of about 130 mg/m² to about 225 mg/m² in combination with Compound A at least 4 times a week.

In some embodiments, the paclitaxel is administered at an amount of about 130 mg/m² to about 220 mg/m² in combination with Compound A at least 4 times a week.

In some embodiments, the paclitaxel is administered at an amount of about 130 mg/m² to about 215 mg/m² in combination with Compound A at least 4 times a week.

In some embodiments, the paclitaxel is administered at an amount of about 130 mg/m² to about 210 mg/m² in combination with Compound A at least 4 times a week.

In some embodiments, the paclitaxel is administered at an amount of about 130 mg/m² to about 205 mg/m² in combination with Compound A at least 4 times a week.

In some embodiments, the paclitaxel is administered at an amount of about 130 mg/m² to about 200 mg/m² in combination with Compound A at least 4 times a week.

In some embodiments, the paclitaxel is administered at an amount of about 130 mg/m² to about 195 mg/m² in combination with Compound A at least 4 times a week.

In some embodiments, the paclitaxel is administered at an amount of about 130 mg/m² to about 190 mg/m² in combination with Compound A at least 4 times a week.

In some embodiments, the paclitaxel is administered at an amount of about 130 mg/m² to about 185 mg/m² in combination with Compound A at least 4 times a week.

In some embodiments, the paclitaxel is administered at an amount of about 130 mg/m² to about 180 mg/m² in combination with Compound A at least 4 times a week.

In some embodiments, the paclitaxel is administered at an amount of about 130 mg/m² to about 175 mg/m² in combination with Compound A at least 4 times a week.

In some embodiments, the paclitaxel is administered at an amount of about 130 mg/m² to about 170 mg/m² in combination with Compound A at least 4 times a week.

In some embodiments, the paclitaxel is administered at an amount of about 130 mg/m² to about 165 mg/m² in combination with Compound A at least 4 times a week.

In some embodiments, the paclitaxel is administered at an amount of about 130 mg/m² to about 160 mg/m² in combination with Compound A at least 4 times a week.

In some embodiments, the paclitaxel is administered at an amount of about 130 mg/m² to about 155 mg/m² in combination with Compound A at least 4 times a week.

In some embodiments, the paclitaxel is administered at an amount of about 130 mg/m² to about 150 mg/m² in combination with Compound A at least 4 times a week.

In some embodiments, the paclitaxel is administered at an amount of about 130 mg/m² to about 145 mg/m² in combination with Compound A at least 4 times a week.

In some embodiments, the paclitaxel is administered at an amount of about 130 mg/m² to about 140 mg/m² in combination with Compound A at least 4 times a week.

In some embodiments, the paclitaxel is administered at an amount of about 130 mg/m² to about 135 mg/m² in combination with Compound A at least 4 times a week.

In some embodiments, the paclitaxel is administered at an amount of about 100 mg/m² in combination with Compound A at least 4 times a week.

In some embodiments, the paclitaxel is administered at an amount of about 105 mg/m² in combination with Compound A at least 4 times a week.

In some embodiments, the paclitaxel is administered at an amount of about 110 mg/m² in combination with Compound A at least 4 times a week.

In some embodiments, the paclitaxel is administered at an amount of about 115 mg/m² in combination with Compound A at least 4 times a week.

In some embodiments, the paclitaxel is administered at an amount of about 120 mg/m² in combination with Compound A at least 4 times a week.

In some embodiments, the paclitaxel is administered at an amount of about 125 mg/m² in combination with Compound A at least 4 times a week.

In some embodiments, the paclitaxel is administered at an amount of about 130 mg/m² in combination with Compound A at least 4 times a week.

In some embodiments, the paclitaxel is administered at an amount of about 135 mg/m² in combination with Compound A at least 4 times a week.

In some embodiments, the paclitaxel is administered at an amount of about 140 mg/m² in combination with Compound A at least 4 times a week.

In some embodiments, the paclitaxel is administered at an amount of about 145 mg/m² in combination with Compound A at least 4 times a week.

In some embodiments, the paclitaxel is administered at an amount of about 150 mg/m² in combination with Compound A at least 4 times a week.

In some embodiments, the paclitaxel is administered at an amount of about 155 mg/m² in combination with Compound A at least 4 times a week.

In some embodiments, the paclitaxel is administered at an amount of about 160 mg/m² in combination with Compound A at least 4 times a week.

In some embodiments, the paclitaxel is administered at an amount of about 165 mg/m² in combination with Compound A at least 4 times a week.

In some embodiments, the paclitaxel is administered at an amount of about 170 mg/m² in combination with Compound A at least 4 times a week.

In some embodiments, the paclitaxel is administered at an amount of about 175 mg/m² in combination with Compound A at least 4 times a week.

In some embodiments, the paclitaxel is administered at an amount of about 180 mg/m² in combination with Compound A at least 4 times a week.

In some embodiments, the paclitaxel is administered at an amount of about 185 mg/m² in combination with Compound A at least 4 times a week.

In some embodiments, the paclitaxel is administered at an amount of about 190 mg/m² in combination with Compound A at least 4 times a week.

In some embodiments, the paclitaxel is administered at an amount of about 195 mg/m² in combination with Compound A at least 4 times a week.

In some embodiments, the paclitaxel is administered at an amount of about 200 mg/m² in combination with Compound A at least 4 times a week.

In some embodiments, the paclitaxel is administered at an amount of about 205 mg/m² in combination with Compound A at least 4 times a week.

In some embodiments, the paclitaxel is administered at an amount of about 210 mg/m² in combination with Compound A at least 4 times a week.

In some embodiments, the paclitaxel is administered at an amount of about 215 mg/m² in combination with Compound A at least 4 times a week.

In some embodiments, the paclitaxel is administered at an amount of about 220 mg/m² in combination with Compound A at least 4 times a week.

In some embodiments, the paclitaxel is administered at an amount of about 225 mg/m² in combination with Compound A at least 4 times a week.

In some embodiments, the paclitaxel is administered at an amount of about 230 mg/m² in combination with Compound A at least 4 times a week.

In some embodiments, the paclitaxel is administered at an amount of about 235 mg/m² in combination with Compound A at least 4 times a week.

In some embodiments, the paclitaxel is administered at an amount of about 240 mg/m² in combination with Compound A at least 4 times a week.

In some embodiments, the paclitaxel is administered at an amount of about 245 mg/m² in combination with Compound A at least 4 times a week.

In some embodiments, the paclitaxel is administered at an amount of about 250 mg/m² in combination with Compound A at least 4 times a week.

In some embodiments, the paclitaxel is administered at an amount of about 255 mg/m² in combination with Compound A at least 4 times a week.

In some embodiments, the paclitaxel is administered at an amount of about 260 mg/m² in combination with Compound A at least 4 times a week.

In some embodiments, the paclitaxel is administered at an amount of about 265 mg/m² in combination with Compound A at least 4 times a week.

In some embodiments, the paclitaxel is administered at an amount of about 270 mg/m² in combination with Compound A at least 4 times a week.

In some embodiments, the paclitaxel is administered at an amount of about 275 mg/m² in combination with Compound A at least 4 times a week.

In some embodiments, the paclitaxel is administered at an amount of about 280 mg/m² in combination with Compound A at least 4 times a week.

In some embodiments, the paclitaxel is administered at an amount of about 285 mg/m² in combination with Compound A at least 4 times a week.

In some embodiments, the paclitaxel is administered at an amount of about 290 mg/m² in combination with Compound A at least 4 times a week.

In some embodiments, the paclitaxel is administered at an amount of about 295 mg/m² in combination with Compound A at least 4 times a week.

In some embodiments, the paclitaxel is administered at an amount of about 300 mg/m² in combination with Compound A at least 4 times a week.

In some embodiments, the paclitaxel is administered at an amount of about 305 mg/m² in combination with Compound A at least 4 times a week.

In some embodiments, the paclitaxel is administered at an amount of about 310 mg/m² in combination with Compound A at least 4 times a week.

In some embodiments, the paclitaxel is administered at an amount of about 315 mg/m² in combination with Compound A at least 4 times a week.

In some embodiments, the paclitaxel is administered at an amount of about 320 mg/m² in combination with Compound A at least 4 times a week.

In some embodiments, the paclitaxel is administered at an amount of about 325 mg/m² in combination with Compound A at least 4 times a week.

In some embodiments, the paclitaxel is administered at an amount of about 330 mg/m² in combination with Compound A at least 4 times a week.

In some embodiments, the paclitaxel is administered at an amount of about 335 mg/m² in combination with Compound A at least 4 times a week.

In some embodiments, the paclitaxel is administered at an amount of about 340 mg/m² in combination with Compound A at least 4 times a week.

In some embodiments, the paclitaxel is administered at an amount of about 345 mg/m² in combination with Compound A at least 4 times a week.

In some embodiments, the paclitaxel is administered at an amount of about 350 mg/m² in combination with Compound A at least 4 times a week.

In some embodiments, the paclitaxel is administered at an amount of about 355 mg/m² in combination with Compound A at least 4 times a week.

In some embodiments, the paclitaxel is administered at an amount of about 360 mg/m² in combination with Compound A at least 4 times a week.

In some embodiments, the paclitaxel is administered at an amount of about 365 mg/m² in combination with Compound A at least 4 times a week.

In some embodiments, the paclitaxel is administered at an amount of about 370 mg/m² in combination with Compound A at least 4 times a week.

In some embodiments, the paclitaxel is administered at an amount of about 375 mg/m² in combination with Compound A at least 4 times a week.

In some embodiments, the paclitaxel is administered at an amount of about 380 mg/m² in combination with Compound A at least 4 times a week.

In some embodiments, the paclitaxel is administered at an amount of about 385 mg/m² in combination with Compound A at least 4 times a week.

In some embodiments, the paclitaxel is administered at an amount of about 390 mg/m² in combination with Compound A at least 4 times a week.

In some embodiments, the paclitaxel is administered at an amount of about 395 mg/m² in combination with Compound A at least 4 times a week.

In some embodiments, the paclitaxel is administered at an amount of about 400 mg/m² in combination with Compound A at least 4 times a week.

In some embodiments, Compound A is administered in combination with paclitaxel at least 4 times a week.

In some embodiments, Compound A is administered at an amount of about 1 mg to about 500 mg in combination with paclitaxel at least 4 times a week.

In some embodiments, Compound A is administered at about 1 mg to about 400 mg in combination with paclitaxel at least 4 times a week.

In some embodiments, Compound A is administered at about 1 mg to about 300 mg in combination with paclitaxel at least 4 times a week.

In some embodiments, Compound A is administered at about 5 mg to about 200 mg in combination with paclitaxel at least 4 times a week.

In some embodiments, Compound A is administered at about 10 mg to about 100 mg in combination with paclitaxel at least 4 times a week.

In some embodiments, Compound A is administered at about 10 mg to about 95 mg in combination with paclitaxel at least 4 times a week.

In some embodiments, Compound A is administered at about 10 mg to about 90 mg in combination with paclitaxel at least 4 times a week.

In some embodiments, Compound A is administered at about 10 mg to about 85 mg in combination with paclitaxel at least 4 times a week.

In some embodiments, Compound A is administered at about 10 mg to about 80 mg in combination with paclitaxel at least 4 times a week.

In some embodiments, Compound A is administered at about 10 mg to about 75 mg in combination with paclitaxel at least 4 times a week.

In some embodiments, Compound A is administered at about 10 mg to about 70 mg in combination with paclitaxel at least 4 times a week.

In some embodiments, Compound A is administered at about 10 mg to about 65 mg in combination with paclitaxel at least 4 times a week.

In some embodiments, Compound A is administered at about 10 mg to about 60 mg in combination with paclitaxel at least 4 times a week.

In some embodiments, Compound A is administered at about 10 mg to about 55 mg in combination with paclitaxel at least 4 times a week.

In some embodiments, Compound A is administered at about 10 mg to about 50 mg in combination with paclitaxel at least 4 times a week.

In some embodiments, Compound A is administered at about 10 mg to about 45 mg in combination with paclitaxel at least 4 times a week.

In some embodiments, Compound A is administered at about 10 mg to about 40 mg in combination with paclitaxel at least 4 times a week.

In some embodiments, Compound A is administered at about 10 mg to about 35 mg in combination with paclitaxel at least 4 times a week.

In some embodiments, Compound A is administered at about 10 mg to about 30 mg in combination with paclitaxel at least 4 times a week.

In some embodiments, Compound A is administered at about 10 mg to about 25 mg in combination with paclitaxel at least 4 times a week.

In some embodiments, Compound A is administered at about 10 mg to about 20 mg in combination with paclitaxel at least 4 times a week.

In some embodiments, Compound A is administered at about 10 mg to about 15 mg in combination with paclitaxel at least 4 times a week.

In some embodiments, Compound A is administered at about 15 mg to about 50 mg in combination with paclitaxel at least 4 times a week.

In some embodiments, Compound A is administered at about 15 mg to about 45 mg in combination with paclitaxel at least 4 times a week.

In some embodiments, Compound A is administered at about 15 mg to about 40 mg in combination with paclitaxel at least 4 times a week.

In some embodiments, Compound A is administered at about 15 mg to about 35 mg in combination with paclitaxel at least 4 times a week.

In some embodiments, Compound A is administered at about 15 mg to about 30 mg in combination with paclitaxel at least 4 times a week.

In some embodiments, Compound A is administered at about 15 mg to about 25 mg in combination with paclitaxel at least 4 times a week.

In some embodiments, Compound A is administered at about 15 mg to about 20 mg in combination with paclitaxel at least 4 times a week.

In some embodiments, Compound A is administered at about 5 mg in combination with paclitaxel at least 4 times a week.

In some embodiments, Compound A is administered at about 10 mg in combination with paclitaxel at least 4 times a week.

In some embodiments, Compound A is administered at about 15 mg in combination with paclitaxel at least 4 times a week.

In some embodiments, Compound A is administered at about 20 mg in combination with paclitaxel at least 4 times a week.

In some embodiments, Compound A is administered at about 25 mg in combination with paclitaxel at least 4 times a week.

In some embodiments, Compound A is administered at about 30 mg in combination with paclitaxel at least 4 times a week.

In some embodiments, Compound A is administered at about 35 mg in combination with paclitaxel at least 4 times a week.

In some embodiments, Compound A is administered at about 40 mg in combination with paclitaxel at least 4 times a week.

In some embodiments, Compound A is administered at about 45 mg in combination with paclitaxel at least 4 times a week.

In some embodiments, Compound A is administered at about 50 mg in combination with paclitaxel at least 4 times a week.

In some embodiments, the paclitaxel is administered in combination with Compound A at least 4 times a week, wherein the at least 4 days are consecutive.

In some embodiments, the paclitaxel is administered in combination with Compound A at least 5 times a week.

In some embodiments, the paclitaxel is administered at an amount of about 100 mg/m² to about 400 mg/m² in combination with Compound A at least 5 times a week.

In some embodiments, the paclitaxel is administered at an amount of about 100 mg/m² to about 350 mg/m² in combination with Compound A at least 5 times a week.

In some embodiments, the paclitaxel is administered at an amount of about 100 mg/m² to about 300 mg/m² in combination with Compound A at least 5 times a week.

In some embodiments, the paclitaxel is administered at an amount of about 150 mg/m² to about 250 mg/m² in combination with Compound A at least 5 times a week.

In some embodiments, the paclitaxel is administered at an amount of about 100 mg/m² to about 245 mg/m² in combination with Compound A at least 5 times a week.

In some embodiments, the paclitaxel is administered at an amount of about 100 mg/m² to about 240 mg/m² in combination with Compound A at least 5 times a week.

In some embodiments, the paclitaxel is administered at an amount of about 100 mg/m² to about 235 mg/m² in combination with Compound A at least 5 times a week.

In some embodiments, the paclitaxel is administered at an amount of about 100 mg/m² to about 230 mg/m² in combination with Compound A at least 5 times a week.

In some embodiments, the paclitaxel is administered at an amount of about 100 mg/m² to about 225 mg/m² in combination with Compound A at least 5 times a week.

In some embodiments, the paclitaxel is administered at an amount of about 100 mg/m² to about 220 mg/m² in combination with Compound A at least 5 times a week.

In some embodiments, the paclitaxel is administered at an amount of about 100 mg/m² to about 215 mg/m² in combination with Compound A at least 5 times a week.

In some embodiments, the paclitaxel is administered at an amount of about 100 mg/m² to about 210 mg/m² in combination with Compound A at least 5 times a week.

In some embodiments, the paclitaxel is administered at an amount of about 100 mg/m² to about 205 mg/m² in combination with Compound A at least 5 times a week.

In some embodiments, the paclitaxel is administered at an amount of about 100 mg/m² to about 200 mg/m² in combination with Compound A at least 5 times a week.

In some embodiments, the paclitaxel is administered at an amount of about 100 mg/m² to about 195 mg/m² in combination with Compound A at least 5 times a week.

In some embodiments, the paclitaxel is administered at an amount of about 100 mg/m² to about 190 mg/m² in combination with Compound A at least 5 times a week.

In some embodiments, the paclitaxel is administered at an amount of about 100 mg/m² to about 185 mg/m² in combination with Compound A at least 5 times a week.

In some embodiments, the paclitaxel is administered at an amount of about 100 mg/m² to about 180 mg/m² in combination with Compound A at least 5 times a week.

In some embodiments, the paclitaxel is administered at an amount of about 100 mg/m² to about 175 mg/m² in combination with Compound A at least 5 times a week.

In some embodiments, the paclitaxel is administered at an amount of about 100 mg/m² to about 170 mg/m² in combination with Compound A at least 5 times a week.

In some embodiments, the paclitaxel is administered at an amount of about 100 mg/m² to about 165 mg/m² in combination with Compound A at least 5 times a week.

In some embodiments, the paclitaxel is administered at an amount of about 100 mg/m² to about 160 mg/m² in combination with Compound A at least 5 times a week.

In some embodiments, the paclitaxel is administered at an amount of about 100 mg/m² to about 155 mg/m² in combination with Compound A at least 5 times a week.

In some embodiments, the paclitaxel is administered at an amount of about 100 mg/m² to about 150 mg/m² in combination with Compound A at least 5 times a week.

In some embodiments, the paclitaxel is administered at an amount of about 100 mg/m² to about 145 mg/m² in combination with Compound A at least 5 times a week.

In some embodiments, the paclitaxel is administered at an amount of about 100 mg/m² to about 140 mg/m² in combination with Compound A at least 5 times a week.

In some embodiments, the paclitaxel is administered at an amount of about 100 mg/m² to about 135 mg/m² in combination with Compound A at least 5 times a week.

In some embodiments, the paclitaxel is administered at an amount of about 100 mg/m² to about 130 mg/m² in combination with Compound A at least 5 times a week.

In some embodiments, the paclitaxel is administered at an amount of about 100 mg/m² to about 125 mg/m² in combination with Compound A at least 5 times a week.

In some embodiments, the paclitaxel is administered at an amount of about 100 mg/m² to about 120 mg/m² in combination with Compound A at least 5 times a week.

In some embodiments, the paclitaxel is administered at an amount of about 100 mg/m² to about 115 mg/m² in combination with Compound A at least 5 times a week.

In some embodiments, the paclitaxel is administered at an amount of about 100 mg/m² to about 110 mg/m² in combination with Compound A at least 5 times a week.

In some embodiments, the paclitaxel is administered at an amount of about 100 mg/m² to about 105 mg/m² in combination with Compound A at least 5 times a week.

In some embodiments, the paclitaxel is administered at an amount of about 150 mg/m² to about 350 mg/m² in combination with Compound A at least 5 times a week.

In some embodiments, the paclitaxel is administered at an amount of about 150 mg/m² to about 300 mg/m² in combination with Compound A at least 5 times a week.

In some embodiments, the paclitaxel is administered at an amount of about 150 mg/m² to about 250 mg/m² in combination with Compound A at least 5 times a week.

In some embodiments, the paclitaxel is administered at an amount of about 150 mg/m² to about 245 mg/m² in combination with Compound A at least 5 times a week.

In some embodiments, the paclitaxel is administered at an amount of about 150 mg/m² to about 240 mg/m² in combination with Compound A at least 5 times a week.

In some embodiments, the paclitaxel is administered at an amount of about 150 mg/m² to about 235 mg/m² in combination with Compound A at least 5 times a week.

In some embodiments, the paclitaxel is administered at an amount of about 150 mg/m² to about 230 mg/m² in combination with Compound A at least 5 times a week.

In some embodiments, the paclitaxel is administered at an amount of about 150 mg/m² to about 225 mg/m² in combination with Compound A at least 5 times a week.

In some embodiments, the paclitaxel is administered at an amount of about 150 mg/m² to about 220 mg/m² in combination with Compound A at least 5 times a week.

In some embodiments, the paclitaxel is administered at an amount of about 150 mg/m² to about 215 mg/m² in combination with Compound A at least 5 times a week.

In some embodiments, the paclitaxel is administered at an amount of about 150 mg/m² to about 210 mg/m² in combination with Compound A at least 5 times a week.

In some embodiments, the paclitaxel is administered at an amount of about 150 mg/m² to about 205 mg/m² in combination with Compound A at least 5 times a week.

In some embodiments, the paclitaxel is administered at an amount of about 150 mg/m² to about 200 mg/m² in combination with Compound A at least 5 times a week.

In some embodiments, the paclitaxel is administered at an amount of about 150 mg/m² to about 195 mg/m² in combination with Compound A at least 5 times a week.

In some embodiments, the paclitaxel is administered at an amount of about 150 mg/m² to about 190 mg/m² in combination with Compound A at least 5 times a week.

In some embodiments, the paclitaxel is administered at an amount of about 150 mg/m² to about 185 mg/m² in combination with Compound A at least 5 times a week.

In some embodiments, the paclitaxel is administered at an amount of about 150 mg/m² to about 180 mg/m² in combination with Compound A at least 5 times a week.

In some embodiments, the paclitaxel is administered at an amount of about 150 mg/m² to about 175 mg/m² in combination with Compound A at least 5 times a week.

In some embodiments, the paclitaxel is administered at an amount of about 150 mg/m² to about 170 mg/m² in combination with Compound A at least 5 times a week.

In some embodiments, the paclitaxel is administered at an amount of about 150 mg/m² to about 165 mg/m² in combination with Compound A at least 5 times a week.

In some embodiments, the paclitaxel is administered at an amount of about 150 mg/m² to about 160 mg/m² in combination with Compound A at least 5 times a week.

In some embodiments, the paclitaxel is administered at an amount of about 150 mg/m² to about 155 mg/m² in combination with Compound A at least 5 times a week.

In some embodiments, the paclitaxel is administered at an amount of about 200 mg/m² to about 300 mg/m² in combination with Compound A at least 5 times a week.

In some embodiments, the paclitaxel is administered at an amount of about 200 mg/m² to about 295 mg/m² in combination with Compound A at least 5 times a week.

In some embodiments, the paclitaxel is administered at an amount of about 200 mg/m² to about 290 mg/m² in combination with Compound A at least 5 times a week.

In some embodiments, the paclitaxel is administered at an amount of about 200 mg/m² to about 285 mg/m² in combination with Compound A at least 5 times a week.

In some embodiments, the paclitaxel is administered at an amount of about 200 mg/m² to about 280 mg/m² in combination with Compound A at least 5 times a week.

In some embodiments, the paclitaxel is administered at an amount of about 200 mg/m² to about 275 mg/m² in combination with Compound A at least 5 times a week.

In some embodiments, the paclitaxel is administered at an amount of about 200 mg/m² to about 270 mg/m² in combination with Compound A at least 5 times a week.

In some embodiments, the paclitaxel is administered at an amount of about 200 mg/m² to about 265 mg/m² in combination with Compound A at least 5 times a week.

In some embodiments, the paclitaxel is administered at an amount of about 200 mg/m² to about 260 mg/m² in combination with Compound A at least 5 times a week.

In some embodiments, the paclitaxel is administered at an amount of about 200 mg/m² to about 255 mg/m² in combination with Compound A at least 5 times a week.

In some embodiments, the paclitaxel is administered at an amount of about 200 mg/m² to about 250 mg/m² in combination with Compound A at least 5 times a week.

In some embodiments, the paclitaxel is administered at an amount of about 200 mg/m² to about 245 mg/m² in combination with Compound A at least 5 times a week.

In some embodiments, the paclitaxel is administered at an amount of about 200 mg/m² to about 240 mg/m² in combination with Compound A at least 5 times a week.

In some embodiments, the paclitaxel is administered at an amount of about 200 mg/m² to about 235 mg/m² in combination with Compound A at least 5 times a week.

In some embodiments, the paclitaxel is administered at an amount of about 200 mg/m² to about 230 mg/m² in combination with Compound A at least 5 times a week.

In some embodiments, the paclitaxel is administered at an amount of about 200 mg/m² to about 225 mg/m² in combination with Compound A at least 5 times a week.

In some embodiments, the paclitaxel is administered at an amount of about 200 mg/m² to about 220 mg/m² in combination with Compound A at least 5 times a week.

In some embodiments, the paclitaxel is administered at an amount of about 200 mg/m² to about 215 mg/m² in combination with Compound A at least 5 times a week.

In some embodiments, the paclitaxel is administered at an amount of about 200 mg/m² to about 210 mg/m² in combination with Compound A at least 5 times a week.

In some embodiments, the paclitaxel is administered at an amount of about 200 mg/m² to about 205 mg/m² in combination with Compound A at least 5 times a week.

In some embodiments, the paclitaxel is administered at an amount of about 250 mg/m² to about 350 mg/m² in combination with Compound A at least 5 times a week.

In some embodiments, the paclitaxel is administered at an amount of about 250 mg/m² to about 300 mg/m² in combination with Compound A at least 5 times a week.

In some embodiments, the paclitaxel is administered at an amount of about 120 mg/m² to about 235 mg/m² in combination with Compound A at least 5 times a week.

In some embodiments, the paclitaxel is administered at an amount of about 120 mg/m² to about 230 mg/m² in combination with Compound A at least 5 times a week.

In some embodiments, the paclitaxel is administered at an amount of about 120 mg/m² to about 225 mg/m² in combination with Compound A at least 5 times a week.

In some embodiments, the paclitaxel is administered at an amount of about 120 mg/m² to about 220 mg/m² in combination with Compound A at least 5 times a week.

In some embodiments, the paclitaxel is administered at an amount of about 120 mg/m² to about 215 mg/m² in combination with Compound A at least 5 times a week.

In some embodiments, the paclitaxel is administered at an amount of about 120 mg/m² to about 210 mg/m² in combination with Compound A at least 5 times a week.

In some embodiments, the paclitaxel is administered at an amount of about 120 mg/m² to about 205 mg/m² in combination with Compound A at least 5 times a week.

In some embodiments, the paclitaxel is administered at an amount of about 120 mg/m² to about 200 mg/m² in combination with Compound A at least 5 times a week.

In some embodiments, the paclitaxel is administered at an amount of about 120 mg/m² to about 195 mg/m² in combination with Compound A at least 5 times a week.

In some embodiments, the paclitaxel is administered at an amount of about 120 mg/m² to about 190 mg/m² in combination with Compound A at least 5 times a week.

In some embodiments, the paclitaxel is administered at an amount of about 120 mg/m² to about 185 mg/m² in combination with Compound A at least 5 times a week.

In some embodiments, the paclitaxel is administered at an amount of about 120 mg/m² to about 180 mg/m² in combination with Compound A at least 5 times a week.

In some embodiments, the paclitaxel is administered at an amount of about 120 mg/m² to about 175 mg/m² in combination with Compound A at least 5 times a week.

In some embodiments, the paclitaxel is administered at an amount of about 120 mg/m² to about 170 mg/m² in combination with Compound A at least 5 times a week.

In some embodiments, the paclitaxel is administered at an amount of about 120 mg/m² to about 165 mg/m² in combination with Compound A at least 5 times a week.

In some embodiments, the paclitaxel is administered at an amount of about 120 mg/m² to about 160 mg/m² in combination with Compound A at least 5 times a week.

In some embodiments, the paclitaxel is administered at an amount of about 120 mg/m² to about 155 mg/m² in combination with Compound A at least 5 times a week.

In some embodiments, the paclitaxel is administered at an amount of about 120 mg/m² to about 150 mg/m² in combination with Compound A at least 5 times a week.

In some embodiments, the paclitaxel is administered at an amount of about 120 mg/m² to about 145 mg/m² in combination with Compound A at least 5 times a week.

In some embodiments, the paclitaxel is administered at an amount of about 120 mg/m² to about 140 mg/m² in combination with Compound A at least 5 times a week.

In some embodiments, the paclitaxel is administered at an amount of about 120 mg/m² to about 135 mg/m² in combination with Compound A at least 5 times a week.

In some embodiments, the paclitaxel is administered at an amount of about 120 mg/m² to about 130 mg/m² in combination with Compound A at least 5 times a week.

In some embodiments, the paclitaxel is administered at an amount of about 120 mg/m² to about 125 mg/m² in combination with Compound A at least 5 times a week.

In some embodiments, the paclitaxel is administered at an amount of about 125 mg/m² to about 235 mg/m² in combination with Compound A at least 5 times a week.

In some embodiments, the paclitaxel is administered at an amount of about 125 mg/m² to about 230 mg/m² in combination with Compound A at least 5 times a week.

In some embodiments, the paclitaxel is administered at an amount of about 125 mg/m² to about 225 mg/m² in combination with Compound A at least 5 times a week.

In some embodiments, the paclitaxel is administered at an amount of about 125 mg/m² to about 220 mg/m² in combination with Compound A at least 5 times a week.

In some embodiments, the paclitaxel is administered at an amount of about 125 mg/m² to about 215 mg/m² in combination with Compound A at least 5 times a week.

In some embodiments, the paclitaxel is administered at an amount of about 125 mg/m² to about 210 mg/m² in combination with Compound A at least 5 times a week.

In some embodiments, the paclitaxel is administered at an amount of about 125 mg/m² to about 205 mg/m² in combination with Compound A at least 5 times a week.

In some embodiments, the paclitaxel is administered at an amount of about 125 mg/m² to about 200 mg/m² in combination with Compound A at least 5 times a week.

In some embodiments, the paclitaxel is administered at an amount of about 125 mg/m² to about 195 mg/m² in combination with Compound A at least 5 times a week.

In some embodiments, the paclitaxel is administered at an amount of about 125 mg/m² to about 190 mg/m² in combination with Compound A at least 5 times a week.

In some embodiments, the paclitaxel is administered at an amount of about 125 mg/m² to about 185 mg/m² in combination with Compound A at least 5 times a week.

In some embodiments, the paclitaxel is administered at an amount of about 125 mg/m² to about 180 mg/m² in combination with Compound A at least 5 times a week.

In some embodiments, the paclitaxel is administered at an amount of about 125 mg/m² to about 175 mg/m² in combination with Compound A at least 5 times a week.

In some embodiments, the paclitaxel is administered at an amount of about 125 mg/m² to about 170 mg/m² in combination with Compound A at least 5 times a week.

In some embodiments, the paclitaxel is administered at an amount of about 125 mg/m² to about 165 mg/m² in combination with Compound A at least 5 times a week.

In some embodiments, the paclitaxel is administered at an amount of about 125 mg/m² to about 160 mg/m² in combination with Compound A at least 5 times a week.

In some embodiments, the paclitaxel is administered at an amount of about 125 mg/m² to about 155 mg/m² in combination with Compound A at least 5 times a week.

In some embodiments, the paclitaxel is administered at an amount of about 125 mg/m² to about 150 mg/m² in combination with Compound A at least 5 times a week.

In some embodiments, the paclitaxel is administered at an amount of about 125 mg/m² to about 145 mg/m² in combination with Compound A at least 5 times a week.

In some embodiments, the paclitaxel is administered at an amount of about 125 mg/m² to about 140 mg/m² in combination with Compound A at least 5 times a week.

In some embodiments, the paclitaxel is administered at an amount of about 125 mg/m² to about 135 mg/m² in combination with Compound A at least 5 times a week.

In some embodiments, the paclitaxel is administered at an amount of about 125 mg/m² to about 130 mg/m² in combination with Compound A at least 5 times a week.

In some embodiments, the paclitaxel is administered at an amount of about 130 mg/m² to about 235 mg/m² in combination with Compound A at least 5 times a week.

In some embodiments, the paclitaxel is administered at an amount of about 130 mg/m² to about 230 mg/m² in combination with Compound A at least 5 times a week.

In some embodiments, the paclitaxel is administered at an amount of about 130 mg/m² to about 225 mg/m² in combination with Compound A at least 5 times a week.

In some embodiments, the paclitaxel is administered at an amount of about 130 mg/m² to about 220 mg/m² in combination with Compound A at least 5 times a week.

In some embodiments, the paclitaxel is administered at an amount of about 130 mg/m² to about 215 mg/m² in combination with Compound A at least 5 times a week.

In some embodiments, the paclitaxel is administered at an amount of about 130 mg/m² to about 210 mg/m² in combination with Compound A at least 5 times a week.

In some embodiments, the paclitaxel is administered at an amount of about 130 mg/m² to about 205 mg/m² in combination with Compound A at least 5 times a week.

In some embodiments, the paclitaxel is administered at an amount of about 130 mg/m² to about 200 mg/m² in combination with Compound A at least 5 times a week.

In some embodiments, the paclitaxel is administered at an amount of about 130 mg/m² to about 195 mg/m² in combination with Compound A at least 5 times a week.

In some embodiments, the paclitaxel is administered at an amount of about 130 mg/m² to about 190 mg/m² in combination with Compound A at least 5 times a week.

In some embodiments, the paclitaxel is administered at an amount of about 130 mg/m² to about 185 mg/m² in combination with Compound A at least 5 times a week.

In some embodiments, the paclitaxel is administered at an amount of about 130 mg/m² to about 180 mg/m² in combination with Compound A at least 5 times a week.

In some embodiments, the paclitaxel is administered at an amount of about 130 mg/m² to about 175 mg/m² in combination with Compound A at least 5 times a week.

In some embodiments, the paclitaxel is administered at an amount of about 130 mg/m² to about 170 mg/m² in combination with Compound A at least 5 times a week.

In some embodiments, the paclitaxel is administered at an amount of about 130 mg/m² to about 165 mg/m² in combination with Compound A at least 5 times a week.

In some embodiments, the paclitaxel is administered at an amount of about 130 mg/m² to about 160 mg/m² in combination with Compound A at least 5 times a week.

In some embodiments, the paclitaxel is administered at an amount of about 130 mg/m² to about 155 mg/m² in combination with Compound A at least 5 times a week.

In some embodiments, the paclitaxel is administered at an amount of about 130 mg/m² to about 150 mg/m² in combination with Compound A at least 5 times a week.

In some embodiments, the paclitaxel is administered at an amount of about 130 mg/m² to about 145 mg/m² in combination with Compound A at least 5 times a week.

In some embodiments, the paclitaxel is administered at an amount of about 130 mg/m² to about 140 mg/m² in combination with Compound A at least 5 times a week.

In some embodiments, the paclitaxel is administered at an amount of about 130 mg/m² to about 135 mg/m² in combination with Compound A at least 5 times a week.

In some embodiments, the paclitaxel is administered at an amount of about 100 mg/m² in combination with Compound A at least 5 times a week.

In some embodiments, the paclitaxel is administered at an amount of about 105 mg/m² in combination with Compound A at least 5 times a week.

In some embodiments, the paclitaxel is administered at an amount of about 110 mg/m² in combination with Compound A at least 5 times a week.

In some embodiments, the paclitaxel is administered at an amount of about 115 mg/m² in combination with Compound A at least 5 times a week.

In some embodiments, the paclitaxel is administered at an amount of about 120 mg/m² in combination with Compound A at least 5 times a week.

In some embodiments, the paclitaxel is administered at an amount of about 125 mg/m² in combination with Compound A at least 5 times a week.

In some embodiments, the paclitaxel is administered at an amount of about 130 mg/m² in combination with Compound A at least 5 times a week.

In some embodiments, the paclitaxel is administered at an amount of about 135 mg/m² in combination with Compound A at least 5 times a week.

In some embodiments, the paclitaxel is administered at an amount of about 140 mg/m² in combination with Compound A at least 5 times a week.

In some embodiments, the paclitaxel is administered at an amount of about 145 mg/m² in combination with Compound A at least 5 times a week.

In some embodiments, the paclitaxel is administered at an amount of about 150 mg/m² in combination with Compound A at least 5 times a week.

In some embodiments, the paclitaxel is administered at an amount of about 155 mg/m² in combination with Compound A at least 5 times a week.

In some embodiments, the paclitaxel is administered at an amount of about 160 mg/m² in combination with Compound A at least 5 times a week.

In some embodiments, the paclitaxel is administered at an amount of about 165 mg/m² in combination with Compound A at least 5 times a week.

In some embodiments, the paclitaxel is administered at an amount of about 170 mg/m² in combination with Compound A at least 5 times a week.

In some embodiments, the paclitaxel is administered at an amount of about 175 mg/m² in combination with Compound A at least 5 times a week.

In some embodiments, the paclitaxel is administered at an amount of about 180 mg/m² in combination with Compound A at least 5 times a week.

In some embodiments, the paclitaxel is administered at an amount of about 185 mg/m² in combination with Compound A at least 5 times a week.

In some embodiments, the paclitaxel is administered at an amount of about 190 mg/m² in combination with Compound A at least 5 times a week.

In some embodiments, the paclitaxel is administered at an amount of about 195 mg/m² in combination with Compound A at least 5 times a week.

In some embodiments, the paclitaxel is administered at an amount of about 200 mg/m² in combination with Compound A at least 5 times a week.

In some embodiments, the paclitaxel is administered at an amount of about 205 mg/m² in combination with Compound A at least 5 times a week.

In some embodiments, the paclitaxel is administered at an amount of about 210 mg/m² in combination with Compound A at least 5 times a week.

In some embodiments, the paclitaxel is administered at an amount of about 215 mg/m² in combination with Compound A at least 5 times a week.

In some embodiments, the paclitaxel is administered at an amount of about 220 mg/m² in combination with Compound A at least 5 times a week.

In some embodiments, the paclitaxel is administered at an amount of about 225 mg/m² in combination with Compound A at least 5 times a week.

In some embodiments, the paclitaxel is administered at an amount of about 230 mg/m² in combination with Compound A at least 5 times a week.

In some embodiments, the paclitaxel is administered at an amount of about 235 mg/m² in combination with Compound A at least 5 times a week.

In some embodiments, the paclitaxel is administered at an amount of about 240 mg/m² in combination with Compound A at least 5 times a week.

In some embodiments, the paclitaxel is administered at an amount of about 245 mg/m² in combination with Compound A at least 5 times a week.

In some embodiments, the paclitaxel is administered at an amount of about 250 mg/m² in combination with Compound A at least 5 times a week.

In some embodiments, the paclitaxel is administered at an amount of about 255 mg/m² in combination with Compound A at least 5 times a week.

In some embodiments, the paclitaxel is administered at an amount of about 260 mg/m² in combination with Compound A at least 5 times a week.

In some embodiments, the paclitaxel is administered at an amount of about 265 mg/m² in combination with Compound A at least 5 times a week.

In some embodiments, the paclitaxel is administered at an amount of about 270 mg/m² in combination with Compound A at least 5 times a week.

In some embodiments, the paclitaxel is administered at an amount of about 275 mg/m² in combination with Compound A at least 5 times a week.

In some embodiments, the paclitaxel is administered at an amount of about 280 mg/m² in combination with Compound A at least 5 times a week.

In some embodiments, the paclitaxel is administered at an amount of about 285 mg/m² in combination with Compound A at least 5 times a week.

In some embodiments, the paclitaxel is administered at an amount of about 290 mg/m² in combination with Compound A at least 5 times a week.

In some embodiments, the paclitaxel is administered at an amount of about 295 mg/m² in combination with Compound A at least 5 times a week.

In some embodiments, the paclitaxel is administered at an amount of about 300 mg/m² in combination with Compound A at least 5 times a week.

In some embodiments, the paclitaxel is administered at an amount of about 305 mg/m² in combination with Compound A at least 5 times a week.

In some embodiments, the paclitaxel is administered at an amount of about 310 mg/m² in combination with Compound A at least 5 times a week.

In some embodiments, the paclitaxel is administered at an amount of about 315 mg/m² in combination with Compound A at least 5 times a week.

In some embodiments, the paclitaxel is administered at an amount of about 320 mg/m² in combination with Compound A at least 5 times a week.

In some embodiments, the paclitaxel is administered at an amount of about 325 mg/m² in combination with Compound A at least 5 times a week.

In some embodiments, the paclitaxel is administered at an amount of about 330 mg/m² in combination with Compound A at least 5 times a week.

In some embodiments, the paclitaxel is administered at an amount of about 335 mg/m² in combination with Compound A at least 5 times a week.

In some embodiments, the paclitaxel is administered at an amount of about 340 mg/m² in combination with Compound A at least 5 times a week.

In some embodiments, the paclitaxel is administered at an amount of about 345 mg/m² in combination with Compound A at least 5 times a week.

In some embodiments, the paclitaxel is administered at an amount of about 350 mg/m² in combination with Compound A at least 5 times a week.

In some embodiments, the paclitaxel is administered at an amount of about 355 mg/m² in combination with Compound A at least 5 times a week.

In some embodiments, the paclitaxel is administered at an amount of about 360 mg/m² in combination with Compound A at least 5 times a week.

In some embodiments, the paclitaxel is administered at an amount of about 365 mg/m² in combination with Compound A at least 5 times a week.

In some embodiments, the paclitaxel is administered at an amount of about 370 mg/m² in combination with Compound A at least 5 times a week.

In some embodiments, the paclitaxel is administered at an amount of about 375 mg/m² in combination with Compound A at least 5 times a week.

In some embodiments, the paclitaxel is administered at an amount of about 380 mg/m² in combination with Compound A at least 5 times a week.

In some embodiments, the paclitaxel is administered at an amount of about 385 mg/m² in combination with Compound A at least 5 times a week.

In some embodiments, the paclitaxel is administered at an amount of about 390 mg/m² in combination with Compound A at least 5 times a week.

In some embodiments, the paclitaxel is administered at an amount of about 395 mg/m² in combination with Compound A at least 5 times a week.

In some embodiments, the paclitaxel is administered at an amount of about 400 mg/m² in combination with Compound A at least 5 times a week.

In some embodiments, Compound A is administered in combination with paclitaxel at least 5 times a week.

In some embodiments, Compound A is administered at an amount of about 1 mg to about 500 mg in combination with paclitaxel at least 5 times a week.

In some embodiments, Compound A is administered at about 1 mg to about 400 mg in combination with paclitaxel at least 5 times a week.

In some embodiments, Compound A is administered at about 1 mg to about 300 mg in combination with paclitaxel at least 5 times a week.

In some embodiments, Compound A is administered at about 5 mg to about 200 mg in combination with paclitaxel at least 5 times a week.

In some embodiments, Compound A is administered at about 10 mg to about 100 mg in combination with paclitaxel at least 5 times a week.

In some embodiments, Compound A is administered at about 10 mg to about 95 mg in combination with paclitaxel at least 5 times a week.

In some embodiments, Compound A is administered at about 10 mg to about 90 mg in combination with paclitaxel at least 5 times a week.

In some embodiments, Compound A is administered at about 10 mg to about 85 mg in combination with paclitaxel at least 5 times a week.

In some embodiments, Compound A is administered at about 10 mg to about 80 mg in combination with paclitaxel at least 5 times a week.

In some embodiments, Compound A is administered at about 10 mg to about 75 mg in combination with paclitaxel at least 5 times a week.

In some embodiments, Compound A is administered at about 10 mg to about 70 mg in combination with paclitaxel at least 5 times a week.

In some embodiments, Compound A is administered at about 10 mg to about 65 mg in combination with paclitaxel at least 5 times a week.

In some embodiments, Compound A is administered at about 10 mg to about 60 mg in combination with paclitaxel at least 5 times a week.

In some embodiments, Compound A is administered at about 10 mg to about 55 mg in combination with paclitaxel at least 5 times a week.

In some embodiments, Compound A is administered at about 10 mg to about 50 mg in combination with paclitaxel at least 5 times a week.

In some embodiments, Compound A is administered at about 10 mg to about 45 mg in combination with paclitaxel at least 5 times a week.

In some embodiments, Compound A is administered at about 10 mg to about 40 mg in combination with paclitaxel at least 5 times a week.

In some embodiments, Compound A is administered at about 10 mg to about 35 mg in combination with paclitaxel at least 5 times a week.

In some embodiments, Compound A is administered at about 10 mg to about 30 mg in combination with paclitaxel at least 5 times a week.

In some embodiments, Compound A is administered at about 10 mg to about 25 mg in combination with paclitaxel at least 5 times a week.

In some embodiments, Compound A is administered at about 10 mg to about 20 mg in combination with paclitaxel at least 5 times a week.

In some embodiments, Compound A is administered at about 10 mg to about 15 mg in combination with paclitaxel at least 5 times a week.

In some embodiments, Compound A is administered at about 15 mg to about 50 mg in combination with paclitaxel at least 5 times a week.

In some embodiments, Compound A is administered at about 15 mg to about 45 mg in combination with paclitaxel at least 5 times a week.

In some embodiments, Compound A is administered at about 15 mg to about 40 mg in combination with paclitaxel at least 5 times a week.

In some embodiments, Compound A is administered at about 15 mg to about 35 mg in combination with paclitaxel at least 5 times a week.

In some embodiments, Compound A is administered at about 15 mg to about 30 mg in combination with paclitaxel at least 5 times a week.

In some embodiments, Compound A is administered at about 15 mg to about 25 mg in combination with paclitaxel at least 5 times a week.

In some embodiments, Compound A is administered at about 15 mg to about 20 mg in combination with paclitaxel at least 5 times a week.

In some embodiments, Compound A is administered at about 5 mg in combination with paclitaxel at least 5 times a week.

In some embodiments, Compound A is administered at about 10 mg in combination with paclitaxel at least 5 times a week.

In some embodiments, Compound A is administered at about 15 mg in combination with paclitaxel at least 5 times a week.

In some embodiments, Compound A is administered at about 20 mg in combination with paclitaxel at least 5 times a week.

In some embodiments, Compound A is administered at about 25 mg in combination with paclitaxel at least 5 times a week.

In some embodiments, Compound A is administered at about 30 mg in combination with paclitaxel at least 5 times a week.

In some embodiments, Compound A is administered at about 35 mg in combination with paclitaxel at least 5 times a week.

In some embodiments, Compound A is administered at about 40 mg in combination with paclitaxel at least 5 times a week.

In some embodiments, Compound A is administered at about 45 mg in combination with paclitaxel at least 5 times a week.

In some embodiments, Compound A is administered at about 50 mg in combination with paclitaxel at least 5 times a week.

In some embodiments, the paclitaxel is administered in combination with Compound A at least 5 times a week, wherein the at least 5 days are consecutive.

In some embodiments, the paclitaxel is administered in combination with Compound A at least 6 times a week.

In some embodiments, the paclitaxel is administered at an amount of about 100 mg/m² to about 400 mg/m² in combination with Compound A at least 6 times a week.

In some embodiments, the paclitaxel is administered at an amount of about 100 mg/m² to about 350 mg/m² in combination with Compound A at least 6 times a week.

In some embodiments, the paclitaxel is administered at an amount of about 100 mg/m² to about 300 mg/m² in combination with Compound A at least 6 times a week.

In some embodiments, the paclitaxel is administered at an amount of about 150 mg/m² to about 250 mg/m² in combination with Compound A at least 6 times a week.

In some embodiments, the paclitaxel is administered at an amount of about 100 mg/m² to about 245 mg/m² in combination with Compound A at least 6 times a week.

In some embodiments, the paclitaxel is administered at an amount of about 100 mg/m² to about 240 mg/m² in combination with Compound A at least 6 times a week.

In some embodiments, the paclitaxel is administered at an amount of about 100 mg/m² to about 235 mg/m² in combination with Compound A at least 6 times a week.

In some embodiments, the paclitaxel is administered at an amount of about 100 mg/m² to about 230 mg/m² in combination with Compound A at least 6 times a week.

In some embodiments, the paclitaxel is administered at an amount of about 100 mg/m² to about 225 mg/m² in combination with Compound A at least 6 times a week.

In some embodiments, the paclitaxel is administered at an amount of about 100 mg/m² to about 220 mg/m² in combination with Compound A at least 6 times a week.

In some embodiments, the paclitaxel is administered at an amount of about 100 mg/m² to about 215 mg/m² in combination with Compound A at least 6 times a week.

In some embodiments, the paclitaxel is administered at an amount of about 100 mg/m² to about 210 mg/m² in combination with Compound A at least 6 times a week.

In some embodiments, the paclitaxel is administered at an amount of about 100 mg/m² to about 205 mg/m² in combination with Compound A at least 6 times a week.

In some embodiments, the paclitaxel is administered at an amount of about 100 mg/m² to about 200 mg/m² in combination with Compound A at least 6 times a week.

In some embodiments, the paclitaxel is administered at an amount of about 100 mg/m² to about 195 mg/m² in combination with Compound A at least 6 times a week.

In some embodiments, the paclitaxel is administered at an amount of about 100 mg/m² to about 190 mg/m² in combination with Compound A at least 6 times a week.

In some embodiments, the paclitaxel is administered at an amount of about 100 mg/m² to about 185 mg/m² in combination with Compound A at least 6 times a week.

In some embodiments, the paclitaxel is administered at an amount of about 100 mg/m² to about 180 mg/m² in combination with Compound A at least 6 times a week.

In some embodiments, the paclitaxel is administered at an amount of about 100 mg/m² to about 175 mg/m² in combination with Compound A at least 6 times a week.

In some embodiments, the paclitaxel is administered at an amount of about 100 mg/m² to about 170 mg/m² in combination with Compound A at least 6 times a week.

In some embodiments, the paclitaxel is administered at an amount of about 100 mg/m² to about 165 mg/m² in combination with Compound A at least 6 times a week.

In some embodiments, the paclitaxel is administered at an amount of about 100 mg/m² to about 160 mg/m² in combination with Compound A at least 6 times a week.

In some embodiments, the paclitaxel is administered at an amount of about 100 mg/m² to about 155 mg/m² in combination with Compound A at least 6 times a week.

In some embodiments, the paclitaxel is administered at an amount of about 100 mg/m² to about 150 mg/m² in combination with Compound A at least 6 times a week.

In some embodiments, the paclitaxel is administered at an amount of about 100 mg/m² to about 145 mg/m² in combination with Compound A at least 6 times a week.

In some embodiments, the paclitaxel is administered at an amount of about 100 mg/m² to about 140 mg/m² in combination with Compound A at least 6 times a week.

In some embodiments, the paclitaxel is administered at an amount of about 100 mg/m² to about 135 mg/m² in combination with Compound A at least 6 times a week.

In some embodiments, the paclitaxel is administered at an amount of about 100 mg/m² to about 130 mg/m² in combination with Compound A at least 6 times a week.

In some embodiments, the paclitaxel is administered at an amount of about 100 mg/m² to about 125 mg/m² in combination with Compound A at least 6 times a week.

In some embodiments, the paclitaxel is administered at an amount of about 100 mg/m² to about 120 mg/m² in combination with Compound A at least 6 times a week.

In some embodiments, the paclitaxel is administered at an amount of about 100 mg/m² to about 115 mg/m² in combination with Compound A at least 6 times a week.

In some embodiments, the paclitaxel is administered at an amount of about 100 mg/m² to about 110 mg/m² in combination with Compound A at least 6 times a week.

In some embodiments, the paclitaxel is administered at an amount of about 100 mg/m² to about 105 mg/m² in combination with Compound A at least 6 times a week.

In some embodiments, the paclitaxel is administered at an amount of about 150 mg/m² to about 350 mg/m² in combination with Compound A at least 6 times a week.

In some embodiments, the paclitaxel is administered at an amount of about 150 mg/m² to about 300 mg/m² in combination with Compound A at least 6 times a week.

In some embodiments, the paclitaxel is administered at an amount of about 150 mg/m² to about 250 mg/m² in combination with Compound A at least 6 times a week.

In some embodiments, the paclitaxel is administered at an amount of about 150 mg/m² to about 245 mg/m² in combination with Compound A at least 6 times a week.

In some embodiments, the paclitaxel is administered at an amount of about 150 mg/m² to about 240 mg/m² in combination with Compound A at least 6 times a week.

In some embodiments, the paclitaxel is administered at an amount of about 150 mg/m² to about 235 mg/m² in combination with Compound A at least 6 times a week.

In some embodiments, the paclitaxel is administered at an amount of about 150 mg/m² to about 230 mg/m² in combination with Compound A at least 6 times a week.

In some embodiments, the paclitaxel is administered at an amount of about 150 mg/m² to about 225 mg/m² in combination with Compound A at least 6 times a week.

In some embodiments, the paclitaxel is administered at an amount of about 150 mg/m² to about 220 mg/m² in combination with Compound A at least 6 times a week.

In some embodiments, the paclitaxel is administered at an amount of about 150 mg/m² to about 215 mg/m² in combination with Compound A at least 6 times a week.

In some embodiments, the paclitaxel is administered at an amount of about 150 mg/m² to about 210 mg/m² in combination with Compound A at least 6 times a week.

In some embodiments, the paclitaxel is administered at an amount of about 150 mg/m² to about 205 mg/m² in combination with Compound A at least 6 times a week.

In some embodiments, the paclitaxel is administered at an amount of about 150 mg/m² to about 200 mg/m² in combination with Compound A at least 6 times a week.

In some embodiments, the paclitaxel is administered at an amount of about 150 mg/m² to about 195 mg/m² in combination with Compound A at least 6 times a week.

In some embodiments, the paclitaxel is administered at an amount of about 150 mg/m² to about 190 mg/m² in combination with Compound A at least 6 times a week.

In some embodiments, the paclitaxel is administered at an amount of about 150 mg/m² to about 185 mg/m² in combination with Compound A at least 6 times a week.

In some embodiments, the paclitaxel is administered at an amount of about 150 mg/m² to about 180 mg/m² in combination with Compound A at least 6 times a week.

In some embodiments, the paclitaxel is administered at an amount of about 150 mg/m² to about 175 mg/m² in combination with Compound A at least 6 times a week.

In some embodiments, the paclitaxel is administered at an amount of about 150 mg/m² to about 170 mg/m² in combination with Compound A at least 6 times a week.

In some embodiments, the paclitaxel is administered at an amount of about 150 mg/m² to about 165 mg/m² in combination with Compound A at least 6 times a week.

In some embodiments, the paclitaxel is administered at an amount of about 150 mg/m² to about 160 mg/m² in combination with Compound A at least 6 times a week.

In some embodiments, the paclitaxel is administered at an amount of about 150 mg/m² to about 155 mg/m² in combination with Compound A at least 6 times a week.

In some embodiments, the paclitaxel is administered at an amount of about 200 mg/m² to about 300 mg/m² in combination with Compound A at least 6 times a week.

In some embodiments, the paclitaxel is administered at an amount of about 200 mg/m² to about 295 mg/m² in combination with Compound A at least 6 times a week.

In some embodiments, the paclitaxel is administered at an amount of about 200 mg/m² to about 290 mg/m² in combination with Compound A at least 6 times a week.

In some embodiments, the paclitaxel is administered at an amount of about 200 mg/m² to about 285 mg/m² in combination with Compound A at least 6 times a week.

In some embodiments, the paclitaxel is administered at an amount of about 200 mg/m² to about 280 mg/m² in combination with Compound A at least 6 times a week.

In some embodiments, the paclitaxel is administered at an amount of about 200 mg/m² to about 275 mg/m² in combination with Compound A at least 6 times a week.

In some embodiments, the paclitaxel is administered at an amount of about 200 mg/m² to about 270 mg/m² in combination with Compound A at least 6 times a week.

In some embodiments, the paclitaxel is administered at an amount of about 200 mg/m² to about 265 mg/m² in combination with Compound A at least 6 times a week.

In some embodiments, the paclitaxel is administered at an amount of about 200 mg/m² to about 260 mg/m² in combination with Compound A at least 6 times a week.

In some embodiments, the paclitaxel is administered at an amount of about 200 mg/m² to about 255 mg/m² in combination with Compound A at least 6 times a week.

In some embodiments, the paclitaxel is administered at an amount of about 200 mg/m² to about 250 mg/m² in combination with Compound A at least 6 times a week.

In some embodiments, the paclitaxel is administered at an amount of about 200 mg/m² to about 245 mg/m² in combination with Compound A at least 6 times a week.

In some embodiments, the paclitaxel is administered at an amount of about 200 mg/m² to about 240 mg/m² in combination with Compound A at least 6 times a week.

In some embodiments, the paclitaxel is administered at an amount of about 200 mg/m² to about 235 mg/m² in combination with Compound A at least 6 times a week.

In some embodiments, the paclitaxel is administered at an amount of about 200 mg/m² to about 230 mg/m² in combination with Compound A at least 6 times a week.

In some embodiments, the paclitaxel is administered at an amount of about 200 mg/m² to about 225 mg/m² in combination with Compound A at least 6 times a week.

In some embodiments, the paclitaxel is administered at an amount of about 200 mg/m² to about 220 mg/m² in combination with Compound A at least 6 times a week.

In some embodiments, the paclitaxel is administered at an amount of about 200 mg/m² to about 215 mg/m² in combination with Compound A at least 6 times a week.

In some embodiments, the paclitaxel is administered at an amount of about 200 mg/m² to about 210 mg/m² in combination with Compound A at least 6 times a week.

In some embodiments, the paclitaxel is administered at an amount of about 200 mg/m² to about 205 mg/m² in combination with Compound A at least 6 times a week.

In some embodiments, the paclitaxel is administered at an amount of about 250 mg/m² to about 350 mg/m² in combination with Compound A at least 6 times a week.

In some embodiments, the paclitaxel is administered at an amount of about 250 mg/m² to about 300 mg/m² in combination with Compound A at least 6 times a week.

In some embodiments, the paclitaxel is administered at an amount of about 120 mg/m² to about 235 mg/m² in combination with Compound A at least 6 times a week.

In some embodiments, the paclitaxel is administered at an amount of about 120 mg/m² to about 230 mg/m² in combination with Compound A at least 6 times a week.

In some embodiments, the paclitaxel is administered at an amount of about 120 mg/m² to about 225 mg/m² in combination with Compound A at least 6 times a week.

In some embodiments, the paclitaxel is administered at an amount of about 120 mg/m² to about 220 mg/m² in combination with Compound A at least 6 times a week.

In some embodiments, the paclitaxel is administered at an amount of about 120 mg/m² to about 215 mg/m² in combination with Compound A at least 6 times a week.

In some embodiments, the paclitaxel is administered at an amount of about 120 mg/m² to about 210 mg/m² in combination with Compound A at least 6 times a week.

In some embodiments, the paclitaxel is administered at an amount of about 120 mg/m² to about 205 mg/m² in combination with Compound A at least 6 times a week.

In some embodiments, the paclitaxel is administered at an amount of about 120 mg/m² to about 200 mg/m² in combination with Compound A at least 6 times a week.

In some embodiments, the paclitaxel is administered at an amount of about 120 mg/m² to about 195 mg/m² in combination with Compound A at least 6 times a week.

In some embodiments, the paclitaxel is administered at an amount of about 120 mg/m² to about 190 mg/m² in combination with Compound A at least 6 times a week.

In some embodiments, the paclitaxel is administered at an amount of about 120 mg/m² to about 185 mg/m² in combination with Compound A at least 6 times a week.

In some embodiments, the paclitaxel is administered at an amount of about 120 mg/m² to about 180 mg/m² in combination with Compound A at least 6 times a week.

In some embodiments, the paclitaxel is administered at an amount of about 120 mg/m² to about 175 mg/m² in combination with Compound A at least 6 times a week.

In some embodiments, the paclitaxel is administered at an amount of about 120 mg/m² to about 170 mg/m² in combination with Compound A at least 6 times a week.

In some embodiments, the paclitaxel is administered at an amount of about 120 mg/m² to about 165 mg/m² in combination with Compound A at least 6 times a week.

In some embodiments, the paclitaxel is administered at an amount of about 120 mg/m² to about 160 mg/m² in combination with Compound A at least 6 times a week.

In some embodiments, the paclitaxel is administered at an amount of about 120 mg/m² to about 155 mg/m² in combination with Compound A at least 6 times a week.

In some embodiments, the paclitaxel is administered at an amount of about 120 mg/m² to about 150 mg/m² in combination with Compound A at least 6 times a week.

In some embodiments, the paclitaxel is administered at an amount of about 120 mg/m² to about 145 mg/m² in combination with Compound A at least 6 times a week.

In some embodiments, the paclitaxel is administered at an amount of about 120 mg/m² to about 140 mg/m² in combination with Compound A at least 6 times a week.

In some embodiments, the paclitaxel is administered at an amount of about 120 mg/m² to about 135 mg/m² in combination with Compound A at least 6 times a week.

In some embodiments, the paclitaxel is administered at an amount of about 120 mg/m² to about 130 mg/m² in combination with Compound A at least 6 times a week.

In some embodiments, the paclitaxel is administered at an amount of about 120 mg/m² to about 125 mg/m² in combination with Compound A at least 6 times a week.

In some embodiments, the paclitaxel is administered at an amount of about 125 mg/m² to about 235 mg/m² in combination with Compound A at least 6 times a week.

In some embodiments, the paclitaxel is administered at an amount of about 125 mg/m² to about 230 mg/m² in combination with Compound A at least 6 times a week.

In some embodiments, the paclitaxel is administered at an amount of about 125 mg/m² to about 225 mg/m² in combination with Compound A at least 6 times a week.

In some embodiments, the paclitaxel is administered at an amount of about 125 mg/m² to about 220 mg/m² in combination with Compound A at least 6 times a week.

In some embodiments, the paclitaxel is administered at an amount of about 125 mg/m² to about 215 mg/m² in combination with Compound A at least 6 times a week.

In some embodiments, the paclitaxel is administered at an amount of about 125 mg/m² to about 210 mg/m² in combination with Compound A at least 6 times a week.

In some embodiments, the paclitaxel is administered at an amount of about 125 mg/m² to about 205 mg/m² in combination with Compound A at least 6 times a week.

In some embodiments, the paclitaxel is administered at an amount of about 125 mg/m² to about 200 mg/m² in combination with Compound A at least 6 times a week.

In some embodiments, the paclitaxel is administered at an amount of about 125 mg/m² to about 195 mg/m² in combination with Compound A at least 6 times a week.

In some embodiments, the paclitaxel is administered at an amount of about 125 mg/m² to about 190 mg/m² in combination with Compound A at least 6 times a week.

In some embodiments, the paclitaxel is administered at an amount of about 125 mg/m² to about 185 mg/m² in combination with Compound A at least 6 times a week.

In some embodiments, the paclitaxel is administered at an amount of about 125 mg/m² to about 180 mg/m² in combination with Compound A at least 6 times a week.

In some embodiments, the paclitaxel is administered at an amount of about 125 mg/m² to about 175 mg/m² in combination with Compound A at least 6 times a week.

In some embodiments, the paclitaxel is administered at an amount of about 125 mg/m² to about 170 mg/m² in combination with Compound A at least 6 times a week.

In some embodiments, the paclitaxel is administered at an amount of about 125 mg/m² to about 165 mg/m² in combination with Compound A at least 6 times a week.

In some embodiments, the paclitaxel is administered at an amount of about 125 mg/m² to about 160 mg/m² in combination with Compound A at least 6 times a week.

In some embodiments, the paclitaxel is administered at an amount of about 125 mg/m² to about 155 mg/m² in combination with Compound A at least 6 times a week.

In some embodiments, the paclitaxel is administered at an amount of about 125 mg/m² to about 150 mg/m² in combination with Compound A at least 6 times a week.

In some embodiments, the paclitaxel is administered at an amount of about 125 mg/m² to about 145 mg/m² in combination with Compound A at least 6 times a week.

In some embodiments, the paclitaxel is administered at an amount of about 125 mg/m² to about 140 mg/m² in combination with Compound A at least 6 times a week.

In some embodiments, the paclitaxel is administered at an amount of about 125 mg/m² to about 135 mg/m² in combination with Compound A at least 6 times a week.

In some embodiments, the paclitaxel is administered at an amount of about 125 mg/m² to about 130 mg/m² in combination with Compound A at least 6 times a week.

In some embodiments, the paclitaxel is administered at an amount of about 130 mg/m² to about 235 mg/m² in combination with Compound A at least 6 times a week.

In some embodiments, the paclitaxel is administered at an amount of about 130 mg/m² to about 230 mg/m² in combination with Compound A at least 6 times a week.

In some embodiments, the paclitaxel is administered at an amount of about 130 mg/m² to about 225 mg/m² in combination with Compound A at least 6 times a week.

In some embodiments, the paclitaxel is administered at an amount of about 130 mg/m² to about 220 mg/m² in combination with Compound A at least 6 times a week.

In some embodiments, the paclitaxel is administered at an amount of about 130 mg/m² to about 215 mg/m² in combination with Compound A at least 6 times a week.

In some embodiments, the paclitaxel is administered at an amount of about 130 mg/m² to about 210 mg/m² in combination with Compound A at least 6 times a week.

In some embodiments, the paclitaxel is administered at an amount of about 130 mg/m² to about 205 mg/m² in combination with Compound A at least 6 times a week.

In some embodiments, the paclitaxel is administered at an amount of about 130 mg/m² to about 200 mg/m² in combination with Compound A at least 6 times a week.

In some embodiments, the paclitaxel is administered at an amount of about 130 mg/m² to about 195 mg/m² in combination with Compound A at least 6 times a week.

In some embodiments, the paclitaxel is administered at an amount of about 130 mg/m² to about 190 mg/m² in combination with Compound A at least 6 times a week.

In some embodiments, the paclitaxel is administered at an amount of about 130 mg/m² to about 185 mg/m² in combination with Compound A at least 6 times a week.

In some embodiments, the paclitaxel is administered at an amount of about 130 mg/m² to about 180 mg/m² in combination with Compound A at least 6 times a week.

In some embodiments, the paclitaxel is administered at an amount of about 130 mg/m² to about 175 mg/m² in combination with Compound A at least 6 times a week.

In some embodiments, the paclitaxel is administered at an amount of about 130 mg/m² to about 170 mg/m² in combination with Compound A at least 6 times a week.

In some embodiments, the paclitaxel is administered at an amount of about 130 mg/m² to about 165 mg/m² in combination with Compound A at least 6 times a week.

In some embodiments, the paclitaxel is administered at an amount of about 130 mg/m² to about 160 mg/m² in combination with Compound A at least 6 times a week.

In some embodiments, the paclitaxel is administered at an amount of about 130 mg/m² to about 155 mg/m² in combination with Compound A at least 6 times a week.

In some embodiments, the paclitaxel is administered at an amount of about 130 mg/m² to about 150 mg/m² in combination with Compound A at least 6 times a week.

In some embodiments, the paclitaxel is administered at an amount of about 130 mg/m² to about 145 mg/m² in combination with Compound A at least 6 times a week.

In some embodiments, the paclitaxel is administered at an amount of about 130 mg/m² to about 140 mg/m² in combination with Compound A at least 6 times a week.

In some embodiments, the paclitaxel is administered at an amount of about 130 mg/m² to about 135 mg/m² in combination with Compound A at least 6 times a week.

In some embodiments, the paclitaxel is administered at an amount of about 100 mg/m² in combination with Compound A at least 6 times a week.

In some embodiments, the paclitaxel is administered at an amount of about 105 mg/m² in combination with Compound A at least 6 times a week.

In some embodiments, the paclitaxel is administered at an amount of about 110 mg/m² in combination with Compound A at least 6 times a week.

In some embodiments, the paclitaxel is administered at an amount of about 115 mg/m² in combination with Compound A at least 6 times a week.

In some embodiments, the paclitaxel is administered at an amount of about 120 mg/m² in combination with Compound A at least 6 times a week.

In some embodiments, the paclitaxel is administered at an amount of about 125 mg/m² in combination with Compound A at least 6 times a week.

In some embodiments, the paclitaxel is administered at an amount of about 130 mg/m² in combination with Compound A at least 6 times a week.

In some embodiments, the paclitaxel is administered at an amount of about 135 mg/m² in combination with Compound A at least 6 times a week.

In some embodiments, the paclitaxel is administered at an amount of about 140 mg/m² in combination with Compound A at least 6 times a week.

In some embodiments, the paclitaxel is administered at an amount of about 145 mg/m² in combination with Compound A at least 6 times a week.

In some embodiments, the paclitaxel is administered at an amount of about 150 mg/m² in combination with Compound A at least 6 times a week.

In some embodiments, the paclitaxel is administered at an amount of about 155 mg/m² in combination with Compound A at least 6 times a week.

In some embodiments, the paclitaxel is administered at an amount of about 160 mg/m² in combination with Compound A at least 6 times a week.

In some embodiments, the paclitaxel is administered at an amount of about 165 mg/m² in combination with Compound A at least 6 times a week.

In some embodiments, the paclitaxel is administered at an amount of about 170 mg/m² in combination with Compound A at least 6 times a week.

In some embodiments, the paclitaxel is administered at an amount of about 175 mg/m² in combination with Compound A at least 6 times a week.

In some embodiments, the paclitaxel is administered at an amount of about 180 mg/m² in combination with Compound A at least 6 times a week.

In some embodiments, the paclitaxel is administered at an amount of about 185 mg/m² in combination with Compound A at least 6 times a week.

In some embodiments, the paclitaxel is administered at an amount of about 190 mg/m² in combination with Compound A at least 6 times a week.

In some embodiments, the paclitaxel is administered at an amount of about 195 mg/m² in combination with Compound A at least 6 times a week.

In some embodiments, the paclitaxel is administered at an amount of about 200 mg/m² in combination with Compound A at least 6 times a week.

In some embodiments, the paclitaxel is administered at an amount of about 205 mg/m² in combination with Compound A at least 6 times a week.

In some embodiments, the paclitaxel is administered at an amount of about 210 mg/m² in combination with Compound A at least 6 times a week.

In some embodiments, the paclitaxel is administered at an amount of about 215 mg/m² in combination with Compound A at least 6 times a week.

In some embodiments, the paclitaxel is administered at an amount of about 220 mg/m² in combination with Compound A at least 6 times a week.

In some embodiments, the paclitaxel is administered at an amount of about 225 mg/m² in combination with Compound A at least 6 times a week.

In some embodiments, the paclitaxel is administered at an amount of about 230 mg/m² in combination with Compound A at least 6 times a week.

In some embodiments, the paclitaxel is administered at an amount of about 235 mg/m² in combination with Compound A at least 6 times a week.

In some embodiments, the paclitaxel is administered at an amount of about 240 mg/m² in combination with Compound A at least 6 times a week.

In some embodiments, the paclitaxel is administered at an amount of about 245 mg/m² in combination with Compound A at least 6 times a week.

In some embodiments, the paclitaxel is administered at an amount of about 250 mg/m² in combination with Compound A at least 6 times a week.

In some embodiments, the paclitaxel is administered at an amount of about 255 mg/m² in combination with Compound A at least 6 times a week.

In some embodiments, the paclitaxel is administered at an amount of about 260 mg/m² in combination with Compound A at least 6 times a week.

In some embodiments, the paclitaxel is administered at an amount of about 265 mg/m² in combination with Compound A at least 6 times a week.

In some embodiments, the paclitaxel is administered at an amount of about 270 mg/m² in combination with Compound A at least 6 times a week.

In some embodiments, the paclitaxel is administered at an amount of about 275 mg/m² in combination with Compound A at least 6 times a week.

In some embodiments, the paclitaxel is administered at an amount of about 280 mg/m² in combination with Compound A at least 6 times a week.

In some embodiments, the paclitaxel is administered at an amount of about 285 mg/m² in combination with Compound A at least 6 times a week.

In some embodiments, the paclitaxel is administered at an amount of about 290 mg/m² in combination with Compound A at least 6 times a week.

In some embodiments, the paclitaxel is administered at an amount of about 295 mg/m² in combination with Compound A at least 6 times a week.

In some embodiments, the paclitaxel is administered at an amount of about 300 mg/m² in combination with Compound A at least 6 times a week.

In some embodiments, the paclitaxel is administered at an amount of about 305 mg/m² in combination with Compound A at least 6 times a week.

In some embodiments, the paclitaxel is administered at an amount of about 310 mg/m² in combination with Compound A at least 6 times a week.

In some embodiments, the paclitaxel is administered at an amount of about 315 mg/m² in combination with Compound A at least 6 times a week.

In some embodiments, the paclitaxel is administered at an amount of about 320 mg/m² in combination with Compound A at least 6 times a week.

In some embodiments, the paclitaxel is administered at an amount of about 325 mg/m² in combination with Compound A at least 6 times a week.

In some embodiments, the paclitaxel is administered at an amount of about 330 mg/m² in combination with Compound A at least 6 times a week.

In some embodiments, the paclitaxel is administered at an amount of about 335 mg/m² in combination with Compound A at least 6 times a week.

In some embodiments, the paclitaxel is administered at an amount of about 340 mg/m² in combination with Compound A at least 6 times a week.

In some embodiments, the paclitaxel is administered at an amount of about 345 mg/m² in combination with Compound A at least 6 times a week.

In some embodiments, the paclitaxel is administered at an amount of about 350 mg/m² in combination with Compound A at least 6 times a week.

In some embodiments, the paclitaxel is administered at an amount of about 355 mg/m² in combination with Compound A at least 6 times a week.

In some embodiments, the paclitaxel is administered at an amount of about 360 mg/m² in combination with Compound A at least 6 times a week.

In some embodiments, the paclitaxel is administered at an amount of about 365 mg/m² in combination with Compound A at least 6 times a week.

In some embodiments, the paclitaxel is administered at an amount of about 370 mg/m² in combination with Compound A at least 6 times a week.

In some embodiments, the paclitaxel is administered at an amount of about 375 mg/m² in combination with Compound A at least 6 times a week.

In some embodiments, the paclitaxel is administered at an amount of about 380 mg/m² in combination with Compound A at least 6 times a week.

In some embodiments, the paclitaxel is administered at an amount of about 385 mg/m² in combination with Compound A at least 6 times a week.

In some embodiments, the paclitaxel is administered at an amount of about 390 mg/m² in combination with Compound A at least 6 times a week.

In some embodiments, the paclitaxel is administered at an amount of about 395 mg/m² in combination with Compound A at least 6 times a week.

In some embodiments, the paclitaxel is administered at an amount of about 400 mg/m² in combination with Compound A at least 6 times a week.

In some embodiments, Compound A is administered in combination with paclitaxel at least 6 times a week.

In some embodiments, Compound A is administered at an amount of about 1 mg to about 500 mg in combination with paclitaxel at least 6 times a week.

In some embodiments, Compound A is administered at about 1 mg to about 400 mg in combination with paclitaxel at least 6 times a week.

In some embodiments, Compound A is administered at about 1 mg to about 300 mg in combination with paclitaxel at least 6 times a week.

In some embodiments, Compound A is administered at about 5 mg to about 200 mg in combination with paclitaxel at least 6 times a week.

In some embodiments, Compound A is administered at about 10 mg to about 100 mg in combination with paclitaxel at least 6 times a week.

In some embodiments, Compound A is administered at about 10 mg to about 95 mg in combination with paclitaxel at least 6 times a week.

In some embodiments, Compound A is administered at about 10 mg to about 90 mg in combination with paclitaxel at least 6 times a week.

In some embodiments, Compound A is administered at about 10 mg to about 85 mg in combination with paclitaxel at least 6 times a week.

In some embodiments, Compound A is administered at about 10 mg to about 80 mg in combination with paclitaxel at least 6 times a week.

In some embodiments, Compound A is administered at about 10 mg to about 75 mg in combination with paclitaxel at least 6 times a week.

In some embodiments, Compound A is administered at about 10 mg to about 70 mg in combination with paclitaxel at least 6 times a week.

In some embodiments, Compound A is administered at about 10 mg to about 65 mg in combination with paclitaxel at least 6 times a week.

In some embodiments, Compound A is administered at about 10 mg to about 60 mg in combination with paclitaxel at least 6 times a week.

In some embodiments, Compound A is administered at about 10 mg to about 55 mg in combination with paclitaxel at least 6 times a week.

In some embodiments, Compound A is administered at about 10 mg to about 50 mg in combination with paclitaxel at least 6 times a week.

In some embodiments, Compound A is administered at about 10 mg to about 45 mg in combination with paclitaxel at least 6 times a week.

In some embodiments, Compound A is administered at about 10 mg to about 40 mg in combination with paclitaxel at least 6 times a week.

In some embodiments, Compound A is administered at about 10 mg to about 35 mg in combination with paclitaxel at least 6 times a week.

In some embodiments, Compound A is administered at about 10 mg to about 30 mg in combination with paclitaxel at least 6 times a week.

In some embodiments, Compound A is administered at about 10 mg to about 25 mg in combination with paclitaxel at least 6 times a week.

In some embodiments, Compound A is administered at about 10 mg to about 20 mg in combination with paclitaxel at least 6 times a week.

In some embodiments, Compound A is administered at about 10 mg to about 15 mg in combination with paclitaxel at least 6 times a week.

In some embodiments, Compound A is administered at about 15 mg to about 50 mg in combination with paclitaxel at least 6 times a week.

In some embodiments, Compound A is administered at about 15 mg to about 45 mg in combination with paclitaxel at least 6 times a week.

In some embodiments, Compound A is administered at about 15 mg to about 40 mg in combination with paclitaxel at least 6 times a week.

In some embodiments, Compound A is administered at about 15 mg to about 35 mg in combination with paclitaxel at least 6 times a week.

In some embodiments, Compound A is administered at about 15 mg to about 30 mg in combination with paclitaxel at least 6 times a week.

In some embodiments, Compound A is administered at about 15 mg to about 25 mg in combination with paclitaxel at least 6 times a week.

In some embodiments, Compound A is administered at about 15 mg to about 20 mg in combination with paclitaxel at least 6 times a week.

In some embodiments, Compound A is administered at about 5 mg in combination with paclitaxel at least 6 times a week.

In some embodiments, Compound A is administered at about 10 mg in combination with paclitaxel at least 6 times a week.

In some embodiments, Compound A is administered at about 15 mg in combination with paclitaxel at least 6 times a week.

In some embodiments, Compound A is administered at about 20 mg in combination with paclitaxel at least 6 times a week.

In some embodiments, Compound A is administered at about 25 mg in combination with paclitaxel at least 6 times a week.

In some embodiments, Compound A is administered at about 30 mg in combination with paclitaxel at least 6 times a week.

In some embodiments, Compound A is administered at about 35 mg in combination with paclitaxel at least 6 times a week.

In some embodiments, Compound A is administered at about 40 mg in combination with paclitaxel at least 6 times a week.

In some embodiments, Compound A is administered at about 45 mg in combination with paclitaxel at least 6 times a week.

In some embodiments, Compound A is administered at about 50 mg in combination with paclitaxel at least 6 times a week.

In some embodiments, the Compound A is administered in combination with paclitaxel at least 6 times a week, wherein the at least 6 days are consecutive.

In some embodiments, the paclitaxel is administered in combination with Compound A, wherein Compound A is administered prior to paclitaxel.

In some embodiments, Compound A is administered at about 15 mg in combination with paclitaxel administered at about 205 mg/m², wherein Compound A is administered prior to paclitaxel.

In some embodiments, Compound A is administered at about 15 mg in combination with paclitaxel administered at about 205 mg/m², wherein Compound A is administered one hour prior to paclitaxel.

In some embodiments, Compound A is administered at about 15 mg in combination with paclitaxel administered at about 205 mg/m², wherein Compound A is administered one hour prior to paclitaxel, wherein the combination is administered 3 days per week.

In some embodiments, Compound A is administered at about 15 mg in combination with paclitaxel administered at about 205 mg/m², wherein Compound A is administered one hour prior to paclitaxel, wherein the combination is administered on 3 consecutive days per week.

In some embodiments, Compound A is administered at about 15 mg in combination with paclitaxel administered at about 165 mg/m², wherein Compound A is administered prior to paclitaxel.

In some embodiments, Compound A is administered at about 15 mg in combination with paclitaxel administered at about 165 mg/m², wherein Compound A is administered one hour prior to paclitaxel.

In some embodiments, Compound A is administered at about 15 mg in combination with paclitaxel administered at about 165 mg/m², wherein Compound A is administered one hour prior to paclitaxel, wherein the combination is administered 3 days per week.

In some embodiments, Compound A is administered at about 15 mg in combination with paclitaxel administered at about 165 mg/m², wherein Compound A is administered one hour prior to paclitaxel, wherein the combination is administered on 3 consecutive days per week.

In some embodiments, Compound A is administered at about 15 mg in combination with paclitaxel administered at about 130 mg/m², wherein Compound A is administered prior to paclitaxel.

In some embodiments, Compound A is administered at about 15 mg in combination with paclitaxel administered at about 130 mg/m², wherein Compound A is administered one hour prior to paclitaxel.

In some embodiments, Compound A is administered at about 15 mg in combination with paclitaxel administered at about 130 mg/m², wherein Compound A is administered one hour prior to paclitaxel, wherein the combination is administered 3 days per week.

In some embodiments, Compound A is administered at about 15 mg in combination with paclitaxel administered at about 130 mg/m², wherein Compound A is administered one hour prior to paclitaxel, wherein the combination is administered on 3 consecutive days per week.

In some embodiments, the paclitaxel is administered at a first dose, followed by a second dose.

In some embodiments, the paclitaxel is administered at a first dose, followed by a second dose, wherein the first dose is reduced to the second dose.

In some embodiments, the paclitaxel is administered at a first dose at an amount of about 100 mg/m² to about 400 mg/m².

In some embodiments, the paclitaxel is administered at a first dose at an amount of about 100 mg/m² to about 350 mg/m².

In some embodiments, the paclitaxel is administered at a first dose at an amount of about 100 mg/m² to about 300 mg/m².

In some embodiments, the paclitaxel is administered at a first dose at an amount of about 150 mg/m² to about 250 mg/m².

In some embodiments, the paclitaxel is administered at a first dose at an amount of about 100 mg/m² to about 245 mg/m².

In some embodiments, the paclitaxel is administered at a first dose at an amount of about 100 mg/m² to about 240 mg/m².

In some embodiments, the paclitaxel is administered at a first dose at an amount of about 100 mg/m² to about 235 mg/m².

In some embodiments, the paclitaxel is administered at a first dose at an amount of about 100 mg/m² to about 230 mg/m².

In some embodiments, the paclitaxel is administered at a first dose at an amount of about 100 mg/m² to about 225 mg/m².

In some embodiments, the paclitaxel is administered at a first dose at an amount of about 100 mg/m² to about 220 mg/m².

In some embodiments, the paclitaxel is administered at a first dose at an amount of about 100 mg/m² to about 215 mg/m².

In some embodiments, the paclitaxel is administered at a first dose at an amount of about 100 mg/m² to about 210 mg/m².

In some embodiments, the paclitaxel is administered at a first dose at an amount of about 100 mg/m² to about 205 mg/m².

In some embodiments, the paclitaxel is administered at a first dose at an amount of about 100 mg/m² to about 200 mg/m².

In some embodiments, the paclitaxel is administered at a first dose at an amount of about 100 mg/m² to about 195 mg/m².

In some embodiments, the paclitaxel is administered at a first dose at an amount of about 100 mg/m² to about 190 mg/m².

In some embodiments, the paclitaxel is administered at a first dose at an amount of about 100 mg/m² to about 185 mg/m².

In some embodiments, the paclitaxel is administered at a first dose at an amount of about 100 mg/m² to about 180 mg/m².

In some embodiments, the paclitaxel is administered at a first dose at an amount of about 100 mg/m² to about 175 mg/m².

In some embodiments, the paclitaxel is administered at a first dose at an amount of about 100 mg/m² to about 170 mg/m².

In some embodiments, the paclitaxel is administered at a first dose at an amount of about 100 mg/m² to about 165 mg/m².

In some embodiments, the paclitaxel is administered at a first dose at an amount of about 100 mg/m² to about 160 mg/m².

In some embodiments, the paclitaxel is administered at a first dose at an amount of about 100 mg/m² to about 155 mg/m².

In some embodiments, the paclitaxel is administered at a first dose at an amount of about 100 mg/m² to about 150 mg/m².

In some embodiments, the paclitaxel is administered at a first dose at an amount of about 100 mg/m² to about 145 mg/m².

In some embodiments, the paclitaxel is administered at a first dose at an amount of about 100 mg/m² to about 140 mg/m².

In some embodiments, the paclitaxel is administered at a first dose at an amount of about 100 mg/m² to about 135 mg/m².

In some embodiments, the paclitaxel is administered at a first dose at an amount of about 100 mg/m² to about 130 mg/m².

In some embodiments, the paclitaxel is administered at a first dose at an amount of about 100 mg/m² to about 125 mg/m².

In some embodiments, the paclitaxel is administered at a first dose at an amount of about 100 mg/m² to about 120 mg/m².

In some embodiments, the paclitaxel is administered at a first dose at an amount of about 100 mg/m² to about 115 mg/m².

In some embodiments, the paclitaxel is administered at a first dose at an amount of about 100 mg/m² to about 110 mg/m².

In some embodiments, the paclitaxel is administered at a first dose at an amount of about 100 mg/m² to about 105 mg/m².

In some embodiments, the paclitaxel is administered at a first dose at an amount of about 150 mg/m² to about 350 mg/m².

In some embodiments, the paclitaxel is administered at a first dose at an amount of about 150 mg/m² to about 300 mg/m².

In some embodiments, the paclitaxel is administered at a first dose at an amount of about 150 mg/m² to about 250 mg/m².

In some embodiments, the paclitaxel is administered at a first dose at an amount of about 150 mg/m² to about 245 mg/m².

In some embodiments, the paclitaxel is administered at a first dose at an amount of about 150 mg/m² to about 240 mg/m².

In some embodiments, the paclitaxel is administered at a first dose at an amount of about 150 mg/m² to about 235 mg/m².

In some embodiments, the paclitaxel is administered at a first dose at an amount of about 150 mg/m² to about 230 mg/m².

In some embodiments, the paclitaxel is administered at a first dose at an amount of about 150 mg/m² to about 225 mg/m².

In some embodiments, the paclitaxel is administered at a first dose at an amount of about 150 mg/m² to about 220 mg/m².

In some embodiments, the paclitaxel is administered at a first dose at an amount of about 150 mg/m² to about 215 mg/m².

In some embodiments, the paclitaxel is administered at a first dose at an amount of about 150 mg/m² to about 210 mg/m².

In some embodiments, the paclitaxel is administered at a first dose at an amount of about 150 mg/m² to about 205 mg/m².

In some embodiments, the paclitaxel is administered at a first dose at an amount of about 150 mg/m² to about 200 mg/m².

In some embodiments, the paclitaxel is administered at a first dose at an amount of about 150 mg/m² to about 195 mg/m².

In some embodiments, the paclitaxel is administered at a first dose at an amount of about 150 mg/m² to about 190 mg/m².

In some embodiments, the paclitaxel is administered at a first dose at an amount of about 150 mg/m² to about 185 mg/m².

In some embodiments, the paclitaxel is administered at a first dose at an amount of about 150 mg/m² to about 180 mg/m².

In some embodiments, the paclitaxel is administered at a first dose at an amount of about 150 mg/m² to about 175 mg/m².

In some embodiments, the paclitaxel is administered at a first dose at an amount of about 150 mg/m² to about 170 mg/m².

In some embodiments, the paclitaxel is administered at a first dose at an amount of about 150 mg/m² to about 165 mg/m².

In some embodiments, the paclitaxel is administered at a first dose at an amount of about 150 mg/m² to about 160 mg/m².

In some embodiments, the paclitaxel is administered at a first dose at an amount of about 150 mg/m² to about 155 mg/m².

In some embodiments, the paclitaxel is administered at a first dose at an amount of about 200 mg/m² to about 300 mg/m².

In some embodiments, the paclitaxel is administered at a first dose at an amount of about 200 mg/m² to about 295 mg/m².

In some embodiments, the paclitaxel is administered at a first dose at an amount of about 200 mg/m² to about 290 mg/m².

In some embodiments, the paclitaxel is administered at a first dose at an amount of about 200 mg/m² to about 285 mg/m².

In some embodiments, the paclitaxel is administered at a first dose at an amount of about 200 mg/m² to about 280 mg/m².

In some embodiments, the paclitaxel is administered at a first dose at an amount of about 200 mg/m² to about 275 mg/m².

In some embodiments, the paclitaxel is administered at a first dose at an amount of about 200 mg/m² to about 270 mg/m².

In some embodiments, the paclitaxel is administered at a first dose at an amount of about 200 mg/m² to about 265 mg/m².

In some embodiments, the paclitaxel is administered at a first dose at an amount of about 200 mg/m² to about 260 mg/m².

In some embodiments, the paclitaxel is administered at a first dose at an amount of about 200 mg/m² to about 255 mg/m².

In some embodiments, the paclitaxel is administered at a first dose at an amount of about 200 mg/m² to about 250 mg/m².

In some embodiments, the paclitaxel is administered at a first dose at an amount of about 200 mg/m² to about 245 mg/m².

In some embodiments, the paclitaxel is administered at a first dose at an amount of about 200 mg/m² to about 240 mg/m².

In some embodiments, the paclitaxel is administered at a first dose at an amount of about 200 mg/m² to about 235 mg/m².

In some embodiments, the paclitaxel is administered at a first dose at an amount of about 200 mg/m² to about 230 mg/m².

In some embodiments, the paclitaxel is administered at a first dose at an amount of about 200 mg/m² to about 225 mg/m².

In some embodiments, the paclitaxel is administered at a first dose at an amount of about 200 mg/m² to about 220 mg/m².

In some embodiments, the paclitaxel is administered at a first dose at an amount of about 200 mg/m² to about 215 mg/m².

In some embodiments, the paclitaxel is administered at a first dose at an amount of about 200 mg/m² to about 210 mg/m².

In some embodiments, the paclitaxel is administered at a first dose at an amount of about 200 mg/m² to about 205 mg/m².

In some embodiments, the paclitaxel is administered at a first dose at an amount of about 250 mg/m² to about 350 mg/m².

In some embodiments, the paclitaxel is administered at a first dose at an amount of about 250 mg/m² to about 300 mg/m².

In some embodiments, the paclitaxel is administered at a first dose at an amount of about 120 mg/m² to about 235 mg/m².

In some embodiments, the paclitaxel is administered at a first dose at an amount of about 120 mg/m² to about 230 mg/m².

In some embodiments, the paclitaxel is administered at a first dose at an amount of about 120 mg/m² to about 225 mg/m².

In some embodiments, the paclitaxel is administered at a first dose at an amount of about 120 mg/m² to about 220 mg/m².

In some embodiments, the paclitaxel is administered at a first dose at an amount of about 120 mg/m² to about 215 mg/m².

In some embodiments, the paclitaxel is administered at a first dose at an amount of about 120 mg/m² to about 210 mg/m².

In some embodiments, the paclitaxel is administered at a first dose at an amount of about 120 mg/m² to about 205 mg/m².

In some embodiments, the paclitaxel is administered at a first dose at an amount of about 120 mg/m² to about 200 mg/m².

In some embodiments, the paclitaxel is administered at a first dose at an amount of about 120 mg/m² to about 195 mg/m².

In some embodiments, the paclitaxel is administered at a first dose at an amount of about 120 mg/m² to about 190 mg/m².

In some embodiments, the paclitaxel is administered at a first dose at an amount of about 120 mg/m² to about 185 mg/m².

In some embodiments, the paclitaxel is administered at a first dose at an amount of about 120 mg/m² to about 180 mg/m².

In some embodiments, the paclitaxel is administered at a first dose at an amount of about 120 mg/m² to about 175 mg/m².

In some embodiments, the paclitaxel is administered at a first dose at an amount of about 120 mg/m² to about 170 mg/m².

In some embodiments, the paclitaxel is administered at a first dose at an amount of about 120 mg/m² to about 165 mg/m².

In some embodiments, the paclitaxel is administered at a first dose at an amount of about 120 mg/m² to about 160 mg/m².

In some embodiments, the paclitaxel is administered at a first dose at an amount of about 120 mg/m² to about 155 mg/m².

In some embodiments, the paclitaxel is administered at a first dose at an amount of about 120 mg/m² to about 150 mg/m².

In some embodiments, the paclitaxel is administered at a first dose at an amount of about 120 mg/m² to about 145 mg/m².

In some embodiments, the paclitaxel is administered at a first dose at an amount of about 120 mg/m² to about 140 mg/m².

In some embodiments, the paclitaxel is administered at a first dose at an amount of about 120 mg/m² to about 135 mg/m².

In some embodiments, the paclitaxel is administered at a first dose at an amount of about 120 mg/m² to about 130 mg/m².

In some embodiments, the paclitaxel is administered at a first dose at an amount of about 120 mg/m² to about 125 mg/m².

In some embodiments, the paclitaxel is administered at a first dose at an amount of about 125 mg/m² to about 235 mg/m².

In some embodiments, the paclitaxel is administered at a first dose at an amount of about 125 mg/m² to about 230 mg/m².

In some embodiments, the paclitaxel is administered at a first dose at an amount of about 125 mg/m² to about 225 mg/m².

In some embodiments, the paclitaxel is administered at a first dose at an amount of about 125 mg/m² to about 220 mg/m².

In some embodiments, the paclitaxel is administered at a first dose at an amount of about 125 mg/m² to about 215 mg/m².

In some embodiments, the paclitaxel is administered at a first dose at an amount of about 125 mg/m² to about 210 mg/m².

In some embodiments, the paclitaxel is administered at a first dose at an amount of about 125 mg/m² to about 205 mg/m².

In some embodiments, the paclitaxel is administered at a first dose at an amount of about 125 mg/m² to about 200 mg/m².

In some embodiments, the paclitaxel is administered at a first dose at an amount of about 125 mg/m² to about 195 mg/m².

In some embodiments, the paclitaxel is administered at a first dose at an amount of about 125 mg/m² to about 190 mg/m².

In some embodiments, the paclitaxel is administered at a first dose at an amount of about 125 mg/m² to about 185 mg/m².

In some embodiments, the paclitaxel is administered at a first dose at an amount of about 125 mg/m² to about 180 mg/m².

In some embodiments, the paclitaxel is administered at a first dose at an amount of about 125 mg/m² to about 175 mg/m².

In some embodiments, the paclitaxel is administered at a first dose at an amount of about 125 mg/m² to about 170 mg/m².

In some embodiments, the paclitaxel is administered at a first dose at an amount of about 125 mg/m² to about 165 mg/m².

In some embodiments, the paclitaxel is administered at a first dose at an amount of about 125 mg/m² to about 160 mg/m².

In some embodiments, the paclitaxel is administered at a first dose at an amount of about 125 mg/m² to about 155 mg/m².

In some embodiments, the paclitaxel is administered at a first dose at an amount of about 125 mg/m² to about 150 mg/m².

In some embodiments, the paclitaxel is administered at a first dose at an amount of about 125 mg/m² to about 145 mg/m².

In some embodiments, the paclitaxel is administered at a first dose at an amount of about 125 mg/m² to about 140 mg/m².

In some embodiments, the paclitaxel is administered at a first dose at an amount of about 125 mg/m² to about 135 mg/m².

In some embodiments, the paclitaxel is administered at a first dose at an amount of about 125 mg/m² to about 130 mg/m².

In some embodiments, the paclitaxel is administered at a first dose at an amount of about 130 mg/m² to about 235 mg/m².

In some embodiments, the paclitaxel is administered at a first dose at an amount of about 130 mg/m² to about 230 mg/m².

In some embodiments, the paclitaxel is administered at a first dose at an amount of about 130 mg/m² to about 225 mg/m².

In some embodiments, the paclitaxel is administered at a first dose at an amount of about 130 mg/m² to about 220 mg/m².

In some embodiments, the paclitaxel is administered at a first dose at an amount of about 130 mg/m² to about 215 mg/m².

In some embodiments, the paclitaxel is administered at a first dose at an amount of about 130 mg/m² to about 210 mg/m².

In some embodiments, the paclitaxel is administered at a first dose at an amount of about 130 mg/m² to about 205 mg/m².

In some embodiments, the paclitaxel is administered at a first dose at an amount of about 130 mg/m² to about 200 mg/m².

In some embodiments, the paclitaxel is administered at a first dose at an amount of about 130 mg/m² to about 195 mg/m².

In some embodiments, the paclitaxel is administered at a first dose at an amount of about 130 mg/m² to about 190 mg/m².

In some embodiments, the paclitaxel is administered at a first dose at an amount of about 130 mg/m² to about 185 mg/m².

In some embodiments, the paclitaxel is administered at a first dose at an amount of about 130 mg/m² to about 180 mg/m².

In some embodiments, the paclitaxel is administered at a first dose at an amount of about 130 mg/m² to about 175 mg/m².

In some embodiments, the paclitaxel is administered at a first dose at an amount of about 130 mg/m² to about 170 mg/m².

In some embodiments, the paclitaxel is administered at a first dose at an amount of about 130 mg/m² to about 165 mg/m².

In some embodiments, the paclitaxel is administered at a first dose at an amount of about 130 mg/m² to about 160 mg/m².

In some embodiments, the paclitaxel is administered at a first dose at an amount of about 130 mg/m² to about 155 mg/m².

In some embodiments, the paclitaxel is administered at a first dose at an amount of about 130 mg/m² to about 150 mg/m².

In some embodiments, the paclitaxel is administered at a first dose at an amount of about 130 mg/m² to about 145 mg/m².

In some embodiments, the paclitaxel is administered at a first dose at an amount of about 130 mg/m² to about 140 mg/m².

In some embodiments, the paclitaxel is administered at a first dose at an amount of about 130 mg/m² to about 135 mg/m².

In some embodiments, the paclitaxel is administered at a first dose at an amount of about 100 mg/m².

In some embodiments, the paclitaxel is administered at a first dose at an amount of about 105 mg/m².

In some embodiments, the paclitaxel is administered at a first dose at an amount of about 110 mg/m².

In some embodiments, the paclitaxel is administered at a first dose at an amount of about 115 mg/m².

In some embodiments, the paclitaxel is administered at a first dose at an amount of about 120 mg/m².

In some embodiments, the paclitaxel is administered at a first dose at an amount of about 125 mg/m².

In some embodiments, the paclitaxel is administered at a first dose at an amount of about 130 mg/m².

In some embodiments, the paclitaxel is administered at a first dose at an amount of about 135 mg/m².

In some embodiments, the paclitaxel is administered at a first dose at an amount of about 140 mg/m².

In some embodiments, the paclitaxel is administered at a first dose at an amount of about 145 mg/m².

In some embodiments, the paclitaxel is administered at a first dose at an amount of about 150 mg/m².

In some embodiments, the paclitaxel is administered at a first dose at an amount of about 155 mg/m².

In some embodiments, the paclitaxel is administered at a first dose at an amount of about 160 mg/m².

In some embodiments, the paclitaxel is administered at a first dose at an amount of about 165 mg/m².

In some embodiments, the paclitaxel is administered at a first dose at an amount of about 170 mg/m².

In some embodiments, the paclitaxel is administered at a first dose at an amount of about 175 mg/m².

In some embodiments, the paclitaxel is administered at a first dose at an amount of about 180 mg/m².

In some embodiments, the paclitaxel is administered at a first dose at an amount of about 185 mg/m².

In some embodiments, the paclitaxel is administered at a first dose at an amount of about 190 mg/m².

In some embodiments, the paclitaxel is administered at a first dose at an amount of about 195 mg/m².

In some embodiments, the paclitaxel is administered at a first dose at an amount of about 200 mg/m².

In some embodiments, the paclitaxel is administered at a first dose at an amount of about 205 mg/m².

In some embodiments, the paclitaxel is administered at a first dose at an amount of about 210 mg/m².

In some embodiments, the paclitaxel is administered at a first dose at an amount of about 215 mg/m².

In some embodiments, the paclitaxel is administered at a first dose at an amount of about 220 mg/m².

In some embodiments, the paclitaxel is administered at a first dose at an amount of about 225 mg/m².

In some embodiments, the paclitaxel is administered at a first dose at an amount of about 230 mg/m².

In some embodiments, the paclitaxel is administered at a first dose at an amount of about 235 mg/m².

In some embodiments, the paclitaxel is administered at a first dose at an amount of about 240 mg/m².

In some embodiments, the paclitaxel is administered at a first dose at an amount of about 245 mg/m².

In some embodiments, the paclitaxel is administered at a first dose at an amount of about 250 mg/m².

In some embodiments, the paclitaxel is administered at a first dose at an amount of about 255 mg/m².

In some embodiments, the paclitaxel is administered at a first dose at an amount of about 260 mg/m².

In some embodiments, the paclitaxel is administered at a first dose at an amount of about 265 mg/m².

In some embodiments, the paclitaxel is administered at a first dose at an amount of about 270 mg/m².

In some embodiments, the paclitaxel is administered at a first dose at an amount of about 275 mg/m².

In some embodiments, the paclitaxel is administered at a first dose at an amount of about 280 mg/m².

In some embodiments, the paclitaxel is administered at a first dose at an amount of about 285 mg/m².

In some embodiments, the paclitaxel is administered at a first dose at an amount of about 290 mg/m².

In some embodiments, the paclitaxel is administered at a first dose at an amount of about 295 mg/m².

In some embodiments, the paclitaxel is administered at a first dose at an amount of about 300 mg/m².

In some embodiments, the paclitaxel is administered at a first dose at an amount of about 305 mg/m².

In some embodiments, the paclitaxel is administered at a first dose at an amount of about 310 mg/m².

In some embodiments, the paclitaxel is administered at a first dose at an amount of about 315 mg/m².

In some embodiments, the paclitaxel is administered at a first dose at an amount of about 320 mg/m².

In some embodiments, the paclitaxel is administered at a first dose at an amount of about 325 mg/m².

In some embodiments, the paclitaxel is administered at a first dose at an amount of about 330 mg/m².

In some embodiments, the paclitaxel is administered at a first dose at an amount of about 335 mg/m².

In some embodiments, the paclitaxel is administered at a first dose at an amount of about 340 mg/m².

In some embodiments, the paclitaxel is administered at a first dose at an amount of about 345 mg/m².

In some embodiments, the paclitaxel is administered at a first dose at an amount of about 350 mg/m².

In some embodiments, the paclitaxel is administered at a first dose at an amount of about 355 mg/m².

In some embodiments, the paclitaxel is administered at a first dose at an amount of about 360 mg/m².

In some embodiments, the paclitaxel is administered at a first dose at an amount of about 365 mg/m².

In some embodiments, the paclitaxel is administered at a first dose at an amount of about 370 mg/m².

In some embodiments, the paclitaxel is administered at a first dose at an amount of about 375 mg/m².

In some embodiments, the paclitaxel is administered at a first dose at an amount of about 380 mg/m².

In some embodiments, the paclitaxel is administered at a first dose at an amount of about 385 mg/m².

In some embodiments, the paclitaxel is administered at a first dose at an amount of about 390 mg/m².

In some embodiments, the paclitaxel is administered at a first dose at an amount of about 395 mg/m².

In some embodiments, the paclitaxel is administered at a first dose at an amount of about 400 mg/m².

In some embodiments, the paclitaxel is administered at a second dose of about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose. In some embodiments, the paclitaxel is administered at a second dose of about 20 mg/m², about 25 mg/m², about 30 mg/m², about 35 mg/m², about 40 mg/m², about 45 mg/m², about 50 mg/m², about 55 mg/m², or about 60 mg/m² less than the first dose. In some embodiments, the paclitaxel is administered at a second dose of about 30 mg/m², about 35 mg/m², about 40 mg/m², about 45 mg/m², or about 50 mg/m² less than the first dose. In some embodiments, the paclitaxel is administered at a second dose of about 40 mg/m² less than the first dose.

In some embodiments, the paclitaxel is administered at a second dose at an amount of about 100 mg/m² to about 400 mg/m².

In some embodiments, the paclitaxel is administered at a second dose at an amount of about 100 mg/m² to about 350 mg/m².

In some embodiments, the paclitaxel is administered at a second dose at an amount of about 100 mg/m² to about 300 mg/m².

In some embodiments, the paclitaxel is administered at a second dose at an amount of about 150 mg/m² to about 250 mg/m².

In some embodiments, the paclitaxel is administered at a second dose at an amount of about 100 mg/m² to about 245 mg/m².

In some embodiments, the paclitaxel is administered at a second dose at an amount of about 100 mg/m² to about 240 mg/m².

In some embodiments, the paclitaxel is administered at a second dose at an amount of about 100 mg/m² to about 235 mg/m².

In some embodiments, the paclitaxel is administered at a second dose at an amount of about 100 mg/m² to about 230 mg/m².

In some embodiments, the paclitaxel is administered at a second dose at an amount of about 100 mg/m² to about 225 mg/m².

In some embodiments, the paclitaxel is administered at a second dose at an amount of about 100 mg/m² to about 220 mg/m².

In some embodiments, the paclitaxel is administered at a second dose at an amount of about 100 mg/m² to about 215 mg/m².

In some embodiments, the paclitaxel is administered at a second dose at an amount of about 100 mg/m² to about 210 mg/m².

In some embodiments, the paclitaxel is administered at a second dose at an amount of about 100 mg/m² to about 205 mg/m².

In some embodiments, the paclitaxel is administered at a second dose at an amount of about 100 mg/m² to about 200 mg/m².

In some embodiments, the paclitaxel is administered at a second dose at an amount of about 100 mg/m² to about 195 mg/m².

In some embodiments, the paclitaxel is administered at a second dose at an amount of about 100 mg/m² to about 190 mg/m².

In some embodiments, the paclitaxel is administered at a second dose at an amount of about 100 mg/m² to about 185 mg/m².

In some embodiments, the paclitaxel is administered at a second dose at an amount of about 100 mg/m² to about 180 mg/m².

In some embodiments, the paclitaxel is administered at a second dose at an amount of about 100 mg/m² to about 175 mg/m².

In some embodiments, the paclitaxel is administered at a second dose at an amount of about 100 mg/m² to about 170 mg/m².

In some embodiments, the paclitaxel is administered at a second dose at an amount of about 100 mg/m² to about 165 mg/m².

In some embodiments, the paclitaxel is administered at a second dose at an amount of about 100 mg/m² to about 160 mg/m².

In some embodiments, the paclitaxel is administered at a second dose at an amount of about 100 mg/m² to about 155 mg/m².

In some embodiments, the paclitaxel is administered at a second dose at an amount of about 100 mg/m² to about 150 mg/m².

In some embodiments, the paclitaxel is administered at a second dose at an amount of about 100 mg/m² to about 145 mg/m².

In some embodiments, the paclitaxel is administered at a second dose at an amount of about 100 mg/m² to about 140 mg/m².

In some embodiments, the paclitaxel is administered at a second dose at an amount of about 100 mg/m² to about 135 mg/m².

In some embodiments, the paclitaxel is administered at a second dose at an amount of about 100 mg/m² to about 130 mg/m².

In some embodiments, the paclitaxel is administered at a second dose at an amount of about 100 mg/m² to about 125 mg/m².

In some embodiments, the paclitaxel is administered at a second dose at an amount of about 100 mg/m² to about 120 mg/m².

In some embodiments, the paclitaxel is administered at a second dose at an amount of about 100 mg/m² to about 115 mg/m².

In some embodiments, the paclitaxel is administered at a second dose at an amount of about 100 mg/m² to about 110 mg/m².

In some embodiments, the paclitaxel is administered at a second dose at an amount of about 100 mg/m² to about 105 mg/m².

In some embodiments, the paclitaxel is administered at a second dose at an amount of about 150 mg/m² to about 350 mg/m².

In some embodiments, the paclitaxel is administered at a second dose at an amount of about 150 mg/m² to about 300 mg/m².

In some embodiments, the paclitaxel is administered at a second dose at an amount of about 150 mg/m² to about 250 mg/m².

In some embodiments, the paclitaxel is administered at a second dose at an amount of about 150 mg/m² to about 245 mg/m².

In some embodiments, the paclitaxel is administered at a second dose at an amount of about 150 mg/m² to about 240 mg/m².

In some embodiments, the paclitaxel is administered at a second dose at an amount of about 150 mg/m² to about 235 mg/m².

In some embodiments, the paclitaxel is administered at a second dose at an amount of about 150 mg/m² to about 230 mg/m².

In some embodiments, the paclitaxel is administered at a second dose at an amount of about 150 mg/m² to about 225 mg/m².

In some embodiments, the paclitaxel is administered at a second dose at an amount of about 150 mg/m² to about 220 mg/m².

In some embodiments, the paclitaxel is administered at a second dose at an amount of about 150 mg/m² to about 215 mg/m².

In some embodiments, the paclitaxel is administered at a second dose at an amount of about 150 mg/m² to about 210 mg/m².

In some embodiments, the paclitaxel is administered at a second dose at an amount of about 150 mg/m² to about 205 mg/m².

In some embodiments, the paclitaxel is administered at a second dose at an amount of about 150 mg/m² to about 200 mg/m².

In some embodiments, the paclitaxel is administered at a second dose at an amount of about 150 mg/m² to about 195 mg/m².

In some embodiments, the paclitaxel is administered at a second dose at an amount of about 150 mg/m² to about 190 mg/m².

In some embodiments, the paclitaxel is administered at a second dose at an amount of about 150 mg/m² to about 185 mg/m².

In some embodiments, the paclitaxel is administered at a second dose at an amount of about 150 mg/m² to about 180 mg/m².

In some embodiments, the paclitaxel is administered at a second dose at an amount of about 150 mg/m² to about 175 mg/m².

In some embodiments, the paclitaxel is administered at a second dose at an amount of about 150 mg/m² to about 170 mg/m².

In some embodiments, the paclitaxel is administered at a second dose at an amount of about 150 mg/m² to about 165 mg/m².

In some embodiments, the paclitaxel is administered at a second dose at an amount of about 150 mg/m² to about 160 mg/m².

In some embodiments, the paclitaxel is administered at a second dose at an amount of about 150 mg/m² to about 155 mg/m².

In some embodiments, the paclitaxel is administered at a second dose at an amount of about 200 mg/m² to about 300 mg/m².

In some embodiments, the paclitaxel is administered at a second dose at an amount of about 200 mg/m² to about 295 mg/m².

In some embodiments, the paclitaxel is administered at a second dose at an amount of about 200 mg/m² to about 290 mg/m².

In some embodiments, the paclitaxel is administered at a second dose at an amount of about 200 mg/m² to about 285 mg/m².

In some embodiments, the paclitaxel is administered at a second dose at an amount of about 200 mg/m² to about 280 mg/m².

In some embodiments, the paclitaxel is administered at a second dose at an amount of about 200 mg/m² to about 275 mg/m².

In some embodiments, the paclitaxel is administered at a second dose at an amount of about 200 mg/m² to about 270 mg/m².

In some embodiments, the paclitaxel is administered at a second dose at an amount of about 200 mg/m² to about 265 mg/m².

In some embodiments, the paclitaxel is administered at a second dose at an amount of about 200 mg/m² to about 260 mg/m².

In some embodiments, the paclitaxel is administered at a second dose at an amount of about 200 mg/m² to about 255 mg/m².

In some embodiments, the paclitaxel is administered at a second dose at an amount of about 200 mg/m² to about 250 mg/m².

In some embodiments, the paclitaxel is administered at a second dose at an amount of about 200 mg/m² to about 245 mg/m².

In some embodiments, the paclitaxel is administered at a second dose at an amount of about 200 mg/m² to about 240 mg/m².

In some embodiments, the paclitaxel is administered at a second dose at an amount of about 200 mg/m² to about 235 mg/m².

In some embodiments, the paclitaxel is administered at a second dose at an amount of about 200 mg/m² to about 230 mg/m².

In some embodiments, the paclitaxel is administered at a second dose at an amount of about 200 mg/m² to about 225 mg/m².

In some embodiments, the paclitaxel is administered at a second dose at an amount of about 200 mg/m² to about 220 mg/m².

In some embodiments, the paclitaxel is administered at a second dose at an amount of about 200 mg/m² to about 215 mg/m².

In some embodiments, the paclitaxel is administered at a second dose at an amount of about 200 mg/m² to about 210 mg/m².

In some embodiments, the paclitaxel is administered at a second dose at an amount of about 200 mg/m² to about 205 mg/m².

In some embodiments, the paclitaxel is administered at a second dose at an amount of about 120 mg/m² to about 235 mg/m².

In some embodiments, the paclitaxel is administered at a second dose at an amount of about 120 mg/m² to about 230 mg/m².

In some embodiments, the paclitaxel is administered at a second dose at an amount of about 120 mg/m² to about 225 mg/m².

In some embodiments, the paclitaxel is administered at a second dose at an amount of about 120 mg/m² to about 220 mg/m².

In some embodiments, the paclitaxel is administered at a second dose at an amount of about 120 mg/m² to about 215 mg/m².

In some embodiments, the paclitaxel is administered at a second dose at an amount of about 120 mg/m² to about 210 mg/m².

In some embodiments, the paclitaxel is administered at a second dose at an amount of about 120 mg/m² to about 205 mg/m².

In some embodiments, the paclitaxel is administered at a second dose at an amount of about 120 mg/m² to about 200 mg/m².

In some embodiments, the paclitaxel is administered at a second dose at an amount of about 120 mg/m² to about 195 mg/m².

In some embodiments, the paclitaxel is administered at a second dose at an amount of about 120 mg/m² to about 190 mg/m².

In some embodiments, the paclitaxel is administered at a second dose at an amount of about 120 mg/m² to about 185 mg/m².

In some embodiments, the paclitaxel is administered at a second dose at an amount of about 120 mg/m² to about 180 mg/m².

In some embodiments, the paclitaxel is administered at a second dose at an amount of about 120 mg/m² to about 175 mg/m².

In some embodiments, the paclitaxel is administered at a second dose at an amount of about 120 mg/m² to about 170 mg/m².

In some embodiments, the paclitaxel is administered at a second dose at an amount of about 120 mg/m² to about 165 mg/m².

In some embodiments, the paclitaxel is administered at a second dose at an amount of about 120 mg/m² to about 160 mg/m².

In some embodiments, the paclitaxel is administered at a second dose at an amount of about 120 mg/m² to about 155 mg/m².

In some embodiments, the paclitaxel is administered at a second dose at an amount of about 120 mg/m² to about 150 mg/m².

In some embodiments, the paclitaxel is administered at a second dose at an amount of about 120 mg/m² to about 145 mg/m².

In some embodiments, the paclitaxel is administered at a second dose at an amount of about 120 mg/m² to about 140 mg/m².

In some embodiments, the paclitaxel is administered at a second dose at an amount of about 120 mg/m² to about 135 mg/m².

In some embodiments, the paclitaxel is administered at a second dose at an amount of about 120 mg/m² to about 130 mg/m².

In some embodiments, the paclitaxel is administered at a second dose at an amount of about 120 mg/m² to about 125 mg/m².

In some embodiments, the paclitaxel is administered at a second dose at an amount of about 125 mg/m² to about 235 mg/m².

In some embodiments, the paclitaxel is administered at a second dose at an amount of about 125 mg/m² to about 230 mg/m².

In some embodiments, the paclitaxel is administered at a second dose at an amount of about 125 mg/m² to about 225 mg/m².

In some embodiments, the paclitaxel is administered at a second dose at an amount of about 125 mg/m² to about 220 mg/m².

In some embodiments, the paclitaxel is administered at a second dose at an amount of about 125 mg/m² to about 215 mg/m².

In some embodiments, the paclitaxel is administered at a second dose at an amount of about 125 mg/m² to about 210 mg/m².

In some embodiments, the paclitaxel is administered at a second dose at an amount of about 125 mg/m² to about 205 mg/m².

In some embodiments, the paclitaxel is administered at a second dose at an amount of about 125 mg/m² to about 200 mg/m².

In some embodiments, the paclitaxel is administered at a second dose at an amount of about 125 mg/m² to about 195 mg/m².

In some embodiments, the paclitaxel is administered at a second dose at an amount of about 125 mg/m² to about 190 mg/m².

In some embodiments, the paclitaxel is administered at a second dose at an amount of about 125 mg/m² to about 185 mg/m².

In some embodiments, the paclitaxel is administered at a second dose at an amount of about 125 mg/m² to about 180 mg/m².

In some embodiments, the paclitaxel is administered at a second dose at an amount of about 125 mg/m² to about 175 mg/m².

In some embodiments, the paclitaxel is administered at a second dose at an amount of about 125 mg/m² to about 170 mg/m².

In some embodiments, the paclitaxel is administered at a second dose at an amount of about 125 mg/m² to about 165 mg/m².

In some embodiments, the paclitaxel is administered at a second dose at an amount of about 125 mg/m² to about 160 mg/m².

In some embodiments, the paclitaxel is administered at a second dose at an amount of about 125 mg/m² to about 155 mg/m².

In some embodiments, the paclitaxel is administered at a second dose at an amount of about 125 mg/m² to about 150 mg/m².

In some embodiments, the paclitaxel is administered at a second dose at an amount of about 125 mg/m² to about 145 mg/m².

In some embodiments, the paclitaxel is administered at a second dose at an amount of about 125 mg/m² to about 140 mg/m².

In some embodiments, the paclitaxel is administered at a second dose at an amount of about 125 mg/m² to about 135 mg/m².

In some embodiments, the paclitaxel is administered at a second dose at an amount of about 125 mg/m² to about 130 mg/m².

In some embodiments, the paclitaxel is administered at a second dose at an amount of about 130 mg/m² to about 235 mg/m².

In some embodiments, the paclitaxel is administered at a second dose at an amount of about 130 mg/m² to about 230 mg/m².

In some embodiments, the paclitaxel is administered at a second dose at an amount of about 130 mg/m² to about 225 mg/m².

In some embodiments, the paclitaxel is administered at a second dose at an amount of about 130 mg/m² to about 220 mg/m².

In some embodiments, the paclitaxel is administered at a second dose at an amount of about 130 mg/m² to about 215 mg/m².

In some embodiments, the paclitaxel is administered at a second dose at an amount of about 130 mg/m² to about 210 mg/m².

In some embodiments, the paclitaxel is administered at a second dose at an amount of about 130 mg/m² to about 205 mg/m².

In some embodiments, the paclitaxel is administered at a second dose at an amount of about 130 mg/m² to about 200 mg/m².

In some embodiments, the paclitaxel is administered at a second dose at an amount of about 130 mg/m² to about 195 mg/m².

In some embodiments, the paclitaxel is administered at a second dose at an amount of about 130 mg/m² to about 190 mg/m².

In some embodiments, the paclitaxel is administered at a second dose at an amount of about 130 mg/m² to about 185 mg/m².

In some embodiments, the paclitaxel is administered at a second dose at an amount of about 130 mg/m² to about 180 mg/m².

In some embodiments, the paclitaxel is administered at a second dose at an amount of about 130 mg/m² to about 175 mg/m².

In some embodiments, the paclitaxel is administered at a second dose at an amount of about 130 mg/m² to about 170 mg/m².

In some embodiments, the paclitaxel is administered at a second dose at an amount of about 130 mg/m² to about 165 mg/m².

In some embodiments, the paclitaxel is administered at a second dose at an amount of about 130 mg/m² to about 160 mg/m².

In some embodiments, the paclitaxel is administered at a second dose at an amount of about 130 mg/m² to about 155 mg/m².

In some embodiments, the paclitaxel is administered at a second dose at an amount of about 130 mg/m² to about 150 mg/m².

In some embodiments, the paclitaxel is administered at a second dose at an amount of about 130 mg/m² to about 145 mg/m².

In some embodiments, the paclitaxel is administered at a second dose at an amount of about 130 mg/m² to about 140 mg/m².

In some embodiments, the paclitaxel is administered at a second dose at an amount of about 130 mg/m² to about 135 mg/m².

In some embodiments, the paclitaxel is administered at a second dose at an amount of about 100 mg/m².

In some embodiments, the paclitaxel is administered at a second dose at an amount of about 105 mg/m².

In some embodiments, the paclitaxel is administered at a second dose at an amount of about 110 mg/m².

In some embodiments, the paclitaxel is administered at a second dose at an amount of about 115 mg/m².

In some embodiments, the paclitaxel is administered at a second dose at an amount of about 120 mg/m².

In some embodiments, the paclitaxel is administered at a second dose at an amount of about 125 mg/m².

In some embodiments, the paclitaxel is administered at a second dose at an amount of about 130 mg/m².

In some embodiments, the paclitaxel is administered at a second dose at an amount of about 135 mg/m².

In some embodiments, the paclitaxel is administered at a second dose at an amount of about 140 mg/m².

In some embodiments, the paclitaxel is administered at a second dose at an amount of about 145 mg/m².

In some embodiments, the paclitaxel is administered at a second dose at an amount of about 150 mg/m².

In some embodiments, the paclitaxel is administered at a second dose at an amount of about 155 mg/m².

In some embodiments, the paclitaxel is administered at a second dose at an amount of about 160 mg/m².

In some embodiments, the paclitaxel is administered at a second dose at an amount of about 165 mg/m².

In some embodiments, the paclitaxel is administered at a second dose at an amount of about 170 mg/m².

In some embodiments, the paclitaxel is administered at a second dose at an amount of about 175 mg/m².

In some embodiments, the paclitaxel is administered at a second dose at an amount of about 180 mg/m².

In some embodiments, the paclitaxel is administered at a second dose at an amount of about 185 mg/m².

In some embodiments, the paclitaxel is administered at a second dose at an amount of about 190 mg/m².

In some embodiments, the paclitaxel is administered at a second dose at an amount of about 195 mg/m².

In some embodiments, the paclitaxel is administered at a second dose at an amount of about 200 mg/m².

In some embodiments, the paclitaxel is administered at a second dose at an amount of about 205 mg/m².

In some embodiments, the paclitaxel is administered at a second dose at an amount of about 210 mg/m².

In some embodiments, the paclitaxel is administered at a second dose at an amount of about 215 mg/m².

In some embodiments, the paclitaxel is administered at a second dose at an amount of about 220 mg/m².

In some embodiments, the paclitaxel is administered at a second dose at an amount of about 225 mg/m².

In some embodiments, the paclitaxel is administered at a second dose at an amount of about 230 mg/m².

In some embodiments, the paclitaxel is administered at a second dose at an amount of about 235 mg/m².

In some embodiments, the paclitaxel is administered at a second dose at an amount of about 240 mg/m².

In some embodiments, the paclitaxel is administered at a second dose at an amount of about 245 mg/m².

In some embodiments, the paclitaxel is administered at a second dose at an amount of about 250 mg/m².

In some embodiments, the paclitaxel is administered at a second dose at an amount of about 255 mg/m².

In some embodiments, the paclitaxel is administered at a second dose at an amount of about 260 mg/m².

In some embodiments, the paclitaxel is administered at a second dose at an amount of about 265 mg/m².

In some embodiments, the paclitaxel is administered at a second dose at an amount of about 270 mg/m².

In some embodiments, the paclitaxel is administered at a second dose at an amount of about 275 mg/m².

In some embodiments, the paclitaxel is administered at a second dose at an amount of about 280 mg/m².

In some embodiments, the paclitaxel is administered at a second dose at an amount of about 285 mg/m².

In some embodiments, the paclitaxel is administered at a second dose at an amount of about 290 mg/m².

In some embodiments, the paclitaxel is administered at a second dose at an amount of about 295 mg/m².

In some embodiments, the paclitaxel is administered at a second dose at an amount of about 300 mg/m².

In some embodiments, the paclitaxel is administered at a second dose at an amount of about 305 mg/m².

In some embodiments, the paclitaxel is administered at a second dose at an amount of about 310 mg/m².

In some embodiments, the paclitaxel is administered at a second dose at an amount of about 315 mg/m².

In some embodiments, the paclitaxel is administered at a second dose at an amount of about 320 mg/m².

In some embodiments, the paclitaxel is administered at a second dose at an amount of about 325 mg/m².

In some embodiments, the paclitaxel is administered at a second dose at an amount of about 330 mg/m².

In some embodiments, the paclitaxel is administered at a second dose at an amount of about 335 mg/m².

In some embodiments, the paclitaxel is administered at a second dose at an amount of about 340 mg/m².

In some embodiments, the paclitaxel is administered at a second dose at an amount of about 345 mg/m².

In some embodiments, the paclitaxel is administered at a second dose at an amount of about 350 mg/m².

In some embodiments, the paclitaxel is administered at a second dose at an amount of about 355 mg/m².

In some embodiments, the paclitaxel is administered at a second dose at an amount of about 360 mg/m².

In some embodiments, the paclitaxel is administered at a second dose at an amount of about 365 mg/m².

In some embodiments, the paclitaxel is administered at a second dose at an amount of about 370 mg/m².

In some embodiments, the paclitaxel is administered at a second dose at an amount of about 375 mg/m².

In some embodiments, the paclitaxel is administered at a second dose at an amount of about 380 mg/m².

In some embodiments, the paclitaxel is administered at a second dose at an amount of about 385 mg/m².

In some embodiments, the paclitaxel is administered at a second dose at an amount of about 390 mg/m².

In some embodiments, the paclitaxel is administered at a second dose at an amount of about 395 mg/m².

In some embodiments, the paclitaxel is administered at a second dose at an amount of about 400 mg/m².

In some embodiments, the paclitaxel is administered at a first dose followed by a second dose, wherein the second dose is followed by a third dose.

In some embodiments, the paclitaxel is administered at a first dose followed by a second dose wherein the second dose is followed be a third dose, wherein the first dose is reduced to a second dose. In some embodiments, the paclitaxel is administered at a first dose followed by a second dose wherein the second dose is followed be a third dose, wherein the second dose is reduced to a third dose.

In some embodiments, the paclitaxel is administered at a second dose followed by a third dose.

In some embodiments, the paclitaxel is administered at a second dose followed by a third dose, wherein the second dose is reduced to a third dose.

In some embodiments, the paclitaxel is administered at a third dose of about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose. In some embodiments, the paclitaxel is administered at a third dose of about 55 mg/m², about 60 mg/m², about 65 mg/m², about 70 mg/m², about 75 mg/m², about 80 mg/m², about 85 mg/m², about 90 mg/m², or about 95 mg/m² less than the first dose. In some embodiments, the paclitaxel is administered at a third dose of about 65 mg/m², about 70 mg/m², about 75 mg/m², about 80 mg/m², or about 85 mg/m² less than the first dose. In some embodiments, the paclitaxel is administered at a third dose of about 75 mg/m² less than the first dose.

In some embodiments, the paclitaxel is administered at a third dose of about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the second dose. In some embodiments, the paclitaxel is administered at a third dose of about 15 mg/m², about 20 mg/m², about 25 mg/m², about 30 mg/m², about 35 mg/m², about 40 mg/m², about 45 mg/m², about 50 mg/m², or about 55 mg/m² less than the second dose. In some embodiments, the paclitaxel is administered at a third dose of about 25 mg/m², about 30 mg/m², about 35 mg/m², about 40 mg/m², or about 45 mg/m² less than the second dose. In some embodiments, the paclitaxel is administered at a third dose of about 35 mg/m² less than the second dose.

In some embodiments, the paclitaxel is administered at a third dose at an amount of about 100 mg/m² to about 400 mg/m².

In some embodiments, the paclitaxel is administered at a third dose at an amount of about 100 mg/m² to about 350 mg/m².

In some embodiments, the paclitaxel is administered at a third dose at an amount of about 100 mg/m² to about 300 mg/m².

In some embodiments, the paclitaxel is administered at a third dose at an amount of about 150 mg/m² to about 250 mg/m².

In some embodiments, the paclitaxel is administered at a third dose at an amount of about 100 mg/m² to about 245 mg/m².

In some embodiments, the paclitaxel is administered at a third dose at an amount of about 100 mg/m² to about 240 mg/m².

In some embodiments, the paclitaxel is administered at a third dose at an amount of about 100 mg/m² to about 235 mg/m².

In some embodiments, the paclitaxel is administered at a third dose at an amount of about 100 mg/m² to about 230 mg/m².

In some embodiments, the paclitaxel is administered at a third dose at an amount of about 100 mg/m² to about 225 mg/m².

In some embodiments, the paclitaxel is administered at a third dose at an amount of about 100 mg/m² to about 220 mg/m².

In some embodiments, the paclitaxel is administered at a third dose at an amount of about 100 mg/m² to about 215 mg/m².

In some embodiments, the paclitaxel is administered at a third dose at an amount of about 100 mg/m² to about 210 mg/m².

In some embodiments, the paclitaxel is administered at a third dose at an amount of about 100 mg/m² to about 205 mg/m².

In some embodiments, the paclitaxel is administered at a third dose at an amount of about 100 mg/m² to about 200 mg/m².

In some embodiments, the paclitaxel is administered at a third dose at an amount of about 100 mg/m² to about 195 mg/m².

In some embodiments, the paclitaxel is administered at a third dose at an amount of about 100 mg/m² to about 190 mg/m².

In some embodiments, the paclitaxel is administered at a third dose at an amount of about 100 mg/m² to about 185 mg/m².

In some embodiments, the paclitaxel is administered at a third dose at an amount of about 100 mg/m² to about 180 mg/m².

In some embodiments, the paclitaxel is administered at a third dose at an amount of about 100 mg/m² to about 175 mg/m².

In some embodiments, the paclitaxel is administered at a third dose at an amount of about 100 mg/m² to about 170 mg/m².

In some embodiments, the paclitaxel is administered at a third dose at an amount of about 100 mg/m² to about 165 mg/m².

In some embodiments, the paclitaxel is administered at a third dose at an amount of about 100 mg/m² to about 160 mg/m².

In some embodiments, the paclitaxel is administered at a third dose at an amount of about 100 mg/m² to about 155 mg/m².

In some embodiments, the paclitaxel is administered at a third dose at an amount of about 100 mg/m² to about 150 mg/m².

In some embodiments, the paclitaxel is administered at a third dose at an amount of about 100 mg/m² to about 145 mg/m².

In some embodiments, the paclitaxel is administered at a third dose at an amount of about 100 mg/m² to about 140 mg/m².

In some embodiments, the paclitaxel is administered at a third dose at an amount of about 100 mg/m² to about 135 mg/m².

In some embodiments, the paclitaxel is administered at a third dose at an amount of about 100 mg/m² to about 130 mg/m².

In some embodiments, the paclitaxel is administered at a third dose at an amount of about 100 mg/m² to about 125 mg/m².

In some embodiments, the paclitaxel is administered at a third dose at an amount of about 100 mg/m² to about 120 mg/m².

In some embodiments, the paclitaxel is administered at a third dose at an amount of about 100 mg/m² to about 115 mg/m².

In some embodiments, the paclitaxel is administered at a third dose at an amount of about 100 mg/m² to about 110 mg/m².

In some embodiments, the paclitaxel is administered at a third dose at an amount of about 100 mg/m² to about 105 mg/m².

In some embodiments, the paclitaxel is administered at a third dose at an amount of about 150 mg/m² to about 350 mg/m².

In some embodiments, the paclitaxel is administered at a third dose at an amount of about 150 mg/m² to about 300 mg/m².

In some embodiments, the paclitaxel is administered at a third dose at an amount of about 150 mg/m² to about 250 mg/m².

In some embodiments, the paclitaxel is administered at a third dose at an amount of about 150 mg/m² to about 245 mg/m².

In some embodiments, the paclitaxel is administered at a third dose at an amount of about 150 mg/m² to about 240 mg/m².

In some embodiments, the paclitaxel is administered at a third dose at an amount of about 150 mg/m² to about 235 mg/m².

In some embodiments, the paclitaxel is administered at a third dose at an amount of about 150 mg/m² to about 230 mg/m².

In some embodiments, the paclitaxel is administered at a third dose at an amount of about 150 mg/m² to about 225 mg/m².

In some embodiments, the paclitaxel is administered at a third dose at an amount of about 150 mg/m² to about 220 mg/m².

In some embodiments, the paclitaxel is administered at a third dose at an amount of about 150 mg/m² to about 215 mg/m².

In some embodiments, the paclitaxel is administered at a third dose at an amount of about 150 mg/m² to about 210 mg/m².

In some embodiments, the paclitaxel is administered at a third dose at an amount of about 150 mg/m² to about 205 mg/m².

In some embodiments, the paclitaxel is administered at a third dose at an amount of about 150 mg/m² to about 200 mg/m².

In some embodiments, the paclitaxel is administered at a third dose at an amount of about 150 mg/m² to about 195 mg/m².

In some embodiments, the paclitaxel is administered at a third dose at an amount of about 150 mg/m² to about 190 mg/m².

In some embodiments, the paclitaxel is administered at a third dose at an amount of about 150 mg/m² to about 185 mg/m².

In some embodiments, the paclitaxel is administered at a third dose at an amount of about 150 mg/m² to about 180 mg/m².

In some embodiments, the paclitaxel is administered at a third dose at an amount of about 150 mg/m² to about 175 mg/m².

In some embodiments, the paclitaxel is administered at a third dose at an amount of about 150 mg/m² to about 170 mg/m².

In some embodiments, the paclitaxel is administered at a third dose at an amount of about 150 mg/m² to about 165 mg/m².

In some embodiments, the paclitaxel is administered at a third dose at an amount of about 150 mg/m² to about 160 mg/m².

In some embodiments, the paclitaxel is administered at a third dose at an amount of about 150 mg/m² to about 155 mg/m².

In some embodiments, the paclitaxel is administered at a third dose at an amount of about 200 mg/m² to about 300 mg/m².

In some embodiments, the paclitaxel is administered at a third dose at an amount of about 200 mg/m² to about 295 mg/m².

In some embodiments, the paclitaxel is administered at a third dose at an amount of about 200 mg/m² to about 290 mg/m².

In some embodiments, the paclitaxel is administered at a third dose at an amount of about 200 mg/m² to about 285 mg/m².

In some embodiments, the paclitaxel is administered at a third dose at an amount of about 200 mg/m² to about 280 mg/m².

In some embodiments, the paclitaxel is administered at a third dose at an amount of about 200 mg/m² to about 275 mg/m².

In some embodiments, the paclitaxel is administered at a third dose at an amount of about 200 mg/m² to about 270 mg/m².

In some embodiments, the paclitaxel is administered at a third dose at an amount of about 200 mg/m² to about 265 mg/m².

In some embodiments, the paclitaxel is administered at a third dose at an amount of about 200 mg/m² to about 260 mg/m².

In some embodiments, the paclitaxel is administered at a third dose at an amount of about 200 mg/m² to about 255 mg/m².

In some embodiments, the paclitaxel is administered at a third dose at an amount of about 200 mg/m² to about 250 mg/m².

In some embodiments, the paclitaxel is administered at a third dose at an amount of about 200 mg/m² to about 245 mg/m².

In some embodiments, the paclitaxel is administered at a third dose at an amount of about 200 mg/m² to about 240 mg/m².

In some embodiments, the paclitaxel is administered at a third dose at an amount of about 200 mg/m² to about 235 mg/m².

In some embodiments, the paclitaxel is administered at a third dose at an amount of about 200 mg/m² to about 230 mg/m².

In some embodiments, the paclitaxel is administered at a third dose at an amount of about 200 mg/m² to about 225 mg/m².

In some embodiments, the paclitaxel is administered at a third dose at an amount of about 200 mg/m² to about 220 mg/m².

In some embodiments, the paclitaxel is administered at a third dose at an amount of about 200 mg/m² to about 215 mg/m².

In some embodiments, the paclitaxel is administered at a third dose at an amount of about 200 mg/m² to about 210 mg/m².

In some embodiments, the paclitaxel is administered at a third dose at an amount of about 200 mg/m² to about 205 mg/m².

In some embodiments, the paclitaxel is administered at a third dose at an amount of about 120 mg/m² to about 235 mg/m².

In some embodiments, the paclitaxel is administered at a third dose at an amount of about 120 mg/m² to about 230 mg/m².

In some embodiments, the paclitaxel is administered at a third dose at an amount of about 120 mg/m² to about 225 mg/m².

In some embodiments, the paclitaxel is administered at a third dose at an amount of about 120 mg/m² to about 220 mg/m².

In some embodiments, the paclitaxel is administered at a third dose at an amount of about 120 mg/m² to about 215 mg/m².

In some embodiments, the paclitaxel is administered at a third dose at an amount of about 120 mg/m² to about 210 mg/m².

In some embodiments, the paclitaxel is administered at a third dose at an amount of about 120 mg/m² to about 205 mg/m².

In some embodiments, the paclitaxel is administered at a third dose at an amount of about 120 mg/m² to about 200 mg/m².

In some embodiments, the paclitaxel is administered at a third dose at an amount of about 120 mg/m² to about 195 mg/m².

In some embodiments, the paclitaxel is administered at a third dose at an amount of about 120 mg/m² to about 190 mg/m².

In some embodiments, the paclitaxel is administered at a third dose at an amount of about 120 mg/m² to about 185 mg/m².

In some embodiments, the paclitaxel is administered at a third dose at an amount of about 120 mg/m² to about 180 mg/m².

In some embodiments, the paclitaxel is administered at a third dose at an amount of about 120 mg/m² to about 175 mg/m².

In some embodiments, the paclitaxel is administered at a third dose at an amount of about 120 mg/m² to about 170 mg/m².

In some embodiments, the paclitaxel is administered at a third dose at an amount of about 120 mg/m² to about 165 mg/m².

In some embodiments, the paclitaxel is administered at a third dose at an amount of about 120 mg/m² to about 160 mg/m².

In some embodiments, the paclitaxel is administered at a third dose at an amount of about 120 mg/m² to about 155 mg/m².

In some embodiments, the paclitaxel is administered at a third dose at an amount of about 120 mg/m² to about 150 mg/m².

In some embodiments, the paclitaxel is administered at a third dose at an amount of about 120 mg/m² to about 145 mg/m².

In some embodiments, the paclitaxel is administered at a third dose at an amount of about 120 mg/m² to about 140 mg/m².

In some embodiments, the paclitaxel is administered at a third dose at an amount of about 120 mg/m² to about 135 mg/m².

In some embodiments, the paclitaxel is administered at a third dose at an amount of about 120 mg/m² to about 130 mg/m².

In some embodiments, the paclitaxel is administered at a third dose at an amount of about 120 mg/m² to about 125 mg/m².

In some embodiments, the paclitaxel is administered at a third dose at an amount of about 125 mg/m² to about 235 mg/m².

In some embodiments, the paclitaxel is administered at a third dose at an amount of about 125 mg/m² to about 230 mg/m².

In some embodiments, the paclitaxel is administered at a third dose at an amount of about 125 mg/m² to about 225 mg/m².

In some embodiments, the paclitaxel is administered at a third dose at an amount of about 125 mg/m² to about 220 mg/m².

In some embodiments, the paclitaxel is administered at a third dose at an amount of about 125 mg/m² to about 215 mg/m².

In some embodiments, the paclitaxel is administered at a third dose at an amount of about 125 mg/m² to about 210 mg/m².

In some embodiments, the paclitaxel is administered at a third dose at an amount of about 125 mg/m² to about 205 mg/m².

In some embodiments, the paclitaxel is administered at a third dose at an amount of about 125 mg/m² to about 200 mg/m².

In some embodiments, the paclitaxel is administered at a third dose at an amount of about 125 mg/m² to about 195 mg/m².

In some embodiments, the paclitaxel is administered at a third dose at an amount of about 125 mg/m² to about 190 mg/m².

In some embodiments, the paclitaxel is administered at a third dose at an amount of about 125 mg/m² to about 185 mg/m².

In some embodiments, the paclitaxel is administered at a third dose at an amount of about 125 mg/m² to about 180 mg/m².

In some embodiments, the paclitaxel is administered at a third dose at an amount of about 125 mg/m² to about 175 mg/m².

In some embodiments, the paclitaxel is administered at a third dose at an amount of about 125 mg/m² to about 170 mg/m².

In some embodiments, the paclitaxel is administered at a third dose at an amount of about 125 mg/m² to about 165 mg/m².

In some embodiments, the paclitaxel is administered at a third dose at an amount of about 125 mg/m² to about 160 mg/m².

In some embodiments, the paclitaxel is administered at a third dose at an amount of about 125 mg/m² to about 155 mg/m².

In some embodiments, the paclitaxel is administered at a third dose at an amount of about 125 mg/m² to about 150 mg/m².

In some embodiments, the paclitaxel is administered at a third dose at an amount of about 125 mg/m² to about 145 mg/m².

In some embodiments, the paclitaxel is administered at a third dose at an amount of about 125 mg/m² to about 140 mg/m².

In some embodiments, the paclitaxel is administered at a third dose at an amount of about 125 mg/m² to about 135 mg/m².

In some embodiments, the paclitaxel is administered at a third dose at an amount of about 125 mg/m² to about 130 mg/m².

In some embodiments, the paclitaxel is administered at a third dose at an amount of about 130 mg/m² to about 235 mg/m².

In some embodiments, the paclitaxel is administered at a third dose at an amount of about 130 mg/m² to about 230 mg/m².

In some embodiments, the paclitaxel is administered at a third dose at an amount of about 130 mg/m² to about 225 mg/m².

In some embodiments, the paclitaxel is administered at a third dose at an amount of about 130 mg/m² to about 220 mg/m².

In some embodiments, the paclitaxel is administered at a third dose at an amount of about 130 mg/m² to about 215 mg/m².

In some embodiments, the paclitaxel is administered at a third dose at an amount of about 130 mg/m² to about 210 mg/m².

In some embodiments, the paclitaxel is administered at a third dose at an amount of about 130 mg/m² to about 205 mg/m².

In some embodiments, the paclitaxel is administered at a third dose at an amount of about 130 mg/m² to about 200 mg/m².

In some embodiments, the paclitaxel is administered at a third dose at an amount of about 130 mg/m² to about 195 mg/m².

In some embodiments, the paclitaxel is administered at a third dose at an amount of about 130 mg/m² to about 190 mg/m².

In some embodiments, the paclitaxel is administered at a third dose at an amount of about 130 mg/m² to about 185 mg/m².

In some embodiments, the paclitaxel is administered at a third dose at an amount of about 130 mg/m² to about 180 mg/m².

In some embodiments, the paclitaxel is administered at a third dose at an amount of about 130 mg/m² to about 175 mg/m².

In some embodiments, the paclitaxel is administered at a third dose at an amount of about 130 mg/m² to about 170 mg/m².

In some embodiments, the paclitaxel is administered at a third dose at an amount of about 130 mg/m² to about 165 mg/m².

In some embodiments, the paclitaxel is administered at a third dose at an amount of about 130 mg/m² to about 160 mg/m².

In some embodiments, the paclitaxel is administered at a third dose at an amount of about 130 mg/m² to about 155 mg/m².

In some embodiments, the paclitaxel is administered at a third dose at an amount of about 130 mg/m² to about 150 mg/m².

In some embodiments, the paclitaxel is administered at a third dose at an amount of about 130 mg/m² to about 145 mg/m².

In some embodiments, the paclitaxel is administered at a third dose at an amount of about 130 mg/m² to about 140 mg/m².

In some embodiments, the paclitaxel is administered at a third dose at an amount of about 130 mg/m² to about 135 mg/m².

In some embodiments, the paclitaxel is administered at a third dose at an amount of about 100 mg/m².

In some embodiments, the paclitaxel is administered at a third dose at an amount of about 105 mg/m².

In some embodiments, the paclitaxel is administered at a third dose at an amount of about 110 mg/m².

In some embodiments, the paclitaxel is administered at a third dose at an amount of about 115 mg/m².

In some embodiments, the paclitaxel is administered at a third dose at an amount of about 120 mg/m².

In some embodiments, the paclitaxel is administered at a third dose at an amount of about 125 mg/m².

In some embodiments, the paclitaxel is administered at a third dose at an amount of about 130 mg/m².

In some embodiments, the paclitaxel is administered at a third dose at an amount of about 135 mg/m².

In some embodiments, the paclitaxel is administered at a third dose at an amount of about 140 mg/m².

In some embodiments, the paclitaxel is administered at a third dose at an amount of about 145 mg/m².

In some embodiments, the paclitaxel is administered at a third dose at an amount of about 150 mg/m².

In some embodiments, the paclitaxel is administered at a third dose at an amount of about 155 mg/m².

In some embodiments, the paclitaxel is administered at a third dose at an amount of about 160 mg/m².

In some embodiments, the paclitaxel is administered at a third dose at an amount of about 165 mg/m².

In some embodiments, the paclitaxel is administered at a third dose at an amount of about 170 mg/m².

In some embodiments, the paclitaxel is administered at a third dose at an amount of about 175 mg/m².

In some embodiments, the paclitaxel is administered at a third dose at an amount of about 180 mg/m².

In some embodiments, the paclitaxel is administered at a third dose at an amount of about 185 mg/m².

In some embodiments, the paclitaxel is administered at a third dose at an amount of about 190 mg/m².

In some embodiments, the paclitaxel is administered at a third dose at an amount of about 195 mg/m².

In some embodiments, the paclitaxel is administered at a third dose at an amount of about 200 mg/m².

In some embodiments, the paclitaxel is administered at a third dose at an amount of about 205 mg/m².

In some embodiments, the paclitaxel is administered at a third dose at an amount of about 210 mg/m².

In some embodiments, the paclitaxel is administered at a third dose at an amount of about 215 mg/m².

In some embodiments, the paclitaxel is administered at a third dose at an amount of about 220 mg/m².

In some embodiments, the paclitaxel is administered at a third dose at an amount of about 225 mg/m².

In some embodiments, the paclitaxel is administered at a third dose at an amount of about 230 mg/m².

In some embodiments, the paclitaxel is administered at a third dose at an amount of about 235 mg/m².

In some embodiments, the paclitaxel is administered at a third dose at an amount of about 240 mg/m².

In some embodiments, the paclitaxel is administered at a third dose at an amount of about 245 mg/m².

In some embodiments, the paclitaxel is administered at a third dose at an amount of about 250 mg/m².

In some embodiments, the paclitaxel is administered at a third dose at an amount of about 255 mg/m².

In some embodiments, the paclitaxel is administered at a third dose at an amount of about 260 mg/m².

In some embodiments, the paclitaxel is administered at a third dose at an amount of about 265 mg/m².

In some embodiments, the paclitaxel is administered at a third dose at an amount of about 270 mg/m².

In some embodiments, the paclitaxel is administered at a third dose at an amount of about 275 mg/m².

In some embodiments, the paclitaxel is administered at a third dose at an amount of about 280 mg/m².

In some embodiments, the paclitaxel is administered at a third dose at an amount of about 285 mg/m².

In some embodiments, the paclitaxel is administered at a third dose at an amount of about 290 mg/m².

In some embodiments, the paclitaxel is administered at a third dose at an amount of about 295 mg/m².

In some embodiments, the paclitaxel is administered at a third dose at an amount of about 300 mg/m².

In some embodiments, the paclitaxel is administered at a third dose at an amount of about 305 mg/m².

In some embodiments, the paclitaxel is administered at a third dose at an amount of about 310 mg/m².

In some embodiments, the paclitaxel is administered at a third dose at an amount of about 315 mg/m².

In some embodiments, the paclitaxel is administered at a third dose at an amount of about 320 mg/m².

In some embodiments, the paclitaxel is administered at a third dose at an amount of about 325 mg/m².

In some embodiments, the paclitaxel is administered at a third dose at an amount of about 330 mg/m².

In some embodiments, the paclitaxel is administered at a third dose at an amount of about 335 mg/m².

In some embodiments, the paclitaxel is administered at a third dose at an amount of about 340 mg/m².

In some embodiments, the paclitaxel is administered at a third dose at an amount of about 345 mg/m².

In some embodiments, the paclitaxel is administered at a third dose at an amount of about 350 mg/m².

In some embodiments, the paclitaxel is administered at a third dose at an amount of about 355 mg/m².

In some embodiments, the paclitaxel is administered at a third dose at an amount of about 360 mg/m².

In some embodiments, the paclitaxel is administered at a third dose at an amount of about 365 mg/m².

In some embodiments, the paclitaxel is administered at a third dose at an amount of about 370 mg/m².

In some embodiments, the paclitaxel is administered at a third dose at an amount of about 375 mg/m².

In some embodiments, the paclitaxel is administered at a third dose at an amount of about 380 mg/m².

In some embodiments, the paclitaxel is administered at a third dose at an amount of about 385 mg/m².

In some embodiments, the paclitaxel is administered at a third dose at an amount of about 390 mg/m².

In some embodiments, the paclitaxel is administered at a third dose at an amount of about 395 mg/m².

In some embodiments, the paclitaxel is administered at a third dose at an amount of about 400 mg/m².

In some embodiments, the first dose is administered at an amount of about 205 mg/m² followed by a second dose.

In some embodiments, the first dose is administered at an amount of about 205 mg/m² followed by a second dose at an amount of 165 mg/m².

In some embodiments, the first dose is administered at an amount of about 205 mg/m² followed by a second dose at an amount of 165 mg/m², wherein the second dose is followed by a third dose.

In some embodiments, the first dose is administered followed by a second dose at an amount of 165 mg/m², wherein the second dose is followed by a third dose.

In some embodiments, the first dose is administered at an amount of about 205 mg/m² followed by a second dose at an amount of 165 mg/m², wherein the second dose is followed by a third dose at an amount of about 130 mg/m² .

In some embodiments, the first dose is administered followed by a second dose at an amount of 165 mg/m², wherein the second dose is followed by a third dose at an amount of about 130 mg/m² .

In some embodiments, the first dose is administered at an amount of about 205 mg/m² followed by a second dose, wherein the second dose is followed by a third dose at an amount of about 130 mg/m² .

In some embodiments, the first dose is administered followed by a second dose, wherein the second dose is followed by a third dose at an amount of about 130 mg/m².

In some embodiments, the first dose is administered in combination with Compound A.

In some embodiments, the second dose is administered in combination with Compound A.

In some embodiments, the third dose is administered in combination with Compound A.

In some embodiments, the first dose is administered until the subject displays one or more adverse reactions, and then the second dose is administered.

In some embodiments, the second dose is administered until the subject displays one or more adverse reactions, and then the third dose is administered.

In some embodiments, the paclitaxel and/or Compound A are admininstered at the various doses and dosing frequencies described herein for a duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks.

In some embodiments, the paclitaxel and/or Compound A are admininstered at the various doses and dosing frequencies described herein for a duration of between 1 week and 2 weeks, between 1 week and 3 weeks, between 1 week and 4 weeks, between 1 week and 5 weeks, between 1 week and 6 weeks, between 1 week and 7 weeks, between 1 week and 8 weeks, between 1 week and 9 weeks, between 1 week and 10 weeks, between 1 week and 11 weeks, between 1 week and 12 weeks, between 1 week and 13 weeks, between 1 week and 14 weeks, between 1 week and 15 weeks, between 1 week and 16 weeks, between 1 week and 17 weeks, between 1 week and 18 weeks, between 1 week and 19 weeks, between 1 week and 20 weeks, between 1 week and 21 weeks, between 1 week and 22 weeks, between 1 week and 23 weeks, between 1 week and 24 weeks, between 1 week and 25 weeks, between 1 week and 26 weeks, between 1 week and 27 weeks, between 1 week and 28 weeks, between 1 week and 29 weeks, or between 1 week and 30 weeks.

In some embodiments, the paclitaxel and/or Compound A are admininstered at the various doses and dosing frequencies described herein for a duration of between 2 weeks and 3 weeks, between 2 weeks and 4 weeks, between 2 weeks and 5 weeks, between 2 weeks and 6 weeks, between 2 weeks and 7 weeks, between 2 weeks and 8 weeks, between 2 weeks and 9 weeks, between 2 weeks and 10 weeks, between 2 weeks and 11 weeks, between 2 weeks and 12 weeks, between 2 weeks and 13 weeks, between 2 weeks and 14 weeks, between 2 weeks and 15 weeks, between 2 weeks and 16 weeks, between 2 weeks and 17 weeks, between 2 weeks and 18 weeks, between 2 weeks and 19 weeks, between 2 weeks and 20 weeks, between 2 weeks and 21 weeks, between 2 weeks and 22 weeks, between 2 weeks and 23 weeks, between 2 weeks and 24 weeks, between 2 weeks and 25 weeks, between 2 weeks and 26 weeks, between 2 weeks and 27 weeks, between 2 weeks and 28 weeks, between 2 weeks and 29 weeks, or between 2 weeks and 30 weeks.

In some embodiments, the paclitaxel and/or Compound A are admininstered at the various doses and dosing frequencies described herein for a duration of between 3 weeks and 4 weeks, between 3 weeks and 5 weeks, between 3 weeks and 6 weeks, between 3 weeks and 7 weeks, between 3 weeks and 8 weeks, between 3 weeks and 9 weeks, between 3 weeks and 10 weeks, between 3 weeks and 11 weeks, between 3 weeks and 12 weeks, between 3 weeks and 13 weeks, between 3 weeks and 14 weeks, between 3 weeks and 15 weeks, between 3 weeks and 16 weeks, between 3 weeks and 17 weeks, between 3 weeks and 18 weeks, between 3 weeks and 19 weeks, between 3 weeks and 20 weeks, between 3 weeks and 21 weeks, between 3 weeks and 22 weeks, between 3 weeks and 23 weeks, between 3 weeks and 24 weeks, between 3 weeks and 25 weeks, between 3 weeks and 26 weeks, between 3 weeks and 27 weeks, between 3 weeks and 28 weeks, between 3 weeks and 29 weeks, or between 3 weeks and 30 weeks.

In some embodiments, the paclitaxel and/or Compound A are admininstered at the various doses and dosing frequencies described herein for a duration of between 4 weeks and 5 weeks, between 4 weeks and 6 weeks, between 4 weeks and 7 weeks, between 4 weeks and 8 weeks, between 4 weeks and 9 weeks, between 4 weeks and 10 weeks, between 4 weeks and 11 weeks, between 4 weeks and 12 weeks, between 4 weeks and 13 weeks, between 4 weeks and 14 weeks, between 4 weeks and 15 weeks, between 4 weeks and 16 weeks, between 4 weeks and 17 weeks, between 4 weeks and 18 weeks, between 4 weeks and 19 weeks, between 4 weeks and 20 weeks, between 4 weeks and 21 weeks, between 4 weeks and 22 weeks, between 4 weeks and 23 weeks, between 4 weeks and 24 weeks, between 4 weeks and 25 weeks, between 4 weeks and 26 weeks, between 4 weeks and 27 weeks, between 4 weeks and 28 weeks, between 4 weeks and 29 weeks, or between 4 weeks and 30 weeks.

In some embodiments, the paclitaxel and/or Compound A are admininstered at the various doses and dosing frequencies described herein for a duration of between 5 weeks and 6 weeks, between 5 weeks and 7 weeks, between 5 weeks and 8 weeks, between 5 weeks and 9 weeks, between 5 weeks and 10 weeks, between 5 weeks and 11 weeks, between 5 weeks and 12 weeks, between 5 weeks and 13 weeks, between 5 weeks and 14 weeks, between 5 weeks and 15 weeks, between 5 weeks and 16 weeks, between 5 weeks and 17 weeks, between 5 weeks and 18 weeks, between 5 weeks and 19 weeks, between 5 weeks and 20 weeks, between 5 weeks and 21 weeks, between 5 weeks and 22 weeks, between 5 weeks and 23 weeks, between 5 weeks and 24 weeks, between 5 weeks and 25 weeks, between 5 weeks and 26 weeks, between 5 weeks and 27 weeks, between 5 weeks and 28 weeks, between 5 weeks and 29 weeks, or between 5 weeks and 30 weeks.

In some embodiments, the paclitaxel and/or Compound A are admininstered at the various doses and dosing frequencies described herein for a duration of between 6 weeks and 7 weeks, between 6 weeks and 8 weeks, between 6 weeks and 9 weeks, between 6 weeks and 10 weeks, between 6 weeks and 11 weeks, between 6 weeks and 12 weeks, between 6 weeks and 13 weeks, between 6 weeks and 14 weeks, between 6 weeks and 15 weeks, between 6 weeks and 16 weeks, between 6 weeks and 17 weeks, between 6 weeks and 18 weeks, between 6 weeks and 19 weeks, between 6 weeks and 20 weeks, between 6 weeks and 21 weeks, between 6 weeks and 22 weeks, between 6 weeks and 23 weeks, between 6 weeks and 24 weeks, between 6 weeks and 25 weeks, between 6 weeks and 26 weeks, between 6 weeks and 27 weeks, between 6 weeks and 28 weeks, between 6 weeks and 29 weeks, or between 6 weeks and 30 weeks.

In some embodiments, the paclitaxel and/or Compound A are admininstered at the various doses and dosing frequencies described herein for a duration of between 7 weeks and 8 weeks, between 7 weeks and 9 weeks, between 7 weeks and 10 weeks, between 7 weeks and 11 weeks, between 7 weeks and 12 weeks, between 7 weeks and 13 weeks, between 7 weeks and 14 weeks, between 7 weeks and 15 weeks, between 7 weeks and 16 weeks, between 7 weeks and 17 weeks, between 7 weeks and 18 weeks, between 7 weeks and 19 weeks, between 7 weeks and 20 weeks, between 7 weeks and 21 weeks, between 7 weeks and 22 weeks, between 7 weeks and 23 weeks, between 7 weeks and 24 weeks, between 7 weeks and 25 weeks, between 7 weeks and 26 weeks, between 7 weeks and 27 weeks, between 7 weeks and 28 weeks, between 7 weeks and 29 weeks, or between 7 weeks and 30 weeks.

In some embodiments, the paclitaxel and/or Compound A are admininstered at the various doses and dosing frequencies described herein for a duration of between 8 weeks and 9 weeks, between 8 weeks and 10 weeks, between 8 weeks and 11 weeks, between 8 weeks and 12 weeks, between 8 weeks and 13 weeks, between 8 weeks and 14 weeks, between 8 weeks and 15 weeks, between 8 weeks and 16 weeks, between 8 weeks and 17 weeks, between 8 weeks and 18 weeks, between 8 weeks and 19 weeks, between 8 weeks and 20 weeks, between 8 weeks and 21 weeks, between 8 weeks and 22 weeks, between 8 weeks and 23 weeks, between 8 weeks and 24 weeks, between 8 weeks and 25 weeks, between 8 weeks and 26 weeks, between 8 weeks and 27 weeks, between 8 weeks and 28 weeks, between 8 weeks and 29 weeks, or between 8 weeks and 30 weeks.

In some embodiments, the paclitaxel and/or Compound A are admininstered at the various doses and dosing frequencies described herein for a duration of between 9 weeks and 10 weeks, between 9 weeks and 11 weeks, between 9 weeks and 12 weeks, between 9 weeks and 13 weeks, between 9 weeks and 14 weeks, between 9 weeks and 15 weeks, between 9 weeks and 16 weeks, between 9 weeks and 17 weeks, between 9 weeks and 18 weeks, between 9 weeks and 19 weeks, between 9 weeks and 20 weeks, between 9 weeks and 21 weeks, between 9 weeks and 22 weeks, between 9 weeks and 23 weeks, between 9 weeks and 24 weeks, between 9 weeks and 25 weeks, between 9 weeks and 26 weeks, between 9 weeks and 27 weeks, between 9 weeks and 28 weeks, between 9 weeks and 29 weeks, or between 9 weeks and 30 weeks.

In some embodiments, the paclitaxel and/or Compound A are admininstered at the various doses and dosing frequencies described herein for a duration of between 10 weeks and 11 weeks, between 10 weeks and 12 weeks, between 10 weeks and 13 weeks, between 10 weeks and 14 weeks, between 10 weeks and 15 weeks, between 10 weeks and 16 weeks, between 10 weeks and 17 weeks, between 10 weeks and 18 weeks, between 10 weeks and 19 weeks, between 10 weeks and 20 weeks, between 10 weeks and 21 weeks, between 10 weeks and 22 weeks, between 10 weeks and 23 weeks, between 10 weeks and 24 weeks, between 10 weeks and 25 weeks, between 10 weeks and 26 weeks, between 10 weeks and 27 weeks, between 10 weeks and 28 weeks, between 10 weeks and 29 weeks, or between 10 weeks and 30 weeks.

In some embodiments, the paclitaxel and/or Compound A are admininstered at the various doses and dosing frequencies described herein for a duration of between 11 weeks and 12 weeks, between 11 weeks and 13 weeks, between 11 weeks and 14 weeks, between 11 weeks and 15 weeks, between 11 weeks and 16 weeks, between 11 weeks and 17 weeks, between 11 weeks and 18 weeks, between 11 weeks and 19 weeks, between 11 weeks and 20 weeks, between 11 weeks and 21 weeks, between 11 weeks and 22 weeks, between 11 weeks and 23 weeks, between 11 weeks and 24 weeks, between 11 weeks and 25 weeks, between 11 weeks and 26 weeks, between 11 weeks and 27 weeks, between 11 weeks and 28 weeks, between 11 weeks and 29 weeks, or between 11 weeks and 30 weeks.

In some embodiments, the paclitaxel and/or Compound A are admininstered at the various doses and dosing frequencies described herein for a duration of between 12 weeks and 13 weeks, between 12 weeks and 14 weeks, between 12 weeks and 15 weeks, between 12 weeks and 16 weeks, between 12 weeks and 17 weeks, between 12 weeks and 18 weeks, between 12 weeks and 19 weeks, between 12 weeks and 20 weeks, between 12 weeks and 21 weeks, between 12 weeks and 22 weeks, between 12 weeks and 23 weeks, between 12 weeks and 24 weeks, between 12 weeks and 25 weeks, between 12 weeks and 26 weeks, between 12 weeks and 27 weeks, between 12 weeks and 28 weeks, between 12 weeks and 29 weeks, or between 12 weeks and 30 weeks.

In some embodiments, the paclitaxel and/or Compound A are admininstered at the various doses and dosing frequencies described herein for a duration of between 13 weeks and 14 weeks, between 13 weeks and 15 weeks, between 13 weeks and 16 weeks, between 13 weeks and 17 weeks, between 13 weeks and 18 weeks, between 13 weeks and 19 weeks, between 13 weeks and 20 weeks, between 13 weeks and 21 weeks, between 13 weeks and 22 weeks, between 13 weeks and 23 weeks, between 13 weeks and 24 weeks, between 13 weeks and 25 weeks, between 13 weeks and 26 weeks, between 13 weeks and 27 weeks, between 13 weeks and 28 weeks, between 13 weeks and 29 weeks, or between 13 weeks and 30 weeks.

In some embodiments, the paclitaxel and/or Compound A are admininstered at the various doses and dosing frequencies described herein for a duration of between 14 weeks and 15 weeks, between 14 weeks and 16 weeks, between 14 weeks and 17 weeks, between 14 weeks and 18 weeks, between 14 weeks and 19 weeks, between 14 weeks and 20 weeks, between 14 weeks and 21 weeks, between 14 weeks and 22 weeks, between 14 weeks and 23 weeks, between 14 weeks and 24 weeks, between 14 weeks and 25 weeks, between 14 weeks and 26 weeks, between 14 weeks and 27 weeks, between 14 weeks and 28 weeks, between 14 weeks and 29 weeks, or between 14 weeks and 30 weeks.

In some embodiments, the paclitaxel and/or Compound A are admininstered at the various doses and dosing frequencies described herein for a duration of between 15 weeks and 16 weeks, between 15 weeks and 17 weeks, between 15 weeks and 18 weeks, between 15 weeks and 19 weeks, between 15 weeks and 20 weeks, between 15 weeks and 21 weeks, between 15 weeks and 22 weeks, between 15 weeks and 23 weeks, between 15 weeks and 24 weeks, between 15 weeks and 25 weeks, between 15 weeks and 26 weeks, between 15 weeks and 27 weeks, between 15 weeks and 28 weeks, between 15 weeks and 29 weeks, or between 15 weeks and 30 weeks.

In some embodiments, the paclitaxel and/or Compound A are admininstered at the various doses and dosing frequencies described herein for a duration of between 16 weeks and 17 weeks, between 16 weeks and 18 weeks, between 16 weeks and 19 weeks, between 16 weeks and 20 weeks, between 16 weeks and 21 weeks, between 16 weeks and 22 weeks, between 16 weeks and 23 weeks, between 16 weeks and 24 weeks, between 16 weeks and 25 weeks, between 16 weeks and 26 weeks, between 16 weeks and 27 weeks, between 16 weeks and 28 weeks, between 16 weeks and 29 weeks, or between 16 weeks and 30 weeks.

In some embodiments, the paclitaxel and/or Compound A are admininstered at the various doses and dosing frequencies described herein for a duration of between 17 weeks and 18 weeks, between 17 weeks and 19 weeks, between 17 weeks and 20 weeks, between 17 weeks and 21 weeks, between 17 weeks and 22 weeks, between 17 weeks and 23 weeks, between 17 weeks and 24 weeks, between 17 weeks and 25 weeks, between 17 weeks and 26 weeks, between 17 weeks and 27 weeks, between 17 weeks and 28 weeks, between 17 weeks and 29 weeks, or between 17 weeks and 30 weeks.

In some embodiments, the paclitaxel and/or Compound A are admininstered at the various doses and dosing frequencies described herein for a duration of between 18 weeks and 19 weeks, between 18 weeks and 20 weeks, between 18 weeks and 21 weeks, between 18 weeks and 22 weeks, between 18 weeks and 23 weeks, between 18 weeks and 24 weeks, between 18 weeks and 25 weeks, between 18 weeks and 26 weeks, between 18 weeks and 27 weeks, between 18 weeks and 28 weeks, between 18 weeks and 29 weeks, or between 18 weeks and 30 weeks.

In some embodiments, the paclitaxel and/or Compound A are admininstered at the various doses and dosing frequencies described herein for a duration of between 19 weeks and 20 weeks, between 19 weeks and 21 weeks, between 19 weeks and 22 weeks, between 19 weeks and 23 weeks, between 19 weeks and 24 weeks, between 19 weeks and 25 weeks, between 19 weeks and 26 weeks, between 19 weeks and 27 weeks, between 19 weeks and 28 weeks, between 19 weeks and 29 weeks, or between 19 weeks and 30 weeks.

In some embodiments, the paclitaxel and/or Compound A are admininstered at the various doses and dosing frequencies described herein for a duration of between 20 weeks and 21 weeks, between 20 weeks and 22 weeks, between 20 weeks and 23 weeks, between 20 weeks and 24 weeks, between 20 weeks and 25 weeks, between 20 weeks and 26 weeks, between 20 weeks and 27 weeks, between 20 weeks and 28 weeks, between 20 weeks and 29 weeks, or between 20 weeks and 30 weeks.

In some embodiments, the paclitaxel and/or Compound A are admininstered at the various doses and dosing frequencies described herein for a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the initial duration of administration of the paclitaxel and/or Compound A at the various doses and dosing frequencies described herein, the pactitaxle and/or Compound A are administered for a second duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration and the second duration is at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration and the second duration is 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the initial duration and the second duration of administration of the paclitaxel and/or Compound A at the various doses and dosing frequencies described herein, the pactitaxle and/or Compound A are administered for a third duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration, the second duration, and the third duration is at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration, the second duration, and the third duration is 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the paclitaxel and/or Compound A are each admininstered at a first dose described herein and at dosing frequencies described herein for a first duration described herein (e.g., at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks), the paclitaxel and/or Compound A are each admininstered at a second dose described herein and at dosing frequencies described herein for a second duration described herein. In some embodiments, the second dose for the paclitaxel and/or Compound A is the same as the first dose for paclitaxel and/or Compound A. In some embodiments, the second dose for the paclitaxel and/or Compound A is different from the first dose for paclitaxel and/or Compound A. In some embodiments, the second dose for the paclitaxel and/or Compound A is reduced from the first dose for paclitaxel and/or Compound A (e.g., for paclitaxel, the second dose is about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose).

In some embodiments, after the paclitaxel and/or Compound A are each admininstered at a first dose described herein and at dosing frequencies described herein for a first duration described herein, followed by a second dose described herein and at dosing frequencies described herein for a second duration described herein, the paclitaxel and/or Compound A are each admininstered at a third dose described herein and at dosing frequencies described herein for a third duration described herein. In some embodiments, the third dose for the paclitaxel and/or Compound A is the same as the second dose for paclitaxel and/or Compound A. In some embodiments, the third dose for the paclitaxel and/or Compound A is different from the second dose for paclitaxel and/or Compound A. In some embodiments, the third dose for the paclitaxel and/or Compound A is reduced from the second dose for paclitaxel and/or Compound A (e.g., for paclitaxel, the second dose is about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose).

In some embodiments, the paclitaxel and/or Compound A are each admininstered for a duration until the subject is treated. In some embodiments, the subject is treated when there is no disease progression (e.g., the cancer is not growing in size and/or severity, and not metastasizing). In some embodiments, the subject is treated when the disease is reversed (e.g., the cancer is reducing size and/or severity).

In some embodiments, the paclitaxel and/or Compound A are each admininstered for a duration until the subject displays one or more adverse reactions.In some embodiments, the paclitaxel and/or Compound A are each admininstered for a duration until the subject displays disease progression (e.g., the cancer is growing in size and/or severity, and/or the cancer is metastasizing).

In some embodiments, the intravenously administered paclitaxel (e.g., Taxol® or paclitaxel formulated with Cremophor®) is infused at an amount of about 135 mg/m² to about 175 mg/m².

In some embodiments, the intravenously administered paclitaxel (e.g., Taxol® or paclitaxel formulated with Cremophor®) is infused over a period of about 3 hours once every 3 weeks.

In some embodiments, the intravenously administered paclitaxel (e.g., Taxol® or paclitaxel formulated with Cremophor®) is infused over a period of about 6 hours once every 3 weeks.

In some embodiments, the intravenously administered paclitaxel (e.g., Taxol® or paclitaxel formulated with Cremophor®) is infused over a period of about 9 hours once every 3 weeks.

In some embodiments, the intravenously administered paclitaxel (e.g., Taxol® or paclitaxel formulated with Cremophor®) is infused over a period of about 12 hours once every 3 weeks.

In some embodiments, the intravenously administered paclitaxel (e.g., Taxol® or paclitaxel formulated with Cremophor®) is infused over a period of about 15 hours once every 3 weeks.

In some embodiments, the intravenously administered paclitaxel (e.g., Taxol® or paclitaxel formulated with Cremophor®) is infused over a period of about 18 hours once every 3 weeks.

In some embodiments, the intravenously administered paclitaxel (e.g., Taxol® or paclitaxel formulated with Cremophor®) is infused over a period of about 21 hours once every 3 weeks.

In some embodiments, the intravenously administered paclitaxel (e.g., Taxol® or paclitaxel formulated with Cremophor®) is infused over a period of about 24 hours once every 3 weeks.

In some embodiments, the intravenously administered paclitaxel (e.g., Taxol® or paclitaxel formulated with Cremophor®) is infused at an amount of about 80 mg/m² over a period of about 1 hour once weekly.

In some embodiments, the hematologic toxicity associated with the intravenous administration of paclitaxel in a subject suffering from an angiosarcoma includes anemia, and myelosuppression. In some embodiments, the myelosuppression may be from leukopenia, neutropenia, thrombocytopenia, or any combination thereof.

In some embodiments, the neurotoxicity associated with the intravenous administration of paclitaxel in a subject suffering from an angiosarcoma includes symptoms such as numbness, tingling, sharp pain, jabbing pain, burning pain, extreme sensitivity, loss of coordination, falling, weakness, paralysis, sweating, heat intolerance, dizziness, changes in blood pressure, bowel problems, bladder problems, or any combination thereof.

In some embodiments, the hypersensitivity-type infusion reactions associated with the intravenous administration of paclitaxel in a subject suffering from an angiosarcoma includes any sign or symptom on the first day of intravenous administration of paclitaxel. In some embodiments, the signs or symptoms in the subject include fever, rash, hives, pruritus, flushing, swelling, dyspnea, bronchospasm, stridor, reduced pulmonary expiratory flow, hypoxia, hypertension, hypotension, hypotonia, syncope, falling, incontinence, abdominal pain, vomiting, urticaria, facial swelling, eye disorders, headache, arrhythmia, tachycardia, nausea, chest pain, anaphylaxis, or any combination thereof.

In some embodiments, the paclitaxel is administered in capsules. In some embodiments, the paclitaxel is administered in 10 mg capsules. In some embodiments, the paclitaxel is administered in 15 mg capsules. In some embodiments, the paclitaxel is administered in 20 mg capsules. In some embodiments, the paclitaxel is administered in 25 mg capsules. In some embodiments, the paclitaxel is administered in 30 mg capsules. In some embodiments, the paclitaxel is administered in 35 mg capsules. In some embodiments, the paclitaxel is administered in 40 mg capsules. In some embodiments, the paclitaxel is administered in 45 mg capsules. In some embodiments, the paclitaxel is administered in 50 mg capsules.

In some embodiments, Compound A and the paclitaxel are administered on the same day.

In some embodiments, Compound A is administered before the paclitaxel is administered.

In some embodiments, Compound A is administered about 5 minutes before the paclitaxel is administered.

In some embodiments, Compound A is administered about 10 minutes before the paclitaxel is administered.

In some embodiments, Compound A is administered about 15 minutes before the paclitaxel is administered.

In some embodiments, Compound A is administered about 30 minutes before the paclitaxel is administered.

In some embodiments, Compound A is administered about 45 minutes before the paclitaxel is administered.

In some embodiments, Compound A is administered about 60 minutes before the paclitaxel is administered.

In some embodiments, Compound A is administered about 65 minutes before the paclitaxel is administered.

In some embodiments, Compound A is administered about 70 minutes before the paclitaxel is administered.

In some embodiments, Compound A is administered about 75 minutes before the paclitaxel is administered.

In some embodiments, Compound A is administered about 80 minutes before the paclitaxel is administered.

In some embodiments, Compound A is administered about 85 minutes before the paclitaxel is administered.

In some embodiments, Compound A is administered about 90 minutes before the paclitaxel is administered.

In some embodiments, Compound A is administered about 2 hours before the paclitaxel is administered.

In some embodiments, Compound A is administered about 3 hours before the paclitaxel is administered.

In some embodiments, Compound A is administered about 4 hours before the paclitaxel is administered.

In some embodiments, Compound A is administered about 6 hours before the paclitaxel is administered.

In some embodiments, Compound A is administered about 8 hours before the paclitaxel is administered.

In some embodiments, about 15 mg of Compound A is administered before the paclitaxel is administered.

In some embodiments, about 15 mg of Compound A is administered about 5 minutes before the paclitaxel is administered.

In some embodiments, about 15 mg of Compound A is administered about 10 minutes before the paclitaxel is administered.

In some embodiments, about 15 mg of Compound A is administered about 15 minutes before the paclitaxel is administered.

In some embodiments, about 15 mg of Compound A is administered about 30 minutes before the paclitaxel is administered.

In some embodiments, about 15 mg of Compound A is administered about 45 minutes before the paclitaxel is administered.

In some embodiments, about 15 mg of Compound A is administered about 60 minutes before the paclitaxel is administered.

In some embodiments, about 15 mg of Compound A is administered about 65 minutes before the paclitaxel is administered.

In some embodiments, about 15 mg of Compound A is administered about 70 minutes before the paclitaxel is administered.

In some embodiments, about 15 mg of Compound A is administered about 75 minutes before the paclitaxel is administered.

In some embodiments, about 15 mg of Compound A is administered about 80 minutes before the paclitaxel is administered.

In some embodiments, about 15 mg of Compound A is administered about 85 minutes before the paclitaxel is administered.

In some embodiments, about 15 mg of Compound A is administered about 90 minutes before the paclitaxel is administered.

In some embodiments, about 15 mg of Compound A is administered about 2 hours before the paclitaxel is administered.

In some embodiments, about 15 mg of Compound A is administered about 3 hours before the paclitaxel is administered.

In some embodiments, about 15 mg of Compound A is administered about 4 hours before the paclitaxel is administered.

In some embodiments, about 15 mg of Compound A is administered about 6 hours before the paclitaxel is administered.

In some embodiments, about 15 mg of Compound A is administered about 8 hours before the paclitaxel is administered.

In some embodiments, an anti-emetic is administered. In some embodiments, the anti-emetic is administered simultaneously with or prior to Compound A. In some embodiments, the anti-emetic is administered simultaneously with Compound A. In some embodiments, the anti-emetic is administered about 5 minutes before Compound A is administered. In some embodiments, the anti-emetic is administered about 10 minutes before Compound A is administered. In some embodiments, the anti-emetic is administered about 15 minutes before Compound A is administered. In some embodiments, the anti-emetic is administered about 30 minutes before Compound A is administered. In some embodiments, the anti-emetic is administered about 45 minutes before Compound A is administered. In some embodiments, the anti-emetic is administered about 60 minutes before Compound A is administered.

In some emdodiments, the anti-emetic is aprepitant. In some emdodiments, the anti-emetic is dexamethasone. In some emdodiments, the anti-emetic is dolasetron. In some emdodiments, the anti-emetic is granisetron. In some emdodiments, the anti-emetic is ondansetron. In some emdodiments, the anti-emetic is palonosetron. In some emdodiments, the anti-emetic is prochlorperazine. In some emdodiments, the anti-emetic is rolapitant. In some emdodiments, the anti-emetic is droperidol. In some emdodiments, the anti-emetic is metoclopramide. In some emdodiments, the anti-emetic is dimenhydrinate. In some emdodiments, the anti-emetic is diphenhydramine. In some emdodiments, the anti-emetic is meclizine. In some emdodiments, the anti-emetic is promethazine.

In some embodiments, a 5-hydroxytryptamine (5-HT3) antagonist is administered. In some embodiments, a 5-HT3 antagonist is administered simultaneously with Compound A. In some embodiments, a 5-HT3 antagonist is administered about 5 minutes before Compound A is administered. In some embodiments, a 5-HT3 antagonist is administered about 10 minutes before Compound A is administered. In some embodiments, a 5-HT3 antagonist is administered about 15 minutes before Compound A is administered. In some embodiments, a 5-HT3 antagonist is administered about 30 minutes before Compound A is administered. In some embodiments, a 5-HT3 antagonist is administered about 45 minutes before Compound A is administered. In some embodiments, a 5-HT3 antagonist is administered about 60 minutes before Compound A is administered. In some embodiment, 5-hydroxytryptamine (5-HT3) antagonist is administered for prophylaxis. In some embodiment, 5-hydroxytryptamine (5-HT3) antagonist is administered for reducing nausea. In some embodiment, a 5-hydroxytryptamine (5-HT3) antagonist is administered for reducing vomitting.

In some embodiments, a steroid antagonist is administered. In some embodiments, a steroid is administered simultaneously with Compound A. In some embodiments, a steroid is administered about 5 minutes before Compound A is administered. In some embodiments, a steroid is administered about 10 minutes before Compound A is administered. In some embodiments, a steroid is administered about 15 minutes before Compound A is administered. In some embodiments, a steroid is administered about 30 minutes before Compound A is administered. In some embodiments, a steroid is administered about 45 minutes before Compound A is administered. In some embodiments, a steroid is administered about 60 minutes before Compound A is administered. In some embodiment, a steroid is administered for reducing hypersensitivity-type reactions.

In some embodiments, a H-1 receptor antagonist is administered. In some embodiments, a H-1 receptor antagonist is administered at the same time as Compound A. In some embodiments, a H-1 receptor antagonist is administered simultaneously with Compound A. In some embodiments, a H-1 receptor antagonist is administered about 5 minutes before Compound A is administered. In some embodiments, a H-1 receptor antagonist is administered about 10 minutes before Compound A is administered. In some embodiments, a H-1 receptor antagonist is administered about 15 minutes before Compound A is administered. In some embodiments, a H-1 receptor antagonist is administered about 30 minutes before Compound A is administered. In some embodiments, a H-1 receptor antagonist is administered about 45 minutes before Compound A is administered. In some embodiments, a H-1 receptor antagonist is administered about 60 minutes before Compound A is administered. In some embodiment, a H-1 receptor antagonist is administered for reducing hypersensitivity-type reactions.

In some embodiments, a potential drug interaction exists between Compound A and moderate to strong inhibitors or inducers of CYP3A4 or CYP2C8. In some embodiments, a subject is not taking a medication which is a moderate to strong inhibitor or inducer of CYP3A4. In some embodiments, a subject is not taking a medication which is a moderate to strong inhibitor or inducer of CYP2C8. In some embodiments, a potential drug interaction exists between Compound A and inhibitors or inducers of P-gp. In some embodiments, a subject is not taking a medication which is a inhibitor or inducer of P-gp.

In some embodiments, a subject is not administered a medication known to be a strong P-gp inhibitor or inducer within 14 days before the oral admistration of paclitaxel. In some embodiments, a subject is not administered an oral medication with a narrow therapeutic index known to be a P-gp substrate (e.g., dabigatran and digoxin) within 24 hours before the oral admistration of paclitaxel. In some embodiments, a subject is not administered a medication known to be a strong CYP3A4 inhibitor (e.g., ketoconazole) or inducer (e.g., rifampin or St. John’s Wort) within 14 days before the oral admistration of paclitaxel. In some embodiments, a subject is not administered a medication known to be a strong inhibitor (e.g., gemfibrozil) or inducers (e.g., rifampin) of CYP2C8 within 14 days before the oral admistration of paclitaxel.

In some embodiments, the plasma exposure of the orally administered paclitaxel, as measured by AUC_{(0→∞),} is equal to or greater than the plasma exposure of intravenously administered paclitaxel (e.g., Taxol® or paclitaxel formulated with Cremophor®) at an amount of about 80 mg/m² over a period of about 60 minutes, as measured by AUC_{(0→∞).}

In some embodiments, the AUC_{(0→∞)} of the orally administered paclitaxel is equal to or greater than the AUC_{(0→∞)} of intravenously administered paclitaxel (e.g., Taxol® or paclitaxel formulated with Cremophor®) at an amount of about 80 mg/m² over a period of about 60 minutes, as measured by AUC_{(0→∞)}, in treating an angiosarcoma.

In some embodiments, the AUC_{(0→∞)} of the orally administered paclitaxel is equal to the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10, 15, 20, 25, 30, 35, 40, 45, 50, 55, 60, 65, 70, 75, 80, 85, 90, 95, or 100% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 10% greater (e.g., at least 10% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 20% greater (e.g., at least 20% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 30% greater (e.g., at least 30% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 40% greater (e.g., at least 40% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 50% greater (e.g., at least 50% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 60% greater (e.g., at least 60% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 70% greater (e.g., at least 70% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 80% greater (e.g., at least 80% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 90% greater (e.g., at least 90% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is at least about 100% greater (e.g., at least 100% greater) than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 100% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 90% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 80% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 70% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 60% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 50% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 40% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 35% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 30% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 25% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 10% greater to about 20% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the AUC_{(0→∞)} of the orally administered paclitaxel is about 15% greater to about 25% greater than the AUC_{(0→∞)} of intravenously administered paclitaxel (e.g., Taxol® or paclitaxel formulated with Cremophor®) at an amount of about 80 mg/m² over a period of about 60 minutes.

In some embodiments, the plasma exposure of the orally administered paclitaxel, as measured by AUC_{(0→∞),} is equal to or greater than the plasma exposure, as measured by AUC_{(0→∞),} of intravenously administered paclitaxel (e.g., Taxol® or paclitaxel formulated with Cremophor®) at an amount of about 125 mg/m² to about 175 mg/m² over a period of about 0.5 hours to about 3 hours once every 3 weeks.

In some embodiments, the plasma exposure of the orally administered paclitaxel, as measured by AUC_{(0→∞),} is equal to or greater than the plasma exposure, as measured by AUC_{(0→∞),} of intravenously administered paclitaxel (e.g., Taxol® or paclitaxel formulated with Cremophor®) at an amount of about 135 mg/m² to about 175 mg/m² over a period of about 3 hours to about 24 hours once every 3 weeks.

In some embodiments, the AUC_{(0→∞)} of the orally administered paclitaxel is about 2,000 ng•h/mL to about 10,000 ng•h/mL.

In some embodiments, the AUC_{(0→∞)} of the orally administered paclitaxel is about 2,000 ng•h/mL to about 9,000 ng•h/mL.

In some embodiments, the AUC_{(0→∞)} of the orally administered paclitaxel is about 3,000 ng•h/mL to about 9,000 ng•h/mL.

In some embodiments, the AUC_{(0→∞)} of the orally administered paclitaxel is about 3,000 ng•h/mL to about 8,000 ng•h/mL.

In some embodiments, the AUC_{(0→∞)} of the orally administered paclitaxel is about 3,000 ng•h/mL to about 7,000 ng•h/mL.

In some embodiments, the AUC_{(0→∞)} of the orally administered paclitaxel is about 4,000 ng•h/mL to about 7,000 ng•h/mL.

In some embodiments, the AUC_{(0→∞)} of the orally administered paclitaxel is about 5,000 ng•h/mL to about 7,000 ng•h/mL.

In some embodiments, the AUC_{(0→∞)} of the orally administered paclitaxel is about 2,000 ng•h/mL.

In some embodiments, the AUC_{(0→∞)} of the orally administered paclitaxel is about 3,000 ng•h/mL.

In some embodiments, the AUC_{(0→∞)} of the orally administered paclitaxel is about 4,000 ng•h/mL.

In some embodiments, the AUC_{(0→∞)} of the orally administered paclitaxel is about 5,000 ng•h/mL.

In some embodiments, the AUC_{(0→∞)} of the orally administered paclitaxel is about 6,000 ng•h/mL.

In some embodiments, the AUC_{(0→∞)} of the orally administered paclitaxel is about 7,000 ng•h/mL.

In some embodiments, the AUC_{(0→∞)} of the orally administered paclitaxel is about 8,000 ng•h/mL.

In some embodiments, the AUC_{(0→∞)} of the orally administered paclitaxel is about 9,000 ng•h/mL.

In some embodiments, the AUC_{(0→∞)} of the orally administered paclitaxel is about 10,000 ng•h/mL.

In some embodiments, the total amount of the paclitaxel orally administered per week is about 300 mg/m² to about 2,000 mg/m².

In some embodiments, the total amount of the paclitaxel orally administered per week is about 300 mg/m² to about 1,900 mg/m².

In some embodiments, the total amount of the paclitaxel orally administered per week is about 300 mg/m² to about 1,800 mg/m².

In some embodiments, the total amount of the paclitaxel orally administered per week is about 300 mg/m² to about 1,700 mg/m².

In some embodiments, the total amount of the paclitaxel orally administered per week is about 300 mg/m² to about 1,600 mg/m².

In some embodiments, the total amount of the paclitaxel orally administered per week is about 300 mg/m² to about 1,500 mg/m².

In some embodiments, the total amount of the paclitaxel orally administered per week is about 300 mg/m² to about 1,400 mg/m².

In some embodiments, the total amount of the paclitaxel orally administered per week is about 300 mg/m² to about 1,300 mg/m².

In some embodiments, the total amount of the paclitaxel orally administered per week is about 300 mg/m² to about 1,200 mg/m².

In some embodiments, the total amount of the paclitaxel orally administered per week is about 300 mg/m² to about 1,100 mg/m².

In some embodiments, the total amount of the paclitaxel orally administered per week is about 300 mg/m² to about 1,000 mg/m².

In some embodiments, the total amount of the paclitaxel orally administered per week is about 300 mg/m² to about 900 mg/m².

In some embodiments, the total amount of the paclitaxel orally administered per week is about 350 mg/m² to about 850 mg/m².

In some embodiments, the total amount of the paclitaxel orally administered per week is about 400 mg/m² to about 800 mg/m².

In some embodiments, the total amount of the paclitaxel orally administered per week is about 400 mg/m² to about 750 mg/m².

In some embodiments, the total amount of the paclitaxel orally administered per week is about 400 mg/m² to about 700 mg/m².

In some embodiments, the total amount of the paclitaxel orally administered per week is about 400 mg/m² to about 650 mg/m².

In some embodiments, the total amount of the paclitaxel orally administered per week is about 400 mg/m² to about 600 mg/m².

In some embodiments, the total amount of the paclitaxel orally administered per week is about 400 mg/m² to about 550 mg/m².

In some embodiments, the total amount of the paclitaxel orally administered per week is about 400 mg/m² to about 500 mg/m².

In some embodiments, the total amount of the paclitaxel orally administered per week is about 450 mg/m² to about 750 mg/m².

In some embodiments, the total amount of the paclitaxel orally administered per week is about 450 mg/m² to about 700 mg/m².

In some embodiments, the total amount of the paclitaxel orally administered per week is about 450 mg/m² to about 650 mg/m².

In some embodiments, the total amount of the paclitaxel orally administered per week is about 450 mg/m² to about 600 mg/m².

In some embodiments, the total amount of the paclitaxel orally administered per week is about 450 mg/m² to about 550 mg/m².

In some embodiments, the total amount of the paclitaxel orally administered per week is about 450 mg/m² to about 500 mg/m².

In some embodiments, the total amount of the paclitaxel orally administered per week is about 500 mg/m² to about 750 mg/m².

In some embodiments, the total amount of the paclitaxel orally administered per week is about 500 mg/m² to about 700 mg/m².

In some embodiments, the total amount of the paclitaxel orally administered per week is about 500 mg/m² to about 650 mg/m².

In some embodiments, the total amount of the paclitaxel orally administered per week is about 500 mg/m² to about 600 mg/m².

In some embodiments, the total amount of the paclitaxel orally administered per week is about 500 mg/m² to about 550 mg/m².

In some embodiments, the total amount of the paclitaxel orally administered per week is about 550 mg/m² to about 650 mg/m².

In some embodiments, the total amount of the paclitaxel orally administered per week is about 550 mg/m² to about 645 mg/m².

In some embodiments, the total amount of the paclitaxel orally administered per week is about 550 mg/m² to about 640 mg/m².

In some embodiments, the total amount of the paclitaxel orally administered per week is about 550 mg/m² to about 635 mg/m².

In some embodiments, the total amount of the paclitaxel orally administered per week is about 550 mg/m² to about 630 mg/m².

In some embodiments, the total amount of the paclitaxel orally administered per week is about 550 mg/m² to about 625 mg/m².

In some embodiments, the total amount of the paclitaxel orally administered per week is about 550 mg/m² to about 620 mg/m².

In some embodiments, the total amount of the paclitaxel orally administered per week is about 550 mg/m² to about 615 mg/m².

In some embodiments, the total amount of the paclitaxel orally administered per week is about 550 mg/m² to about 610 mg/m².

In some embodiments, the total amount of the paclitaxel orally administered per week is about 550 mg/m² to about 605 mg/m².

In some embodiments, the total amount of the paclitaxel orally administered per week is about 550 mg/m² to about 600 mg/m².

In some embodiments, the total amount of the paclitaxel orally administered per week is about 550 mg/m² to about 595 mg/m².

In some embodiments, the total amount of the paclitaxel orally administered per week is about 550 mg/m² to about 590 mg/m².

In some embodiments, the total amount of the paclitaxel orally administered per week is about 550 mg/m² to about 585 mg/m².

In some embodiments, the total amount of the paclitaxel orally administered per week is about 550 mg/m² to about 580 mg/m².

In some embodiments, the total amount of the paclitaxel orally administered per week is about 550 mg/m² to about 575 mg/m².

In some embodiments, the total amount of the paclitaxel orally administered per week is about 550 mg/m² to about 570 mg/m².

In some embodiments, the total amount of the paclitaxel orally administered per week is about 550 mg/m² to about 565 mg/m².

In some embodiments, the total amount of the paclitaxel orally administered per week is about 550 mg/m² to about 560 mg/m².

In some embodiments, the total amount of the paclitaxel orally administered per week is about 550 mg/m² to about 555 mg/m².

In some embodiments, the total amount of the paclitaxel orally administered per week is about 450 mg/m² to about 550 mg/m².

In some embodiments, the total amount of the paclitaxel orally administered per week is about 450 mg/m² to about 545 mg/m².

In some embodiments, the total amount of the paclitaxel orally administered per week is about 450 mg/m² to about 540 mg/m².

In some embodiments, the total amount of the paclitaxel orally administered per week is about 450 mg/m² to about 535 mg/m².

In some embodiments, the total amount of the paclitaxel orally administered per week is about 450 mg/m² to about 530 mg/m².

In some embodiments, the total amount of the paclitaxel orally administered per week is about 450 mg/m² to about 525 mg/m².

In some embodiments, the total amount of the paclitaxel orally administered per week is about 450 mg/m² to about 520 mg/m².

In some embodiments, the total amount of the paclitaxel orally administered per week is about 450 mg/m² to about 515 mg/m².

In some embodiments, the total amount of the paclitaxel orally administered per week is about 450 mg/m² to about 510 mg/m².

In some embodiments, the total amount of the paclitaxel orally administered per week is about 450 mg/m² to about 505 mg/m².

In some embodiments, the total amount of the paclitaxel orally administered per week is about 450 mg/m² to about 500 mg/m².

In some embodiments, the total amount of the paclitaxel orally administered per week is about 450 mg/m² to about 495 mg/m².

In some embodiments, the total amount of the paclitaxel orally administered per week is about 450 mg/m² to about 490 mg/m².

In some embodiments, the total amount of the paclitaxel orally administered per week is about 450 mg/m² to about 485 mg/m².

In some embodiments, the total amount of the paclitaxel orally administered per week is about 450 mg/m² to about 480 mg/m².

In some embodiments, the total amount of the paclitaxel orally administered per week is about 450 mg/m² to about 475 mg/m².

In some embodiments, the total amount of the paclitaxel orally administered per week is about 450 mg/m² to about 470 mg/m².

In some embodiments, the total amount of the paclitaxel orally administered per week is about 450 mg/m² to about 465 mg/m².

In some embodiments, the total amount of the paclitaxel orally administered per week is about 450 mg/m² to about 460 mg/m².

In some embodiments, the total amount of the paclitaxel orally administered per week is about 450 mg/m² to about 455 mg/m².

In some embodiments, the total amount of the paclitaxel orally administered per week is about 350 mg/m² to about 450 mg/m².

In some embodiments, the total amount of the paclitaxel orally administered per week is about 350 mg/m² to about 445 mg/m².

In some embodiments, the total amount of the paclitaxel orally administered per week is about 350 mg/m² to about 440 mg/m².

In some embodiments, the total amount of the paclitaxel orally administered per week is about 350 mg/m² to about 435 mg/m².

In some embodiments, the total amount of the paclitaxel orally administered per week is about 350 mg/m² to about 430 mg/m².

In some embodiments, the total amount of the paclitaxel orally administered per week is about 350 mg/m² to about 425 mg/m².

In some embodiments, the total amount of the paclitaxel orally administered per week is about 350 mg/m² to about 420 mg/m².

In some embodiments, the total amount of the paclitaxel orally administered per week is about 350 mg/m² to about 415 mg/m².

In some embodiments, the total amount of the paclitaxel orally administered per week is about 350 mg/m² to about 410 mg/m².

In some embodiments, the total amount of the paclitaxel orally administered per week is about 350 mg/m² to about 405 mg/m².

In some embodiments, the total amount of the paclitaxel orally administered per week is about 350 mg/m² to about 400 mg/m².

In some embodiments, the total amount of the paclitaxel orally administered per week is about 350 mg/m² to about 395 mg/m².

In some embodiments, the total amount of the paclitaxel orally administered per week is about 350 mg/m² to about 390 mg/m².

In some embodiments, the total amount of the paclitaxel orally administered per week is about 350 mg/m² to about 385 mg/m².

In some embodiments, the total amount of the paclitaxel orally administered per week is about 350 mg/m² to about 380 mg/m².

In some embodiments, the total amount of the paclitaxel orally administered per week is about 350 mg/m² to about 375 mg/m².

In some embodiments, the total amount of the paclitaxel orally administered per week is about 350 mg/m² to about 370 mg/m².

In some embodiments, the total amount of the paclitaxel orally administered per week is about 350 mg/m² to about 365 mg/m².

In some embodiments, the total amount of the paclitaxel orally administered per week is about 350 mg/m² to about 360 mg/m².

In some embodiments, the total amount of the paclitaxel orally administered per week is about 350 mg/m² to about 355 mg/m².

In some embodiments, the total amount of the paclitaxel orally administered per week is about 300, 325, 350, 375, 400, 425, 450, 475, 500, 525, 550, 575, 600, 605, 610, 615, 620, 625, 630, 635, 640, 645, 650, 655, 660, 665, 670, 675, 680, 685, 690, 695, 700, 725, 750, 775, 800, 825, 850, 875, 900, 925, 950, 975, 1,000, 1,025, 1,050, 1,075, 1,100, 1,125, 1,150, 1,175, 1,200, 1,225, 1,250, 1,275, 1,300, 1,325, 1,350, 1,375, 1,400, 1,425, 1,450, 1,475, 1,500, 1,525, 1,550, 1,575, 1,600, 1,625, 1,650, 1,675, 1,700, 1,725, 1,750, 1,775, or 1,800 mg/m². In some embodiments, the total amount of the paclitaxel orally administered per week is about 375, 400, 425, 450, 475, 500, 525, 550, 575, 600, 605, 610, 615, 620, 625, 630, 635, 640, 645, or 650 mg/m².

In some embodiments, the total amount of the paclitaxel orally administered per week is at least about 300 mg/m² (e.g., at least 300 mg/m²).

In some embodiments, the total amount of the paclitaxel orally administered per week is at least about 350 mg/m² (e.g., at least 350 mg/m²).

In some embodiments, the total amount of the paclitaxel orally administered per week is at least about 355 mg/m² (e.g., at least 355 mg/m²).

In some embodiments, the total amount of the paclitaxel orally administered per week is at least about 360 mg/m² (e.g., at least 360 mg/m²).

In some embodiments, the total amount of the paclitaxel orally administered per week is at least about 365 mg/m² (e.g., at least 365 mg/m²).

In some embodiments, the total amount of the paclitaxel orally administered per week is at least about 370 mg/m² (e.g., at least 370 mg/m²).

In some embodiments, the total amount of the paclitaxel orally administered per week is at least about 375 mg/m² (e.g., at least 375 mg/m²).

In some embodiments, the total amount of the paclitaxel orally administered per week is at least about 380 mg/m² (e.g., at least 380 mg/m²).

In some embodiments, the total amount of the paclitaxel orally administered per week is at least about 385 mg/m² (e.g., at least 385 mg/m²).

In some embodiments, the total amount of the paclitaxel orally administered per week is at least about 390 mg/m² (e.g., at least 390 mg/m²).

In some embodiments, the total amount of the paclitaxel orally administered per week is at least about 395 mg/m² (e.g., at least 395 mg/m²).

In some embodiments, the total amount of the paclitaxel orally administered per week is at least about 400 mg/m² (e.g., at least 400 mg/m²).

In some embodiments, the total amount of the paclitaxel orally administered per week is at least about 405 mg/m² (e.g., at least 405 mg/m²).

In some embodiments, the total amount of the paclitaxel orally administered per week is at least about 410 mg/m² (e.g., at least 410 mg/m²).

In some embodiments, the total amount of the paclitaxel orally administered per week is at least about 415 mg/m² (e.g., at least 415 mg/m²).

In some embodiments, the total amount of the paclitaxel orally administered per week is at least about 420 mg/m² (e.g., at least 420 mg/m²).

In some embodiments, the total amount of the paclitaxel orally administered per week is at least about 425 mg/m² (e.g., at least 425 mg/m²).

In some embodiments, the total amount of the paclitaxel orally administered per week is at least about 430 mg/m² (e.g., at least 430 mg/m²).

In some embodiments, the total amount of the paclitaxel orally administered per week is at least about 435 mg/m² (e.g., at least 435 mg/m²).

In some embodiments, the total amount of the paclitaxel orally administered per week is at least about 440 mg/m² (e.g., at least 440 mg/m²).

In some embodiments, the total amount of the paclitaxel orally administered per week is at least about 445 mg/m² (e.g., at least 445 mg/m²).

In some embodiments, the total amount of the paclitaxel orally administered per week is at least about 450 mg/m² (e.g., at least 450 mg/m²).

In some embodiments, the total amount of the paclitaxel orally administered per week is at least about 455 mg/m² (e.g., at least 455 mg/m²).

In some embodiments, the total amount of the paclitaxel orally administered per week is at least about 460 mg/m² (e.g., at least 460 mg/m²).

In some embodiments, the total amount of the paclitaxel orally administered per week is at least about 465 mg/m² (e.g., at least 465 mg/m²).

In some embodiments, the total amount of the paclitaxel orally administered per week is at least about 470 mg/m² (e.g., at least 470 mg/m²).

In some embodiments, the total amount of the paclitaxel orally administered per week is at least about 475 mg/m² (e.g., at least 475 mg/m²).

In some embodiments, the total amount of the paclitaxel orally administered per week is at least about 480 mg/m² (e.g., at least 480 mg/m²).

In some embodiments, the total amount of the paclitaxel orally administered per week is at least about 485 mg/m² (e.g., at least 485 mg/m²).

In some embodiments, the total amount of the paclitaxel orally administered per week is at least about 490 mg/m² (e.g., at least 490 mg/m²).

In some embodiments, the total amount of the paclitaxel orally administered per week is at least about 495 mg/m² (e.g., at least 495 mg/m²).

In some embodiments, the total amount of the paclitaxel orally administered per week is at least about 500 mg/m² (e.g., at least 500 mg/m²).

In some embodiments, the total amount of the paclitaxel orally administered per week is at least about 505 mg/m² (e.g., at least 505 mg/m²).

In some embodiments, the total amount of the paclitaxel orally administered per week is at least about 510 mg/m² (e.g., at least 510 mg/m²).

In some embodiments, the total amount of the paclitaxel orally administered per week is at least about 515 mg/m² (e.g., at least 515 mg/m²).

In some embodiments, the total amount of the paclitaxel orally administered per week is at least about 520 mg/m² (e.g., at least 520 mg/m²).

In some embodiments, the total amount of the paclitaxel orally administered per week is at least about 525 mg/m² (e.g., at least 525 mg/m²).

In some embodiments, the total amount of the paclitaxel orally administered per week is at least about 530 mg/m² (e.g., at least 530 mg/m²).

In some embodiments, the total amount of the paclitaxel orally administered per week is at least about 535 mg/m² (e.g., at least 535 mg/m²).

In some embodiments, the total amount of the paclitaxel orally administered per week is at least about 540 mg/m² (e.g., at least 540 mg/m²).

In some embodiments, the total amount of the paclitaxel orally administered per week is at least about 545 mg/m² (e.g., at least 545 mg/m²).

In some embodiments, the total amount of the paclitaxel orally administered per week is at least about 550 mg/m² (e.g., at least 550 mg/m²).

In some embodiments, the total amount of the paclitaxel orally administered per week is at least about 555 mg/m² (e.g., at least 555 mg/m²).

In some embodiments, the total amount of the paclitaxel orally administered per week is at least about 560 mg/m² (e.g., at least 560 mg/m²).

In some embodiments, the total amount of the paclitaxel orally administered per week is at least about 565 mg/m² (e.g., at least 565 mg/m²).

In some embodiments, the total amount of the paclitaxel orally administered per week is at least about 570 mg/m² (e.g., at least 570 mg/m²).

In some embodiments, the total amount of the paclitaxel orally administered per week is at least about 575 mg/m² (e.g., at least 575 mg/m²).

In some embodiments, the total amount of the paclitaxel orally administered per week is at least about 580 mg/m² (e.g., at least 580 mg/m²).

In some embodiments, the total amount of the paclitaxel orally administered per week is at least about 585 mg/m² (e.g., at least 585 mg/m²).

In some embodiments, the total amount of the paclitaxel orally administered per week is at least about 590 mg/m² (e.g., at least 590 mg/m²).

In some embodiments, the total amount of the paclitaxel orally administered per week is at least about 595 mg/m² (e.g., at least 595 mg/m²).

In some embodiments, the total amount of the paclitaxel orally administered per week is at least about 600 mg/m² (e.g., at least 600 mg/m²).

In some embodiments, the total amount of the paclitaxel orally administered per week is at least about 605 mg/m² (e.g., at least 605 mg/m²).

In some embodiments, the total amount of the paclitaxel orally administered per week is at least about 610 mg/m² (e.g., at least 610 mg/m²).

In some embodiments, the total amount of the paclitaxel orally administered per week is at least about 615 mg/m² (e.g., at least 615 mg/m²).

In some embodiments, the total amount of the paclitaxel orally administered per week is at least about 620 mg/m² (e.g., at least 620 mg/m²).

In some embodiments, the total amount of the paclitaxel orally administered per week is at least about 625 mg/m² (e.g., at least 625 mg/m²).

In some embodiments, the total amount of the paclitaxel orally administered per week is at least about 630 mg/m² (e.g., at least 630 mg/m²).

In some embodiments, the total amount of the paclitaxel orally administered per week is at least about 635 mg/m² (e.g., at least 635 mg/m²).

In some embodiments, the total amount of the paclitaxel orally administered per week is at least about 640 mg/m² (e.g., at least 640 mg/m²).

In some embodiments, the total amount of the paclitaxel orally administered per week is at least about 645 mg/m² (e.g., at least 645 mg/m²).

In some embodiments, the total amount of the paclitaxel orally administered per week is at least about 650 mg/m² (e.g., at least 650 mg/m²).

In some embodiments, the total amount of the paclitaxel orally administered per week is at least about 655 mg/m² (e.g., at least 655 mg/m²).

In some embodiments, the total amount of the paclitaxel orally administered per week is at least about 660 mg/m² (e.g., at least 660 mg/m²).

In some embodiments, the total amount of the paclitaxel orally administered per week is at least about 675 mg/m² (e.g., at least 675 mg/m²).

In some embodiments, the total amount of the paclitaxel orally administered per week is at least about 700 mg/m² (e.g., at least 700 mg/m²).

In some embodiments, the total amount of the paclitaxel orally administered per week is at least about 750 mg/m² (e.g., at least 750 mg/m²).

In some embodiments, the total amount of the paclitaxel orally administered per week is at least about 800 mg/m² (e.g., at least 800 mg/m²).

In some embodiments, the total amount of the paclitaxel orally administered per week is at least about 825 mg/m² (e.g., at least 825 mg/m²).

In some embodiments, the total amount of the paclitaxel orally administered per week is at least about 875 mg/m² (e.g., at least 875 mg/m²).

In some embodiments, the total amount of the paclitaxel orally administered per week is at least about 900 mg/m² (e.g., at least 900 mg/m²).

In some embodiments, the total amount of the paclitaxel orally administered per week is at least about 975 mg/m² (e.g., at least 975 mg/m²).

In some embodiments, the total amount of the paclitaxel orally administered per week is at least about 1,000 mg/m² (e.g., at least 1,000 mg/m²).

In some embodiments, the total amount of the paclitaxel orally administered per week is at least about 1,050 mg/m² (e.g., at least 1,050 mg/m²).

In some embodiments, the total amount of the paclitaxel orally administered per week is at least about 1,100 mg/m² (e.g., at least 1,100 mg/m²).

In some embodiments, the total amount of the paclitaxel orally administered per week is at least about 1,125 mg/m² (e.g., at least 1,125 mg/m²).

In some embodiments, the total amount of the paclitaxel orally administered per week is at least about 1,200 mg/m² (e.g., at least 1,200 mg/m²).

In some embodiments, the total amount of the paclitaxel orally administered per week is at least about 1,250 mg/m² (e.g., at least 1,250 mg/m²).

In some embodiments, the total amount of the paclitaxel orally administered per week is at least about 1,300 mg/m² (e.g., at least 1,300 mg/m²).

In some embodiments, the total amount of the paclitaxel orally administered per week is at least about 1,350 mg/m² (e.g., at least 1,350 mg/m²).

In some embodiments, the total amount of the paclitaxel orally administered per week is at least about 1,375 mg/m² (e.g., at least 1,375 mg/m²).

In some embodiments, the total amount of the paclitaxel orally administered per week is at least about 1,400 mg/m² (e.g., at least 1,400 mg/m²).

In some embodiments, the total amount of the paclitaxel orally administered per week is at least about 1,500 mg/m² (e.g., at least 1,500 mg/m²).

In some embodiments, the total amount of the paclitaxel orally administered per week is at least about 1,625 mg/m² (e.g., at least 1,625 mg/m²).

In some embodiments, the total amount of the paclitaxel orally administered per week is at least about 1,650 mg/m² (e.g., at least 1,650 mg/m²).

In some embodiments, the total amount of the paclitaxel orally administered per week is at least about 1,750 mg/m² (e.g., at least 1,750 mg/m²).

In some embodiments, the total amount of the paclitaxel orally administered per week is at least about 1,800 mg/m² (e.g., at least 1,800 mg/m²).

In some embodiments, the intravenously administered paclitaxel is formulated in compositions that comprise paclitaxel and a pharmaceutical excipient or carrier that facilitates the intravenous administration of paclitaxel. In some embodiments, the intravenously administered paclitaxel is formulated with polyethoxylated castor oil, e.g., Cremophor®. In some embodiments, the intravenously administered paclitaxel is Taxol®, or a generic version thereof. In some embodiments, the intravenously administered paclitaxel is formulated with a protein carrier. In some embodiments, the intravenously administered paclitaxel is formulated in a composition comprising protein-bound paclitaxel, i.e., Abraxane®.

In some embodiments, the angiosarcoma is a disease that involves abnormal cell growth with the potential to invade or spread to other parts of the body.

In some embodiments, the angiosarcoma is a cutaneous angiosarcoma, i.e., angiosarcoma of the skin. In some embodiments, the angiosarcoma is a subcutaneous angiosarcoma, i.e., angiosarcoma appearing underneath the surface of the skin. In some embodiments, the angiosarcoma is an angiosarcoma of the limbs, trunk, head and neck, bone, or organ. In some embodiments, the angiosarcoma is an angiosarcoma of the deep tissue. In some embodiments, the angiosarcoma is an angiosarcoma of the limbs. In some embodiments, the angiosarcoma is an angiosarcoma of the arm. In some embodiments, the angiosarcoma is an angiosarcoma of the upper arm. In some embodiments, the angiosarcoma is an angiosarcoma of the lower arm. In some embodiments, the angiosarcoma is an angiosarcoma of the leg. In some embodiments, the angiosarcoma is an angiosarcoma of the upper leg. In some embodiments, the angiosarcoma is an angiosarcoma of the lower leg. In some embodiments, the angiosarcoma is an angiosarcoma of the hand. In some embodiments, the angiosarcoma is an angiosarcoma of the foot. In some embodiments, the angiosarcoma is an angiosarcoma of the trunk. In some embodiments, the angiosarcoma is an angiosarcoma of the torso. In some embodiments, the angiosarcoma is an angiosarcoma of the abdominal area. In some embodiments, the angiosarcoma is an angiosarcoma of the chest. In some embodiments, the angiosarcoma is an angiosarcoma of the back. In some embodiments, the angiosarcoma is an angiosarcoma of the breast. In some embodiments, the angiosarcoma is an angiosarcoma of the head and neck. In some embodiments, the angiosarcoma is an angiosarcoma of the head. In some embodiments, the angiosarcoma is an angiosarcoma of the scalp. In some embodiments, the angiosarcoma is an angiosarcoma of the nose. In some embodiments, the angiosarcoma is an angiosarcoma of the ears. In some embodiments, the angiosarcoma is an angiosarcoma of the mouth. In some embodiments, the angiosarcoma is an angiosarcoma of the eyes. In some embodiments, the angiosarcoma is an angiosarcoma of the neck. In some embodiments, the angiosarcoma is an angiosarcoma of the bone. In some embodiments, the angiosarcoma is an angiosarcoma of the organs. In some embodiments, the angiosarcoma is an angiosarcoma of the liver or spleen. In some embodiments, the angiosarcoma is an angiosarcoma of the liver. In some embodiments, the angiosarcoma is an angiosarcoma of the spleen. In some embodiments, the angiosarcoma is an angiosarcoma of the breast, liver, spleen, or deep tissue. In some embodiments, the angiosarcoma is an angiosarcoma of the head and neck or breast. In some embodiments, the angiosarcoma is an angiosarcoma of the scalp and breast. In some embodiments, the angiosarcoma is an angiosarcoma of the nose.

In some embodiments, the subject is fasted before paclitaxel and/or Compound A is orally administered. In some embodiments, the subject is fasted for at least 3 hours, at least 4 hours, at least 6 hours, at least 8 hours, at least 12 hours, at least 18 hours, or at least 24 hours before treatment. In some embodiments, the subject is fasted for at least 3 hours before treatment. In some embodiments, the subject is fasted for at least 4 hours before treatment. In some embodiments, the subject is fasted for at least 5 hours before treatment. In some embodiments, the subject is fasted for at least 6 hours before treatment. In some embodiments, the subject is fasted for at least 7 hours before treatment. In some embodiments, the subject is fasted for at least 8 hours before treatment. In some embodiments, the subject is fasted for at least 9 hours before treatment. In some embodiments, the subject is fasted for at least 10 hours before treatment. In some embodiments, the subject is fasted for at least 12 hours, at least 18 hours, or at least 24 hours before treatment.

In some embodiments, the subject is fasted after paclitaxel and/or Compound A is orally administered. In some embodiments, the subject is fasted for at least 30 minutes, at least 45 minutes, at least 60 minutes, at least 75 minutes, at least 90 minutes, at least 105 minutes, at least 120 minutes, at least 135 minutes, at least 150 minutes, at least 165 minutes, at least 180 minutes, at least 210 minutes, or at least 240 minutes after treatment. In some embodiments, the subject is fasted for at least 30 minutes after treatment. In some embodiments, the subject is fasted for at least 45 minutes after treatment. In some embodiments, the subject is fasted for at least 60 minutes after treatment. In some embodiments, the subject is fasted for at least 75 minutes after treatment. In some embodiments, the subject is fasted for at least 90 minutes after treatment. In some embodiments, the subject is fasted for at least 105 minutes after treatment. In some embodiments, the subject is fasted for at least 120 minutes after treatment. In some embodiments, the subject is fasted for at least 135 minutes after treatment. In some embodiments, the subject is fasted for at least 150 minutes after treatment. In some embodiments, the subject is fasted for at least 165 minutes after treatment. In some embodiments, the subject is fasted for at least 180 minutes after treatment. In some embodiments, the subject is fasted for at least 210 minutes after treatment. In some embodiments, the subject is fasted for at least 240 minutes after treatment.

In some embodiments, the size of the angiosarcoma descreases for at least 10% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 20% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 30% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 40% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 50% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 60% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 70% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 80% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 90% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma descreases for at least 95% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel.

In some embodiments, the size of the angiosarcoma is measured according to RECIST v 1.1. In some embodiments, the size of the angiosarcoma is measured in at least 1 dimension and the longest diameter in the plane of measurement is recorded.

In some embodiments, there is no adverse reactions within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, within 20 weeks, within 25 weeks, within 30 weeks, within 35 weeks, or within 40 weeks, of the administration of paclitaxel. In some embodiments, there is no adverse reactions during the entire duration of the administration of paclitaxel.

In some embodiments, there is no severe adverse reactions within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, within 20 weeks, within 25 weeks, within 30 weeks, within 35 weeks, or within 40 weeks, of the administration of paclitaxel. In some embodiments, there is no severe adverse reactions during the entire duration of the administration of paclitaxel.

An adverse reaction, as used herein, is any reaction caused by or associated with the oral administration of paclitaxel and/or Compound A that is or is considered to be undesirable or harmful. An adverse reaction can be mild, moderate, or severe, depending on the type of the adverse reaction and/or the level/amount of the adverse reaction. In some embodiments, the adverse reaction is gastrointestinal (e.g., vomiting, nausea, abdominal discomfort, or diarrhea). In some embodiments, the adverse reaction is neurotoxicity (e.g., peripheral neuropathy). In some embodiments, the adverse reaction is hematological in nature (e.g., hematological toxicities associated with IV paclitaxel). In some embodiments, the adverse reaction is leucopenia. In some embodiments, the adverse reaction is febrile neutropenia. In some embodiments, the adverse reaction is neutropenia. In some embodiments, the adverse reaction is neutropenia with infection. In some embodiments, the adverse reaction is neutropenia without infection. In some embodiments, the adverse reaction is any unfavorable or unintended sign. In some embodiments, the adverse reaction is any new disease or exacerbation of an existing disease. In some embodiments, the adverse reaction is any deterioration in nonprotocol-required measurements of a laboratory value or other clinical test. In some embodiments, the adverse reaction is recurrence of an intermittent medical condition. In some embodiments, the adverse reaction is headache. In some embodiments, the adverse reaction is life threatening.

In some embodiments, an adverse reaction includes neutropenia, alopecia, anemia, arthralgia/myalgia, diarrhea, leukopenia, nausea/vomiting, opportunistic infections, peripheral neuropathy, thrombocytopenia, mucositis, hypersensitivity, renal impairment, hypotension, bradycardia, grand mal seizures, cardiac conduction abnormalities, Pyrexia, dehydration, pancytopenia, congestive heart failure, left ventricular dysfunction, Stevens-Johnson syndrome, toxic epidermal necrolysis, and extravasation.

In some embodiments, the subject meets the Inclusion Criteria for treatment. In some embodiments, the subject is 18 years or older. In some embodiments, the subject has histologically-confirmed cutaneous angiosarcoma that is not amenable to curative intent surgery (e.g., locally advanced disease and disease for which surgical resection would carry an unacceptable risk of recurrence or morbidity to the subject). In some embodiments, the subject has not had previous treatment of angiosarcoma with taxanes. In some embodiments, the disease is measurable per RECIST v. 1.1. In some embodiments, the subject has an Eastern Cooperative Oncology Group (ECOG11) performance status <1. In some embodiments, the subject has resolution of all acute AEs from prior cancer therapeies to NCI CTCAE v4.03 Grade ≤ 1 or to that subject’s baseline.

In some embodiments, the subject has adequate organ function. In some embodiments, adequate organ function is renal function as evidenced by serum creatinine ≤ 1.5 x upper limit of normal (ULN) or calculated creatinine clearance ≥ 50 mL/ min per the Cockcroft and Gault formula. In some embodiments, adequate organ function is bone marrow function as evidenced by absolute neutrophil count, hemoglobin, and platelet count. In some embodiments, adequate organ function is liver function as evidenced by total bilirubin within normal limits, alanine aminotransferase (ALT) ≤_3 × ULN, and aspartate aminotransferase (AST) ≤3 × ULN, gamma-glutamyl transferase (GGT) ≤ 10 x ULN, and alkaline phosphatase ≤3 × ULN.

In some embodiments, the subject is male. In some embodiments, the subject is male and sterile (including vasectomy confirmed by post vasectomy semen analysis).

In some embodiments, the subject is female. In some embodiments, the female is of non-child bearing potential due to surgical sterilization confirmed by medical history or menopause. In some embodiments, the female is of child-bearing potential and tests negative for pregnancy based on serum pregnancy test.

In some embodiments, the subject has a life expectancy of at least 3 months.

In some embodiments, the subject is between the ages of about 0 years old to about 110 years old. In some embodiments, the subject is between the ages of about 0 years old to about 10 years old. In some embodiments, the subject is between the ages of about 10 years old to about 20 years old. In some embodiments, the subject is between the ages of about 20 years old to about 30 years old. In some embodiments, the subject is between the ages of about 30 years old to about 40 years old. In some embodiments, the subject is between the ages of about 40 years old to about 50 years old. In some embodiments, the subject is between the ages of about 50 years old to about 60 years old. In some embodiments, the subject is between the ages of about 60 years old to about 70 years old. In some embodiments, the subject is between the ages of about 70 years old to about 80 years old. In some embodiments, the subject is between the ages of about 80 years old to about 90 years old. In some embodiments, the subject is between the ages of about 90 years old to about 100 years old. In some embodiments, the subject is between the ages of about 90 years old to about 110 years old. In some embodiments, the subject is between the ages of about 60 years old to about 110 years old. In some embodiments, the subject is between the ages of about 60 years old to about 100 years old. In some embodiments, the subject is between the ages of about 60 years old to about 90 years old. In some embodiments, the subject is between the ages of about 60 years old to about 80 years old. In some embodiments, the subject is between the ages of about 60 years old to about 70 years old.

Unless explicitly indicated otherwise, the terms “approximately” and “about” are synonymous. In some embodiments, “approximately” and “about” refer to the recited amount, dose, value (for example, ∞AUC_{(0→}∞₎), or duration ± 20%, ± 15%, ± 10%, ± 8%, ± 6%, ± 5%, ± 4%, ± 2%, ± 1%, or ± 0.5%. In another embodiment, “approximately” and “about” refer to the listed amount, dose, value, or duration ± 10%, ± 8%, ± 6%, ± 5%, ± 4%, or ± 2%. In yet another embodiment, “approximately” and “about” refer to the listed amount, dose, value, or duration ± 5%. In yet another embodiment, “approximately” and “about” refer to the listed amount, dose, value, or duration ± 2%. In yet another embodiment, “approximately” and “about” refer to the listed amount, dose, value, or duration ± 1%. When the terms “approximately” and “about” are used when reciting temperature or temperature range, these terms refer to the recited temperature or temperature range ± 5° C., ± 2° C., or ± 1° C. In another embodiment, the terms “approximately” and “about” refer to the recited temperature or temperature range ± 2° C.

The term “paclitaxel” refers to 5,20-Epoxy-1,2,4,7,10-13-hexahydroxytax-11-en-9-one-4,10-diacetate 2-benzoate 13-ester with (2R,3S)-N-benzoyl-3-phenylisoserine, i.e., the compound with the following structure:

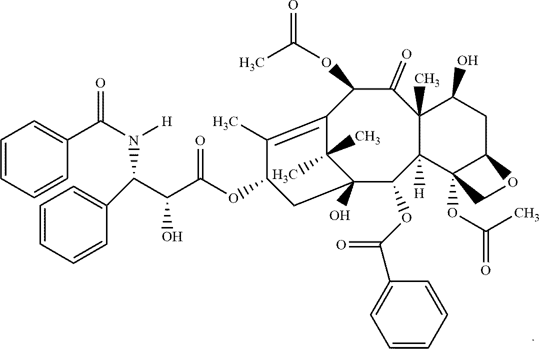

Unless otherwise indicated, the term “paclitaxel” includes pharmaceutically acceptable salts and/or solvates thereof.

Paclitaxel suitable for intravenous administration or intravenously administered paclitaxel includes compositions that comprise paclitaxel and a pharmaceutical excipient or carrier that facilitates the intravenous administration of paclitaxel. Such pharmaceutical excipient or carrier includes polyethoxylated castor oil, e.g., Cremophor ®. In some embodiments, paclitaxel suitable for intravenous administration or intravenously administered paclitaxel includes the brand name product, TAXOL®, and generic versions thereof. In some embodiments, paclitaxel suitable for intravenous administration or intravenously administered paclitaxel includes a composition comprising protein-bound paclitaxel, i.e., Abraxane®. In some embodiments, paclitaxel suitable for intravenous administration exhibits clinical toxicities. In some embodiments, paclitaxel suitable for intravenous administration exhibits clinical toxicities due to the excipient or carrier. In some embodiments, paclitaxel suitable for intravenous administration exhibits clinical toxicities due to the polyethoxylated castor oil excipient or carrier.

Paclitaxel suitable for oral administration or orally administered paclitaxel refers to a formulation of paclitaxel that is administered orally. In some embodiments, the orally administered paclitaxel is in capsule form. In some embodiments, each capsule contains paclitaxel and a surfactant, e.g., polysorbate 80. In some embodiments, each capsule contains 30 mg-200 mg of paclitaxel. In some embodiments, each capsule contains 30 mg, 45 mg, 60 mg, 75 mg, 90 mg, 100 mg, 125 mg, 150 mg, 175 mg, or 200 mg of paclitaxel. In some embodiments, each capsule contains 30 mg of paclitaxel. In some embodiments, each capsule contains 500 mg of polysorbate 80. In some embodiments each capsule contains 30 mg of paclitaxel and 500 mg of polysorbate 80. In some embodiments, the orally administered paclitaxel is in tablet form. In some embodiments, each tablet contains 30 mg - 200 mg of paclitaxel. In some embodiments, each tablet contains 30 mg, 45 mg, 60 mg, 75 mg, 90 mg, 100 mg, 125 mg, 150 mg, 175 mg, or 200 mg of paclitaxel. In some embodiments, each tablet contains 30 mg of paclitaxel.

Oral formulation of paclitaxel (e.g., capsule or tablet) may be formulated by any suitable methods known in the art.

The term “Compound A” refers to a compound, or a pharmaceutically acceptable salt and/or solvate thereof, which is a P-gp pump inhibitor and has the following structure:

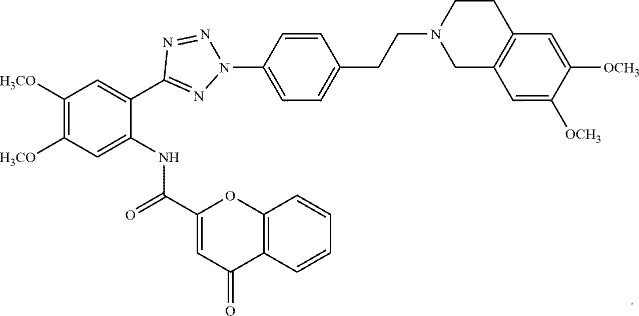

Unless indicated otherwise, the terms “Compound A,” “HM30181 methanesulfonate monohydrate,” “HM30181A,” “HM30181AK,” and “HM30181AK-US” are all equivalent and are used interchangeably. In some embodiments, Compound A refers to a methanesulfonate salt monohydrate of Compound A:

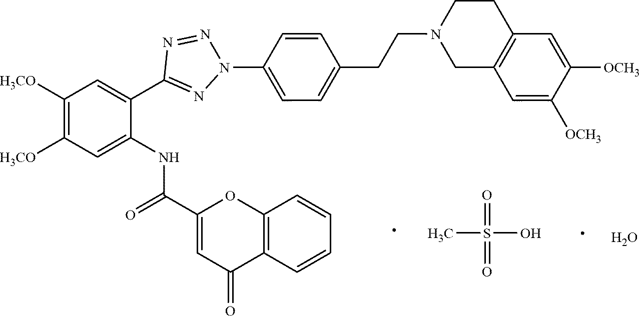

In some embodiments, Compound A refers to a methanesulfonate salt monohydrate of Compound A. Compound A is commercially available, e.g., in tablet form suitable for oral administration. In some embodiments, Compound A is administered in 15 mg tablets suitable for oral administration.

Compound A may be formulated by any suitable methods known in the art.

Angiosarcoma is defined as a distinct subset of soft tissue sarcomas (Toro et al., Int. J. Cancer 119(12): 2922-30, 2006). Angiosarcoma can be definitively diagnosed histologically with the use of tumor biomarkers (Lahat et al., Clin. Mol. Insights. Ann. Surg. 251(6): 1098-106, 2010), and can be clearly differentiated from Kaposi’s sarcoma. Angiosarcomas express the typical vascular biomarkers: CD34, CD31, Fli1, ERG and podoplanin (D2-40, lymphatic marker). Sometimes co-expressed are epithelial antigens: EMA, Cam5.2 and AE⅓. Immunostaining for the Kaposi’s sarcoma herpes virus is negative (Antonescu, Modern Pathol. 27: S30-S38, 2014).

The term “subject” includes any living organism that has an angiosarcoma or is at a risk of developing an angiosarcoma. In some embodiments, the term “subject” refers to a mammal that has an angiosarcoma or is at a risk of developing an angiosarcoma. In some embodiments, the term subject refers to a human being that has an angiosarcoma or is at a risk of developing an angiosarcoma. In some embodiments, the term subject refers to an angiosarcoma patient, i.e., a patient.

The term “AUC(0→∞)” refers to the total exposure to a drug and is expressed in unit of concentration time. In some embodiments, it is the concentration of a drug over a time interval circulating in the body, e.g., in the plasma, blood, or serum. In some embodiments, AUC(0→∞) = AUC(₀→_{Tlast}) + (CTlast/Kelim), where C_{Tlast} is the last measureable measurable drug concentration and Kₑₗᵢₘ is the terminal elimination rate constant, expressed in time⁻¹ units.

In some embodiments, C_{Tlast} may be determined from about 1 day to about 21 days after oral administration of paclitaxel.

In some embodiments, C_{Tlast} may be determined from about 2 days to about 14 days after oral administration of paclitaxel.

In some embodiments, C_{Tlast} may be determined from about 3 days to about 7 days after oral administration of paclitaxel.

In some embodiments, C_{Tlast} may be determined at about 3 days after oral administration of paclitaxel.

In some embodiments, C_{Tlast} may be determined at about 4 days after oral administration of paclitaxel.

In some embodiments, C_{Tlast} may be determined at about 5 days after oral administration of paclitaxel.

In some embodiments, C_{Tlast} may be determined at about 6 days after oral administration of paclitaxel.

In some embodiments, C_{Tlast} may be determined at about 7 days after oral administration of paclitaxel.

Hematologic toxicity associated with the intravenous administration of paclitaxel in a subject suffering from an angiosarcoma can be assessed by a medical professional or health care worker by analyzing blood samples in a subject, i.e., determining cell counts, including white blood cells, absolute neutrophils, platelets, and hemoglobin.

Hypersensitivity-type infusion reactions, and symptoms associated with hematologic toxicity and/or neurotoxicity associated with the intravenous administration of paclitaxel in a subject suffering from an angiosarcoma can be assessed by a medical professional or health care worker.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject depends upon the subject’s body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active ingredient or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing paclitaxel may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active ingredient into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. The composition is stable under the conditions of manufacture and storage and is preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating paclitaxel in the required amount in an appropriate solvent with one or a combination of ingredients enumerated above, as required, followed by filtered sterilization. Generally, dispersions are prepared by incorporating form paclitaxel into a sterile vehicle that contains a basic dispersion medium and the required other ingredients from those enumerated above. In the case of sterile powders for the preparation of sterile injectable solutions, methods of preparation are vacuum drying and freeze-drying that yields a powder of paclitaxel plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, form can be incorporated with excipients and used in the form of tablets, troches, or capsules. Oral compositions can also be prepared using a fluid carrier for use as a mouthwash, wherein in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The pharmaceutical compositions described herein can be included in a container, pack, or dispenser together with instructions for administration.

All percentages and ratios used herein, unless otherwise indicated, are by weight.

Other features and advantages of the present application are apparent from the different examples. The provided examples illustrate different components and methodology useful in practicing the present application.

The examples do not limit the claimed application. Based on the present disclosure the skilled artisan can identify and employ other components and methodology useful for practicing the present application.

### EXAMPLES

### Example 1 - In Vivo Anti-Cancer Activity in Tumor Cell Implant Mouse Models of Orally Administered Paclitaxel in Combination with Compound A

In seven in vivo experiments in mice, the ability of orally administered paclitaxel in combination with Compound A to inhibit the growth of various human tumor transplant cell lines was studied. Paclitaxel alone, administered IV or intraperitoneally (IP), was used as control. Summary and results of the study are presented in the Table 1 below.

TABLE 1

Summary and Results of In vivo Anti-Cancer Activity in Tumor Cell Implant Mouse Models of Orally Administered Paclitaxel in combination with Compound A

| Mouse strain | Implant: human cancer cell line | Paclitaxel together with Compound A | | Paclitaxel Alone | |
|---|---|---|---|---|---|
| | | Paclitaxel Dose | Effect | Dose | Effect |
| NU/Nu Balb/C | SKOV-3 ovarian | 30 mg/kg 2x weekly for 3 weeks | 91.52 IR% | | |
| | | 40 mg 1x weekly for 3 weeks | 58.21 IR% | 30 mg/kg IV, 1x weekly for 3 weeks | 72.79 IR% |
| NU/Nu Balb/C | A431 vaginal epidermoid | 30 mg/kg 2x weekly for 3 weeks | 91.78 IR% | 30 mg/kg IV, 1x weekly for 3 weeks | 45.41 IR% |
| NU/Nu Balb/C | HT-29 colon | 50 mg/kg every 4 days for 4 doses | 96.28 IR% | 25 mg/kg IP every 4 days for 4 doses | 65.82 IR% |
| NU/Nu Balb/C | HT-29 colon | 40 mg/kg every 4 days for 4 doses | 40.21% tumor regression | 20 mg/kg IV every 4 days for 4 doses | 163.09% tumor regression |
| NCr-nu (athymic) | SK-OV-3 ovarian | 30 mg/kg every other day for 4 doses | >78.6 days growth delay | 18 mg/kg IV, every other day for 4 doses | 43.8 days growth delay |
| NCr-nu (athymic) | MDA-MB-231 mammary | 30 mg/kg every other day for 4 doses | >55.9 days growth delay | 18 mg/kg IV, every other day for 4 doses | >55.9 days growth delay |
| Athymic nude | NCI-H460 lung | 50 mg/kg every other day for 4 doses | 12.7 days growth delay | 24 mg/kg IV, every other day for 4 doses | 16.3 days growth delay |

In these studies, tumor inhibition rate (IR%) was calculated as ([1-relative tumor growth in treated group/relative tumor growth in control group] x 100). Tumor regression was calculated as (Mean Tumor WeightEnd of treatment/Mean tumor WeightBeginning of treatment) x100. Therefore, a value <100% for tumor regression indicates a decrease in tumor weight over the course of the study and a value >100% indicates an increase in tumor weight. Tumor growth delay was calculated as the median number of days to reach two tumor mass doublings.

In all studies, orally administered paclitaxel in combination with Compound A was active against transplanted human tumor cell lines. Paclitaxel administered IP or IV was generally less active. Because of differences in dose levels, comparative activity of the orally administered paclitaxel administered with IV or IP administered paclitaxel may not be reliably estimated in the absence of plasma or tissue exposure levels.

These results showed that oral administration of paclitaxel in combination with Compound A is effective in inhibiting the growth of various human tumor cell lines after implantation into mice. In addition, no adverse effects in a battery of CNS, respiratory and cardiovascular safety pharmacology studies were observed. Oral administration of paclitaxel in combination with Compound A caused minor reductions in body temperature in a CNS safety pharmacology study in rats, and reductions in PR and QRS intervals in a cardiovascular safety pharmacology in dogs. These effects are interpreted as being caused by paclitaxel, due to their absence when Compound A was administered alone.

### Example 2 - Dose Dependency of Orally Administered Paclitaxel

In rats and dogs administered with paclitaxel orally in combination with Compound A, Cₘₐₓ and AUC for paclitaxel increased in a greater than dose-proportional manner as shown in the Table 2 and Table 3.

TABLE 2

Dose-Dependency of Paclitaxel Pharmacokinetics in Rats After Single Dose Oral Administration in Combination with Compound A

| Dose Paclitaxel (mg/kg) | AUClast (ng.hr/mL) | Cmax (ng/mL) | Tmax (Hr) | t½ (Hr) |
|---|---|---|---|---|
| | Paclitaxel | Paclitaxel | Paclitaxel | Paclitaxel |
| 5 | 333 | 103 | 0.6 | 8.2 |
| 10 | 1027 | 356 | 0.7 | 8.7 |
| 20 | 3009 | 852 | 1.2 | 7.5 |
| 30 | 6638 | 1761 | 1.2 | 6.0 |
| 40 | 11052 | 2488 | 1.5 | 5.5 |
| 50 | 13124 | 3067 | 1.4 | 5.3 |

TABLE 3

Dose-Dependency of Paclitaxel Pharmacokinetics in Dogs After Single Dose Oral Administration in Combination with Compound A

| Dose Paclitaxel (mg/kg) | AUClast (ng.hr/mL) | Cmax (ng/mL) | Tmax (Hr) | t_{½} (Hr) |
|---|---|---|---|---|
| | Paclitaxel | Paclitaxel | Paclitaxel | Paclitaxel |
| 2 | 413 | 128 | 0.5 | 7.4 |
| 4 | 1331 | 661 | 0.7 | 8.1 |
| 6 | 2123 | 1109 | 0.5 | 7.3 |

### Example 3 - Determination of the Bioavailability of Orally Administered Paclitaxel in Combination with Compound A in Subjects Suffering from Cancers

A study was designed to determine the absolute bioavailability of orally administered paclitaxel in combination with Compound A in subjects suffering from cancer. Eligible subjects were adults for whom therapy with intravenously administered paclitaxel (e.g., Taxol® or paclitaxel formulated with Cremophor®) at a dose of 80 mg/m² over 1 hour once per week is indicated.

The first 6 subjects received the following treatments approximately 1 week apart: 80 mg/m² intravenously administered paclitaxel (as Taxol® or generic) infused over 1 hour (on day 1 or day 8) plus premedication per standard local practice; or 15 mg Compound A plus 270 mg (about 150 mg/m²) orally administered paclitaxel (days 1 and 2 or days 8 and 9). Compound A was administered 1 hour before oral paclitaxel. No premedication was allowed during this treatment sequence.

Based on preliminary results from the first 6 subjects, the protocol was amended to compare the extent of absorption of orally administered paclitaxel to that of 80 mg/m² intravenously administered paclitaxel (e.g., Taxol® or paclitaxel formulated with Cremophor®) at doses that were considered likely to meet criteria for bioequivalence by AUC(0→∞).

The amendment allowed for studying 2 additional subjects in a crossover design comparing oral paclitaxel at a daily dose of 274 mg/m² for 2 consecutive days and 80 mg/m² intravenously administered paclitaxel (e.g., Taxol® or paclitaxel formulated with Cremophor®) over 1 hour. Following the completion of the 6^{th} subject in the original 270 mg dose group, the protocol was further amended to compare the exposure of up to 2 additional cohorts of 2 subjects each at doses of orally administered paclitaxel that were increased by up to 25%, or decreased, if needed, to achieve a paclitaxel exposure similar to that of 80 mg/m² intravenously administered paclitaxel (e.g., Taxol® or paclitaxel formulated with Cremophor®)over 1 hour.

Based on preliminary data from 6 subjects at 270 mg per day for 2 days and 2 subjects at a daily dose of 274 mg/m² for 2 consecutive days, it was estimated that the bioavailability of orally administered paclitaxel is approximately 13.4%. Therefore, orally administered paclitaxel at a dose of 313 mg/m² daily for 2 consecutive days per week, i.e., a weekly dose of 626 mg/m² orally administered paclitaxel, is expected to produce similar exposure to 80 mg/m² intravenously administered paclitaxel (e.g., Taxol® or paclitaxel formulated with Cremophor®) over 1 hour (within 80% to 125%).

### Example 4 - Determination of Maximum Tolerated Dose (MTD) for Orally Administered Paclitaxel in Combination with Compound A

To determine the MTD for orally administered paclitaxel, a Phase 1 study was conducted in 24 subjects with advanced solid cancer. An oral liquid formulation of paclitaxel was given on days 1, 8 and 15 in a cycle of 28 days. Paclitaxel doses ranged from 60 to 420 mg/m², and Compound A in tablet was dosed at half of paclitaxel doses (30 to 210 mg/m²) and administered 1 hour before paclitaxel.

TABLE 4

Dose Level to Determination of Maximum Tolerated Dose (MTD) for Orally Administered Paclitaxel in Combination with Compound A

| Dose Level | Paclitaxel Dose (mg/m²) | Compound A Dose (mg/m²) |
|---|---|---|
| 1 | 60 | 30 |
| 2 | 120 | 60 |
| 3 | 180 | 90 |
| 4 | 240 | 120 |
| 5 | 300 | 150 |
| 6 | 360 | 180 |
| 7 | 420 | 210 |

The MTD was not reached in this study and dose escalation was stopped because of PK nonlinearity at paclitaxel doses above 300 mg/m². The results showed that paclitaxel Cₘₐₓ and AUCinf increased with dose up to 300 mg/m² (Table 5). At doses above 300 mg/m², both Cₘₐₓ and AUCinf plateaued. Clearance and elimination half-life of paclitaxel did not show significant change with increase in dose level. Mean apparent clearance (CL/F) ranged from 131.7 L/h to 178.4 at paclitaxel doses from 60 to 300 mg/m², but increased up to 244.9 L/h at higher doses. T_{½} ranged from 19.9-32.1 hours, consistent with published values for paclitaxel. Metabolic ratios of p-3-hydroxy paclitaxel and 6α-hydroxy paclitaxel metabolites were 0.1 ~ 0.25 and 0.04 ~ 0.13 respectively. Paclitaxel pharmacokinetics was linear and proportional with doses up to 300 mg/m². The overall toxicity profile of paclitaxel, when orally administered in combination with Compound A, was consistent with that of paclitaxel, with neutropenia being the adverse event associated with temporary or permanent discontinuation of treatment.

TABLE 5

Pharmacokinetic Parameters of Paclitaxel by Dose Level

| Dose (mg/m²) | Statistic | tmax^{a} (µg/L) | Cmax (µg/L) | AUClast (ug*h/L) | AUCinf (µg*h/L) | Vd/F (L) | CL/F (L/h) | t½ (h) | MRTinf (h) | CL_{R} (L/h) |
|---|---|---|---|---|---|---|---|---|---|---|
| 60 (n=2) | Mean | 1.5 | 149.6 | 584.1 | 727.1 | 9128.7 | 178.4 | 32.1 | 28.6 | - |
| 120 (n=2) | Mean | 1.0 | 655.3 | 1611.1 | 1840.1 | 4036.0 | 131.7 | 22.0 | 17.0 | 3.0 |
| 180 (n=2) | Mean | 1.1 | 432.0 | 1673.9 | 1985.0 | 6030.7 | 173.4 | 24.4 | 21.9 | 1.8 |
| 240 (n=3) | Mean | 1.0 | 509.6 | 2547.4 | 2971.2 | 6011.5 | 147.6 | 25.7 | 23.4 | 2.4 |
| | SD | 0.5 | 177.8 | 1000.3 | 941.1 | 4651.5 | 56.1 | 11.2 | 9.5 | 1.0 |
| | CV (%) | 1.0 | 34.9 | 39.3 | 31.7 | 77.4 | 38.0 | 43.6 | 40.7 | 39.4 |
| 300 (n=3) | Mean | 0.5 | 919.7 | 3135.0 | 3481.1 | 4927.3 | 162.2 | 21.2 | 16.2 | 2.0 |
| | SD | 0.5 | 261.1 | 1450.1 | 1580.0 | 2900.7 | 98.4 | 1.4 | 0.5 | - |
| | CV (%) | 1.0 | 28.4 | 46.3 | 45.4 | 58.9 | 60.7 | 6.6 | 3.0 | - |
| 360 (n=3) | Mean | 0.5 | 655.0 | 2512.1 | 2851.7 | 6837.8 | 228.7 | 20.8 | 18.7 | 7.3 |
| | SD | 0.5 | 464.8 | 809.9 | 923.1 | 1668.4 | 59.8 | 1.9 | 1.0 | 9.8 |
| | CV (%) | 2.0 | 71.0 | 32.2 | 32.4 | 24.4 | 26.1 | 9.0 | 5.5 | 134.3 |
| 420 (n=2) | Mean | 0.5 | 707.5 | 3407.1 | 3731.5 | 7175.1 | 244.9 | 19.9 | 14.7 | 2.9 |

### Example 5 - Determination of MTD for Orally Administered Paclitaxel in Combination with Compound A

A study to determine the MTD for orally administered paclitaxel in subjects with advanced malignancies is being conducted. Successive cohorts are being treated with oral doses of 270 mg (approximately 150 mg/m²) paclitaxel per day. The initial cohort was treated with 270 mg per day 2 days per week for 3 weeks of a 4-week cycle. Subsequent cohorts received an additional day of treatment for each dosing week. Subjects in the first 2 cohorts (Arm 1) received 15 mg Compound A concomitantly with oral paclitaxel on Day 1 only, and were not required to fast prior to taking study medication. Subsequent cohorts (Arm 2) received Compound A concomitantly with each dose of paclitaxel and were explicitly instructed to fast prior to dosing. To date, the first 3 subjects have completed 5 days of dosing in Arm 2 (total dose 1350 mg or approximately 750 mg/m² paclitaxel per week for 3 out of 4 weeks).

### Example 6 - Clinical Study of Orally Administered Paclitaxel in Combination with Compound A

A 2-part study was conducted in subjects with advanced/metastatic or recurrent gastric cancer. The initial stage included a 28-day cycle MTD assessment of orally administered paclitaxel (as liquid filled capsules at doses of 90, 120, or 150 mg/m² per day for 2 days), with Compound A given concomitantly at a dose of 15 mg on Day 1. In the second part of this study, the selected dose of paclitaxel, 150 mg/m² per day, was given for 2 days per week, every 3 weeks out of a 4-week cycle. In some cases, an additional 15 mg tablet of Compound A on Day 2 was given in each dosing week. A total of 56 subjects enrolled in this study: 10 subjects with advanced malignant tumors in Part 1 and 46 subjects with advanced gastric cancer in Part 2.

Pharmacokinetic results from these 2 studies showed that paclitaxel maximum concentration (Cₘₐₓ) and area under the curve extrapolated to infinity (AUCinf) increased with dose up to 300 mg/m² following administration of paclitaxel. At doses above 300 mg/m², both Cₘₐₓ and AUC_{inf} plateaued. Half-life (t_{½}) ranged from 19.9 to 32.1 hours, consistent with published values for paclitaxel. Metabolic ratios of p-3-hydroxy paclitaxel and 6α-hydroxy paclitaxel metabolites were 0.1 ~ 0.25 and 0.04 ~ 0.13, respectively. Following 2 consecutive daily doses of paclitaxel at doses of 60, 90, or 150 mg/m², minimal to no accumulation occurred in paclitaxel Cₘₐₓ and AUC. Cₘₐₓ and AUC₀₋₂₄ ranged from 202 - 280 (Day 1) and 159 - 315 ng/mL (Day 2), and 611 - 894 (Day 1) and 735 - 1081 ng·h/mL (Day 2), respectively, with minimal increase in Cₘₐₓ across the dose levels. Overall exposure on Day 2 was about 20 to 30% higher. T_{½} ranged from 18.3 to 19.7 hours, consistent with published values for paclitaxel. The results are shown below in Tables 6 and 7.

TABLE 6

Paclitaxel Cₘₐₓ following Two Consecutive Daily Oral Doses

| Dose (mg/m²) | Day | N | Mean (SD) (ng/mL) |
|---|---|---|---|
| 90 | 1 | 2 | 202 (111) |
| 120 | 1 | 3 | 280 (65.3) |
| 150 | 1 | 3 | 251 (100) |
| 90 | 2 | 2 | 315 (83.6) |
| 120 | 2 | 3 | 159 (114) |
| 150 | 2 | 3 | 276 (73.3) |

TABLE 7

Paclitaxel AUC₀₋₂₄ Following Two Consecutive Daily Oral Doses

| Dose (mg/m²) | Day | N | Mean (SD) (ng-hr/mL) |
|---|---|---|---|
| 90 | 1 | 2 | 611 (124) |
| 120 | 1 | 3 | 627 (207) |
| 150 | 1 | 3 | 894 (159) |
| 90 | 2 | 2 | 735 (171) |
| 120 | 2 | 3 | 904 (137) |
| 150 | 2 | 3 | 1081 (234) |

### Example 7: Food Effect

In dogs, absorption of orally administered paclitaxel was inhibited by food. Paclitaxel Cₘₐₓ and AUC were approximately a 2-fold higher in the fasted group as compared to the fed group.

TABLE 8

Food Effect in Dogs After a Single Oral Dose of Paclitaxel in Combination With Compound A

| | | AUCₗₐₛₜ (ng.hr/mL) | Cₘₐₓ(ng/mL) | Tₘₐₓ (hr) | t_{½} (hr) | Vd/F (L) | CL/F (L/hr) | AUC_{Fed}/ AUC_{Fasted} Ratio |
|---|---|---|---|---|---|---|---|---|
| Paclitaxel (6 mg/kg) | Fed | 849.1±119.9 | 299.0±54.6 | 0.7±0.2 | 9.9±2.7 | 88.0±19.6 | 6.4±1.3 | 53.1 |
| | Fasted | 1599.8±416.5 | 683.6±174.8 | 0.5±0.0 | 9.4±0.3 | 57.9±17.4 | 4.2±1.2 | |

Dose: Paclitaxel, 6 mg/kg in combination with Compound A 3 mg/kg

### Example 8 - Clinical Study of Orally Administered Paclitaxel in Combination with Compound A in the Treatment of Cutaneous Angiosarcoma

A non-blinded, multi-center, open-label, pilot study to evaluate the activity, safety, and tolerability of orally administered paclitaxel in combination with Compound A in subjects with cutaneous angiosarcoma is being conducted. The study contains five periods: Screening, Baseline, Treatment, Treatment Extension, and Follow-up. Paclitaxel (205 mg/m²) is administered orally in capsules and Compound A (15 mg) orally in tablets, once daily for 3 consecutive days every week during the treatment period from weeks 1 through 25. Compound A is administered one hour before paclitaxel on all dosing days. The calculated paclitaxel dose (based on body surface area [BSA]) for each subject is converted into the closest number of 30-mg paclitaxel capsules.

Anti-emetics are given to subjects on each day of administration, with the first dose of anti-emetic given at the same time as Compound A. 5-hydroxytryptamine (5-HT3) antagonists are used for prophylaxis or initial treatment of nausea or vomiting. Steroids or H-1 receptor antagonists are given only if hypersensitivity-type reactions occur (including dyspnea with or without bronchospasm, urticaria, flushing or rashes, blood pressure changes, or angioedema). For subjects who may experience hypersensitivity-type reactions, premedication with steroids or H-1 antagonists may be given before subsequent doses, if clinically indicated.

Subjects who do not have documented disease progression by the end of the Treatment Period are eligible to receive therapy in the Treatment Extension Period; additional doses of oral paclitaxel in combination with Compound A are administered from Week 26 onwards. Subjects may receive oral paclitaxel in combination with Compound A until they meet 1 of the criteria for withdrawal from the study. Safety assessments, including clinical and laboratory assessments and AEs, is conducted throughout the study. Tumor response is evaluated according to RECIST v1. 1 criteria at specified timepoints during the study. A Final Visit takes place after completion of treatment, or if a subject is discontinued within 28 days after the last dose of study treatment, preferably before the subject receives additional chemotherapy.

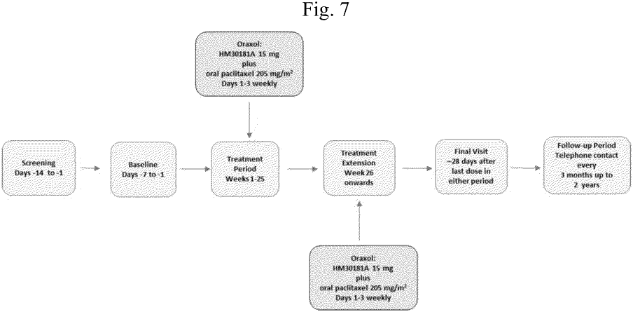

After the Final Visit, subjects are contacted by telephone every 3 months for up to 2 years, to assess overall survival (Follow-up Period). This study has an optimal two-stage design as outlined in FIG. 7. At the end of Stage I, the primary endpoint RR₆ months is examined. The study is stopped if 2 or fewer of the first 12 evaluable subjects respond during their first 6 months of treatment. When 3 or more responders are observed, an additional 13 evaluable subjects are enrolled. Non-evaluable subjects are replaced.

Primary response rate is determined within 6 months after initiation of treatment. Secondary outcomes include determination of adverse events (AEs), overall safety and tolerability, progression free survival, overall survival, duration of response, and time to best response.

### Study Design

Subject eligibility is established during the Screening/Baseline Period which lasts no longer than 14 days. Subjects are screened within 14 days of the first dose of study drug. All screening/baseline assessments/evaluations, as presented in Table 11, are performed after eligibility criteria are met. All baseline assessments/evaluations are performed prior to dosing.

The Treatment Period begins on Day 1, Week 1 and continues through Day 7 of Week 25. All subjects are fasted for 6 hours before treatment and for 2 hours after treatment (water, coffee, and tea (with sugar) is allowed during the fasting period. At 2 hours after the dose, subjects may have a light meal and may resume full meals at 4 hours after the dose. Safety and efficacy assessments evaluations are performed during the Treatment Period according to Table 11. If a subject discontinues the study at any time before Week 26, an end of treatment visit is conducted within 28 days after the last dose of study treatment. If possible, this visit is scheduled before the subject receives additional chemotherapy. Table 9 presents the treatments administered in the study.

TABLE 9

Treatments Administered in Study

| | | | | |
|---|---|---|---|---|
| | Strength | Oral Dose Form | Amount Dispensed and Frequency | Days Administered |
| Compound A | 15 mg | Tablet | Single dose: 1 x 15-mg tablet, 1 hour before oral paclitaxel | Days 1 to 3 of each week |
| Oral paclitaxel | 30 mg | Capsules | Single dose: number of capsules dispensed based on calculated doses | Days 1 to 3 of each week |

A subject is withdrawn from study treatment if it is medically necessary, or if it is the wish of the subject. In addition, subjects may be discontinued early from the study treatment due to one or more of the following reasons:
Death
Progression of disease
   radiologic progression of disease not associated with AEs
   radiologic progression of disease associated with AEs
   other clinical findings indicating disease progression, e.g., pain with bone metastases or seizure with brain metastases
AEs not associated with progression of disease
Withdrawal of consent - is qualified as with or without permission to obtain additional assessments after the time of withdrawal
Unacceptable toxicity at the lowest dose level
Noncompliance
Termination of the study
Other

### Subject Eligibility

### Inclusion Criteria

Willingness and ability to give informed consent, prior to any study-specific procedures and willingness to comply with scheduled visits, treatment plan, laboratory tests, and other study procedures.
Age of 18 years or older.
Histologically-confirmed cutaneous angiosarcoma that is not amenable to curative intent surgery (e.g., locally advanced disease and disease for which surgical resection would carry an unacceptable risk of recurrence or morbidity to the subject).
No previous treatment of angiosarcoma with taxanes.
Measurable disease per RECIST v.1.1.
Eastern Cooperative Oncology Group (ECOG11) performance status ≤ 1
Resolution of all acute AEs resulting from prior cancer therapies to NCI CTCAE v4.03 Grade ≤ 1 or to that subject’s baseline
Adequate organ function as defined by the following criteria:
   Adequate renal function as evidenced by serum creatinine <1.5 x upper limit of normal (ULN) or calculated creatinine clearance ≥50 mL/ min per the Cockcroft and Gault formula.
   Adequate bone marrow function as evidenced by absolute neutrophil count (ANC) ≥ 1.5 × 10⁹/L, hemoglobin >9.0 g/dL (<9.0 g/dL is acceptable if it is corrected by transfusion), and platelet count ≥100 × 10⁹/L.
   Adequate liver function as evidenced by total bilirubin within normal limits, alanine aminotransferase (ALT) ≤3 xULN, aspartate aminotransferase (AST) ≧3xULN, gamma-glutamyl transferase (GGT) ≧10xULN, and alkaline phosphatase ≧3xULN.
Able to swallow pills whole and retain oral medication
Men who are sterile (including vasectomy confirmed by post vasectomy semen analysis), or agree to use a condom with spermicide and to not donate sperm during the study and for at least 30 days following last dose of treatment.
Woman of non-child bearing potential due to surgical sterilization (at least 6 weeks following surgical bilateral oophorectomy with or without hysterectomy or tubal ligation) confirmed by medical history or menopause (i.e., no menstrual bleeding for more than 12 months in a women aged ≧ 45 years), or woman of child-bearing potential who test negative for pregnancy at time of enrollment based on serum pregnancy test and agree to use at least 2 acceptable methods of birth control, one of which is highly effective, during the study and for at least 30 days after stopping treatment.
Life expectancy of at least 3 months.

### Exclusion Criteria

Subjects with metastases outside of local lymph node involvement.
Concurrent treatment or participation on other therapeutic clinical trial for angiosarcoma. Participation in companion studies sponsored by local institutions, including biological correlates, is permitted.
Women who are pregnant or breastfeeding.
Receipt of systemic cytotoxic therapy, including investigational agents, within 14 days or 5 half-lives of the first study dosing day, whichever is longer.
Major surgery or trauma within 28 days prior to first dose of investigational product. The following are not considered to be major procedures and are permitted before treatment administration: thoracentesis, paracentesis, catheter placement, port placement, laparoscopy, thoracoscopy, tube thoracostomy, bronchoscopy, endoscopic ultrasonographic procedures, mediastinoscopy, skin biopsies, and imaging-guided biopsy for diagnostic purposes.
Subjects who have received wide-field radiotherapy to the pelvis ≤3 months (defined as >50% of volume of pelvic bones or equivalent) or limited-field radiation for palliation ≤3 months prior to treatment administration. Angiosarcoma lesions in the radiation field are not evaluable unless they have developed progressive disease following radiation.
History of brain involvement with cancer, spinal cord compression, or carcinomatous meningitis, or new evidence of brain or leptomeningeal disease.
Angina, myocardial infarction, symptomatic congestive heart failure, cerebrovascular accident, transient ischemic attack, arterial embolism, pulmonary embolism, percutaneous transluminal coronary angioplasty (PTCA) or coronary artery bypass graft (CABG) within 3 months prior to treatment administration.
Active bleeding or bleeding diathesis actively requiring transfusions. Subjects with cutaneous ulcers from angiosarcoma or who have skin lesions with bleeding are allowed to participate.
Thrombolytic use (except to maintain IV catheters) within 10 days prior to treatment administration.
Presence of a malabsorption syndrome or major resection of the stomach or small bowel that could affect the absorption of the drug.
Known active viral or nonviral hepatitis or cirrhosis.
Known human immunodeficiency virus (HIV) or acquired immunodeficiency syndrome (AIDS) related illness.
Active infection that requires systemic treatment.
Concurrent use of a strong cytochrome P450 (CYP) 3A4 inducer (e.g., rifampin or St. John’s Wort) or a strong CYP3A4 inhibitor (e.g., ketoconazole) within 14 days prior to treatment administration.
Concurrent use of a strong CYP2C8 inhibitor (e.g., gemfibrozil) or inducer (e.g., rifampin) within 14 days prior to treatment administration.
Concurrent use of an oral medication with a narrow therapeutic index known to be a P-glycoprotein (P-gp) substrate within 24 hours prior to treatment administration.
Concurrent use of a medication known to be a strong P-gp inhibitor or inducer within 14 days prior to treatment administration.
History of hypersensitivity to paclitaxel, not attributed to a hypersensitivity type reaction to Cremophor® or history of hypersensitivity-type reaction to polysorbate 80 or other components of the formulation of the drug.
Other severe acute or chronic medical (including bone marrow suppressive diseases) or psychiatric condition or laboratory abnormality that may increase the risk associated with study participation, impede the ability of the subject to complete all protocol-specified activities, or may interfere with the interpretation of study results and make the subject inappropriate for this study.

### Response Evaluation

The primary determination of activity is based on objective tumor assessments made according to RECIST v1.1. Tumor assessments is done at Baseline and every 6 weeks after starting treatment during the Treatment Period and the Treatment Extension Period according to Table 10. Tumor assessments may include the following evaluations:
2D color photography - Subjects undergo 2D color photography of all visible cutaneous lesions, including a ruler to estimate the size of the lesions. In addition to the photos taken for endpoint assessment purposes, photos are taken when a significant change is observed since the last visit (scheduled or unscheduled).
Physical examination - Lesions detected by physical examination are only considered measurable if superficial, e.g., skin nodules and palpable lymph nodes. They are assessed using calipers. Documentation by color photography including ruler is recommended.
Cross-sectional imaging using CT or MRI scan - All subjects should receive CT or MRI scans of site of the primary tumor and location of known metastases. Imaging of chest and abdomen (with pelvis) is required at baseline and follow-up due to high likelihood of development of metastases at these sites. The preferred method of disease assessment is CT with contrast. If subjects are unable to receive CT contrast due to CT contrast medium allergy or renal insufficiency, enhanced MRI scans may be used. A combination of non-contrast CT and MRI studies (such as chest CT without contrast and abdominal MRI with contrast) may be used. CT or MRI scan of the brain is performed at screening as needed if metastases are suspected or the subject is neurologically symptomatic. CT scans are performed with contiguous cuts in slice thickness of 5 mm or less. Spiral CT are performed using a 5 mm contiguous reconstruction algorithm.
All other known areas of disease are consistently followed throughout the study. Imaging based evaluation rather than clinical examination is the required technique when both could be used to assess the antitumor effect of the treatment. For a given patient, the same method of assessment is used throughout the course of the study thereafter. The same method and technique are used to characterize each identified and reported lesion at Baseline, during the Treatment Period, the Treatment Extension Period, and at the end of study visit. All lesions are classified as target or non-target lesions at the screening visit. Each lesion designation is maintained through the course of the study.

Tumor assessments are performed as outlined in the schedule of assessments (Table 11 and Table 12) and whenever disease progression is suspected.
Measurable: are accurately measured in at least 1 dimension (longest diameter in the plane of measurement is to be recorded) with a minimum size of:
   10 mm by CT scan or MRI (CT scan slice thickness no greater than 5 mm).
   10 mm cutaneous lesion measured by digital 2D color photography; the measurements are reported by longest diameter.
   Malignant lymph nodes: To be considered pathologically enlarged and measurable, a lymph node is ≥ 15 mm in short axis when assessed by CT scan (CT scan slice thickness recommended to be no greater than 5 mm). At baseline and follow-up, only the short axis is measured and followed.

Lytic bone lesions, with an identifiable soft tissue component, evaluated by CT or MRI, are considered measurable lesions if the soft tissue component otherwise meets the definition of measurability previously described. Blastic bone lesions are non-measurable. Lesions in previously irradiated areas (or areas treated with local therapy) are not selected as target lesions, unless there has been demonstrated progression in the lesion. Prior radiation for other cancers or history of palliative radiation with documented subsequent progression is permitted.
Non-Measurable: All other lesions, including small lesions (longest diameter <10 mm or pathological lymph nodes with ≥10 to < 15 mm short axis) as well as truly nonmeasurable lesions. Lesions considered to be truly non-measurable are bone lesions (lytic lesions or mixed osteolytic-osteoblastic lesions without identifiable soft tissue components, and osteoblastic lesions), leptomeningeal disease, ascites, pleural/pericardial effusions, abdominal masses not confirmed by imaging techniques, and cystic lesions.

TABLE 10

Response Evaluation Criteria in Solid Tumors Assessed per RECIST 1.1 Criteria

| | | | |
|---|---|---|---|
| Target Lesions^{a} | Non-Target Lesions^{b} | New Lesion^{c} | Overall Response |
| CR | CR | No | CR |
| CR | Non-CR/non-PD | No | PR |
| CR | Not evaluated | No | PR |
| PR | Non-PD or not all evaluated | No | PR |
| SD | Non-PD or not all evaluated | No | SD |
| Not all evaluated | Non-PD | No | Not Evaluable |
| PD | Any Response | Yes or No | PD |
| Any Response | PD | Yes or No | PD |
| Any Response | Any Response | Yes | PD |

CR = complete response; PD = progressive disease; PR = partial response; SD = stable disease.
a Measurable lesions only.
b May include measurable lesions not followed as target lesions or non-measurable lesions.
c Measurable or non-measurable lesion.

### Target Lesions

Complete response (CR): Disappearance of all target lesions. Any pathological lymph nodes (whether target or non-target) must have reduction in short axis to <10 mm.
Partial response (PR): A least a 30% decrease in the sum of diameters of the target lesions, taking as a reference the baseline sum diameters.
Progressive disease (PD)— At least 20% increase in the sum of diameters of target lesions; taking as a reference the smallest sum on study (this includes the baseline sum if that is the smallest on study). In addition to the relative increase of 20%, the sum must also demonstrate an absolute increase of at least 5 mm. The appearance of 1 or more new lesions is also considered progression.
Stable disease: Neither sufficient shrinkage to qualify for PR nor sufficient increase to qualify for PD, taking as reference the smallest sum diameters while on study.

### Nontarget Lesions

Complete response (CR): Disappearance of all non-target lesions. All lymph nodes must be non-pathological in size (<10 mm short axis).
Non-CR/non-PD: Persistence of ≥1 non-target lesion(s).
Progressive disease (PD): Unequivocal progression of existing non-target lesions, or the appearance of ≥1 new lesion.

### Adverse Events

An Adverse Event (AE) is an untoward medical occurrence in a patient or clinical investigation subject. An adverse event (AE) does not necessarily have a causal relationship with the medicinal product. For this study, the criteria for identifying AEs are:
Any unfavorable and unintended sign (including an abnormal laboratory finding), symptom, or disease temporally associated with the use of an investigational product, whether or not considered related to the investigational product
Any new disease or exacerbation of an existing disease (Note: For subjects with disease progression, where medically feasible the actual clinical diagnoses manifesting disease progression are listed on the AE eCRF in addition to disease progression)
Any deterioration in nonprotocol-required measurements of a laboratory value or other clinical test (eg, ECG or x-ray) that results in symptoms, a change in treatment, or discontinuation from study drug
Recurrence of an intermittent medical condition (eg, headache) not present at Baseline Adverse events are graded on a 5-point scale according to CTCAE v4.03 or later as follows:
   Grade 1 = Mild: asymptomatic or mild symptoms or clinical or diagnostic observations only or intervention not indicated
   Grade 2 = Moderate: minimal, local, or noninvasive intervention indicated or limiting age-appropriate instrumental activities of daily living
   Grade 3 = Severe or medically significant but not immediately life-threatening: hospitalization or prolongation of hospitalization indicated or disabling or limiting self-care activities of daily living
   Grade 4 = Life-threatening consequences or urgent intervention indicated
   Grade 5 = Death related to AE

The subject’s performance status is assessed according to ECOG criteria. ECOG performance status is assessed at Screening, Weeks 1, 4, 7, 10, 13, 16, 19, 22, and 25 of the initial Treatment Period, every 6 weeks during the Treatment Extension (Weeks 31, 37, 43, etc) and at the Final Visit. A listing of the subjects enrolled can be seen in Table 13.

TABLE 11

Schedule of Procedures/Assessments for Screening/Baseline, Treatment, and Follow-up Periods

| Period^{a} | Screening^{b} | Baseline | Treatment (Weeks) | | | | | | | | | | | | | | | | | | Follow up^{c} |
|---|---|---|---|---|---|---|---|---|---|---|---|---|---|---|---|---|---|---|---|---|---|
| Day/Week or Visit^{d} | Day -14 to-1 | Day -7 to-1 | 1 | 2 3 | 4 | 5 6 | 7 | 8 9 | 10 | 11 12 | 13 | 14 15 | 16 | 17 18 | 19 | 20 21 | 22 | 23 24 | 25 | Final Visit^{e} within 28 days after last dose | Every 3 months up to 2 years |
| Time Window (Days) | | | | +2 | +3 | +3 | +3 | +3 | +3 | +3 | +3 | +3 | +3 | +3 | +3 | +3 | +3 | +3 | +3 | +3 | ±14 |
| Clinic visits^{f} | X | X | X | X | X | | X | | X | | X | | X | | X | | X | | X | X | |
| Non-clinic visit telephone follow-up^{g} | | | | | | X | | X | | X | | X | | X | | X | | X | | | X |
| Informed consent | X | | | | | | | | | | | | | | | | | | | | |
| Inc/Exc criteria | X | | | | | | | | | | | | | | | | | | | | |
| Demography | X | | | | | | | | | | | | | | | | | | | | |
| Height | X | | | | | | | | | | | | | | | | | | | | |
| Weight | X | | X | | X | | X | | X | | X | | X | | X | | X | | X | X | |
| BSA^{h} | X | | X | | X | | X | | X | | X | | X | | X | | X | | X | | |
| Medical/surgical hx | X | | | | | | | | | | | | | | | | | | | | |
| Oncology hx | X | | | | | | | | | | | | | | | | | | | | |
| Oncology tx hx | X | | | | | | | | | | | | | | | | | | | | |
| Prior and/or conmedsⁱ | X | X | X | X | X | X | X | X | X | X | X | X | X | X | X | X | X | X | X | X | |
| Vital signs | X | | | | X | | X | | X | | X | | X | | X | | X | | X | X | |
| 12-lead ECG^{j} | X | | X | | | | | | X | | | | | | X | | | | X | X | |
| Pregnancy test^{k} | X | | X | | | | | | X | | | | | | X | | | | X | X | |
| Urinalysis^{l} | X | | X | | X | | X | | X | | X | | X | | X | | X | | X | X | |
| Blood chemistry^{l} | X | | X | X | X | X | X | | X | | X | | X | | X | | X | | X | X | |
| Hematology^{l} | X | | X | X | X | X | X | X | X | X | X | X | X | X | X | X | X | X | X | X | |
| ECOG status^{m} | X | | X | | X | | X | | X | | X | | X | | X | | X | | X | X | |
| Physical examination | X | Xⁿ | X | | X | | Xⁿ | | X | | Xⁿ | | X | | Xⁿ | | X | | Xⁿ | X | |
| 2D photographyⁿ | | X | | X | X | | X | | | | X | | | | X | | | | X | X | |
| CT/ MRI chest, abdomen (with pelvis)ⁿ | | X | | | | | X | | | | X | | | | X | | | | X | X | |
| Period^{a} | Screening^{b} | Baseline | Treatment (Weeks) | | | | | | | | | | | | | | | | | | Follow up^{c} |
| Day/Week or Visit^{d} | Day -14 to-1 | Day -7 to-1 | 1 | 2 3 | 4 | 5 6 | 7 | 8 9 | 10 | 11 12 | 13 | 14 15 | 16 | 17 18 | 19 | 20 21 | 22 | 23 24 | 25 | Final Visit^{e} within 28 days after last dose | Every 3 months up to 2 years |
| Time Window (Days) | | | | +2 | +3 | +3 | +3 | +3 | +3 | +3 | +3 | +3 | +3 | +3 | +3 | +3 | +3 | +3 | +3 | +3 | ±14 |
| Tumor assessmentⁿ | | X | | | | | X | | | | X | | | | X | | | | X | X | |
| Biopsy^{o} | | | | | | | X^{o} | | | | x^{o} | | | | x^{o} | | | | x^{o} | x^{o} | |
| Administer Oraxol^{p} | | | X | X | X | X | X | X | X | X | X | X | X | X | X | X | X | X | X | | |
| Dispense Oraxol for home use^{q} | | | X | | X | | X | | X | | X | | X | | X | | X | | X^{r} | | |
| Adverse events | X | X | X | X | X | X | X | X | X | X | X | X | X | X | X | X | X | X | X | X | |

BSA = body surface area; Conmeds = concomitant medications; CT = computed tomography; ECG = electrocardiogram; ECOG = Eastern Cooperative Oncology Group; hx = history; Inc/Exc = Inclusion/Exclusion (criteria); MRI = magnetic resonance imaging; RECIST = Response Evaluation Criteria in Solid Tumors; tx = treatment.
a All assessments is performed as scheduled.
b Screening procedures/assessments may be performed within 14 days prior to Day 1, Week 1. If screening assessments are performed within 4 days prior to dosing, the Day 1 Week 1 assessments do not need to be repeated and the screening assessments can be used as Day 1, Week 1 assessments.
c A follow-up telephone contact is made every 3 months after the Final Visit for up to 2 years to assess overall survival.
d Days are denoted for the Screening Period; week numbers are shown for the Treatment Period.
e The Final Visit occurs after completion of treatment or if a subject is discontinued within 28 days after the last dose, preferably before the subject receives additional chemotherapy.
f If there is a dosing delay, eg, for unacceptable toxicity, subjects continue weekly visit assessments until dosing resumes.
g Within 48 hours prior to Day 1 of non-clinic visit weeks, qualified designee contacts the subject to discuss possible adverse events, concomitant medications used, and any other safety concerns. If the subject is visiting the clinic to have their weekly laboratory assessment collected, the study coordinator may conduct the follow-up at that time or the site may contact the subject via telephone.
h BSA is calculated based on pretreatment body weight and height using the site’s institutional standard. Sites ensure that the same method for BSA calculation is used throughout the course of the study. After Week 1, BSA is recalculated if weight changes by ≥10% and the dose is adjusted if clinically indicated.
i The site reviews prior and concomitant medications reported during Screening and Baseline and confirms if any new non-oncological medications have been taken since the last clinic visit. Concomitant medications includes nonprescription remedies, vitamins, etc. For the study weeks where no study visit is planned, concomitant medications is assessed at weekly intervals (within 48 hours before dosing for non-clinic weeks) via telephone, or at clinic if subject is visiting the clinic to have weekly laboratory samples collected.
j A 12-lead ECG is performed at Screening and Weeks 1, 10, 19, 25 on the day of the clinic visit, and at the Final Visit. Subjects are in the recumbent position for a period of 5 minutes prior to the ECG.
k Serum pregnancy test to be performed at Screening, Day 1 Week 1, and Weeks 10, 19, 25, and at the Final Visit in females of childbearing potential. Additional pregnancy testing is performed as needed.
1 Hematology, chemistry, and urinalysis tests are to be obtained within 2 days before Day 1 dosing on designated weeks. Laboratory results are reviewed and acceptable prior to dosing on Day 1 of every dosing week and following any treatment delays for unacceptable toxicity. Subjects are fasted for Screening and Final Visit laboratory assessments. It is recommended, but not required, that subjects be fasted for the remaining laboratory assessments. It is noted if subjects were in a fasted or fed state at the time of blood collection.
m Refer to ECOG criteria to assess subject status. Subjects with an ECOG >1 at Screening or Day 1 Week 1 is excluded from the study. ECOG assessment occurs before dosing at each clinic visit noted in the schedule.
n A tumor assessment, including physical examination, 2D photography, and imaging (CT/MRI) of chest and abdomen (with pelvis) based on RECIST v1.1 criteria to determine eligibility is conducted at Baseline (within 7 days prior to the first dose and as close as possible to Day 1 Week 1). CT or MRI scan of the brain is performed at Baseline if metastases are suspected or the subject is neurologically symptomatic. Subsequent scans and tumor assessments occurs every 6 weeks ±7 days. Imaging is done at the Final Visit if no imaging was performed within 6 weeks prior to the Final Visit. Unscheduled scans are conducted if clinically indicated. In addition to the photos taken for endpoint assessment purposes, photos are taken when a significant change is observed since the last visit (scheduled or unscheduled).
o Optional, if needed to confirm complete response.
p Oral paclitaxel in combination with Compound A is taken for 3 consecutive days of every week, ie, on Days 1, 2, and 3 of every week. Compound A is administered 1 hour before oral paclitaxel on all dosing days.
q Starting at Day 1 Week 1, on Day 1 of every 3rd week, subjects is dispensed oral paclitaxel in combination with Compound A treatment for at home administration.
r Dispensation is done only if a subject continues to the Treatment Extension Period.

TABLE 12

Schedule of Procedures/Assessments for Treatment Extension and Follow-up Periods

| Period^{a} | Treatment Extension | | | | | | Follow-up^{b} |
|---|---|---|---|---|---|---|---|
| Week or Visit | 26 27 | 28 | 29 30 | 31 then every 6 weeks | 32 and 33 then weekly on non-clinic weeks | Final Visitc within 28 days after last dose | Every 3 months up to 2 years |
| Time Window (Days) | +3 | +3 | +3 | +3 | +3 | +3 | ±14 |
| Clinic visits^{d} | | | | X | | X | |
| Non-clinic visit telephone follow-up^{e} | X | X | X | | X | | X |
| Weight | | | | X | | X | |
| BSA^{f} | | | | X | | | |
| Prior and/or conmeds^{g} | X | X | X | X | X | X | |
| Vital signs | | | | X | | X | |
| 12-lead ECG^{h} | | | | X | | X | |
| Pregnancy testⁱ | | | | X | | X | |
| Urinalysis ^{j} | | | | X | | X | |
| Blood chemistry ^{j} | | | | X | | X | |
| Hematology^{j} | X | X | X | X | X | X | |
| ECOG Status^{k} | | | | X | | X | |
| Physical examination | | | | Xl | | X^{l} | |
| 2D photography^{l} | | | | X | | X | |
| CT/ MRI chest, abdomen (with pelvis)^{l} | | | | X | | X | |
| Tumor assessment^{l} | | | | X | | X | |
| Biopsy^{m} | | | | X^{m} | | X^{m} | |
| Administer Oraxolⁿ | X | X | X | X | X | | |
| Dispense Oraxol for home use^{o} | | | | X | | | |
| Adverse Events | X | X | X | X | X | X | |

BSA = body surface area; Conmeds = concomitant medications; CT = computed tomography; ECG = electrocardiogram; ECOG = Eastern Cooperative Oncology Group; MRI = magnetic resonance imaging; RECIST = Response Evaluation Criteria in Solid Tumors.
a All assessments are performed as scheduled.
b A follow-up telephone contact is made every 3 months after the Final Visit for up to 2 years to assess overall survival.
c The Final Visit occurs after completion of treatment or if a subject is discontinued within 28 days after the last dose, preferably before the subject receives additional chemotherapy.
d If there is a dosing delay, eg, for unacceptable toxicity, subjects continue weekly visit assessments until dosing resumes.
e Within 48 hours prior to Day 1 of non-clinic visit weeks, qualified designee contacts the subject to discuss possible adverse events, concomitant medications used, and any other safety concerns. If the subject is visiting the clinic to have their weekly laboratory assessment collected, the study coordinator may conduct the follow-up at that time or the site may contact the subject via telephone.
f BSA is calculated based on pretreatment body weight and height using the site’s institutional standard. Sites ensures that the same method for BSA calculation is used throughout the course of the study. BSA is recalculated if weight changes by ≥10% and the dose is adjusted if clinically indicated.
g The site confirms if any new non-oncological medications have been taken since the last clinic visit. Concomitant medications includes nonprescription remedies, vitamins, etc. For the study weeks where no study visit is planned, concomitant medications is assessed at weekly intervals (within 48 hours before dosing for non-clinic weeks) via telephone, or at clinic if subject is visiting the clinic to have weekly laboratory samples collected. h A 12-lead ECG is performed every 6 weeks on treatment on the day of the clinic visit and at the Final Visit. Subjects are in the recumbent position for a period of 5 minutes prior to the ECG.
i Serum pregnancy test to be performed every 6 weeks and at the Final Visit in females of childbearing potential. Additional pregnancy testing is performed as needed.
j Hematology, chemistry, and urinalysis tests are to be obtained within 2 days before Day 1 dosing on designated weeks. Laboratory results are reviewed and acceptable prior to dosing on Day 1 of every dosing week and following any treatment delays for unacceptable toxicity. Subjects are fasted for Final Visit laboratory assessments. It is recommended, but not required, that subjects be fasted for the remaining laboratory assessments. It is noted if subjects were in a fasted or fed state at the time of blood collection.
k Refer to ECOG criteria to assess subject status. ECOG assessment occurs before dosing at each clinic visit noted in the schedule.
l A tumor assessment, including physical examination, 2D photography, imaging (CT/MRI) of chest and abdomen (with pelvis) based on RECIST v1.1 criteria to determine eligibility is conducted every 6 weeks ±7 days. Imaging is done at the Final Visit if no imaging was performed within 6 weeks prior to the Final Visit. Unscheduled scans are conducted if clinically indicated. In addition to the photos taken for endpoint assessment purposes, photos are taken when a significant change is observed since the last visit (scheduled or unscheduled).
m Optional, if needed to confirm complete response.
n Oral paclitaxel in combination with Compound A is taken for 3 consecutive days of every week, ie, on Days 1, 2, and 3 of every week. Compound A is administered 1 hour before oral paclitaxel on all dosing days.
o On Day 1 of every 6^{th} week, subjects is dispensed oral paclitaxel in combination with Compound A treatment for at home administration.

TABLE 13

Subjects Enrolled in Study

| Subject | Location | Age | Race | Gender | Angiosarcoma primary lesion location | Metastatic Site | Type of Diagnoses | Oncological History |
|---|---|---|---|---|---|---|---|---|
| 1 | USA | 69 | White | Female | Right areola | Right axilla | Secondary, radiotherapy | Bilateral breast cancer |
| 2 | USA | 72 | White | Male | Nasal dorsum | None | Idiopathic | Basal cell carcinoma of skin (left nostril) |
| 3 | USA | 82 | White | Male0 | Scalp | None | Idiopathic | Kidney cancer |
| 4 | USA | 76 | White | Female | Right breast | None | Idiopathic | Breast cancer |
| 5 | USA | 88 | White | Male | Scalp | None | Idiopathic | Prostate cancer |
| 6 | Hong Kong | 93 | Asian | Male | Scalp, left parietal scalp angio | None | Idiopathic | None |
| 7 | Hong Kong | 85 | Asian | Female | Scalp | Lymph nodes | Idiopathic | None |

### Treatment Extension

Subjects who do not have progression of disease during the Treatment Period may enter a Treatment Extension Period. During the Treatment Extension Period, subjects may receive additional treatment, starting at the final dose level received during the Treatment Period (i.e., at the reduced dose, if dose reduction is necessary). Subjects may receive additional treatment at their assigned dose or at one of the dose reduction levels if dose reduction is warranted due to unacceptable toxicities, until any of the discontinuation criteria are met. Subjects in the Treatment Extension Period may be given dose reductions as described for unacceptable toxicity. A total of two dose reductions are permitted. Safety and efficacy assessment evaluations, as detailed in Table 12 are performed during the Treatment Extension Period. If a subject discontinues the study at any time during the Treatment Extension Period, an end of treatment visit is conducted within 28 days after the last dose of study treatment. If possible, this visit is scheduled before the subject receives additional chemotherapy.

### Dose Delays and Reduction

An unacceptable toxicity occurs when any of the events listed below are considered at least possibly related to the administration of paclitaxel in combination with Compound A. The grading of toxicity is based on the NCI CTCAE v4.03 criteria.
$ 1 & FM~ x 10⁹/L
Grade 3 or 4 ANC plus fever or Grade 3 or 4 ANC with bacteremia or sepsis
Grade 3 thrombocytopenia (<50 x 10⁹/L platelets) for more than 7 days, or accompanied by clinically significant bleeding
Grade 4 thrombocytopenia (<25 x 10⁹/L platelets) regardless of duration or clinical manifestations
Grade ≥3 nausea, vomiting, or diarrhea persisting for more than 48 hours despite optimal medical management
Grade ≥3 clinically important toxicities not listed above. This does not include:
   laboratory abnormalities not considered to be SAEs and which resolve back to Grade 1 or baseline within 7 days
   alopecia
   anorexia or asthenia which resolves within 7 days
Toxicities not listed above that cause a dose delay of >7 days
Grade >_3 neuropathy

If a subject’s ANC is >0.8 x 10⁹/L and <1.5 x 10⁹/L or platelet count is <100 x 10⁹/L prior to dosing, then the dose is delayed until counts recover to above these values at which time treatment is restarted at the same dose level.

Subjects experiencing ≥ Grade 3 neuropathy is discontinued from treatment. For subjects experiencing other unacceptable toxicity treatment is delayed until the toxicity improves to Grade 1 or baseline at which point they may resume treatment at Dose Reduction Level 1. If toxicity occurs at Dose Reduction Level 1, the dose is further reduced to Dose Reduction Level 2.

Dose Reduction Level 1-15 mg oral Compound A plus oral paclitaxel 165 mg/m²/day, 3 times per week.

Dose Reduction Level 2 - 15 mg oral Compound A plus oral paclitaxel 130 mg/m²/day, 3 times per week.

Decisions concerning dose reduction due to results of clinical laboratory testing are based on the most recent local laboratory results available prior to dosing. Subjects who continue to experience unacceptable toxicity at the lowest dose level are discontinued from the protocol. Study AEs are presented in Table 14 and 15 below.

TABLE 14

Overview of AEs and SAEs

| | | | |
|---|---|---|---|
| Deaths | Disease Progression | Withdrawal due to AEs | SAEs |
| None | None | None | 1 (unrelated) |

### Subject 1

Subject 1, a 69-year-old white female, had a primary angiosarcoma lesion on the right areola and metastatic site on the right axilla. The angiosarcoma was secondary from radiotherapy. The subject had prior bilareral breast cancer. A breast biopsy was performed to confirm angiosarcoma, and no histological confirmation was performed on the axilla lesion. The subject was monitored according to the protocol. The sum of measurement at baseline of the primary lesion on the right areola was 71 mm (FIG. 8A). By week 7, the sum of the primary lesion on the right areola was 0 mm (FIG. 8C). An additional measurement was taken at week 2 (FIG. 8B). The sum of measurement at baseline of the secondary lesion on the right axilla was 16 mm. On week 7, the sum of the secondary lesion on the right axilla was 17 mm as determined by radiology (CT).

The subject had a SAE of pneumonia unrelated to the treatment on week 4 and was treated with antibiotics and antiviral medication. The SAE was completely resolved in one week and the subject continued treatment with oral paclitazel in combination with Compound A.

### Subject 2

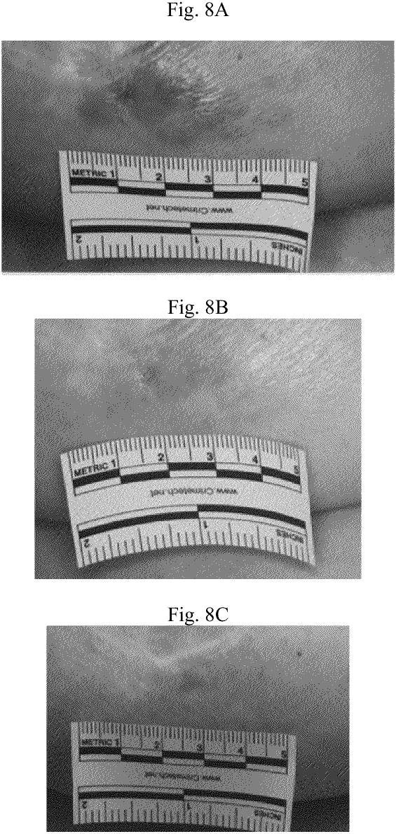

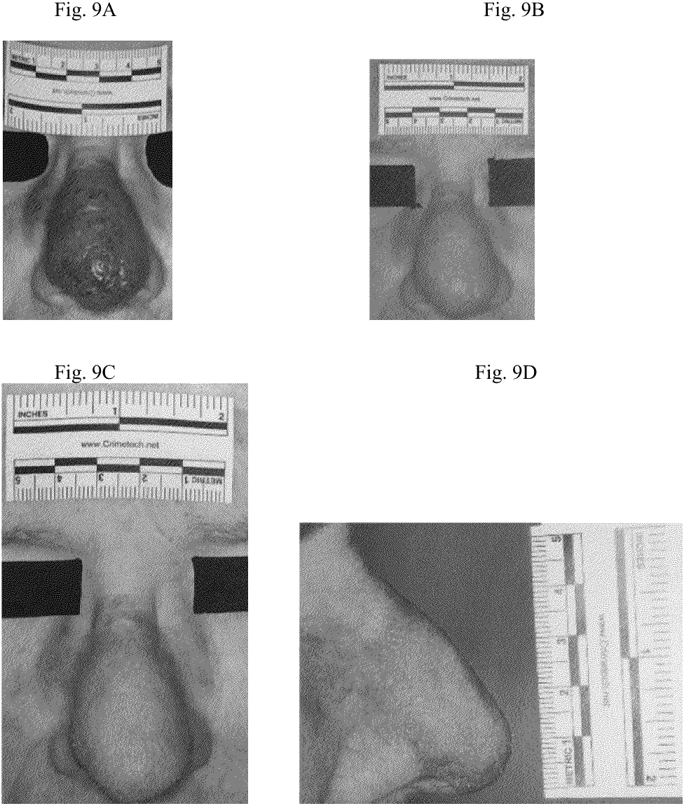

Subject 2, a 72-year-old white male, had a primary angiosarcoma lesion on the nasal dorsum. The angiosarcoma was idiopathic. The subject had prior basal cell carcinoma of the skin on the left nostril. Histology was performed on archival tissue. The subject was monitored according to the protocol. The sum of measurement at baseline of the primary lesion on the nasal dorsum was 50 mm (FIG. 9A). On week 7, the sum of the primary lesion on the nasal dorsum was 45 mm (FIG. 9B). The subject reported darkening in color and dryness of the lesion during weeks 1 and 2 of treatment. The subject’s skin started to return to a normal skin tone at week 3. Week 10 photographs were also taken from a front and side view (FIG. 9C and FIG. 9D).

Dosage was held at week 9 due to Grade 3 absolute neutrophil count.

### Subject 3

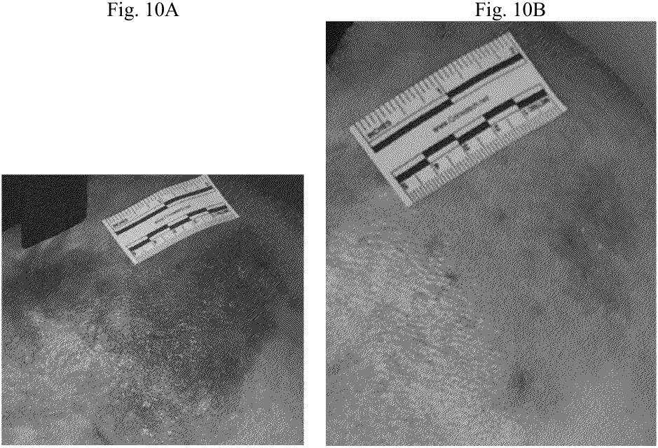

Subject 3, a 82-year-old white male with one kidney, had a primary angiosarcoma lesion on the scalp. The angiosarcoma was idiopathic. The subject had prior kidney cancer. A biopsy was performed to confirm angiosarcoma. The subject was monitored according to the protocol. The sum of measurement at baseline of the primary lesion on the scalp was 145 mm (FIG. 10A). On week 6, day 5, the sum of the primary lesion on the scalp was 0 mm (FIG. 10B). PET scan images from baseline (FIG. 11A) to week 6, day 5 (FIG. 11B) were taken to monitor the response by radiology, showing the no visible lesion at week 6, day 5.

The subjected had high creatinine levels since baseline and was prescrived IV fluids prior to week 4 dosing. Dosage was held at week 9 due to Grade 3 absolute neutrophil count.

### Subject 4

Subject 4, a 76-year-old white female, had a primary angiosarcoma lesion on the right breast. The angiosarcoma was idiopathic. The subject had prior breast cancer. A mastectomy was performed to confirm angiosarcoma. The subject was monitored according to the protocol. The sum of measurement at baseline of the primary lesion on the right breast was 58 mm. On week 2, the sum of the primary lesion on the right breast was 53 mm.

### Subject 5

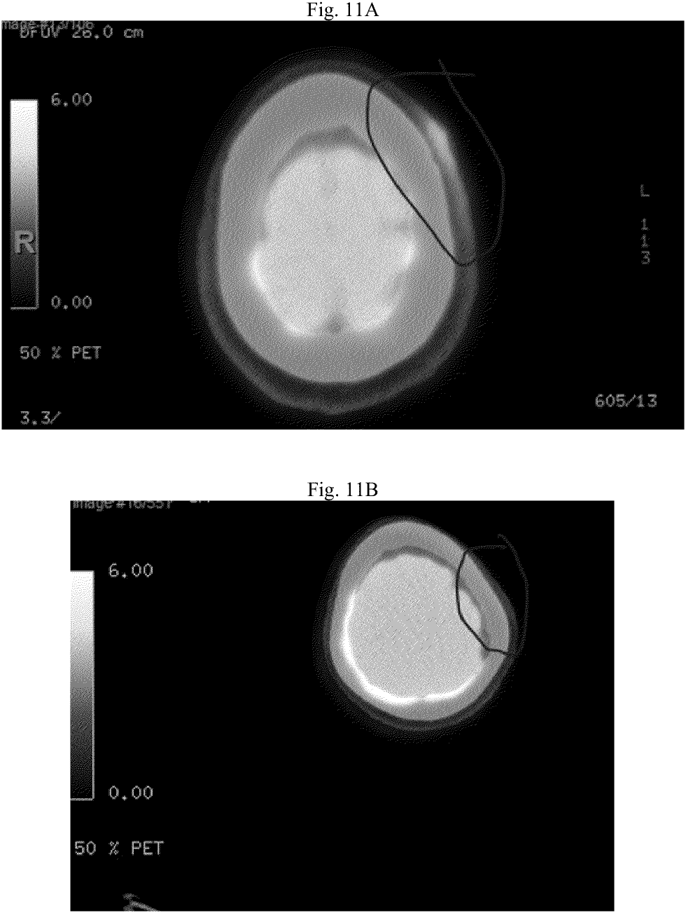

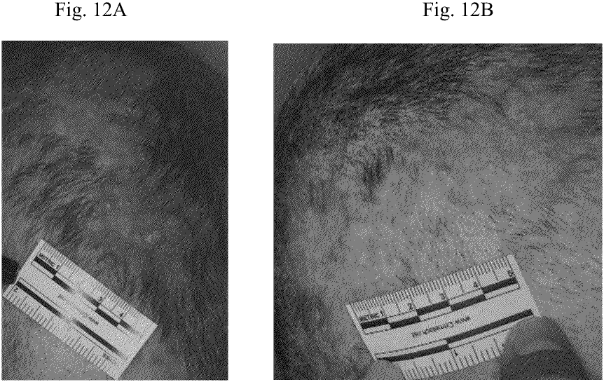

Subject 5, a 88-year-old white male, had a primary angiosarcoma lesion on the scalp. The angiosarcoma was idiopathic. The subject had prior prostate cancer. The subject was monitored according to the protocol. The sum of measurement at baseline of the primary lesion on the scalpe was 100 mm (FIG. 12A). The sum of measuremebnt at baseline of the primary lesion on the scalp and week 3 are the same with the lesion lighter in color (FIG. 12B).

### Subject 6

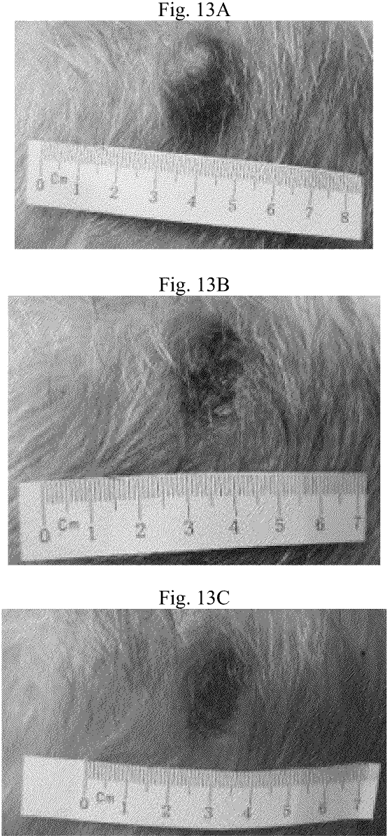

Subject 6, a 93-year-old Asian male, had a primary angiosarcoma lesion on the left parietal scalp. The angiosarcoma was idiopathic. The subject had no prior cancer history. The subject was monitored according to the protocol. There was a 44% improvement in lesion size based on CT scans from baseline to Week 7. Measurements at baseline (FIG. 13A), week 2 (FIG. 13B), and week 7 (FIG. 13C) show improvement in lesion size.

Two dose reductions were implemented due to absolute neutrophil counts of 500 at week 3 and 600 at week 7. Dosage at week 3 was reduced to 165 mg/m² and dosage at week 7 was reduced to 130 mg/m². Efficacy data shows partial remission.

### Subject 7

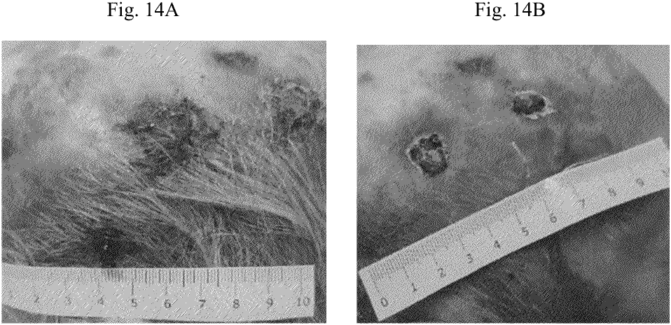

Subject 7, a 85-year-old Asian female, had a primary angiosarcoma lesion on the scalp and a metastatic site on the lymph nodes. The angiosarcoma was idiopathic. The subject had prior vertex radion and surgeries of both right and left auricular. The subject was monitored according to the protocol. Measurements at baseline (FIG. 14A) and week 7 (FIG. 14C) show improvement in lesion size.

One dose reductions was implemented due to an absolute neutrophil count of 700 at week 2. Dosage at week 2 was reduced to 165 mg/m².

### Adverse Events

No Grade 4 adverse events were observed. The most frequent Grade 3 adverse events are decrease in neutrophil count. However, the subjects recovered from the adverse events either without any change in treatment or through dose reduction. Other Grade 3 adverse events that may be attributable to the treatment include diarrhea, increase in GGT, rash Maculo-Papular, decrease in white blood cell count, and hyponatremia.

### Example 9 - In Vitro Effects of Paclitaxel on Angiosarcoma Cells

Three commercially available murine cell lines capable of forming subcutaneous angiosarcomas in mice have been generated. These include endothelial SVRbag4, an endothelial cell line derived from the SVR cell line (mouse endothelial cell line containing temperature sensitive SV40 large T-antigen and H-RAS oncogene), and SVRA221a (SVR cell line infected with a retrovirus encoding a dominant negative allele of the Mitogen-Activated Protein Kinase Kinase, MAPKK, gene A221a). The third cell line reported in the angiosarcoma literature, Mile Sven 1 (MS1), was derived from islet of Langerhans cells transduced with temperature sensitive SV40 large T antigen (tsA-58-3), but possesses many properties of endothelial cells.

### Methods

### In Vitro Cytotoxicity of Paclitaxel in Angiosarcoma Cell Lines

MS1, SVRA221a, SVRbag4, and HMEC-1 cells were seeded in 96-well plates (1,500 cells per well) in culture medium supplemented with 1 µM Compound A. After 24 hours incubation at 37° C., cells were washed once then treated with paclitaxel at a range of concentrations (0.3 - 2,187 nM) in 100 µL of culture medium for 72 hours. Three independent experiments of two replicates were completed. A 10 µL aliqout of 3-(4,5-dimethylthiazol-2-yl)-2,5-diphenyltetrazolium bromide (MTT) solution (5 mg/mL in PBS) was added to each well and incubated at 37° C. for 2 hours. The culture medium was removed followed by addition of 100 µL of DMSO to each well. The absorbance in each well was measured at 540 nm using a SpectraMax microplate reader. A sigmoid Eₘₐₓ model using nonlinear regression (Equation 1) with least squares fit was used to estimate Eₘₐₓ and GI₅₀ values using GraphPad Prism 6 for each experiment:

$$\left(I\right) \% i n h i b i t i o n = \frac{{E}_{m a x} ⋅ {C}^{γ}}{{C}^{γ} + G {I}_{50}}$$

where Eₘₐₓ is the maximal effect on cell proliferation caused by drug treatment, GI₅₀ is the drug concentration that causes 50% of the maximal growth inhibition, C is the concentration of drug, and γ is the Hill coefficient, which is a measure of steepness of slope. The average Eₘₐₓ and GI₅₀ values and standard deviated from three individual experiments were calculated and reported. R-squared of all curve fits exceeded 0.9.

### Immunofluorescence Microscopy of Tubulin Disruption

Coverslips (22-mm) were pre-coated with rat tail collagen for 1 hour then seeded with cells in 6-well plates for 24 hours before treatment. Murine angiosarcoma cells were treated with paclitaxel for 3 hours, followed by fixation with cold methanol at -20° C. for 20 min. After blocking with 2% BSA in PBS for 30 min, cells were incubated with anti α-tubulin antibody (Sigma 6199) diluted in blocking buffer (2% BSA in PBS) for 1.5 hours at room temperature. Cells were washed three times with PBS and incubated with Alexa 598 anti-Mouse IgG (Invitrogen) for 1 hours at room temperature. Following three PBS washes, coverslips were mounted on glass slides in ProLong antifade reagent (Invitrogen). Fluorescent images were captured using a Nikon TE2000-E inverted microscope for two independent experiments.

### Results

Murine angiosarcoma cell lines, MS1, SVRA221a, SVRbag4, and a human endothelium cell line, HMEC-1, were utilized as model systems to study paclitaxel anti-proliferative potency and activity in angiosarcoma. MS1 is a pancreatic islet endothelial cell line which is known to give rise to benign hemangiomas. SVRA221a and SVRbag4 cells are endothelial cells derived from the SVR cell line. HMEC-1 cells are human dermal microvascular endothelial cells.

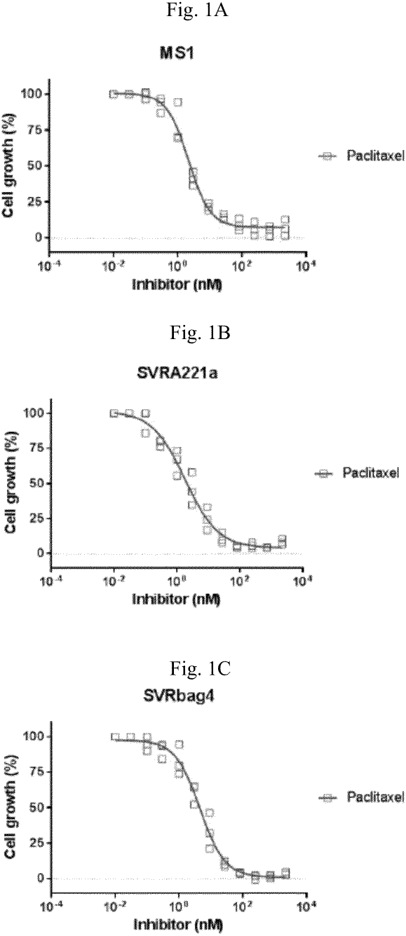

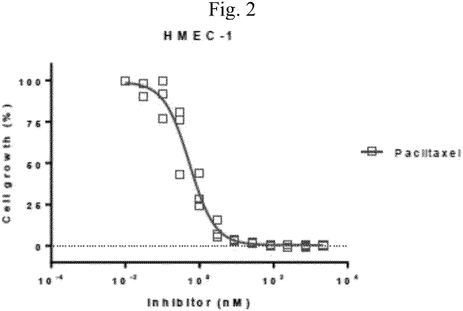

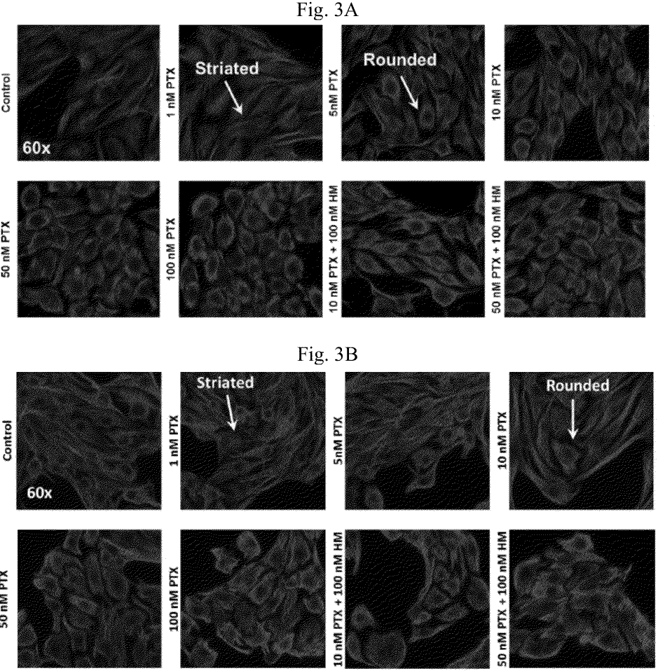

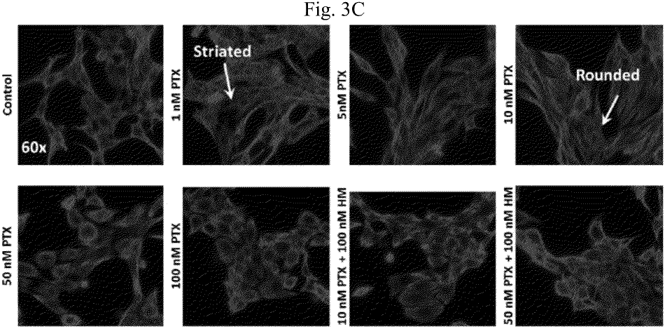

To evaluate the antiproliferative activity of paclitaxel in angiosarcoma, an in vitro MTT assay was used to assess the antiproliferative effects in three murine cell lines and a human endothelium cell line. The overall potency (GI₅₀) of paclitaxel was estimated to be 1.8 ± 0.36 ng/mL for MS1, 1.8 ±1.0 ng/mL for SVRA221a, 3.9 ± 1.0 ng/mL for SVRbag4 (FIG. 1A - FIG. 1C and Table 15), and 0.46± 0.23 ng/mL for HMEC-1 (FIG. 2 and Table 15). Observed GI₅₀ values ranged from 0.46- 3.9 ng/mL with a mean value of 2.0 ng/mL. The mechanism of action of paclitaxel on microtubule polymerization activity in the angiosarcoma cell lines was also examined. MS1, SVRA221a, and SVRbag4 cell lines were treated with 1 - 100 nM paclitaxel. Paclitaxel induced condensed and rounded morphology as well as polymerization of microtubules starting at 5 - 10 nM (4.2 - 8.5 ng/mL; FIG. 3A - FIG. 3C). These data support the ability of paclitaxel to inhibit angiosarcoma cell proliferation at low concentrations and that an overall plasma concentration in the range of 0.46 - 3.9 ng/mL paclitaxel causes cell death.

TABLE 15

Inhibition of Angiosarcoma Cell Proliferation by Treatment with Paclitaxel

| | Species | GI₅₀ (ng/mL) | Eₘₐₓ (% inhibition) |
|---|---|---|---|
| MS1 | Mouse | 1.8 ± 0.36 | 93 ± 4.5 |
| SVRA221a | Mouse | 1.8 ± 1.0 | 96 ± 1.1 |
| SVRbag4 | Mouse | 3.9 ± 1.0 | 99 ± 1.4 |
| HMEC-1* | Human | 0.46 ± 0.32 | 99 ± 0.10 |

Mean ± SD, n-3
* Human dermal microvascular endothelial cells

### Example 10 - In Vivo Effects of Combination of Paclitaxel and Compound A on Angiosarcoma

SVRA221a cells (1 x 10⁶) in 50% Matrigel were inoculated into one shaved flank of female SCID mice (70 total, divided into 5 groups of 14 mice each). When the majority of the tumors reached a tumor volume of 150 to 200 mm³, mice were sorted into treatment groups such that the mean tumor volume across study groups was equivalent. Mice received 20 mg/kg PO Compound A followed 30 minutes later by 10, 20, 30, or 40 mg/kg PO paclitaxel. The study design is shown in Table 16. The treatment schedule was administered QDx3/week for three weeks. Tumors and body weights were measured three times per week for up to four weeks. Mice were euthanized four weeks post treatment initiation, when tumors reached 1500 mm³, or when they became necrotic or ulcerated.

TABLE 16

Study Design for In Vivo Effects of Combination of Paclitaxel and Compound A on Angiosarcoma

| Group No. | Group Size | Treatment 1 | Treatment 2 | Schedule | Evaluation |
|---|---|---|---|---|---|
| 1 | 14 | Vehicle | Vehicle | QDx3/week for 3 weeks | |
| 2 | | | 10 mg/kg Paclitaxel, PO | | |
| 3 | | 20 mg/kg Compound A PO | 20 mg/kg Paclitaxel, PO | | Tumor volume & body weight 3x/week Daily overall condition |
| 4 | | | 30 mg/kg Paclitaxel, PO | | |
| 5 | | | 40 mg/kg Paclitaxel, PO | | |

A vehicle of 50% Tween80 + 50% PEG200 was utilized for paclitaxel. Equal volumes of Tween80 and PEG200 were combined in a 50 ml conical tube and gently mixed on a rotating mixer at 37° C. until the solution was uniform. The vehicle was prepared fresh on each dosing day.

For Compound A administration, a vehicle of DMSO + Diluent A (30% propylene glycol + 70% purified water + 0.02% conc. HCl) was prepared. Diluent A was produced by combining propylene glycol (216 ml), concentrated (37%) HCl (144 µl) and purified water (504 ml) in a polypropylene tube and gently stirred. One batch of Diluent A was prepared for the duration of the study. Compound A was prepared in 5% DMSO + 95% Diluent A. The amount of Compound A required to produce a 5 mg/ml solution was adjusted based on the batch correction factor of 1.195. Compound A powder was dissolved in DMSO (final concentration, 5%) by heating to 70° C. for approximately 20 sec. Diluent A was then added to yield a Compound A solution with a final concentration of 5 mg/ml.

Paclitaxel dosing solutions were prepared within 24 h of treatment in 50% Tween80 + 50% PEG200. The appropriate amount of paclitaxel power was added to the required volume of vehicle (final paclitaxel concentration of 10 mg/ml) in glass vial containing a stir bar. After brief stirring to suspend the powder, the solution was sonicated for 10 min at 55° C. Approximately 5 min after the start of sonication, the vial was briefly removed from the sonicator and gently stirred to dislodge any paclitaxel that had adhered to the side of the vial. The solution was then returned to the water bath to complete sonication. If small aggregates remained in the solution, an additional 5 min of sonication was performed. For each treatment day, a 10 mg/ml paclitaxel solution was diluted with the appropriate volume of vehicle to generate 2.5, 5, and 7.5 mg/ml paclitaxel solutions.

Compound A or its vehicle was administered by oral gavage in a volume of 100 µl per 25 g mouse on Days 1-3, 8-10, and 15-17. Paclitaxel or its vehicle was administered by oral gavage 30 minutes later in a volume of 100 µl per 25 g mouse body weight on Days 1-3, 8-10, and 15-17.

After tumor cell inoculation, mice were observed daily for overall condition, behavior, and the appearance of visible or palpable tumors. Once treatment was initiated a Total Condition Scoring system was used to monitor changes in mouse condition and identify adverse effects of treatment or tumor growth. The semi-quantitative scoring system takes into consideration body condition or appearance, posture and activity. Each parameter is scored based on the descriptions in Table 17 below and the scores were totaled to yield the “Total Condition Score (TCS).” The maximum attainable TCS is 12 and indicates no abnormal findings. A TCS of ≤ 3 indicates the observation of very strong adverse effects and is grounds for euthanasia. Abnormal findings not considered by the scoring system include tumor appearance, necrosis, and ulceration were. Mice with tumors that developed ulcerated or necrotic regions extending beyond a small scab were euthanized, as required by IACUC regulations.

TABLE 17

Total Condition Scoring system used to monitor changes in mouse condition and identify adverse effects of treatment or tumor growth

| Parameter | Score | Observation | Description |
|---|---|---|---|
| Body Condition | 4 | Normal | |
| | 3 | Slightly Abnormal | Slightly loose skin, some far but less than normal or slight abdominal distention |
| | 2 | Abnormal | Loos skin and thin, palpable iliac crests and vertebrae, moderate abdominal distention |
| | 1 | Severly Abnormal | No palpable fat, severly reduced muscle mass or severe abdominal distention |
| Posture | 4 | Normal | |
| | 3 | Slightly Hunched | |
| | 2 | Moderately Hunched | |
| | 1 | Severly Hunched | |
| Activity | 4 | Normal | |
| | 3 | Slightly Lethargic | Less interest in surroundings than normal, but still responsive |
| | 2 | Moderately Lethargic | Not interested in surroundings, but still responsive |
| | 1 | Severly Lethargic | Not interested in surroundings, not responsive |

Mice were weighed three times per week during the course of the study. During treatment periods, mice were weighed on dosing days prior to treatment to determine the appropriate treatment volume. The D1 weight was used as a baseline to calculate changes in body weight over the course of the study. Upon the development of visible, palpable tumors, tumors were measured daily until treatment commencement and then three times per week thereafter. More frequent tumor measurements were performed as a tumor approached the size endpoint (1500 mm³) to prevent the tumor volume from significantly passing the endpoint between measurements. Tumors were measured in two dimensions using digital calipers. Tumor volume was calculated using the equation (L x W^2)/2 where L (maximum length) was the longest dimension in mm and W (maximum width) was the longest dimension measured perpendicular to L. Tumor measurements on D1 were used as a baseline to calculate changes in tumor size over the course of the study.

Analysis of tumor growth parameters were performed for D13 and D15 of the study. D15 represented the last tumor measurement day with a sufficient number of control tumors for statistical analysis. As tumor regrowth between treatment cycles was evident, analysis was also performed on D13 representing the last tumor measurement before regrowth began after the second treatment cycle. Outliers in the dataset were excluded based on the ROUT test (GraphPad Prism). The mean tumor volume, fold tumor growth, and percentage of growth inhibition were calculated. These parameters were compared to the vehicle control using ANOVA. T/C%, a standard oncology parameter, was calculated as follows: (Tx-T1)/(Cx-C1)*100 where T and C represent the median tumor volume of treated and control tumors on Day X and Day 1. This value represents the size of treated tumors relative to control tumors. Thus, T/C% = 40 means that the treated tumor is 40% of the size of the control tumor at the designated time point.

The most frequent observations were related to changes in tumor appearance, including a darkened appearance with some bruising around the tumor and the development of necrotic or ulcerated areas. There was no significant weight loss observed. A dose-dependent decrease in SVRA221a tumor growth appeared to plateau between 30 and 40 mg/kg paclitaxel. Maximum growth inhibition (61.1%) was achieved on D13 with 30 mg/kg paclitaxel. Tumor growth resumed after completion of the treatment period.

### Group 1 (Vehicle Control)

Mice had a TCS ranging from 9-12 during the course of the study. Two mice with a TCS of 9 were likely anemic since they were both described as pale with one having a bleeding tumor. Mice euthanized prior to the end of the study due to necrosis, ulceration, or bleeding (11/14) on days 10 to 17 or reaching the tumor size endpoint (3/14) on days 17 to 28. No mice appeared ruffled during the course of the study.

### Group 2 (Compound A + 10 Mg/kg Paclitaxel)

Mice had a TCS of 11-12 during the course of the study. Some of the mice (8/14) appeared ruffled from days 1 to 5, which coincided with the first week of treatment. However, the appearance of the mice returned to normal and a ruffled appearance did not occur with subsequent weeks of treatment. Mice were euthanized prior to the end of the study due to necrosis, ulceration, or bleeding (9/14) on days 9 to 24 or reaching the tumor size endpoint (2/14) on days 15 and 17. Three mice reached the scheduled end of the study.

### Group 3 (Compound A + 20 Mg/kg Paclitaxel)

All but one mouse had a TCS of 11-12 during the course of the study. One mouse was euthanized on day 7 with a TCS of 7 due to a ruptured tumor and bleeding that resulted in a moribund condition and pale appearance. The majority of mice appeared ruffled for 2 to 6 days during each treatment week. Three mice were slightly lethargic after the first round of treatment. Mice were euthanized prior to the end of the study due to necrosis, ulceration, or bleeding (10/14) on days 10 to 27. One mouse was found dead on day 15, the cause of death unclear; the tumor had not ulcerated and the TCS score was 12. Three mice reached the scheduled end of the study.

### Group 4 (Compound A + 30 Mg/kg Paclitaxel)

Mice had TCS of 10-12 during the course of the study. The majority of the mice appeared ruffled for 3 to 6 days during each treatment week. Two mice were slightly hunched for two days after the first round of treatment. Mice were euthanized prior to the end of the study due to necrosis, ulceration, or bleeding (8/14) on days 10 to 25. Six mice reached the scheduled end of the study.

### Group 5 (Compound A + 40 Mg/kg Paclitaxel)

All but one mouse had TCS of 10-12 during the course of the study. The majority of the mice appeared ruffled for 3 to 6 days after the last treatment of each treatment week. Mice in this group were euthanized prior to the end of the study due to necrosis, ulceration, or bleeding (11/14) on days 10 to 27 or reaching the tumor size end point (1/14). One mouse was found dead on the morning of day 13, the cause of death unclear; the tumor had not ulcerated and the TCS score was 11 the prior day with a ruffled appearance. One mouse reached the scheduled end of the study.

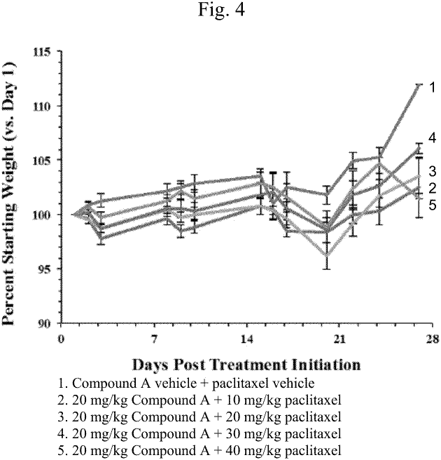

Treatment associated toxicity was not observed as illustrated by Total Condition Scores of 10 - 12 and weight loss of < 10% for the majority of the mice, regardless of treatment (FIG. 4). Tumors appeared dark in color with some bruising, indicative of the vascular nature of angiosarcomas, often leading to ulceration and necrosis. Oral paclitaxel administered 30 minutes after Compound A resulted in a dose-dependent effect on SVRA221a tumor growth as demonstrated by both increased survival times and decreased tumor volumes.

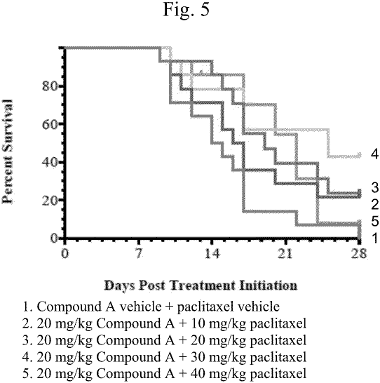

Mice survival did not appear to be correlated to treatment toxicity. Survival was effected by tumor growth and significantly influenced by ulceration and necrosis. As shown in FIG. 5 and Table 18, the median survival time increased with increasing doses of paclitaxel and reached a plateau between 30 and 40 mg/kg.

TABLE 18

Median survival time of groups according to treatment

| Group No. | Treatment | MST (days) | P-value |
|---|---|---|---|
| 1 | Compound A vehicle + paclitaxel vehicle | 14.5 | - |
| 2 | 20 mg/kg Compound A + 10 mg/kg paclitaxel | 16.5 | 0.136 |
| 3 | 20 mg/kg Compound A + 20 mg/kg paclitaxel | 19.0 | 0.018 |
| 4 | 20 mg/kg Compound A + 30 mg/kg paclitaxel | 25.0 | 0.002 |
| 5 | 20 mg/kg Compound A + 40 mg/kg paclitaxel | 22.0 | 0.015 |

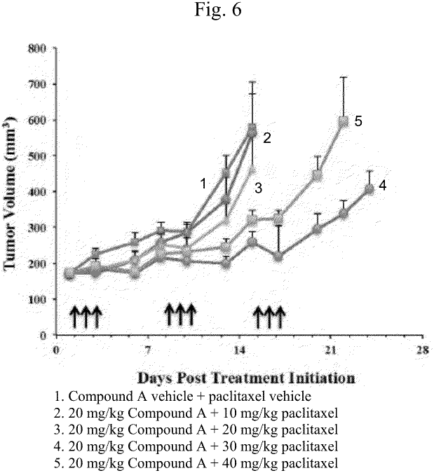

The effect of treatment on tumor growth is shown in FIG. 6. A dose-dependent decrease in tumor growth following treatment was observed. In general, tumor growth appeared to resume in between treatment cycles (~3 days following the last treatment in a cycle) but was retarded during additional treatment cycles. Tumor growth returned to normal upon completion of the final treatment.

Tumor growth was compared across the treatment groups on D13 and D15. Due to the tendency of angiosarcoma tumors to necrose/ulcerate, there were insufficient tumors remaining in the control group beyond D15 to perform additional analysis across groups. A comparison of tumor growth parameters between the control and treated groups is presented in Table 19 and Table 20.

TABLE 19

Effect of Treatment on Tumor Growth Parameters on D13

| Group No. | Treatments | Day 13 | | | | | | |
|---|---|---|---|---|---|---|---|---|
| | | Tumor Volume | | | Fold Tumor Growth (vs. DI) | | Growth Inhibition (%) | T/C (%) |
| | | Mean ± SEM | P-value | n | Mean ± SEM | P-value | | |
| 1 | Compound A vehicle + paclitaxel vehicle | 454.2 ± 45.3 | - | 9 | 3.1 ± 0.6 | - | 0.0 | - |
| 2 | 20 mg/kg Compound A + 10 mg/kg paclitaxel | 376.0 ± 63.9 | 0.562 | 10 | 2.4 ± 0.6 | 0.785 | 23.2 | 36.0 |
| 3 | 20 mg/kg Compound A + 20 mg/kg paclitaxel | 323.0 ± 45.6 | 0.112 | 13 | 1.9 ± 0.3 | 0.388 | 39.9 | 35.3 |
| 4 | 20 mg/kg Compound A + 30 mg/kg paclitaxel | 198.1 ± 20.6 | 0.001 | 11 | 1.2 ±0.2 | 0.146 | 61.1 | 5.2 |
| 5 | 20 mg/kg Compound A + 40 mg/kg paclitaxel | 246.5 ± 22.6 | 0.007 | 11 | 1.4 ± 0.2 | 0.207 | 54.3 | 16.2 |

TABLE 20

Effect of Treatment on Tumor Growth Parameters on D15

| Group No. | Treatments | Day 15 | | | | | | |
|---|---|---|---|---|---|---|---|---|
| | | Tumor Volume | | | Fold Tumor Growth (vs. DI) | | Growth Inhibition (%) | T/C (%) |
| | | Mean ± SEM | P-value | n | Mean ± SEM | P-value | | |
| 1 | Compound A vehicle + paclitaxel vehicle | 579.7 ± 92.6 | - | 7 | 3.9 ± 0.8 | - | 0.0 | - |
| 2 | 20 mg/kg Compound A + 10 mg/kg paclitaxel | 562.0 ± 142.9 | 1.000 | 8 | 3.7 ± 1.3 | 1.000 | 4.2 | 61.1 |
| 3 | 20 mg/kg Compound A + 20 mg/kg paclitaxel | 465.1 ± 90.0 | 0.789 | 11 | 2.6 ± 0.5 | 0.561 | 32.3 | 72.8 |
| 4 | 20 mg/kg Compound A + 30 mg/kg paclitaxel | 260.2 ± 26.7 | 0.107 | 11 | 1.6 ± 0.3 | 0.195 | 57.5 | 26.5 |
| 5 | 20 mg/kg Compound A + 40 mg/kg paclitaxel | 322.0 ± 24.3 | 0.172 | 11 | 1.9 ± 0.2 | 0.187 | 51.6 | 33.0 |

Dose-dependent decreases in tumor volume and fold tumor growth was observed on D13 and D15 of the study. The greatest tumor growth inhibition (61.1%) was observed with Study Group 4 (20 mg/kg Compound A + 30 mg/kg paclitaxel) on D13, which was statistically different from the control (P = 0.001). This inhibition remained at 57.5% on D15. Treatment with 20 mg/kg Compound A + 40 mg/kg paclitaxel (Study Group 5) also caused significant growth inhibition on D13 (54.3%, P = 0.007), which remained at 51.6% on D15. There was no significant difference between the effects of 30 mg/kg and 40 mg/kg paclitaxel.

The T/C% parameter is also used to evaluate tumor growth inhibition, which compares the median difference in the tumor volumes on a designated study day to D1 to the median difference of the control tumor volumes. A T/C% < 40 is indicative of efficacy. The T/C% for the two lowest paclitaxel doses of 10 mg/kg and 20 mg/kg (Study Groups 2 and 3) fell below 40% on D13, but increased to 61.1 and 72.8% by D15, respectively. In contrast, the two higher doses of paclitaxel demonstrated significant anti-tumor efficacy on both D13 (T/C% < 20) and D15 (T/C% < 35).

### INCORPORATION BY REFERENCE

The entire disclosure of each of the patent documents and scientific articles referred to herein is incorporated by reference for all purposes.

### EQUIVALENTS

The disclosure of the application can be embodied in other specific forms without departing from the spirit or essential characteristics thereof. The foregoing embodiments are therefore to be considered in all respects illustrative rather than limiting on the disclosure of the application described herein. Scope of the disclosure of the application is thus indicated by the appended claims rather than by the foregoing description, and all changes that come within the meaning and range of equivalency of the claims are intended to be embraced therein.

1. A method for treating an angiosarcoma in a subject in need thereof, comprising:
a. oral administration of paclitaxel at an amount of about 205 mg/m² to the subject once a day and for 3 times a week; and
b. oral administration of Compound A: 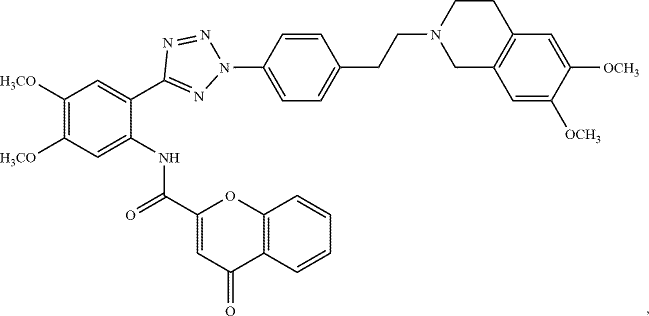
to the subject once a day and for 1-3 times a week, and
wherein Compound A is administered prior to the paclitaxel.

2. The method of claim 1, comprising oral administration of paclitaxel on consecutive days.

3. The method of claim 1, comprising oral administration of Compound A at an amount of about 15 mg.

4. The method of claim 1, comprising oral administration of Compound A for 3 times a week.

5. The method of claim 1, comprising oral administration of Compound A on consecutive days.

6. The method of claim 1, comprising oral administration of Compound A at an amount of about 15 mg and for 3 times a week.

7. The method of claim 6, comprising oral administration of paclitaxel on consecutive days.

8. The method of claim 6, comprising oral administration of Compound A on consecutive days.

9. The method of claim 6, wherein Compound A is administered about an hour prior to the paclitaxel.

10. The method of claim 1, wherein the angiosarcoma is cutaneous angiosarcoma.

11. A method for treating an angiosarcoma in a subject in need thereof, comprising:
a. oral administration of paclitaxel at an amount of about 205 mg/m² to the subject once a day and for 3 times a week for a first duration, and at a second amount less than 205 mg/m² to the subject once a day and for 3 times a week for a second duration;
b. oral administration of Compound A: 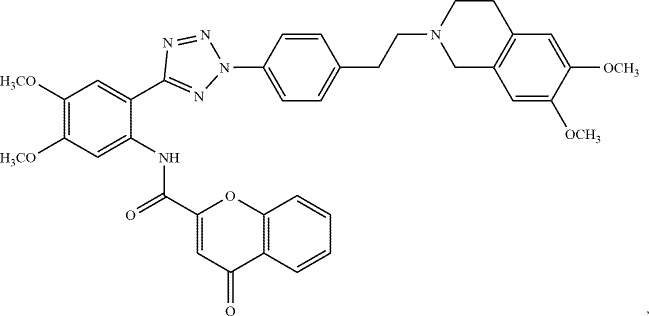
to the subject once a day and for 1-3 times a week, and
wherein Compound A is administered prior to the paclitaxel.

12. The method of claim 11, wherein the second amount is about 165 mg/m².

13. The method of claim 11, wherein the second amount is about 130 mg/m².

14. The method of claim 11, comprising oral administration of Compound A at an amount of about 15 mg and for 3 times a week.

15. The method of claim 11, wherein the angiosarcoma is cutaneous angiosarcoma.

16. A method for treating an angiosarcoma in a subject in need thereof, comprising:
a. oral administration of paclitaxel at an amount of about 205 mg/m² to the subject once a day and for 3 times a week for a first duration, at a second amount less than 205 mg/m² to the subject once a day and for 3 times a week for a second duration, and at a third amount less than the second amount to the subject once a day and for 3 times a week;
b. oral administration of Compound A: 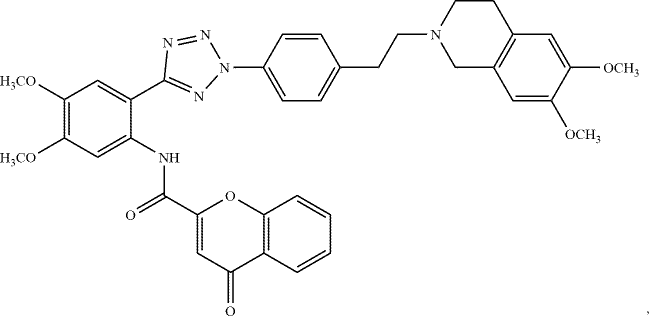
to the subject once a day and for 1-3 times a week, and
wherein Compound A is administered prior to the paclitaxel.

17. The method of claim 16, wherein the second amount is about 165 mg/m².

18. The method of claim 16, wherein the third amount is about 130 mg/m².

19. The method of claim 16, comprising oral administration of Compound A at an amount of about 15 mg and for 3 times a week.

20. The method of claim 16, wherein the angiosarcoma is cutaneous angiosarcoma.
